(19) United States
(12) Patent Application Publication
(10) Pub. No.: US 2025/0084374 A1
(43) Pub. Date: Mar. 13, 2025

(54) Compositions for reprogramming cells into hemogenic and/or hematopoietic stem cell-like cells, methods and uses thereof

(71) Applicant: Centro de Neurociências e Biologia Celular, COIMBRA (PT)

(72) Inventors: Carlos Filipe RIBEIRO LEMOS PEREIRA, CAMINHA (PT); Rita Alexandra SILVÉRIO ALVES, FERRAGUDO (PT)

(73) Assignee: Centro de Neurociências e Biologia Celular, COIMBRA (PT)

(21) Appl. No.: 18/725,902

(22) PCT Filed: Dec. 30, 2022
(86) PCT No.: PCT/IB2022/062924

§ 371 Date: Jul. 1, 2024

(30) Foreign Application Priority Data
Dec. 31, 2021 (PT) 117725

(51) Int. Cl. C12N 5/0797; C12N 15/86

(52) U.S. Cl. CPC C12N 5/0623; C12N 15/86; C12N 2506/00; C12N 2740/15043

(57) ABSTRACT

The present disclosure relates to a construct or a vector for reprogramming stem cells, differentiated cells, or mixtures thereof into hemogenic and/or hematopoietic stem cell-like cells, wherein the construct or the vector encodes a peptide comprising a combination of two isolated or synthetic transcription factors, preferably at least three isolated or synthetic transcription factors. The disclosure also relates to a composition comprising said construct or vector, to a method for reprogramming or inducing a stem cell or a differentiated cell into hemogenic and/or hematopoietic stem cell-like cells comprising a step of transducing a cell with at least one of said vectors; to a induced hemogenic and/or hematopoietic stem cell-like cell obtained by said method; to a composition comprising said induced hemogenic and/or hematopoietic stem cell-like cell; and to a kit comprising at least one of the following components: the induced hemogenic and/or hematopoietic stem cell-like cell; the composition as described in any of the previous claims; the vector or the construct as disclosed; or mixtures thereof.

### TECHNICAL FIELD

The present disclosure relates to a construct or vector for reprogramming cells, in particular differentiated cells or stem cells, into hemogenic and/or hematopoietic stem cell-like cells.

### BACKGROUND

Polycistronic vectors are able to efficiently reprogram to pluripotency (Carey et al., 2009; Sommer et al., 2009) or towards differentiated cell-types (Rosa et al., 2018; Wang et al., 2015) by combining several transcription factors (TFs) into a single viral vector. This way, it is possible to reduce the amount of random integrations and increase reprogramming efficiency by promoting the delivery of all factors at the same time in the same cell. Also, the order of the TFs in the polycistronic vector matters as this impacts the stoichiometry of the factors. The relative expression levels of the reprogramming factors is critical for an efficient process (Rosa et al., 2018; Wang et al., 2015). GATA2, GF11B and FOS reprogram mouse (Pereira et al., 2016, 2013) and human fibroblasts (Gomes et al., 2018; Silverio-Alves, Gomes, Kurochkin, Moore, & Pereira, 2019) to hematopoietic stem-like cells that undergo a dynamic hemogenic process.

Recently, CD9 was reported as a novel marker of hemogenic reprogramming, as it is expressed early in the reprogramming process (Silverio-Alves et al., 2019), besides being a positive marker of murine bone-marrow HSCs (Karlsson et al., 2013).

GATA2, GF11B and FOS transcription factors (TF) induce the activation of a hematopoietic stem cell (HSC)-specific reporter (hCD34/H2BGFP) in mouse embryonic fibroblasts and the expression HSC markers in human dermal fibroblasts (HDFs), namely CD9, CD49f, CD143 and the well stablished hemogenic and hematopoietic marker CD34, albeit with low efficiency (<1%). Ensuring the expression of GATA2, GF11B and FOS in all transduced cells and in optimal relative levels may be critical.

These facts are disclosed in order to illustrate the technical problem addressed by the present disclosure.

### GENERAL DESCRIPTION

The present disclosure relates to a method for generating hemogenic and/or hematopoietic stem cell-like cells from differentiated cell types by expression of transcription factors in a polycistronic vector.

In an embodiment, three transcription factors are used to induce hemogenic/hematopoietic reprogramming of differentiate cells. Polycistronic vectors include the coding sequences of the three transcription factors in six different orders, each vector encoding a first, a second and a third transcription factor separated by a self-cleavage peptide (SEQ ID No. 9 or SEQ ID No. 10) in a single open reading frame.

The present disclosure relates to a construct or a vector for reprogramming stem cells, differentiated cells, or mixtures thereof into hemogenic and/or hematopoietic stem cell-like cells, encoding a peptide comprising a combination of two isolated or synthetic transcription factors wherein the peptide sequence is at least 90% identical to and selected from a list comprising: SEQ ID No. 11, SEQ ID No. 12, SEQ ID No. 13, SEQ ID No. 14, SEQ ID No. 15, SEQ ID No. 16, and mixtures thereof; preferably at least three isolated or synthetic transcription factors. Surprisingly it was found that the construct or the vector of the present disclosure may be used as a reprograming agent of stem cells, differentiated cells, or mixtures thereof into hemogenic and/or hematopoietic stem cell-like cells; preferably differentiated cells into hemogenic and/or hematopoietic stem cell-like cells.

In an embodiment, the peptide sequence is at least 95% identical to and selected from a list consisting of: SEQ ID No. 11, SEQ ID No. 12, SEQ ID No. 13, SEQ ID No. 14, SEQ ID No. 15, SEQ ID No. 16, and mixtures thereof. In a further embodiment, the peptide sequence is identical to and selected from a list consisting of: SEQ ID No. 11, SEQ ID No. 12, SEQ ID No. 13, SEQ ID No. 14, SEQ ID No. 15, SEQ ID No. 16, and mixtures thereof. In a preferred embodiment, the peptide sequence is at least 90% identical to SEQ ID No. 12, preferably at least 95% identical to SEQ ID No. 12; more preferably identical to SEQ ID No. 12.

In an embodiment, the disclosed construct or the vector comprises a sequence at least 90% identical to and selected from a list consisting of: SEQ ID No. 44, SEQ ID No. 45, SEQ ID No. 46, SEQ ID No. 47, SEQ ID No. 48; SEQ ID No. 49. In a further embodiment, the construct or the vector comprises a sequence at least 95% identical to and selected from a list consisting of: SEQ ID No. 44, SEQ ID No. 45, SEQ ID No. 46, SEQ ID No. 47, SEQ ID No. 48; SEQ ID No. 49. In a yet further embodiment, the construct or the vector comprises a sequence identical to and selected from a list consisting of: SEQ ID No. 44, SEQ ID No. 45, SEQ ID No. 46, SEQ ID No. 47, SEQ ID No. 48, SEQ ID No. 49.

In an embodiment, the construct or the vector has a sequence at least 90% identical to and selected from a list consisting of: SEQ ID No. 20, SEQ ID. No 21, SEQ ID No. 22, SEQ ID No. 23, SEQ ID No. 24, SEQ ID No. 25, or combinations thereof; preferably at least 95% identical to and selected from a list consisting of: SEQ ID No. 20, SEQ ID. No 21, SEQ ID No. 22, SEQ ID No. 23, SEQ ID No. 24, SEQ ID No. 25, or combinations thereof; more preferably, identical to and selected from a list consisting of: SEQ ID No. 20, SEQ ID. No 21, SEQ ID No. 22, SEQ ID No. 23, SEQ ID No. 24, SEQ ID No. 25, or combinations thereof.

In a preferred embodiment, the construct or the vector has a sequence at least 90% identical to SEQ ID No. 21; preferably at least 95% identical to SEQ ID No. 21; more preferably identical to SEQ ID No. 21.

In an embodiment, the combination of three isolated or synthetic transcription factors is encoded in the following sequential order from 5′ to 3′: GATA2, GF11B, FOS; or GATA2, FOS, GF11B; or GF11B, GATA2, FOS; or GF11B, FOS, GATA2; FOS, GATA2, GF11B; or FOS, GF11B, GATA2.

In an embodiment, the vector is a viral vector; in particular a retrovirus, an adenovirus, a lentivirus, a herpes virus, a pox virus, or adeno-associated virus vectors.

In an embodiment, the disclosed construct or the disclosed vector may be used in medicine or as medicament, preferably in the treatment or therapy of diseases related to stem cell or bone marrow transplantation; or immunotherapy; or hemotherapy; or in the treatment or therapy of neurodegenerative diseases; drug screening; or disease modelling of blood-related diseases; or in the treatment or therapy of autoimmune diseases; or in the treatment or therapy of immunodeficiency; or in the treatment or therapy of cancer; or in the treatment or therapy of an infectious diseases.

The present disclosure also relates to a composition comprising the disclosed construct or vector for reprogramming stem cells or differentiated cells, or mixtures thereof into hemogenic and/or hematopoietic stem cell-like cells, preferably for reprogramming differentiated cells into hemogenic and/or hematopoietic stem cell-like cells.

In an embodiment, the composition comprises a combination of at least two transcription factors encoded by an isolated or synthetic sequence at least 90% identical to and selected from a list consisting of: SEQ ID No. 44, SEQ ID No. 45, SEQ ID No. 46, SEQ ID No. 47, SEQ ID No. 48, SEQ ID No. 49, and mixtures thereof; preferably at least three transcription factors.

In an embodiment, the composition comprises a combination of at least three transcription factors encoded by an isolated or synthetic sequence at least 95% identical and selected from a list consisting of: SEQ ID No. 44, SEQ ID No. 45, SEQ ID No. 46, SEQ ID No. 47, SEQ ID No. 48, SEQ ID No. 49, and mixtures thereof.

In an embodiment, the composition comprises a combination of at least three transcription factors encoded by an isolated or synthetic sequence identical and selected from a list consisting of: SEQ ID No. 44, SEQ ID No. 45, SEQ ID No. 46, SEQ ID No. 47, SEQ ID No. 48, SEQ ID No. 49, and mixtures thereof.

In an embodiment, the stem cells or differentiated cells are selected from a list consisting of: pluripotent stem cell, multipotent stem cell, fibroblast, cancer cell, and mixtures thereof.

In a further embodiment, the cell may be selected from a list consisting of: pluripotent stem cell, multipotent stem cell, differentiated cell, fibroblast, cancer cell, and mixtures thereof; preferably differentiated cell or fibroblast.

In an embodiment, the cell may be selected from a list consisting of: fibroblast, differentiated cell, and mixtures thereof.

In an embodiment, composition may be used in veterinary or human medicine, in particular for bone marrow and stem cell transplantation, in immunotherapy, or in autoimmune diseases, immunodeficiency, or in neurodegenerative or ageing diseases, or in cancer or in infectious diseases, or as a drug screening or disease model.

In an embodiment, the pluripotent stem cell, multipotent stem cell or differentiated cell is a mammalian pluripotent stem cell, multipotent stem cell or differentiated cell, in particular a mouse or a human cell.

The present disclosure also relates to a method for reprogramming or inducing a stem cell or a differentiated cell into hemogenic and/or hematopoietic stem cell-like cells, comprising the following steps:
transducing a cell selected from a list consisting of: stem cell or a differentiated cell, and mixtures thereof, with one or more vectors comprising a nucleic acid sequence encoding an aminoacid sequence at least 90% identical, preferably at least 95% identical, to a sequence from a list comprising SEQ ID No. 11, SEQ ID No. 12, SEQ ID No. 13, SEQ ID No. 14, SEQ ID No. 15, SEQ ID No. 16, and mixtures thereof;
culturing the transduced cell in a cell media that supports growth of hemogenic and/or hematopoietic stem cell-like cells.

In an embodiment, the aminoacid sequence is at least 90% identical to SEQ ID No. 12, preferably at least 95% identical to SEQ ID No. 12, even more preferably identical to SEQ ID No. 12.

In an embodiment, the vector comprises a sequence at least 90% identical to and selected from a list consisting of: SEQ ID No. 44, SEQ ID No. 45, SEQ ID No. 46, SEQ ID No. 47, SEQ ID No. 48, SEQ ID No. 49

In an embodiment, the vector has a sequence at least 90% identical to SEQ ID No. 45, preferably at least 95% identical to SEQ ID No. 45, even more preferably identical to SEQ ID No. 45.

In an embodiment, the culturing of the cell transduced with a plurality of isolated and synthetic transcription factors takes at least 2 days, preferably at least 5 days, more preferably at least 8 days, even more preferably at least 9 days.

In an embodiment, the cell is selected from a list consisting of: pluripotent stem cell, or multipotent stem cell, differentiated cell, and mixtures thereof.

In an embodiment, the cell is a mammalian cell.

In an embodiment, the pluripotent stem cell, multipotent stem cell, or differentiated cell, is selected from the group consisting of: an endoderm derived cell, a mesoderm derived cell, or an ectoderm derived cell, a multipotent stem cell including mesenchymal stem cell, a hematopoietic stem cell, an intestinal stem cell, a cell line, or fibroblast cell; in particular a fibroblast.

In an embodiment, the cell is a non-human cell. In a further embodiment, the cell is a mouse cell.

In an embodiment, the cell is a human cell.

In an embodiment, the cell is a human or mouse fibroblast, or a mammalian umbilical cord blood stem cell.

An aspect of the present disclosure relates to an induced hemogenic and/or hematopoietic stem cell-like cell obtained by the disclosed method.

The present disclosure also relates to a composition comprising the disclosed induced hemogenic and/or hematopoietic stem cell-like cell, or mixtures thereof, in a therapeutically effective amount and a pharmaceutically acceptable excipient. In an embodiment, the composition may be used in veterinary or human medicine.

In an embodiment, the composition may be used in stem cell or bone marrow transplantation; or immunotherapy; or hemotherapy; or in the treatment or therapy of neurodegenerative diseases; drug screening; or disease modelling of blood-related diseases; or in the treatment or therapy of autoimmune diseases; or in the treatment or therapy of immunodeficiency; or in the treatment or therapy of cancer; or in the treatment or therapy of an infectious diseases.

In an embodiment, the composition further comprises an anti-viral, an analgesic, an anti-inflammatory agent, a chemotherapy agent, a radiotherapy agent, an antibiotic, a diuretic, or mixtures thereof.

In an embodiment, the composition further comprises a filler, a binder, a disintegrant, or a lubricant, or mixtures thereof.

In an embodiment, the composition is an injectable formulation, in particular an in-situ injection.

In an embodiment, the composition may be used in veterinary or human medicine, in particular in immunotherapy, hemotherapy, drug screening, disease modelling of blood-related diseases, neurodegenerative or ageing diseases, or in cancer or in infectious diseases.

In an embodiment, the composition may be used in the treatment, therapy or diagnostic of a blood disorder.

In an embodiment, the composition may be used in the treatment therapy or diagnostic of neoplasia in particular cancer, namely solid or haematological tumours.

In an embodiment, the composition may be used in the treatment, therapy or diagnostic of a fungal, viral, chlamydial, bacterial, nanobacterial or parasitic infectious disease.

An aspect of the present disclosure relates to a kit comprising at least one of the following components:
the disclosed induced hemogenic and/or hematopoietic stem cell-like cell;
the disclosed compositions;
the disclosed vector or the disclosed construct;
or mixtures thereof.

### BRIEF DESCRIPTION OF THE DRAWINGS

The following figures provide preferred embodiments for illustrating the disclosure and should not be seen as limiting the scope of invention.

FIG. 1: (A) Schematic representation of an embodiment of the three transcription factors (3TFs) in individual delivery vectors. (B) Schematic representation of polycistronic delivery vectors encoding GATA2, GFI1B and FOS in six different orders separated by P2A and T2A self-cleaving peptides. Ga-GATA2, Gi-GF11B, Fo-FOS.

FIG. 2: Embodiment of western blot data show the expression of GATA2, GFI1B and FOS proteins 5 days after transduction of human dermal fibroblasts with the six polycistronic vectors. Calnexin (CANX) was used as loading control.

FIG. 3: Embodiment of quantification (%) of fully reprogrammed (CD9+CD49f+CD34+CD143+) cells inside the live-cell population, 15 days after human dermal fibroblasts were transduced with individual factors (3TFs) or polycistronic vectors. GaFoGi was the vector (SEQ ID No. 21) that lead to the highest increase in reprogramming efficiency (n=6-7). *p<0.05, **** p<0.0001. Mean±SD is represented.

FIG. 4: Representative flow cytometry plots of CD34+CD143+ cells inside the CD9+CD49f+ population when the three transcription factors (3TFs) are expressed in human dermal fibroblasts individually (A) or in the same polycistronic vector in the GATA2-FOS-GF11B (GaFoGi, SEQ ID No. 21) order (B), 15 days after transduction. A prominent CD34+CD143+ population is evident in the GaFoGi condition.

### DETAILED DESCRIPTION

The present disclosure relates to a construct or a vector for reprogramming stem cells, differentiated cells, or mixtures thereof into hemogenic and/or hematopoietic stem cell-like cells, wherein the construct or the vector encodes a peptide comprising a combination of two isolated or synthetic transcription factors, preferably at least three isolated or synthetic transcription factors. The disclosure also relates to a composition comprising said construct or vector, to a method for reprogramming or inducing a stem cell or a differentiated cell into hemogenic and/or hematopoietic stem cell-like cells comprising a step of transducing a cell with at least one of said vectors; to a induced hemogenic and/or hematopoietic stem cell-like cell obtained by said method; to a composition comprising said induced hemogenic and/or hematopoietic stem cell-like cell; and to a kit comprising at least one of the following components: the induced hemogenic and/or hematopoietic stem cell-like cell; the composition as described in any of the previous claims; the vector or the construct as disclosed; or mixtures thereof.

Also, the present disclosure provides methods to generate hemogenic and/or hematopoietic stem cell-like cells from differentiated cell types by expression of transcription factors in a polycistronic vector. Three transcription factors are sufficient to induce hemogenic/hematopoietic reprogramming of differentiate cells. Polycistronic vectors include the coding sequences of the three transcription factors in six different orders, each vector encoding a first, a second and a third transcription factor separated by a self-cleavage peptide in a single open reading frame.

In an embodiment, human embryonic kidney (HEK) 293T cells, and human dermal fibroblast (HDFs) (ScienCell) were maintained in DMEM (Dulbecco's modified Eagle's medium) supplemented with 10% (v/v) fetal bovine serum (FBS), 2 mM L-glutamine and antibiotics (penicillin and streptomycin, 10 μg/ml). All cells were maintained at 37° C. and 5% (v/v) CO₂. HDFs undergoing reprogramming were cultured in MyeloCult H5100 (StemCell Technologies) supplemented with 1 mM of hydrocortisone (StemCell Technologies) and antibiotic-antimycotic (1λ).

In an embodiment, polycistronic vectors for the human factors GATA2 (SEQ ID No. 6), GF11B (SEQ ID No. 7), and FOS (SEQ ID No. 8), with the National Centre for Biotechnology Information (NCBI) consensus sequence references CCDS3049.1, CCDS6957.1 and CCDS9841.1, respectively, were codon optimized and synthesized by Twist Bioscience in six different orders (SEQ ID No. 44, SEQ ID No. 45, SEQ ID No. 46, SEQ ID No. 47, SEQ ID No. 48, SEQ ID No. 49) and subcloned by enzymatic restriction into a constitutive lentiviral vector (FUW) with a human ubiquitin (UbC) promoter (FUW-UbC, SEQ ID No. 17). Each transcription factor is separated by a self-cleavage peptide (P2A or T2A) in a single open reading frame. The first two coding sequences lack the stop codon. Individual human factors (SEQ ID No. 1, SEQ ID No. 2, SEQ ID No. 3) were also subcloned into the FUW lentiviral vector using In-Fusion HD Cloning Kit (Takara) according to manufacturer's instructions.

In an embodiment, HEK293T cells were transfected with a mixture of 10 μg transfer plasmid (polycistronic or individual vectors), 7.5 μg of psPAX2 packaging vector encoding the Gag, Pol, Tat and Rev genes (SEQ ID No. 18, Addgene #12260) and 2.5 μg of pMD2.G envelope vector encoding the VSV-G gene (SEQ ID No. 19, Addgene #12259). Viral supernatants were harvested after 36, 48, and 72 hours of cell culture, filtered (0.45 μm) and concentrated 100-fold with with Lenti-X Virus Concentrator (Takara). GATA2, GF11B and FOS individual vectors (3TFs) were pool-produced. Different viruses used in the same experiment were produced in the same batch.

In an embodiment, 150000 HDFs were plated in 6-well plates (Falcon) prior to transduction. The next day, cells were incubated overnight with 10 L of FUW lentiviral particles (polycistronics [SEQ ID No. 20, SEQ ID No. 21, SEQ ID No. 22, SEQ ID No. 23, SEQ ID No. 24, SEQ ID No. 25] or individual vectors) in media supplemented with polybrene (8 μg/ml). Cells were transduced twice in consecutive days in two independent experiments. Day 0 was considered the day of the first transduction. Cells were split 1:2 at day 4 and cultured in MyeloCult H5100 (StemCell Technologies) supplemented with 1 mM of hydrocortisone (StemCell Technologies) and antibiotic-antimycotic (1λ) until the end of the experiment at day 15. Media was changed twice a week.

In an embodiment, up to 1 million transduced HDFs were submitted to whole cell lysis with radioimmunoprecipitation assay (RIPA) buffer (Thermo Fisher Scientific) supplemented with 100 mM phenylmethylsulfonyl fluoride (PMSF), 1M sodium fluoride (NaF) and 1× Protease Inhibitor Cocktail (Thermo Fisher Scientific). Protein fractions were diluted 1:2 in Laemmli buffer (Bio-Rad) with 5% 2-ME (Sigma) and boiled at 98° C. for 10 min. Samples were run in Bolt 4 to 12%, Bis-Tris SDS-PAGE gels (Invitrogen) using Mini Gel Tank (Thermo Fisher Scientific) and Bolt running buffer (Invitrogen). Transfer was done in an iBlot 2 (Thermo Fisher Scientific) dry system for 7 min. Membranes were incubated overnight at 4° C. with unconjugated primary antibodies against GATA2, GF11B, FOS or Calnexin diluted according to manufacturer's instructions, washed and incubated for 45 min at room temperature with donkey anti-rabbit horseradish peroxidase-conjugated secondary antibody diluted 1:10000. Membranes were incubated with ECL prime (Amersham) for 5 min and revealed in a ChemiDoc (Bio-Rad). A similar number of cells was used in all conditions.

In an embodiment, cells were dissociated, pelleted and incubated with anti-human CD49f-PECy7, CD9-PE, CD34-AF488 antibodies diluted (1:100), together with CD143-APC (3:100) in staining buffer (phosphate-buffered saline (PBS) with 2% FBS) at 4° C. for 20 min, in the presence of mouse serum 1% (v/v). Cells were washed once, resuspended in staining buffer, stained for cell viability with 4′,6-diamidino-2-phenylindole (DAPI, 1:100) and analyzed in LSR FORTESSA. Live cells were defined as DAPI negative. Flow cytometry results were analyzed using Flowjo software (version 10.6.1).

In an embodiment, comparison of means between groups was performed by one-way ANOVA followed by Bonferroni's multiple comparisons test.

In an embodiment, GATA2, GF11B and FOS transcription factors can be delivered in a single polycistronic vector. Six polycistronic vectors were generated (SEQ ID No. 20, SEQ ID No. 21, SEQ ID No. 22, SEQ ID No. 23, SEQ ID No. 24, SEQ ID No. 25), where GATA2 (Ga), GF11B (Gi) and FOS (Fo) were positioned in different orders: GaGiFo (SEQ ID No. 44), GaFoGi (SEQ ID No. 45), GiGaFo (SEQ ID No. 46), GiFoGa (SEQ ID No. 47), FoGaGi (SEQ ID No. 48) and FoGiGa (SEQ ID No. 49) (FIG. 1).

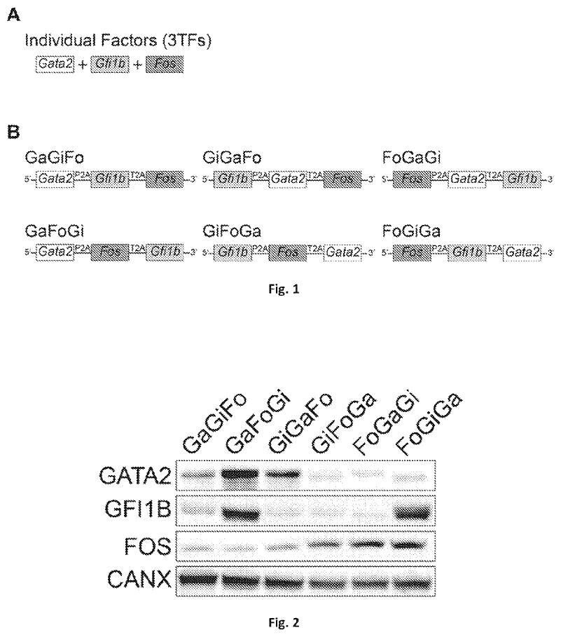

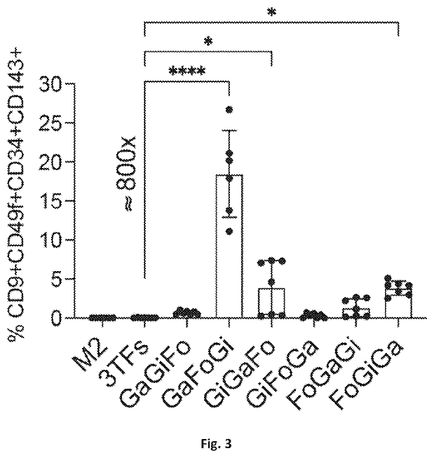

In an embodiment, western blot analysis of the expression levels of each transcription factor (TF) 5 days post-transduction, with each polycistronic vector, show that the three proteins are expressed at different levels depending on the vector used (FIG. 2). Interestingly, vector GaFoGi (SEQ ID No. 21) that lead to high levels of GATA2 and GF11B, together with low levels of FOS, resulted in an 800-fold increase in the percentage of fully reprogrammed cells, when compared to the individual factors (3TFs) (FIG. 3). Higher levels of GATA2 alone (found for SEQ ID No. 22, GiGaFo) or high levels of GFI1B plus FOS (found for SEQ ID No. 25, FoGiGa) also improved reprograming efficiency, but to a lower extent, stressing the need for higher expression levels of both GATA2 and GF11B for optimal reprogramming efficiency (FIGS. 2 and 3).

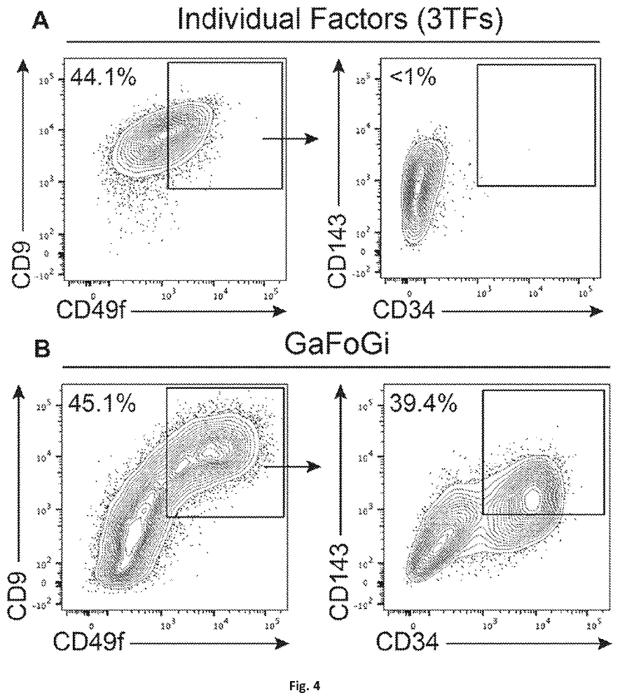

In an embodiment, reprogramming efficiency was assessed by the percentage of quadruple positive cells for CD9, CD49f, CD34 and CD143 hemogenic markers, 15 days after transduction of HDFs with 3TFs or with each of the polycistronic vectors (FIGS. 3 and 4). The obtained data indicates that even though the 3TFs condition is able to generate CD9+CD49f+ cells, these lack the CD34 marker (FIG. 4A), suggesting partial reprogramming. On the contrary, transduction with the GaFoGi polycistronic vector (SEQ ID No. 21) leads to the emergence of a cell population that is both CD143 and CD34 positive (FIG. 4B). This population accounts for approximately 40% of the CD9+CD49+ population, with the expression of CD34 being the most striking difference between conditions.

Surprisingly, the expression of GATA2, GF11B and FOS in a single polycistronic vector substantially improves hemogenic reprogramming efficiency, especially wherein the vector comprises a sequence when GATA2 is followed by FOS and then GF11B (SEQ ID No. 45, GaFoGi). Using the GaFoGi vector, as SEQ ID No. 21, leads to high expression levels of both GATA2 and GF11B proteins, which translates into the combined expression of CD9, CD49f, CD143 and, in particular, CD34 markers.

In an embodiment, the increased efficiency may be accompanied by improvement in the lineage fidelity and long-term engraftment, as assessed by transcriptional and functional levels.

Methods for the alignment of sequences for comparison are well known in the art, such methods include BLAST, FASTA and TFASTA. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). The sequence identity values, which are indicated in the present subject matter as a percentage were determined over the entire amino acid sequence, using BLAST with the default parameters.

The term “comprising” whenever used in this document is intended to indicate the presence of stated features, integers, steps, components, but not to preclude the presence or addition of one or more other features, integers, steps, components or groups thereof.

Those skilled in the art will recognize or be able to ascertain using no more than routine experimentation, many equivalents to the specific embodiments of the invention described herein. The scope of the present invention is not intended to be limited to the above description, but rather is as set forth in the appended claims.

Where singular forms of elements or features are used in the specification of the claims, the plural form is also included, and vice versa, if not specifically excluded. For example, the term “a polysaccharide” or “the polysaccharide” also includes the plural forms “polysaccharides” or “the polysaccharides,” and vice versa. In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim.

Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skilled in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

The disclosure should not be seen in any way restricted to the embodiments described and a person with ordinary skill in the art will foresee many possibilities to modifications thereof. The above described embodiments are combinable.

The following claims further set out particular embodiments of the disclosure.

### REFERENCES

Carey, B. W., Markoulaki, S., Hanna, J., Saha, K., Gao, Q., Mitalipova, M., & Jaenisch, R. (2009). Reprogramming of murine and human somatic cells using a single polycistronic vector. PNAS, 106(1), 2-7.
Gomes, A. M., Kurochkin, I., Chang, B., Daniel, M., Law, K., Satija, N., . . . Pereira, C.-F. (2018). Cooperative Transcription Factor Induction Mediates Hemogenic Reprogramming. Cell Reports, 25(10), 2821-2835. https://doi.org/10.1016/J.celrep.2018.11.032
Karlsson, G., Rörby, E., Pina, C., Soneji, S., Reckzeh, K., Miharada, K., . . . Enver, T. (2013). Report The Tetraspanin CD9 Affords High-Purity Capture of All Murine Hematopoietic Stem Cells. Cell Reports, 4(4), 642-648. https://doi.org/10.1016/j.celrep.2013.07.020
Pereira, C. F., Chang, B., Gomes, A., Bernitz, J., Papatsenko, D., Niu, X., . . . Moore, K. A. (2016). Hematopoietic Reprogramming In Vitro Informs In Vivo Identification of Hemogenic Precursors to Definitive Hematopoietic Stem Cells. Developmental Cell, 36(5), 525-539. https://doi.org/10.1016/j.devcel.2016.02.011
Pereira, C. F., Chang, B., Qiu, J., Niu, X., Papatsenko, D., Hendry, C. E., . . . Moore, K. (2013). Induction of a Hemogenic Program in Mouse Fibroblasts. Cell Stem Cell, 13(2), 205-218. https://doi.org/10.1016/j.stem.2013.05.024
Rosa, F. F., Pires, C. F., Kurochkin, I., Ferreira, A. G., Gomes, A. M., Palma, L. G., . . . Pereira, C. (2018). Direct reprogramming of fibroblasts into antigen-presenting dendritic cells. Science Immunology, 3(30), eaau4292.
Silvério-Alves, R., Gomes, A. M., Kurochkin, I., Moore, K. A., & Pereira, C.-F. (2019). Hemogenic Reprogramming of Human Fibroblasts by Enforced Expression of Transcription Factors. Journal of Visualized Experiments, (e60112).
Sommer, C. A., Stadtfeld, M., Murphy, G. J., Hochedlinger, K., Kotton, D. N., & Mostoslavsky, G. (2009). Induced Pluripotent Stem Cell Generation Using a Single Lentiviral Stem Cell Cassette. Stem Cells, 27(3), 543-549. https://doi.org/10.1634/stemcells.2008-1075
Wang, L., Liu, Z., Yin, C., Asfour, H., Chen, O., Li, Y., & Bursac, N. (2015). Stoichiometry of Gata4, Mef2c, and Tbx5 Influences the Efficiency and Quality of Induced Cardiac Myocyte Reprogramming. Circulation Research, 116(2), 237-244. https://doi.org/10.1161/CIRCRESAHA.116.305547

SEQUENCE LISTING

Nucleotide and aminoacid sequences used were the following (* represents a stop codon):
SEQ ID No. 1-GATA2
ATGGAGGTGGCGCCCGAGCAGCCGCGCTGGATGGCGCACCCGGCCGTGCTGAATGCGCAGCACCCCGACTCACACCACCCGG
GCCTGGCGCACAACTACATGGAACCCGCGCAGCTGCTGCCTCCAGACGAGGTGGACGTCTTCTTCAATCACCTCGACTCGCAG
GGCAACCCCTACTATGCCAACCCCGCTCACGCGCGGGCGCGCGTCTCCTACAGCCCCGCGCACGCCCGCCTGACCGGAGGCCA
GATGTGCCGCCCACACTTGTTGCACAGCCCGGGTTTGCCCTGGCTGGACGGGGGCAAAGCAGCCCTCTCTGCCGCTGCGGCCC
ACCACCACAACCCCTGGACCGTGAGCCCCTTCTCCAAGACGCCACTGCACCCCTCAGCTGCTGGAGGCCCTGGAGGCCCACTCT
CTGTGTACCCAGGGGCTGGGGGGGGAGCGGGGGAGGCAGCGGGAGCTCAGTGGCCTCCCTCACCCCTACAGCAGCCCACT
CTGGCTCCCACCTTTTCGGCTTCCCACCCACGCCACCCAAAGAAGTGTCTCCTGACCCTAGCACCACGGGGGCTGCGTCTCCAG
CCTCATCTTCCGCGGGGGGTAGTGCAGCCCGAGGAGAGGACAAGGACGGCGTCAAGTACCAGGTGTCACTGACGGAGAGCA
TGAAGATGGAAAGTGGCAGTCCCCTGCGCCCAGGCCTAGCTACTATGGGCACCCAGCCTGCTACACACCACCCCATCCCCACC
TACCCCTCCTATGTGCCGGCGGCTGCCCACGACTACAGCAGCGGACTCTTCCACCCCGGAGGCTTCCTGGGGGGACCGGCCTC
CAGCTTCACCCCTAAGCAGCGCAGCAAGGCTCGTTCCTGTTCAGAAGGCCGGGAGTGTGTCAACTGTGGGGCCACAGCCACCC
CTCTCTGGCGGCGGGACGGCACCGGCCACTACCTGTGCAATGCCTGTGGCCTCTACCACAAGATGAATGGGCAGAACCGACC
ACTCATCAAGCCCAAGCGAAGACTGTCGGCCGCCAGAAGAGCCGGCACCTGTTGTGCAAATTGTCAGACGACAACCACCACCT
TATGGCGCCGAAACGCCAACGGGGACCCTGTCTGCAACGCCTGTGGCCTCTACTACAAGCTGCACAATGTTAACAGGCCACTG
ACCATGAAGAAGGAAGGGATCCAGACTCGGAACCGGAAGATGTCCAACAAGTCCAAGAAGAGCAAGAAAGGGGCGGAGTG
CTTCGAGGAGCTGTCAAAGTGCATGCAGGAGAAGTCATCCCCCTTCAGTGCAGCTGCCCTGGCTGGACACATGGCACCTGTGG
GCCACCTCCCGCCCTTCAGCCACTCCGGACACATCCTGCCCACTCCGACGCCCATCCACCCCTCCTCCAGCCTCTCCTTCGGCCA
CCCCCACCCGTCCAGCATGGTGACCGCCATGGGCTAG
SEQ ID No. 2-GFI1B
ATGCCACGCTCCTTCCTGGTGAAGAGCAAGAAGGCTCACACCTACCACCAGCCCCGTGTGCAGGAAGATGAACCGCTCTGGCC
TCCTGCCCTTACCCCGGTGCCCAGAGACCAGGCTCCAAGCAACAGCCCTGTCCTTAGCACTCTATTCCCAAACCAGTGCCTGGA
CTGGACCAACCTCAAACGAGAGCCGGAGCTGGAGCAGGACCAGAACTTGGCCAGGATGGCCCCGGCACCAGAGGGCCCCAT
TGTGCTGTCCCGACCCCAGGATGGGGACTCTCCACTGTCCGACTCACCCCCATTCTACAAGCCTAGCTTCTCCTGGGACACCTT
GGCCACAACCTATGGCCACAGCTACCGGCAGGCCCCCTCCACCATGCAGTCAGCCTTCCTGGAGCACTCCGTCAGCCTGTACG
GCAGTCCTCTTGTGCCCAGCACTGAGCCCGCCTTGGACTTCAGCCTCCGCTACTCCCCAGGCATGGATGCGTACCACTGTGTGA
AGTGCAACAAGGTCTTCTCCACCCCTCACGGGCTCGAAGTGCATGTGCGACGCTCCCATAGTGGGACCCGGCCCTTCGCCTGT
GACATCTGCGGCAAAACCTTCGGCCACGCTGTGAGCCTGGAGCAGCACACGCACGTCCACTCCCAGGAGCGCAGCTTCGAGT
GCCGCATGTGCGGCAAGGCCTTCAAGCGCTCGTCCACGCTGTCCACCCACCTGCTCATCCACTCAGACACGCGGCCCTACCCCT
GCCAGTTCTGCGGCAAGCGTTTCCACCAGAAGTCCGACATGAAGAAGCACACCTACATCCACACAGGTGAGAAGCCGCACAA
GTGCCAGGTGTGCGGAAAGGCCTTCAGCCAGAGCTCCAACCTCATCACCCACAGCCGCAAGCACACAGGCTTCAAGCCCTTCA
GCTGTGAGCTGTGCACCAAAGGCTTCCAGCGCAAGGTGGACCTGCGGCGGCACCGCGAGAGCCAGCACAATCTCAAGTGA
SEQ ID No. 3-FOS
ATGATGTTCTCGGGCTTCAACGCAGACTACGAGGCGTCATCCTCCCGCTGCAGCAGCGCGTCCCCGGCCGGGGATAGCCTCTC
TTACTACCACTCACCCGCAGACTCCTTCTCCAGCATGGGCTCGCCTGTCAACGCGCAGGACTTCTGCACGGACCTGGCCGTCTC
CAGTGCCAACTTCATTCCCACGGTCACTGCCATCTCGACCAGTCCGGACCTGCAGTGGCTGGTGCAGCCCGCCCTCGTCTCCTC
CGTGGCCCCATCGCAGACCAGAGCCCCTCACCCTTTCGGAGTCCCCGCCCCCTCCGCTGGGGCTTACTCCAGGGCTGGCGTTGT
GAAGACCATGACAGGAGGCCGAGCGCAGAGCATTGGCAGGAGGGGCAAGGTGGAACAGTTATCTCCAGAAGAAGAAGAGA
AAAGGAGAATCCGAAGGGAAAGGAATAAGATGGCTGCAGCCAAATGCCGCAACCGGAGGAGGGAGCTGACTGATACACTCC
AAGCGGAGACAGACCAACTAGAAGATGAGAAGTCTGCTTTGCAGACCGAGATTGCCAACCTGCTGAAGGAGAAGGAAAAAC
TAGAGTTCATCCTGGCAGCTCACCGACCTGCCTGCAAGATCCCTGATGACCTGGGCTTCCCAGAAGAGATGTCTGTGGCTTCCC
TTGATCTGACTGGGGGCCTGCCAGAGGTTGCCACCCCGGAGTCTGAGGAGGCCTTCACCCTGCCTCTCCTCAATGACCCTGAG
CCCAAGCCCTCAGTGGAACCTGTCAAGAGCATCAGCAGCATGGAGCTGAAGACCGAGCCCTTTGATGACTTCCTGTTCCCAGC
ATCATCCAGGCCCAGTGGCTCTGAGACAGCCCGCTCCGTGCCAGACATGGACCTATCTGGGTCCTTCTATGCAGCAGACTGGG
AGCCTCTGCACAGTGGCTCCCTGGGGATGGGGCCCATGGCCACAGAGCTGGAGCCCCTGTGCACTCCGGTGGTCACCTGTACT
CCCAGCTGCACTGCTTACACGTCTTCCTTCGTCTTCACCTACCCCGAGGCTGACTCCTTCCCCAGCTGTGCAGCTGCCCACCGCA
AGGGCAGCAGCAGCAATGAGCCTTCCTCTGACTCGCTCAGCTCACCCACGCTGCTGGCCCTGTGA
SEQ ID No. 4-P2A
GCCACAAACTTCTCTCTGCTAAAGCAAGCAGGTGATGTTGAAGAAAACCCCGGGCCT
SEQ ID No. 5-T2A
GAGGGCAGGGGAAGTCTTCTAACATGCGGGGACGTGGAGGAAAATCCCGGCCCA
SEQ ID No. 6-GATA 2 peptide
MEVAPEQPRWMAHPAVLNAQHPDSHHPGLAHNYMEPAQLLPPDEVDVFFNHLDSQGNPYYANPAHARARVSYSPAHARLTGG
QMCRPHLLHSPGLPWLDGGKAALSAAAAHHHNPWTVSPFSKTPLHPSAAGGPGGPLSVYPGAGGGSGGGSGSSVASLTPTAAHS
GSHLFGFPPTPPKEVSPDPSTTGAASPASSSAGGSAARGEDKDGVKYQVSLTESMKMESGSPLRPGLATMGTQPATHHPIPTYPSY
VPAAAHDYSSGLFHPGGFLGGPASSFTPKQRSKARSCSEGRECVNCGATATPLWRRDGTGHYLCNACGLYHKMNGQNRPLIKPKR
RLSAARRAGTCCANCQTTTTTLWRRNANGDPVCNACGLYYKLHNVNRPLTMKKEGIQTRNRKMSNKSKKSKKGAECFEELSKCM
QEKSSPFSAAALAGHMAPVGHLPPFSHSGHILPTPTPIHPSSSLSFGHPHPSSMVTAMG*
SEQ ID No. 7-GFI1B peptide
MPRSFLVKSKKAHTYHQPRVQEDEPLWPPALTPVPRDQAPSNSPVLSTLFPNQCLDWTNLKREPELEQDQNLARMAP
APEGPIVLSRPQDGDSPLSDSPPFYKPSFSWDTLATTYGHSYRQAPSTMQSAFLEHSVSLYGSPLVPSTEPALDFSLRYSP
GMDAYHCVKCNKVFSTPHGLEVHVRRSHSGTRPFACDICGKTFGHAVSLEQHTHVHSQERSFECRMCGKAFKRSSTLS
THLLIHSDTRPYPCQFCGKRFHQKSDMKKHTYIHTGEKPHKCQVCGKAFSQSSNLITHSRKHTGFKPFSCELCTKGFQRK
VDLRRHRESQHNLK*
SEQ ID No. 8-FOS peptide
MMFSGFNADYEASSSRCSSASPAGDSLSYYHSPADSFSSMGSPVNAQDFCTDLAVSSANFIPTVTAISTSPDLQWLVQP
ALVSSVAPSQTRAPHPFGVPAPSAGAYSRAGVVKTMTGGRAQSIGRRGKVEQLSPEEEEKRRIRRERNKMAAAKCRNR
RRELTDTLQAETDQLEDEKSALQTEIANLLKEKEKLEFILAAHRPACKIPDDLGFPEEMSVASLDLTGGLPEVATPESEEAF
TLPLLNDPEPKPSVEPVKSISSMELKTEPFDDFLFPASSRPSGSETARSVPDMDLSGSFYAADWEPLHSGSLGMGPMAT
ELEPLCTPVVTCTPSCTAYTSSFVFTYPEADSFPSCAAAHRKGSSSNEPSSDSLSSPTLLAL*
SEQ ID No. 9-P2A self-cleavage peptide
ATNFSLLKQAGDVEENPGP
SEQ ID No. 10-T2A self-cleavage peptide
EGRGSLLTCGDVEENPGP
SEQ ID No. 11-GaGiFo peptide
MEVAPEQPRWMAHPAVLNAQHPDSHHPGLAHNYMEPAQLLPPDEVDVFFNHLDSQGNPYYANPAHARARVSYSP
AHARLTGGQMCRPHLLHSPGLPWLDGGKAALSAAAAHHHNPWTVSPFSKTPLHPSAAGGPGGPLSVYPGAGGGSG
GGSGSSVASLTPTAAHSGSHLFGFPPTPPKEVSPDPSTTGAASPASSSAGGSAARGEDKDGVKYQVSLTESMKMESGSP
LRPGLATMGTQPATHHPIPTYPSYVPAAAHDYSSGLFHPGGFLGGPASSFTPKQRSKARSCSEGRECVNCGATATPLW
RRDGTGHYLCNACGLYHKMNGQNRPLIKPKRRLSAARRAGTCCANCQTTTTTLWRRNANGDPVCNACGLYYKLHNV
NRPLTMKKEGIQTRNRKMSNKSKKSKKGAECFEELSKCMQEKSSPFSAAALAGHMAPVGHLPPFSHSGHILPTPTPIHP
SSSLSFGHPHPSSMVTAMGGSGATNFSLLKQAGDVEENPGPMPRSFLVKSKKAHTYHQPRVQEDEPLWPPALTPVPR
DQAPSNSPVLSTLFPNQCLDWTNLKREPELEQDQNLARMAPAPEGPIVLSRPQDGDSPLSDSPPFYKPSFSWDTLATTY
GHSYRQAPSTMQSAFLEHSVSLYGSPLVPSTEPALDFSLRYSPGMDAYHCVKCNKVFSTPHGLEVHVRRSHSGTRPFAC
DICGKTFGHAVSLEQHTHVHSQERSFECRMCGKAFKRSSTLSTHLLIHSDTRPYPCQFCGKRFHQKSDMKKHTYIHTGE
KPHKCQVCGKAFSQSSNLITHSRKHTGFKPFSCELCTKGFQRKVDLRRHRESQHNLKGSGEGRGSLLTCGDVEENPGP
MMFSGFNADYEASSSRCSSASPAGDSLSYYHSPADSFSSMGSPVNAQDFCTDLAVSSANFIPTVTAISTSPDLQWLVQP
ALVSSVAPSQTRAPHPFGVPAPSAGAYSRAGVVKTMTGGRAQSIGRRGKVEQLSPEEEEKRRIRRERNKMAAAKCRNR
RRELTDTLQAETDQLEDEKSALQTEIANLLKEKEKLEFILAAHRPACKIPDDLGFPEEMSVASLDLTGGLPEVATPESEEAF
TLPLLNDPEPKPSVEPVKSISSMELKTEPFDDFLFPASSRPSGSETARSVPDMDLSGSFYAADWEPLHSGSLGMGPMAT
ELEPLCTPVVTCTPSCTAYTSSFVFTYPEADSFPSCAAAHRKGSSSNEPSSDSLSSPTLLAL*
SEQ ID No. 12-GaFoGi peptide
MEVAPEQPRWMAHPAVLNAQHPDSHHPGLAHNYMEPAQLLPPDEVDVFFNHLDSQGNPYYANPAHARARVSYSP
AHARLTGGQMCRPHLLHSPGLPWLDGGKAALSAAAAHHHNPWTVSPFSKTPLHPSAAGGPGGPLSVYPGAGGGSG
GGSGSSVASLTPTAAHSGSHLFGFPPTPPKEVSPDPSTTGAASPASSSAGGSAARGEDKDGVKYQVSLTESMKMESGSP
LRPGLATMGTQPATHHPIPTYPSYVPAAAHDYSSGLFHPGGFLGGPASSFTPKQRSKARSCSEGRECVNCGATATPLW
RRDGTGHYLCNACGLYHKMNGQNRPLIKPKRRLSAARRAGTCCANCQTTTTTLWRRNANGDPVCNACGLYYKLHNV
NRPLTMKKEGIQTRNRKMSNKSKKSKKGAECFEELSKCMQEKSSPFSAAALAGHMAPVGHLPPFSHSGHILPTPTPIHP
SSSLSFGHPHPSSMVTAMGGSGATNFSLLKQAGDVEENPGPMMFSGFNADYEASSSRCSSASPAGDSLSYYHSPADSF
SSMGSPVNAQDFCTDLAVSSANFIPTVTAISTSPDLQWLVQPALVSSVAPSQTRAPHPFGVPAPSAGAYSRAGVVKTM
TGGRAQSIGRRGKVEQLSPEEEEKRRIRRERNKMAAAKCRNRRRELTDTLQAETDQLEDEKSALQTEIANLLKEKEKLEFI
LAAHRPACKIPDDLGFPEEMSVASLDLTGGLPEVATPESEEAFTLPLLNDPEPKPSVEPVKSISSMELKTEPFDDFLFPASS
RPSGSETARSVPDMDLSGSFYAADWEPLHSGSLGMGPMATELEPLCTPVVTCTPSCTAYTSSFVFTYPEADSFPSCAAA
HRKGSSSNEPSSDSLSSPTLLALGSGEGRGSLLTCGDVEENPGPMPRSFLVKSKKAHTYHQPRVQEDEPLWPPALTPVP
RDQAPSNSPVLSTLFPNQCLDWTNLKREPELEQDQNLARMAPAPEGPIVLSRPQDGDSPLSDSPPFYKPSFSWDTLAT
TYGHSYRQAPSTMQSAFLEHSVSLYGSPLVPSTEPALDFSLRYSPGMDAYHCVKCNKVFSTPHGLEVHVRRSHSGTRPF
ACDICGKTFGHAVSLEQHTHVHSQERSFECRMCGKAFKRSSTLSTHLLIHSDTRPYPCQFCGKRFHQKSDMKKHTYIHT
GEKPHKCQVCGKAFSQSSNLITHSRKHTGFKPFSCELCTKGFQRKVDLRRHRESQHNLK*
SEQ ID No. 13-GiGaFo peptide
MPRSFLVKSKKAHTYHQPRVQEDEPLWPPALTPVPRDQAPSNSPVLSTLFPNQCLDWTNLKREPELEQDQNLARMAP
APEGPIVLSRPQDGDSPLSDSPPFYKPSFSWDTLATTYGHSYRQAPSTMQSAFLEHSVSLYGSPLVPSTEPALDFSLRYSP
GMDAYHCVKCNKVFSTPHGLEVHVRRSHSGTRPFACDICGKTFGHAVSLEQHTHVHSQERSFECRMCGKAFKRSSTLS
THLLIHSDTRPYPCQFCGKRFHQKSDMKKHTYIHTGEKPHKCQVCGKAFSQSSNLITHSRKHTGFKPFSCELCTKGFQRK
VDLRRHRESQHNLKGSGATNFSLLKQAGDVEENPGPMEVAPEQPRWMAHPAVLNAQHPDSHHPGLAHNYMEPAQ
LLPPDEVDVFFNHLDSQGNPYYANPAHARARVSYSPAHARLTGGQMCRPHLLHSPGLPWLDGGKAALSAAAAHHHN
PWTVSPFSKTPLHPSAAGGPGGPLSVYPGAGGGSGGGSGSSVASLTPTAAHSGSHLFGFPPTPPKEVSPDPSTTGAASP
ASSSAGGSAARGEDKDGVKYQVSLTESMKMESGSPLRPGLATMGTQPATHHPIPTYPSYVPAAAHDYSSGLFHPGGFL
GGPASSFTPKQRSKARSCSEGRECVNCGATATPLWRRDGTGHYLCNACGLYHKMNGQNRPLIKPKRRLSAARRAGTC
CANCQTTTTTLWRRNANGDPVCNACGLYYKLHNVNRPLTMKKEGIQTRNRKMSNKSKKSKKGAECFEELSKCMQEKS
SPFSAAALAGHMAPVGHLPPFSHSGHILPTPTPIHPSSSLSFGHPHPSSMVTAMGGSGEGRGSLLTCGDVEENPGPM
MFSGFNADYEASSSRCSSASPAGDSLSYYHSPADSFSSMGSPVNAQDFCTDLAVSSANFIPTVTAISTSPDLQWLVQPA
LVSSVAPSQTRAPHPFGVPAPSAGAYSRAGVVKTMTGGRAQSIGRRGKVEQLSPEEEEKRRIRRERNKMAAAKCRNRR
RELTDTLQAETDQLEDEKSALQTEIANLLKEKEKLEFILAAHRPACKIPDDLGFPEEMSVASLDLTGGLPEVATPESEEAFT
LPLLNDPEPKPSVEPVKSISSMELKTEPFDDFLFPASSRPSGSETARSVPDMDLSGSFYAADWEPLHSGSLGMGPMATE
LEPLCTPVVTCTPSCTAYTSSFVFTYPEADSFPSCAAAHRKGSSSNEPSSDSLSSPTLLAL*
SEQ ID No. 14-GiFoGa peptide
MPRSFLVKSKKAHTYHQPRVQEDEPLWPPALTPVPRDQAPSNSPVLSTLFPNQCLDWTNLKREPELEQDQNLARMAP
APEGPIVLSRPQDGDSPLSDSPPFYKPSFSWDTLATTYGHSYRQAPSTMQSAFLEHSVSLYGSPLVPSTEPALDFSLRYSP
GMDAYHCVKCNKVFSTPHGLEVHVRRSHSGTRPFACDICGKTFGHAVSLEQHTHVHSQERSFECRMCGKAFKRSSTLS
THLLIHSDTRPYPCQFCGKRFHQKSDMKKHTYIHTGEKPHKCQVCGKAFSQSSNLITHSRKHTGFKPFSCELCTKGFQRK
VDLRRHRESQHNLKGSGATNFSLLKQAGDVEENPGPMMFSGFNADYEASSSRCSSASPAGDSLSYYHSPADSFSSMGS
PVNAQDFCTDLAVSSANFIPTVTAISTSPDLQWLVQPALVSSVAPSQTRAPHPFGVPAPSAGAYSRAGVVKTMTGGRA
QSIGRRGKVEQLSPEEEEKRRIRRERNKMAAAKCRNRRRELTDTLQAETDQLEDEKSALQTEIANLLKEKEKLEFILAAHR
PACKIPDDLGFPEEMSVASLDLTGGLPEVATPESEEAFTLPLLNDPEPKPSVEPVKSISSMELKTEPFDDFLFPASSRPSGS
ETARSVPDMDLSGSFYAADWEPLHSGSLGMGPMATELEPLCTPVVTCTPSCTAYTSSFVFTYPEADSFPSCAAAHRKGS
SSNEPSSDSLSSPTLLALGSGEGRGSLLTCGDVEENPGPMEVAPEQPRWMAHPAVLNAQHPDSHHPGLAHNYMEPA
QLLPPDEVDVFFNHLDSQGNPYYANPAHARARVSYSPAHARLTGGQMCRPHLLHSPGLPWLDGGKAALSAAAAHHH
NPWTVSPFSKTPLHPSAAGGPGGPLSVYPGAGGGSGGGSGSSVASLTPTAAHSGSHLFGFPPTPPKEVSPDPSTTGAA
SPASSSAGGSAARGEDKDGVKYQVSLTESMKMESGSPLRPGLATMGTQPATHHPIPTYPSYVPAAAHDYSSGLFHPGG
FLGGPASSFTPKQRSKARSCSEGRECVNCGATATPLWRRDGTGHYLCNACGLYHKMNGQNRPLIKPKRRLSAARRAGT
CCANCQTTTTTLWRRNANGDPVCNACGLYYKLHNVNRPLTMKKEGIQTRNRKMSNKSKKSKKGAECFEELSKCMQEK
SSPFSAAALAGHMAPVGHLPPFSHSGHILPTPTPIHPSSSLSFGHPHPSSMVTAMG*
SEQ ID No. 15-FoGaGi peptide
MMFSGFNADYEASSSRCSSASPAGDSLSYYHSPADSFSSMGSPVNAQDFCTDLAVSSANFIPTVTAISTSPDLQWLVQP
ALVSSVAPSQTRAPHPFGVPAPSAGAYSRAGVVKTMTGGRAQSIGRRGKVEQLSPEEEEKRRIRRERNKMAAAKCRNR
RRELTDTLQAETDQLEDEKSALQTEIANLLKEKEKLEFILAAHRPACKIPDDLGFPEEMSVASLDLTGGLPEVATPESEEAF
TLPLLNDPEPKPSVEPVKSISSMELKTEPFDDFLFPASSRPSGSETARSVPDMDLSGSFYAADWEPLHSGSLGMGPMAT
ELEPLCTPVVTCTPSCTAYTSSFVFTYPEADSFPSCAAAHRKGSSSNEPSSDSLSSPTLLALGSGATNFSLLKQAGDVEENP
GPMEVAPEQPRWMAHPAVLNAQHPDSHHPGLAHNYMEPAQLLPPDEVDVFFNHLDSQGNPYYANPAHARARVSY
SPAHARLTGGQMCRPHLLHSPGLPWLDGGKAALSAAAAHHHNPWTVSPFSKTPLHPSAAGGPGGPLSVYPGAGGGS
GGGSGSSVASLTPTAAHSGSHLFGFPPTPPKEVSPDPSTTGAASPASSSAGGSAARGEDKDGVKYQVSLTESMKMESG
SPLRPGLATMGTQPATHHPIPTYPSYVPAAAHDYSSGLFHPGGFLGGPASSFTPKQRSKARSCSEGRECVNCGATATPL
WRRDGTGHYLCNACGLYHKMNGQNRPLIKPKRRLSAARRAGTCCANCQTTTTTLWRRNANGDPVCNACGLYYKLH
NVNRPLTMKKEGIQTRNRKMSNKSKKSKKGAECFEELSKCMQEKSSPFSAAALAGHMAPVGHLPPFSHSGHILPTPTPI
HPSSSLSFGHPHPSSMVTAMGGSGEGRGSLLTCGDVEENPGPMPRSFLVKSKKAHTYHQPRVQEDEPLWPPALTPVP
RDQAPSNSPVLSTLFPNQCLDWTNLKREPELEQDQNLARMAPAPEGPIVLSRPQDGDSPLSDSPPFYKPSFSWDTLAT
TYGHSYRQAPSTMQSAFLEHSVSLYGSPLVPSTEPALDFSLRYSPGMDAYHCVKCNKVFSTPHGLEVHVRRSHSGTRPF
ACDICGKTFGHAVSLEQHTHVHSQERSFECRMCGKAFKRSSTLSTHLLIHSDTRPYPCQFCGKRFHQKSDMKKHTYIHT
GEKPHKCQVCGKAFSQSSNLITHSRKHTGFKPFSCELCTKGFQRKVDLRRHRESQHNLK*
SEQ ID No. 16-FoGiGa peptide
MMFSGFNADYEASSSRCSSASPAGDSLSYYHSPADSFSSMGSPVNAQDFCTDLAVSSANFIPTVTAISTSPDLQWLVQP
ALVSSVAPSQTRAPHPFGVPAPSAGAYSRAGVVKTMTGGRAQSIGRRGKVEQLSPEEEEKRRIRRERNKMAAAKCRNR
RRELTDTLQAETDQLEDEKSALQTEIANLLKEKEKLEFILAAHRPACKIPDDLGFPEEMSVASLDLTGGLPEVATPESEEAF
TLPLLNDPEPKPSVEPVKSISSMELKTEPFDDFLFPASSRPSGSETARSVPDMDLSGSFYAADWEPLHSGSLGMGPMAT
ELEPLCTPVVTCTPSCTAYTSSFVFTYPEADSFPSCAAAHRKGSSSNEPSSDSLSSPTLLALGSGATNFSLLKQAGDVEENP
GPMPRSFLVKSKKAHTYHQPRVQEDEPLWPPALTPVPRDQAPSNSPVLSTLFPNQCLDWTNLKREPELEQDQNLARM
APAPEGPIVLSRPQDGDSPLSDSPPFYKPSFSWDTLATTYGHSYRQAPSTMQSAFLEHSVSLYGSPLVPSTEPALDFSLRY
SPGMDAYHCVKCNKVFSTPHGLEVHVRRSHSGTRPFACDICGKTFGHAVSLEQHTHVHSQERSFECRMCGKAFKRSST
LSTHLLIHSDTRPYPCQFCGKRFHQKSDMKKHTYIHTGEKPHKCQVCGKAFSQSSNLITHSRKHTGFKPFSCELCTKGFQ
RKVDLRRHRESQHNLKGSGEGRGSLLTCGDVEENPGPMEVAPEQPRWMAHPAVLNAQHPDSHHPGLAHNYMEPA
QLLPPDEVDVFFNHLDSQGNPYYANPAHARARVSYSPAHARLTGGQMCRPHLLHSPGLPWLDGGKAALSAAAAHHH
NPWTVSPFSKTPLHPSAAGGPGGPLSVYPGAGGGSGGGSGSSVASLTPTAAHSGSHLFGFPPTPPKEVSPDPSTTGAA
SPASSSAGGSAARGEDKDGVKYQVSLTESMKMESGSPLRPGLATMGTQPATHHPIPTYPSYVPAAAHDYSSGLFHPGG
FLGGPASSFTPKQRSKARSCSEGRECVNCGATATPLWRRDGTGHYLCNACGLYHKMNGQNRPLIKPKRRLSAARRAGT
CCANCQTTTTTLWRRNANGDPVCNACGLYYKLHNVNRPLTMKKEGIQTRNRKMSNKSKKSKKGAECFEELSKCMQEK
SSPFSAAALAGHMAPVGHLPPFSHSGHILPTPTPIHPSSSLSFGHPHPSSMVTAMG*
SEQ ID No. 17-FUW-UbC
TCGACGGATCGGGAGATCTCCCGATCCCCTATGGTGCACTCTCAGTACAATCTGCTCTGATGCCGCATAGTTAAGCCAGTATCT
GCTCCCTGCTTGTGTGTTGGAGGTCGCTGAGTAGTGCGCGAGCAAAATTTAAGCTACAACAAGGCAAGGCTTGACCGACAATT
GCATGAAGAATCTGCTTAGGGTTAGGCGTTTTGCGCTGCTTCGCGATGTACGGGCCAGATATACGCGTTGACATTGATTATTG
ACTAGTTATTAATAGTAATCAATTACGGGGTCATTAGTTCATAGCCCATATATGGAGTTCCGCGTTACATAACTTACGGTAAAT
GGCCCGCCTGGCTGACCGCCCAACGACCCCCGCCCATTGACGTCAATAATGACGTATGTTCCCATAGTAACGCCAATAGGGAC
TTTCCATTGACGTCAATGGGTGGAGTATTTACGGTAAACTGCCCACTTGGCAGTACATCAAGTGTATCATATGCCAAGTACGCC
CCCTATTGACGTCAATGACGGTAAATGGCCCGCCTGGCATTATGCCCAGTACATGACCTTATGGGACTTTCCTACTTGGCAGTA
CATCTACGTATTAGTCATCGCTATTACCATGGTGATGCGGTTTTGGCAGTACATCAATGGGCGTGGATAGCGGTTTGACTCACG
GGGATTTCCAAGTCTCCACCCCATTGACGTCAATGGGAGTTTGTTTTGGCACCAAAATCAACGGGACTTTCCAAAATGTCGTAA
CAACTCCGCCCCATTGACGCAAATGGGCGGTAGGCGTGTACGGTGGGAGGTCTATATAAGCAGCGCGTTTTGCCTGTACTGG
GTCTCTCTGGTTAGACCAGATCTGAGCCTGGGAGCTCTCTGGCTAACTAGGGAACCCACTGCTTAAGCCTCAATAAAGCTTGCC
TTGAGTGCTTCAAGTAGTGTGTGCCCGTCTGTTGTGTGACTCTGGTAACTAGAGATCCCTCAGACCCTTTTAGTCAGTGTGGAA
AATCTCTAGCAGTGGCGCCCGAACAGGGACTTGAAAGCGAAAGGGAAACCAGAGGAGCTCTCTCGACGCAGGACTCGGCTTG
CTGAAGCGCGCACGGCAAGAGGCGAGGGGCGGCGACTGGTGAGTACGCCAAAAATTTTGACTAGCGGAGGCTAGAAGGAG
AGAGATGGGTGCGAGAGCGTCAGTATTAAGCGGGGGAGAATTAGATCGCGATGGGAAAAAATTCGGTTAAGGCCAGGGGG
AAAGAAAAAATATAAATTAAAACATATAGTATGGGCAAGCAGGGAGCTAGAACGATTCGCAGTTAATCCTGGCCTGTTAGAA
ACATCAGAAGGCTGTAGACAAATACTGGGACAGCTACAACCATCCCTTCAGACAGGATCAGAAGAACTTAGATCATTATATAA
TACAGTAGCAACCCTCTATTGTGTGCATCAAAGGATAGAGATAAAAGACACCAAGGAAGCTTTAGACAAGATAGAGGAAGAG
CAAAACAAAAGTAAGACCACCGCACAGCAAGCGGCCGCTGATCTTCAGACCTGGAGGAGGAGATATGAGGGACAATTGGAG
AAGTGAATTATATAAATATAAAGTAGTAAAAATTGAACCATTAGGAGTAGCACCCACCAAGGCAAAGAGAAGAGTGGTGCAG
AGAGAAAAAAGAGCAGTGGGAATAGGAGCTTTGTTCCTTGGGTTCTTGGGAGCAGCAGGAAGCACTATGGGCGCAGCGTCA
ATGACGCTGACGGTACAGGCCAGACAATTATTGTCTGGTATAGTGCAGCAGCAGAACAATTTGCTGAGGGCTATTGAGGCGC
AACAGCATCTGTTGCAACTCACAGTCTGGGGCATCAAGCAGCTCCAGGCAAGAATCCTGGCTGTGGAAAGATACCTAAAGGAT
CAACAGCTCCTGGGGATTTGGGGTTGCTCTGGAAAACTCATTTGCACCACTGCTGTGCCTTGGAATGCTAGTTGGAGTAATAA
ATCTCTGGAACAGATTTGGAATCACACGACCTGGATGGAGTGGGACAGAGAAATTAACAATTACACAAGCTTAATACACTCCT
TAATTGAAGAATCGCAAAACCAGCAAGAAAAGAATGAACAAGAATTATTGGAATTAGATAAATGGGCAAGTTTGTGGAATTG
GTTTAACATAACAAATTGGCTGTGGTATATAAAATTATTCATAATGATAGTAGGAGGCTTGGTAGGTTTAAGAATAGTTTTTGC
TGTACTTTCTATAGTGAATAGAGTTAGGCAGGGATATTCACCATTATCGTTTCAGACCCACCTCCCAACCCCGAGGGGACCCGA
CAGGCCCGAAGGAATAGAAGAAGAAGGTGGAGAGAGAGACAGAGACAGATCCATTCGATTAGTGAACGGATCGGCACTGC
GTGCGCCAATTCTGCAGACAAATGGCAGTATTCATCCACAATTTTAAAAGAAAAGGGGGGATTGGGGGGTACAGTGCAGGGG
AAAGAATAGTAGACATAATAGCAACAGACATACAAACTAAAGAATTACAAAAACAAATTACAAAAATTCAAAATTTTCGGGTT
TATTACAGGGACAGCAGAGATCCAGTTTGGTTAATTAACCCGTGTCGGCTCCAGATCTGGCCTCCGCGCCGGGTTTTGGCGCC
TCCCGCGGGCGCCCCCCTCCTCACGGCGAGCGCTGCCACGTCAGACGAAGGGCGCAGCGAGCGTCCTGATCCTTCCGCCCGG
ACGCTCAGGACAGCGGCCCGCTGCTCATAAGACTCGGCCTTAGAACCCCAGTATCAGCAGAAGGACATTTTAGGACGGGACTT
GGGTGACTCTAGGGCACTGGTTTTCTTTCCAGAGAGCGGAACAGGCGAGGAAAAGTAGTCCCTTCTCGGCGATTCTGCGGAG
GGATCTCCGTGGGGCGGTGAACGCCGATGATTATATAAGGACGCGCCGGGTGTGGCACAGCTAGTTCCGTCGCAGCCGGGAT
TTGGGTCGCGGTTCTTGTTTGTGGATCGCTGTGATCGTCACTTGGTGAGTAGCGGGCTGCTGGGCTGGCCGGGGCTTTCGTGG
CCGCCGGGCCGCTCGGTGGGACGGAAGCGTGTGGAGAGACCGCCAAGGGCTGTAGTCTGGGTCCGCGAGCAAGGTTGCCCT
GAACTGGGGGTTGGGGGGAGCGCAGCAAAATGGCGGCTGTTCCCGAGTCTTGAATGGAAGACGCTTGTGAGGCGGGCTGTG
AGGTCGTTGAAACAAGGTGGGGGGCATGGTGGGCGGCAAGAACCCAAGGTCTTGAGGCCTTCGCTAATGCGGGAAAGCTCT
TATTCGGGTGAGATGGGCTGGGGCACCATCTGGGGACCCTGACGTGAAGTTTGTCACTGACTGGAGAACTCGGTTTGTCGTCT
GTTGCGGGGGGGGCAGTTATGGCGGTGCCGTTGGGCAGTGCACCCGTACCTTTGGGAGCGCGCGCCCTCGTCGTGTCGTGAC
GTCACCCGTTCTGTTGGCTTATAATGCAGGGTGGGGCCACCTGCCGGTAGGTGTGCGGTAGGCTTTTCTCCGTCGCAGGACGC
AGGGTTCGGGCCTAGGGTAGGCTCTCCTGAATCGACAGGCGCCGGACCTCTGGTGAGGGGAGGGATAAGTGAGGCGTCAGT
TTCTTTGGTCGGTTTTATGTACCTATCTTCTTAAGTAGCTGAAGCTCCGGTTTTGAACTATGCGCTCGGGGTTGGCGAGTGTGTT
TTGTGAAGTTTTTTAGGCACCTTTTGAAATGTAATCATTTGGGTCAATATGTAATTTTCAGTGTTAGACTAGTAAATTGTCCGCT
AAATTCTGGCCGTTTTTGGCTTTTTTGTTAGACGAAGCTTGGGCCCGGGATCCATTCGATATCAAGCTTATCGATAATCAACCTC
TGGATTACAAAATTTGTGAAAGATTGACTGGTATTCTTAACTATGTTGCTCCTTTTACGCTATGTGGATACGCTGCTTTAATGCC
TTTGTATCATGCTATTGCTTCCCGTATGGCTTTCATTTTCTCCTCCTTGTATAAATCCTGGTTGCTGTCTCTTTATGAGGAGTTGTG
GCCCGTTGTCAGGCAACGTGGCGTGGTGTGCACTGTGTTTGCTGACGCAACCCCCACTGGTTGGGGCATTGCCACCACCTGTC
AGCTCCTTTCCGGGACTTTCGCTTTCCCCCTCCCTATTGCCACGGCGGAACTCATCGCCGCCTGCCTTGCCCGCTGCTGGACAGG
GGCTCGGCTGTTGGGCACTGACAATTCCGTGGTGTTGTCGGGGAAATCATCGTCCTTTCCTTGGCTGCTCGCCTGTGTTGCCAC
CTGGATTCTGCGCGGGACGTCCTTCTGCTACGTCCCTTCGGCCCTCAATCCAGCGGACCTTCCTTCCCGCGGCCTGCTGCCGGC
TCTGCGGCCTCTTCCGCGTCTTCGCCTTCGCCCTCAGACGAGTCGGATCTCCCTTTGGGCCGCCTCCCCGCATCGATACCGTCGA
CCTCGAGACCTAGAAAAACATGGAGCAATCACAAGTAGCAATACAGCAGCTACCAATGCTGATTGTGCCTGGCTAGAAGCACA
AGAGGAGGAGGAGGTGGGTTTTCCAGTCACACCTCAGGTACCTTTAAGACCAATGACTTACAAGGCAGCTGTAGATCTTAGCC
ACTTTTTAAAAGAAAAGGGGGGACTGGAAGGGCTAATTCACTCCCAACGAAGACAAGATATCCTTGATCTGTGGATCTACCAC
ACACAAGGCTACTTCCCTGATTGGCAGAACTACACACCAGGGCCAGGGATCAGATATCCACTGACCTTTGGATGGTGCTACAA
GCTAGTACCAGTTGAGCAAGAGAAGGTAGAAGAAGCCAATGAAGGAGAGAACACCCGCTTGTTACACCCTGTGAGCCTGCAT
GGGATGGATGACCCGGAGAGAGAAGTATTAGAGTGGAGGTTTGACAGCCGCCTAGCATTTCATCACATGGCCCGAGAGCTGC
ATCCGGACTGTACTGGGTCTCTCTGGTTAGACCAGATCTGAGCCTGGGAGCTCTCTGGCTAACTAGGGAACCCACTGCTTAAG
CCTCAATAAAGCTTGCCTTGAGTGCTTCAAGTAGTGTGTGCCCGTCTGTTGTGTGACTCTGGTAACTAGAGATCCCTCAGACCC
TTTTAGTCAGTGTGGAAAATCTCTAGCAGGGCCCGTTTAAACCCGCTGATCAGCCTCGACTGTGCCTTCTAGTTGCCAGCCATC
TGTTGTTTGCCCCTCCCCCGTGCCTTCCTTGACCCTGGAAGGTGCCACTCCCACTGTCCTTTCCTAATAAAATGAGGAAATTGCA
TCGCATTGTCTGAGTAGGTGTCATTCTATTCTGGGGGGTGGGGTGGGGCAGGACAGCAAGGGGGAGGATTGGGAAGACAAT
AGCAGGCATGTGAGCAAAAGGCCAGCAAAAGGCCAGGAACCGTAAAAAGGCCGCGTTGCTGGCGTTTTTCCATAGGCTCCGC
CCCCCTGACGAGCATCACAAAAATCGACGCTCAAGTCAGAGGTGGCGAAACCCGACAGGACTATAAAGATACCAGGCGTTTC
CCCCTGGAAGCTCCCTCGTGCGCTCTCCTGTTCCGACCCTGCCGCTTACCGGATACCTGTCCGCCTTTCTCCCTTCGGGAAGCGT
GGCGCTTTCTCATAGCTCACGCTGTAGGTATCTCAGTTCGGTGTAGGTCGTTCGCTCCAAGCTGGGCTGTGTGCACGAACCCCC
CGTTCAGCCCGACCGCTGCGCCTTATCCGGTAACTATCGTCTTGAGTCCAACCCGGTAAGACACGACTTATCGCCACTGGCAGC
AGCCACTGGTAACAGGATTAGCAGAGCGAGGTATGTAGGCGGTGCTACAGAGTTCTTGAAGTGGTGGCCTAACTACGGCTAC
ACTAGAAGAACAGTATTTGGTATCTGCGCTCTGCTGAAGCCAGTTACCTTCGGAAAAAGAGTTGGTAGCTCTTGATCCGGCAA
ACAAACCACCGCTGGTAGCGGTGGTTTTTTTGTTTGCAAGCAGCAGATTACGCGCAGAAAAAAAGGATCTCAAGAAGATCCTT
TGATCTTTTCTACGGGGTCTGACGCTCAGTGGAACGAAAACTCACGTTAAGGGATTTTGGTCATGAGATTATCAAAAAGGATC
TTCACCTAGATCCTTTTAAATTAAAAATGAAGTTTTAAATCAATCTAAAGTATATATGAGTAAACTTGGTCTGACAGTTACCAAT
GCTTAATCAGTGAGGCACCTATCTCAGCGATCTGTCTATTTCGTTCATCCATAGTTGCCTGACTCCCCGTCGTGTAGATAACTAC
GATACGGGAGGGCTTACCATCTGGCCCCAGTGCTGCAATGATACCGCGAGACCCACGCTCACCGGCTCCAGATTTATCAGCAA
TAAACCAGCCAGCCGGAAGGGCCGAGCGCAGAAGTGGTCCTGCAACTTTATCCGCCTCCATCCAGTCTATTAATTGTTGCCGG
GAAGCTAGAGTAAGTAGTTCGCCAGTTAATAGTTTGCGCAACGTTGTTGCCATTGCTACAGGCATCGTGGTGTCACGCTCGTC
GTTTGGTATGGCTTCATTCAGCTCCGGTTCCCAACGATCAAGGCGAGTTACATGATCCCCCATGTTGTGCAAAAAAGCGGTTAG
CTCCTTCGGTCCTCCGATCGTTGTCAGAAGTAAGTTGGCCGCAGTGTTATCACTCATGGTTATGGCAGCACTGCATAATTCTCTT
ACTGTCATGCCATCCGTAAGATGCTTTTCTGTGACTGGTGAGTACTCAACCAAGTCATTCTGAGAATAGTGTATGCGGCGACCG
AGTTGCTCTTGCCCGGCGTCAATACGGGATAATACCGCGCCACATAGCAGAACTTTAAAAGTGCTCATCATTGGAAAACGTTCT
TCGGGGCGAAAACTCTCAAGGATCTTACCGCTGTTGAGATCCAGTTCGATGTAACCCACTCGTGCACCCAACTGATCTTCAGCA
TCTTTTACTTTCACCAGCGTTTCTGGGTGAGCAAAAACAGGAAGGCAAAATGCCGCAAAAAAGGGAATAAGGGCGACACGGA
AATGTTGAATACTCATACTCTTCCTTTTTCAATATTATTGAAGCATTTATCAGGGTTATTGTCTCATGAGCGGATACATATTTGAA
TGTATTTAGAAAAATAAACAAATAGGGGTTCCGCGCACATTTCCCCGAAAAGTGCCACCTGACG
SEQ ID No. 18-packaging vector encoding the Gag, Pol, Tat and Rev genes
TTGATTATTGACTAGTTATTAATAGTAATCAATTACGGGGTCATTAGTTCATAGCCCATATATGGAGTTCCGCGTTACATAACTT
ACGGTAAATGGCCCGCCTGGCTGACCGCCCAACGACCCCCGCCCATTGACGTCAATAATGACGTATGTTCCCATAGTAACGCC
AATAGGGACTTTCCATTGACGTCAATGGGTGGAGTATTTACGGTAAACTGCCCACTTGGCAGTACATCAAGTGTATCATATGCC
AAGTACGCCCCCTATTGACGTCAATGACGGTAAATGGCCCGCCTGGCATTATGCCCAGTACATGACCTTATGGGACTTTCCTAC
TTGGCAGTACATCTACGTATTAGTCATCGCTATTACCATGGTCGAGGTGAGCCCCACGTTCTGCTTCACTCTCCCCATCTCCCCC
CCCTCCCCACCCCCAATTTTGTATTTATTTATTTTTTAATTATTTTGTGCAGCGATGGGGGCGGGGGGGGGGGGGGGGCGCGC
CRGGGGGGGGGGGGGGGGGGGGGGGGGGGGGGGGGGSGRGGSGGRGRGGKGCGGCGGCAGCCAATCAGAGCGGCGCG
CTCCGAAAGTTTCCTTTTATGGCGAGGCGGCGGCGGCGGCGGCCCTATAAAAAGCGAAGCGCGCGGCGGGGGGAGTCGCT
GCGCGCTGCCTTCGCCCCGTGCCCCGCTCCGCCGCCGCCTCGCGCCGCCCGCCCCGGCTCTGACTGACCGCGTTACTCCCACAG
GTGAGCGGGCGGGACGGCCCTTCTCCTCCGGGCTGTAATTAGCGCTTGGTTTAATGACGGCTTGTTTCTTTTCTGTGGCTGCGT
GAAAGCCTTGAGGGGCTCCGGGAGGGCCCTTTGTGCGGGGGGAGCGGCTCGGGGGGTGCGTGCGTGTGTGTGTGCGTGGG
GAGCGCCGCGTGCGGCTCCGCGCTGCCCGGCGGCTGTGAGCGCTGCGGGCGCGGCGCGGGGCTTTGTGCGCTCCGCAGTGT
GCGCGAGGGGAGCGCGGCCGGGGGCGGTGCCCCGCGGTGCGGGGGGGGCTGCGAGGGGAACAAAGGCTGCGTGCGGGGT
GTGTGCGTGGGGGGGTGAGCAGGGGGTGTGGGCGCGTCGGTCGGGCTGCAACCCCCCCTGCACCCCCCTCCCCGAGTTGCTG
AGCACGGCCCGGCTTCGGGTGCGGGGCTCCGTACGGGGCGTGGCGCGGGGCTCGCCGTGCCGGGCGGGGGGTGGCGGCAG
GTGGGGGTGCCGGGCGGGGGGGGGCCGCCYCGGGCCGGGGRGGGCTCGGGGGRGGGGCGCGGCGGCCCCCGGAGCGCCG
GCGGCTGTCGAGGCGCGGCGAGCCGCAGCCATTGCCTTTTATGGTAATCGTGCGAGAGGGCGCAGGGACTTCCTTTGTCCCA
AATCTGTGCGGAGCCGAAATCTGGGAGGCGCCGCCGCACCCCCTCTAGCGGGCGCGGGGCGAAGCGGTGCGGCGCCGGCAG
GAAGGAAATGGGGGGGGAGGGCCTTCGTGCGTCGCCGCGCCGCCGTCCCCTTCTCCCTCTCCAGCCTCGGGGCTGTCCGCGG
GGGGACGGCTGCCTTCGGGGGGGACGGGGCAGGGGGGGGTTCGGCTTCTGGCGTGTGACCGGCGGCTCTAGAGCCTCTGCT
AACCATGTTCATGCCTTCTTCTTTTTCCTACAGCTCCTGGGCAACGTGCTGGTTATTGTGCTGTCTCATCATTTTGGCAAAGAATT
CGGGCCGCGTTGACGCGCACGGCAAGAGGCGAGGGGCGGCGACTGGTGAGAGATGGGTGCGAGAGCGTCAGTATTAAGCG
GGGGAGAATTAGATCGATGGGAAAAAATTCGGTTAAGGCCAGGGGGAAAGAAAAAATATAAATTAAAACATATAGTATGGG
CAAGCAGGGAGCTAGAACGATTCGCAGTTAATCCTGGCCTGTTAGAAACATCAGAAGGCTGTAGACAAATACTGGGACAGCT
ACAACCATCCCTTCAGACAGGATCAGAAGAACTTAGATCATTATATAATACAGTAGCAACCCTCTATTGTGTGCATCAAAGGAT
AGAGATAAAAGACACCAAGGAAGCTTTAGACAAGATAGAGGAAGAGCAAAACAAAAGTAAGAAAAAAGCACAGCAAGCAGC
AGCTGACACAGGACACAGCAATCAGGTCAGCCAAAATTACCCTATAGTGCAGAACATCCAGGGGCAAATGGTACATCAGGCC
ATATCACCTAGAACTTTAAATGCATGGGTAAAAGTAGTAGAAGAGAAGGCTTTCAGCCCAGAAGTGATACCCATGTTTTCAGC
ATTATCAGAAGGAGCCACCCCACAAGATTTAAACACCATGCTAAACACAGTGGGGGGACATCAAGCAGCCATGCAAATGTTAA
AAGAGACCATCAATGAGGAAGCTGCAGAATGGGATAGAGTGCATCCAGTGCATGCAGGGCCTATTGCACCAGGCCAGATGA
GAGAACCAAGGGGAAGTGACATAGCAGGAACTACTAGTACCCTTCAGGAACAAATAGGATGGATGACACATAATCCACCTAT
CCCAGTAGGAGAAATCTATAAAAGATGGATAATCCTGGGATTAAATAAAATAGTAAGAATGTATAGCCCTACCAGCATTCTGG
ACATAAGACAAGGACCAAAGGAACCCTTTAGAGACTATGTAGACCGATTCTATAAAACTCTAAGAGCCGAGCAAGCTTCACAA
GAGGTAAAAAATTGGATGACAGAAACCTTGTTGGTCCAAAATGCGAACCCAGATTGTAAGACTATTTTAAAAGCATTGGGACC
AGGAGCGACACTAGAAGAAATGATGACAGCATGTCAGGGAGTGGGGGGACCCGGCCATAAAGCAAGAGTTTTGGCTGAAGC
AATGAGCCAAGTAACAAATCCAGCTACCATAATGATACAGAAAGGCAATTTTAGGAACCAAAGAAAGACTGTTAAGTGTTTCA
ATTGTGGCAAAGAAGGGCACATAGCCAAAAATTGCAGGGCCCCTAGGAAAAAGGGCTGTTGGAAATGTGGAAAGGAAGGAC
ACCAAATGAAAGATTGTACTGAGAGACAGGCTAATTTTTTAGGGAAGATCTGGCCTTCCCACAAGGGAAGGCCAGGGAATTTT
CTTCAGAGCAGACCAGAGCCAACAGCCCCACCAGAAGAGAGCTTCAGGTTTGGGGAAGAGACAACAACTCCCTCTCAGAAGC
AGGAGCCGATAGACAAGGAACTGTATCCTTTAGCTTCCCTCAGATCACTCTTTGGCAGCGACCCCTCGTCACAATAAAGATAGG
GGGGCAATTAAAGGAAGCTCTATTAGATACAGGAGCAGATGATACAGTATTAGAAGAAATGAATTTGCCAGGAAGATGGAAA
CCAAAAATGATAGGGGGAATTGGAGGTTTTATCAAAGTAAGACAGTATGATCAGATACTCATAGAAATCTGCGGACATAAAG
CTATAGGTACAGTATTAGTAGGACCTACACCTGTCAACATAATTGGAAGAAATCTGTTGACTCAGATTGGCTGCACTTTAAATT
TTCCCATTAGTCCTATTGAGACTGTACCAGTAAAATTAAAGCCAGGAATGGATGGCCCAAAAGTTAAACAATGGCCATTGACA
GAAGAAAAAATAAAAGCATTAGTAGAAATTTGTACAGAAATGGAAAAGGAAGGAAAAATTTCAAAAATTGGGCCTGAAAATC
CATACAATACTCCAGTATTTGCCATAAAGAAAAAAGACAGTACTAAATGGAGAAAATTAGTAGATTTCAGAGAACTTAATAAG
AGAACTCAAGATTTCTGGGAAGTTCAATTAGGAATACCACATCCTGCAGGGTTAAAACAGAAAAAATCAGTAACAGTACTGGA
TGTGGGCGATGCATATTTTTCAGTTCCCTTAGATAAAGACTTCAGGAAGTATACTGCATTTACCATACCTAGTATAAACAATGA
GACACCAGGGATTAGATATCAGTACAATGTGCTTCCACAGGGATGGAAAGGATCACCAGCAATATTCCAGTGTAGCATGACAA
AAATCTTAGAGCCTTTTAGAAAACAAAATCCAGACATAGTCATCTATCAATACATGGATGATTTGTATGTAGGATCTGACTTAG
AAATAGGGCAGCATAGAACAAAAATAGAGGAACTGAGACAACATCTGTTGAGGTGGGGATTTACCACACCAGACAAAAAACA
TCAGAAAGAACCTCCATTCCTTTGGATGGGTTATGAACTCCATCCTGATAAATGGACAGTACAGCCTATAGTGCTGCCAGAAAA
GGACAGCTGGACTGTCAATGACATACAGAAATTAGTGGGAAAATTGAATTGGGCAAGTCAGATTTATGCAGGGATTAAAGTA
AGGCAATTATGTAAACTTCTTAGGGGAACCAAAGCACTAACAGAAGTAGTACCACTAACAGAAGAAGCAGAGCTAGAACTGG
CAGAAAACAGGGAGATTCTAAAAGAACCGGTACATGGAGTGTATTATGACCCATCAAAAGACTTAATAGCAGAAATACAGAA
GCAGGGGCAAGGCCAATGGACATATCAAATTTATCAAGAGCCATTTAAAAATCTGAAAACAGGAAAGTATGCAAGAATGAAG
GGTGCCCACACTAATGATGTGAAACAATTAACAGAGGCAGTACAAAAAATAGCCACAGAAAGCATAGTAATATGGGGAAAGA
CTCCTAAATTTAAATTACCCATACAAAAGGAAACATGGGAAGCATGGTGGACAGAGTATTGGCAAGCCACCTGGATTCCTGAG
TGGGAGTTTGTCAATACCCCTCCCTTAGTGAAGTTATGGTACCAGTTAGAGAAAGAACCCATAATAGGAGCAGAAACTTTCTAT
GTAGATGGGGCAGCCAATAGGGAAACTAAATTAGGAAAAGCAGGATATGTAACTGACAGAGGAAGACAAAAAGTTGTCCCC
CTAACGGACACAACAAATCAGAAGACTGAGTTACAAGCAATTCATCTAGCTTTGCAGGATTCGGGATTAGAAGTAAACATAGT
GACAGACTCACAATATGCATTGGGAATCATTCAAGCACAACCAGATAAGAGTGAATCAGAGTTAGTCAGTCAAATAATAGAGC
AGTTAATAAAAAAGGAAAAAGTCTACCTGGCATGGGTACCAGCACACAAAGGAATTGGAGGAAATGAACAAGTAGATAAATT
GGTCAGTGCTGGAATCAGGAAAGTACTATTTTTAGATGGAATAGATAAGGCCCAAGAAGAACATGAGAAATATCACAGTAAT
TGGAGAGCAATGGCTAGTGATTTTAACCTACCACCTGTAGTAGCAAAAGAAATAGTAGCCAGCTGTGATAAATGTCAGCTAAA
AGGGGAAGCCATGCATGGACAAGTAGACTGTAGCCCAGGAATATGGCAGCTAGATTGTACACATTTAGAAGGAAAAGTTATC
TTGGTAGCAGTTCATGTAGCCAGTGGATATATAGAAGCAGAAGTAATTCCAGCAGAGACAGGGCAAGAAACAGCATACTTCC
TCTTAAAATTAGCAGGAAGATGGCCAGTAAAAACAGTACATACAGACAATGGCAGCAATTTCACCAGTACTACAGTTAAGGCC
GCCTGTTGGTGGGCGGGGATCAAGCAGGAATTTGGCATTCCCTACAATCCCCAAAGTCAAGGAGTAATAGAATCTATGAATAA
AGAATTAAAGAAAATTATAGGACAGGTAAGAGATCAGGCTGAACATCTTAAGACAGCAGTACAAATGGCAGTATTCATCCACA
ATTTTAAAAGAAAAGGGGGGATTGGGGGGTACAGTGCAGGGGAAAGAATAGTAGACATAATAGCAACAGACATACAAACTA
AAGAATTACAAAAACAAATTACAAAAATTCAAAATTTTCGGGTTTATTACAGGGACAGCAGAGATCCAGTTTGGAAAGGACCA
GCAAAGCTCCTCTGGAAAGGTGAAGGGGCAGTAGTAATACAAGATAATAGTGACATAAAAGTAGTGCCAAGAAGAAAAGCA
AAGATCATCAGGGATTATGGAAAACAGATGGCAGGTGATGATTGTGTGGCAAGTAGACAGGATGAGGATTAACACATGGAA
TTCTGCAACAACTGCTGTTTATCCATTTCAGAATTGGGTGTCGACATAGCAGAATAGGCGTTACTCGACAGAGGAGAGCAAGA
AATGGAGCCAGTAGATCCTAGACTAGAGCCCTGGAAGCATCCAGGAAGTCAGCCTAAAACTGCTTGTACCAATTGCTATTGTA
AAAAGTGTTGCTTTCATTGCCAAGTTTGTTTCATGACAAAAGCCTTAGGCATCTCCTATGGCAGGAAGAAGCGGAGACAGCGA
CGAAGAGCTCATCAGAACAGTCAGACTCATCAAGCTTCTCTATCAAAGCAGTAAGTAGTACATGTAATGCAACCTATAATAGTA
GCAATAGTAGCATTAGTAGTAGCAATAATAATAGCAATAGTTGTGTGGTCCATAGTAATCATAGAATATAGGAAAATGGCCGC
TGATCTTCAGACCTGGAGGAGGAGATATGAGGGACAATTGGAGAAGTGAATTATATAAATATAAAGTAGTAAAAATTGAACC
ATTAGGAGTAGCACCCACCAAGGCAAAGAGAAGAGTGGTGCAGAGAGAAAAAAGAGCAGTGGGAATAGGAGCTTTGTTCCT
TGGGTTCTTGGGAGCAGCAGGAAGCACTATGGGCGCAGCGTCAATGACGCTGACGGTACAGGCCAGACAATTATTGTCTGGT
ATAGTGCAGCAGCAGAACAATTTGCTGAGGGCTATTGAGGCGCAACAGCATCTGTTGCAACTCACAGTCTGGGGCATCAAGC
AGCTCCAGGCAAGAATCCTGGCTGTGGAAAGATACCTAAAGGATCAACAGCTCCTGGGGATTTGGGGTTGCTCTGGAAAACT
CATTTGCACCACTGCTGTGCCTTGGAATGCTAGTTGGAGTAATAAATCTCTGGAACAGATTTGGAATCACACGACCTGGATGG
AGTGGGACAGAGAAATTAACAATTACACAAGCTTAATACACTCCTTAATTGAAGAATCGCAAAACCAGCAAGAAAAGAATGAA
CAAGAATTATTGGAATTAGATAAATGGGCAAGTTTGTGGAATTGGTTTAACATAACAAATTGGCTGTGGTATATAAAATTATTC
ATAATGATAGTAGGAGGCTTGGTAGGTTTAAGAATAGTTTTTGCTGTACTTTCTATAGTGAATAGAGTTAGGCAGGGATATTC
ACCATTATCGTTTCAGACCCACCTCCCAACCCCGAGGGGACCCGACAGGCCCGAAGGAATAGAAGAAGAAGGTGGAGAGAG
AGACAGAGACAGATCCATTCGATTAGTGAACGGATCCTTGGCACTTATCTGGGACGATCTGCGGAGCCTGTGCCTCTTCAGCT
ACCACCGCTTGAGAGACTTACTCTTGATTGTAACGAGGATTGTGGAACTTCTGGGACGCAGGGGGGGGAAGCCCTCAAATAT
TGGTGGAATCTCCTACAATATTGGAGTCAGGAGCTAAAGAATAGTGCTGTTAGCTTGCTCAATGCCACAGCCATAGCAGTAGC
TGAGGGGACAGATAGGGTTATAGAAGTAGTACAAGGAGCTTGTAGAGCTATTCGCCACATACCTAGAAGAATAAGACAGGGC
TTGGAAAGGATTTTGCTATAAGCTCGAAACAACCGGTACCTCTAGAACTATAGCTAGCAGATCTTTTTCCCTCTGCCAAAAATTA
TGGGGACATCATGAAGCCCCTTGAGCATCTGACTTCTGGCTAATAAAGGAAATTTATTTTCATTGCAATAGTGTGTTGGAATTT
TTTGTGTCTCTCACTCGGAAGGACATATGGGAGGGCAAATCATTTAAAACATCAGAATGAGTATTTGGTTTAGAGTTTGGCAA
CATATGCCCATATGCTGGCTGCCATGAACAAAGGTTGGCTATAAAGAGGTCATCAGTATATGAAACAGCCCCCTGCTGTCCATT
CCTTATTCCATAGAAAAGCCTTGACTTGAGGTTAGATTTTTTTTATATTTTGTTTTGTGTTATTTTTTTCTTTAACATCCCTAAAAT
TTTCCTTACATGTTTTACTAGCCAGATTTTTCCTCCTCTCCTGACTACTCCCAGTCATAGCTGTCCCTCTTCTCTTATGGAGATCCC
TCGACCTGCAGCCCAAGCTTGGCGTAATCATGGTCATAGCTGTTTCCTGTGTGAAATTGTTATCCGCTCACAATTCCACACAACA
TACGAGCCGGAAGCATAAAGTGTAAAGCCTGGGGTGCCTAATGAGTGAGCTAACTCACATTAATTGCGTTGCGCTCACTGCCC
GCTTTCCAGTCGGGAAACCTGTCGTGCCAGCGGATCCGCATCTCAATTAGTCAGCAACCATAGTCCCGCCCCTAACTCCGCCCA
TCCCGCCCCTAACTCCGCCCAGTTCCGCCCATTCTCCGCCCCATGGCTGACTAATTTTTTTTATTTATGCAGAGGCCGAGGCCGC
CTCGGCCTCTGAGCTATTCCAGAAGTAGTGAGGAGGCTTTTTTGGAGGCCTAGGCTTTTGCAAAAAGCTAACTTGTTTATTGCA
GCTTATAATGGTTACAAATAAAGCAATAGCATCACAAATTTCACAAATAAAGCATTTTTTTCACTGCATTCTAGTTGTGGTTTGT
CCAAACTCATCAATGTATCTTATCATGTCTGGATCCGCTGCATTAATGAATCGGCCAACGCGCGGGGAGAGGCGGTTTGCGTA
TTGGGCGCTCTTCCGCTTCCTCGCTCACTGACTCGCTGCGCTCGGTCGTTCGGCTGCGGCGAGCGGTATCAGCTCACTCAAAGG
CGGTAATACGGTTATCCACAGAATCAGGGGATAACGCAGGAAAGAACATGTGAGCAAAAGGCCAGCAAAAGGCCAGGAACC
GTAAAAAGGCCGCGTTGCTGGCGTTTTTCCATAGGCTCCGCCCCCCTGACGAGCATCACAAAAATCGACGCTCAAGTCAGAGG
TGGCGAAACCCGACAGGACTATAAAGATACCAGGCGTTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTTTCTCATAGCTCACGCTGTAGGTATCTCAGTTCGGTGT
AGGTCGTTCGCTCCAAGCTGGGCTGTGTGCACGAACCCCCCGTTCAGCCCGACCGCTGCGCCTTATCCGGTAACTATCGTCTTG
AGTCCAACCCGGTAAGACACGACTTATCGCCACTGGCAGCAGCCACTGGTAACAGGATTAGCAGAGCGAGGTATGTAGGCGG
TGCTACAGAGTTCTTGAAGTGGTGGCCTAACTACGGCTACACTAGAAGAACAGTATTTGGTATCTGCGCTCTGCTGAAGCCAG
TTACCTTCGGAAAAAGAGTTGGTAGCTCTTGATCCGGCAAACAAACCACCGCTGGTAGCGGTGGTTTTTTTGTTTGCAAGCAGC
AGATTACGCGCAGAAAAAAAGGATCTCAAGAAGATCCTTTGATCTTTTCTACGGGGTCTGACGCTCAGTGGAACGAAAACTCA
CGTTAAGGGATTTTGGTCATGAGATTATCAAAAAGGATCTTCACCTAGATCCTTTTAAATTAAAAATGAAGTTTTAAATCAATCT
AAAGTATATATGAGTAAACTTGGTCTGACAGTTACCAATGCTTAATCAGTGAGGCACCTATCTCAGCGATCTGTCTATTTCGTTC
ATCCATAGTTGCCTGACTCCCCGTCGTGTAGATAACTACGATACGGGAGGGCTTACCATCTGGCCCCAGTGCTGCAATGATACC
GCGAGACCCACGCTCACCGGCTCCAGATTTATCAGCAATAAACCAGCCAGCCGGAAGGGCCGAGCGCAGAAGTGGTCCTGCA
ACTTTATCCGCCTCCATCCAGTCTATTAATTGTTGCCGGGAAGCTAGAGTAAGTAGTTCGCCAGTTAATAGTTTGCGCAACGTT
GTTGCCATTGCTACAGGCATCGTGGTGTCACGCTCGTCGTTTGGTATGGCTTCATTCAGCTCCGGTTCCCAACGATCAAGGCGA
GTTACATGATCCCCCATGTTGTGCAAAAAAGCGGTTAGCTCCTTCGGTCCTCCGATCGTTGTCAGAAGTAAGTTGGCCGCAGTG
TTATCACTCATGGTTATGGCAGCACTGCATAATTCTCTTACTGTCATGCCATCCGTAAGATGCTTTTCTGTGACTGGTGAGTACT
CAACCAAGTCATTCTGAGAATAGTGTATGCGGCGACCGAGTTGCTCTTGCCCGGCGTCAATACGGGATAATACCGCGCCACAT
AGCAGAACTTTAAAAGTGCTCATCATTGGAAAACGTTCTTCGGGGCGAAAACTCTCAAGGATCTTACCGCTGTTGAGATCCAGT
TCGATGTAACCCACTCGTGCACCCAACTGATCTTCAGCATCTTTTACTTTCACCAGCGTTTCTGGGTGAGCAAAAACAGGAAGG
CAAAATGCCGCAAAAAAGGGAATAAGGGCGACACGGAAATGTTGAATACTCATACTCTTCCTTTTTCAATATTATTGAAGCATT
TATCAGGGTTATTGTCTCATGAGCGGATACATATTTGAATGTATTTAGAAAAATAAACAAATAGGGGTTCCGCGCACATTTCCC
CGAAAAGTGCCACCTGGGTCGACA
SEQ ID No. 19-pMD2.G envelope vector encoding the VSV-G gene
GGATCCCCTGAGGGGGCCCCCATGGGCTAGAGGATCCGGCCTCGGCCTCTGCATAAATAAAAAAAATTAGTCAGCCATGAGC
TTGGCCCATTGCATACGTTGTATCCATATCATAATATGTACATTTATATTGGCTCATGTCCAACATTACCGCCATGTTGACATTGA
TTATTGACTAGTTATTAATAGTAATCAATTACGGGGTCATTAGTTCATAGCCCATATATGGAGTTCCGCGTTACATAACTTACGG
TAAATGGCCCGCCTGGCTGACCGCCCAACGACCCCCGCCCATTGACGTCAATAATGACGTATGTTCCCATAGTAACGCCAATAG
GGACTTTCCATTGACGTCAATGGGTGGAGTATTTACGGTAAACTGCCCACTTGGCAGTACATCAAGTGTATCATATGCCAAGTA
CGCCCCCTATTGACGTCAATGACGGTAAATGGCCCGCCTGGCATTATGCCCAGTACATGACCTTATGGGACTTTCCTACTTGGC
AGTACATCTACGTATTAGTCATCGCTATTACCATGGTGATGCGGTTTTGGCAGTACATCAATGGGCGTGGATAGCGGTTTGACT
CACGGGGATTTCCAAGTCTCCACCCCATTGACGTCAATGGGAGTTTGTTTTGGCACCAAAATCAACGGGACTTTCCAAAATGTC
GTAACAACTCCGCCCCATTGACGCAAATGGGCGGTAGGCGTGTACGGTGGGAGGTCTATATAAGCAGAGCTCGTTTAGTGAA
CCGTCAGATCGCCTGGAGACGCCATCCACGCTGTTTTGACCTCCATAGAAGACACCGGGACCGATCCAGCCTCCCCTCGAAGCT
TACATGTGGTACCGAGCTCGGATCCTGAGAACTTCAGGGTGAGTCTATGGGACCCTTGATGTTTTCTTTCCCCTTCTTTTCTATG
GTTAAGTTCATGTCATAGGAAGGGGAGAAGTAACAGGGTACACATATTGACCAAATCAGGGTAATTTTGCATTTGTAATTTTA
AAAAATGCTTTCTTCTTTTAATATACTTTTTTGTTTATCTTATTTCTAATACTTTCCCTAATCTCTTTCTTTCAGGGCAATAATGATA
CAATGTATCATGCCTCTTTGCACCATTCTAAAGAATAACAGTGATAATTTCTGGGTTAAGGCAATAGCAATATTTCTGCATATAA
ATATTTCTGCATATAAATTGTAACTGATGTAAGAGGTTTCATATTGCTAATAGCAGCTACAATCCAGCTACCATTCTGCTTTTATT
TTATGGTTGGGATAAGGCTGGATTATTCTGAGTCCAAGCTAGGCCCTTTTGCTAATCATGTTCATACCTCTTATCTTCCTCCCAC
AGCTCCTGGGCAACGTGCTGGTCTGTGTGCTGGCCCATCACTTTGGCAAAGCACGTGAGATCTGAATTCTGACACTATGAAGT
GCCTTTTGTACTTAGCCTTTTTATTCATTGGGGTGAATTGCAAGTTCACCATAGTTTTTCCACACAACCAAAAAGGAAACTGGAA
AAATGTTCCTTCTAATTACCATTATTGCCCGTCAAGCTCAGATTTAAATTGGCATAATGACTTAATAGGCACAGCCTTACAAGTC
AAAATGCCCAAGAGTCACAAGGCTATTCAAGCAGACGGTTGGATGTGTCATGCTTCCAAATGGGTCACTACTTGTGATTTCCG
CTGGTATGGACCGAAGTATATAACACATTCCATCCGATCCTTCACTCCATCTGTAGAACAATGCAAGGAAAGCATTGAACAAAC
GAAACAAGGAACTTGGCTGAATCCAGGCTTCCCTCCTCAAAGTTGTGGATATGCAACTGTGACGGATGCCGAAGCAGTGATTG
TCCAGGTGACTCCTCACCATGTGCTGGTTGATGAATACACAGGAGAATGGGTTGATTCACAGTTCATCAACGGAAAATGCAGC
AATTACATATGCCCCACTGTCCATAACTCTACAACCTGGCATTCTGACTATAAGGTCAAAGGGCTATGTGATTCTAACCTCATTT
CCATGGACATCACCTTCTTCTCAGAGGACGGAGAGCTATCATCCCTGGGAAAGGAGGGCACAGGGTTCAGAAGTAACTACTTT
GCTTATGAAACTGGAGGCAAGGCCTGCAAAATGCAATACTGCAAGCATTGGGGAGTCAGACTCCCATCAGGTGTCTGGTTCG
AGATGGCTGATAAGGATCTCTTTGCTGCAGCCAGATTCCCTGAATGCCCAGAAGGGTCAAGTATCTCTGCTCCATCTCAGACCT
CAGTGGATGTAAGTCTAATTCAGGACGTTGAGAGGATCTTGGATTATTCCCTCTGCCAAGAAACCTGGAGCAAAATCAGAGCG
GGTCTTCCAATCTCTCCAGTGGATCTCAGCTATCTTGCTCCTAAAAACCCAGGAACCGGTCCTGCTTTCACCATAATCAATGGTA
CCCTAAAATACTTTGAGACCAGATACATCAGAGTCGATATTGCTGCTCCAATCCTCTCAAGAATGGTCGGAATGATCAGTGGAA
CTACCACAGAAAGGGAACTGTGGGATGACTGGGCACCATATGAAGACGTGGAAATTGGACCCAATGGAGTTCTGAGGACCA
GTTCAGGATATAAGTTTCCTTTATACATGATTGGACATGGTATGTTGGACTCCGATCTTCATCTTAGCTCAAAGGCTCAGGTGTT
CGAACATCCTCACATTCAAGACGCTGCTTCGCAACTTCCTGATGATGAGAGTTTATTTTTTGGTGATACTGGGCTATCCAAAAAT
CCAATCGAGCTTGTAGAAGGTTGGTTCAGTAGTTGGAAAAGCTCTATTGCCTCTTTTTTCTTTATCATAGGGTTAATCATTGGAC
TATTCTTGGTTCTCCGAGTTGGTATCCATCTTTGCATTAAATTAAAGCACACCAAGAAAAGACAGATTTATACAGACATAGAGA
TGAACCGACTTGGAAAGTAACTCAAATCCTGCACAACAGATTCTTCATGTTTGGACCAAATCAACTTGTGATACCATGCTCAAA
GAGGCCTCAATTATATTTGAGTTTTTAATTTTTATGAAAAAAAAAAAAAAAAACGGAATTCACCCCACCAGTGCAGGCTGCCTA
TCAGAAAGTGGTGGCTGGTGTGGCTAATGCCCTGGCCCACAAGTATCACTAAGCTCGCTTTCTTGCTGTCCAATTTCTATTAAA
GGTTCCTTTGTTCCCTAAGTCCAACTACTAAACTGGGGGATATTATGAAGGGCCTTGAGCATCTGGATTCTGCCTAATAAAAAA
CATTTATTTTCATTGCAATGATGTATTTAAATTATTTCTGAATATTTTACTAAAAAGGGAATGTGGGAGGTCAGTGCATTTAAAA
CATAAAGAAATGAAGAGCTAGTTCAAACCTTGGGAAAATACACTATATCTTAAACTCCATGAAAGAAGGTGAGGCTGCAAACA
GCTAATGCACATTGGCAACAGCCCCTGATGCCTATGCCTTATTCATCCCTCAGAAAAGGATTCAAGTAGAGGCTTGATTTGGAG
GTTAAAGTTTTGCTATGCTGTATTTTACATTACTTATTGTTTTAGCTGTCCTCATGAATGTCTTTTCACTACCCATTTGCTTATCCT
GCATCTCTCAGCCTTGACTCCACTCAGTTCTCTTGCTTAGAGATACCACCTTTCCCCTGAAGTGTTCCTTCCATGTTTTACGGCGA
GATGGTTTCTCCTCGCCTGGCCACTCAGCCTTAGTTGTCTCTGTTGTCTTATAGAGGTCTACTTGAAGAAGGAAAAACAGGGGG
CATGGTTTGACTGTCCTGTGAGCCCTTCTTCCCTGCCTCCCCCACTCACAGTGACCCGGAATCCCTCGACATGGCAGTCTAGCAC
TAGTGCGGCCGCAGATCTGCTTCCTCGCTCACTGACTCGCTGCGCTCGGTCGTTCGGCTGCGGCGAGCGGTATCAGCTCACTCA
AAGGCGGTAATACGGTTATCCACAGAATCAGGGGATAACGCAGGAAAGAACATGTGAGCAAAAGGCCAGCAAAAGGCCAGG
AACCGTAAAAAGGCCGCGTTGCTGGCGTTTTTCCATAGGCTCCGCCCCCCTGACGAGCATCACAAAAATCGACGCTCAAGTCA
GAGGTGGCGAAACCCGACAGGACTATAAAGATACCAGGCGTTTCCCCCTGGAAGCTCCCTCGTGCGCTCTCCTGTTCCGACCC
TGCCGCTTACCGGATACCTGTCCGCCTTTCTCCCTTCGGGAAGCGTGGCGCTTTCTCATAGCTCACGCTGTAGGTATCTCAGTTC
GGTGTAGGTCGTTCGCTCCAAGCTGGGCTGTGTGCACGAACCCCCCGTTCAGCCCGACCGCTGCGCCTTATCCGGTAACTATC
GTCTTGAGTCCAACCCGGTAAGACACGACTTATCGCCACTGGCAGCAGCCACTGGTAACAGGATTAGCAGAGCGAGGTATGT
AGGCGGTGCTACAGAGTTCTTGAAGTGGTGGCCTAACTACGGCTACACTAGAAGAACAGTATTTGGTATCTGCGCTCTGCTGA
AGCCAGTTACCTTCGGAAAAAGAGTTGGTAGCTCTTGATCCGGCAAACAAACCACCGCTGGTAGCGGTGGTTTTTTTGTTTGCA
AGCAGCAGATTACGCGCAGAAAAAAAGGATCTCAAGAAGATCCTTTGATCTTTTCTACGGGGTCTGACGCTCAGTGGAACGAA
AACTCACGTTAAGGGATTTTGGTCATGAGATTATCAAAAAGGATCTTCACCTAGATCCTTTTAAATTAAAAATGAAGTTTTAAAT
CAATCTAAAGTATATATGAGTAAACTTGGTCTGACAGTTACCAATGCTTAATCAGTGAGGCACCTATCTCAGCGATCTGTCTATT
TCGTTCATCCATAGTTGCCTGACTCCCCGTCGTGTAGATAACTACGATACGGGAGGGCTTACCATCTGGCCCCAGTGCTGCAAT
GATACCGCGAGACCCACGCTCACCGGCTCCAGATTTATCAGCAATAAACCAGCCAGCCGGAAGGGCCGAGCGCAGAAGTGGT
CCTGCAACTTTATCCGCCTCCATCCAGTCTATTAATTGTTGCCGGGAAGCTAGAGTAAGTAGTTCGCCAGTTAATAGTTTGCGC
AACGTTGTTGCCATTGCTACAGGCATCGTGGTGTCACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTTCGGTCCTCCGATCGTTGTCAGAAGTAAGTTGGC
CGCAGTGTTATCACTCATGGTTATGGCAGCACTGCATAATTCTCTTACTGTCATGCCATCCGTAAGATGCTTTTCTGTGACTGGT
GAGTACTCAACCAAGTCATTCTGAGAATAGTGTATGCGGCGACCGAGTTGCTCTTGCCCGGCGTCAATACGGGATAATACCGC
GCCACATAGCAGAACTTTAAAAGTGCTCATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTTTCACCAGCGTTTCTGGGTGAGCAAAAAC
AGGAAGGCAAAATGCCGCAAAAAAGGGAATAAGGGCGACACGGAAATGTTGAATACTCATACTCTTCCTTTTTCAATATTATT
GAAGCATTTATCAGGGTTATTGTCTCATGAGCGGATACATATTTGAATGTATTTAGAAAAATAAACAAATAGGGGTTCCGCGC
ACATTTCCCCGAAAAGTGCCACCTGACGT
SEQ ID No. 20-FUW-GaGiFo (vector 1)
TCGACGGATCGGGAGATCTCCCGATCCCCTATGGTGCACTCTCAGTACAATCTGCTCTGATGCCGCATAGTTAAGCCAGTATCT
GCTCCCTGCTTGTGTGTTGGAGGTCGCTGAGTAGTGCGCGAGCAAAATTTAAGCTACAACAAGGCAAGGCTTGACCGACAATT
GCATGAAGAATCTGCTTAGGGTTAGGCGTTTTGCGCTGCTTCGCGATGTACGGGCCAGATATACGCGTTGACATTGATTATTG
ACTAGTTATTAATAGTAATCAATTACGGGGTCATTAGTTCATAGCCCATATATGGAGTTCCGCGTTACATAACTTACGGTAAAT
GGCCCGCCTGGCTGACCGCCCAACGACCCCCGCCCATTGACGTCAATAATGACGTATGTTCCCATAGTAACGCCAATAGGGAC
TTTCCATTGACGTCAATGGGTGGAGTATTTACGGTAAACTGCCCACTTGGCAGTACATCAAGTGTATCATATGCCAAGTACGCC
CCCTATTGACGTCAATGACGGTAAATGGCCCGCCTGGCATTATGCCCAGTACATGACCTTATGGGACTTTCCTACTTGGCAGTA
CATCTACGTATTAGTCATCGCTATTACCATGGTGATGCGGTTTTGGCAGTACATCAATGGGCGTGGATAGCGGTTTGACTCACG
GGGATTTCCAAGTCTCCACCCCATTGACGTCAATGGGAGTTTGTTTTGGCACCAAAATCAACGGGACTTTCCAAAATGTCGTAA
CAACTCCGCCCCATTGACGCAAATGGGCGGTAGGCGTGTACGGTGGGAGGTCTATATAAGCAGCGCGTTTTGCCTGTACTGG
GTCTCTCTGGTTAGACCAGATCTGAGCCTGGGAGCTCTCTGGCTAACTAGGGAACCCACTGCTTAAGCCTCAATAAAGCTTGCC
TTGAGTGCTTCAAGTAGTGTGTGCCCGTCTGTTGTGTGACTCTGGTAACTAGAGATCCCTCAGACCCTTTTAGTCAGTGTGGAA
AATCTCTAGCAGTGGCGCCCGAACAGGGACTTGAAAGCGAAAGGGAAACCAGAGGAGCTCTCTCGACGCAGGACTCGGCTTG
CTGAAGCGCGCACGGCAAGAGGCGAGGGGCGGCGACTGGTGAGTACGCCAAAAATTTTGACTAGCGGAGGCTAGAAGGAG
AGAGATGGGTGCGAGAGCGTCAGTATTAAGCGGGGGAGAATTAGATCGCGATGGGAAAAAATTCGGTTAAGGCCAGGGGG
AAAGAAAAAATATAAATTAAAACATATAGTATGGGCAAGCAGGGAGCTAGAACGATTCGCAGTTAATCCTGGCCTGTTAGAA
ACATCAGAAGGCTGTAGACAAATACTGGGACAGCTACAACCATCCCTTCAGACAGGATCAGAAGAACTTAGATCATTATATAA
TACAGTAGCAACCCTCTATTGTGTGCATCAAAGGATAGAGATAAAAGACACCAAGGAAGCTTTAGACAAGATAGAGGAAGAG
CAAAACAAAAGTAAGACCACCGCACAGCAAGCGGCCGCTGATCTTCAGACCTGGAGGAGGAGATATGAGGGACAATTGGAG
AAGTGAATTATATAAATATAAAGTAGTAAAAATTGAACCATTAGGAGTAGCACCCACCAAGGCAAAGAGAAGAGTGGTGCAG
AGAGAAAAAAGAGCAGTGGGAATAGGAGCTTTGTTCCTTGGGTTCTTGGGAGCAGCAGGAAGCACTATGGGCGCAGCGTCA
ATGACGCTGACGGTACAGGCCAGACAATTATTGTCTGGTATAGTGCAGCAGCAGAACAATTTGCTGAGGGCTATTGAGGCGC
AACAGCATCTGTTGCAACTCACAGTCTGGGGCATCAAGCAGCTCCAGGCAAGAATCCTGGCTGTGGAAAGATACCTAAAGGAT
CAACAGCTCCTGGGGATTTGGGGTTGCTCTGGAAAACTCATTTGCACCACTGCTGTGCCTTGGAATGCTAGTTGGAGTAATAA
ATCTCTGGAACAGATTTGGAATCACACGACCTGGATGGAGTGGGACAGAGAAATTAACAATTACACAAGCTTAATACACTCCT
TAATTGAAGAATCGCAAAACCAGCAAGAAAAGAATGAACAAGAATTATTGGAATTAGATAAATGGGCAAGTTTGTGGAATTG
GTTTAACATAACAAATTGGCTGTGGTATATAAAATTATTCATAATGATAGTAGGAGGCTTGGTAGGTTTAAGAATAGTTTTTGC
TGTACTTTCTATAGTGAATAGAGTTAGGCAGGGATATTCACCATTATCGTTTCAGACCCACCTCCCAACCCCGAGGGGACCCGA
CAGGCCCGAAGGAATAGAAGAAGAAGGTGGAGAGAGAGACAGAGACAGATCCATTCGATTAGTGAACGGATCGGCACTGC
GTGCGCCAATTCTGCAGACAAATGGCAGTATTCATCCACAATTTTAAAAGAAAAGGGGGGATTGGGGGGTACAGTGCAGGGG
AAAGAATAGTAGACATAATAGCAACAGACATACAAACTAAAGAATTACAAAAACAAATTACAAAAATTCAAAATTTTCGGGTT
TATTACAGGGACAGCAGAGATCCAGTTTGGTTAATTAACCCGTGTCGGCTCCAGATCTGGCCTCCGCGCCGGGTTTTGGCGCC
TCCCGCGGGCGCCCCCCTCCTCACGGCGAGCGCTGCCACGTCAGACGAAGGGCGCAGCGAGCGTCCTGATCCTTCCGCCCGG
ACGCTCAGGACAGCGGCCCGCTGCTCATAAGACTCGGCCTTAGAACCCCAGTATCAGCAGAAGGACATTTTAGGACGGGACTT
GGGTGACTCTAGGGCACTGGTTTTCTTTCCAGAGAGCGGAACAGGCGAGGAAAAGTAGTCCCTTCTCGGCGATTCTGCGGAG
GGATCTCCGTGGGGCGGTGAACGCCGATGATTATATAAGGACGCGCCGGGTGTGGCACAGCTAGTTCCGTCGCAGCCGGGAT
TTGGGTCGCGGTTCTTGTTTGTGGATCGCTGTGATCGTCACTTGGTGAGTAGCGGGCTGCTGGGCTGGCCGGGGCTTTCGTGG
CCGCCGGGCCGCTCGGTGGGACGGAAGCGTGTGGAGAGACCGCCAAGGGCTGTAGTCTGGGTCCGCGAGCAAGGTTGCCCT
GAACTGGGGGTTGGGGGGAGCGCAGCAAAATGGCGGCTGTTCCCGAGTCTTGAATGGAAGACGCTTGTGAGGCGGGCTGTG
AGGTCGTTGAAACAAGGTGGGGGGCATGGTGGGCGGCAAGAACCCAAGGTCTTGAGGCCTTCGCTAATGCGGGAAAGCTCT
TATTCGGGTGAGATGGGCTGGGGCACCATCTGGGGACCCTGACGTGAAGTTTGTCACTGACTGGAGAACTCGGTTTGTCGTCT
GTTGCGGGGGCGGCAGTTATGGCGGTGCCGTTGGGCAGTGCACCCGTACCTTTGGGAGCGCGCGCCCTCGTCGTGTCGTGAC
GTCACCCGTTCTGTTGGCTTATAATGCAGGGTGGGGCCACCTGCCGGTAGGTGTGCGGTAGGCTTTTCTCCGTCGCAGGACGC
AGGGTTCGGGCCTAGGGTAGGCTCTCCTGAATCGACAGGCGCCGGACCTCTGGTGAGGGGAGGGATAAGTGAGGCGTCAGT
TTCTTTGGTCGGTTTTATGTACCTATCTTCTTAAGTAGCTGAAGCTCCGGTTTTGAACTATGCGCTCGGGGTTGGCGAGTGTGTT
TTGTGAAGTTTTTTAGGCACCTTTTGAAATGTAATCATTTGGGTCAATATGTAATTTTCAGTGTTAGACTAGTAAATTGTCCGCT
AAATTCTGGCCGTTTTTGGCTTTTTTGTTAGACGAAGCTTGGGCCCGGGATCCATGGAAGTCGCTCCTGAACAACCACGGTGG
ATGGCTCATCCTGCAGTTCTTAACGCTCAACATCCAGATAGCCATCATCCTGGGCTCGCTCATAATTATATGGAGCCTGCCCAAC
TCCTTCCCCCAGATGAAGTAGATGTATTCTTTAACCATCTTGATAGCCAAGGAAATCCTTATTACGCAAATCCTGCCCATGCCAG
AGCCAGAGTAAGCTATTCACCAGCCCATGCAAGGCTTACTGGTGGACAAATGTGTCGGCCCCATCTGCTGCATTCCCCCGGCCT
GCCGTGGTTGGATGGCGGAAAGGCGGCTCTGAGTGCGGCGGCTGCACATCATCATAATCCTTGGACAGTCTCACCTTTCTCTA
AAACTCCCCTTCATCCTAGTGCAGCCGGTGGTCCAGGTGGTCCGCTGTCCGTTTATCCCGGCGCCGGCGGCGGAAGTGGCGGC
GGAAGTGGCAGTAGCGTAGCTTCATTGACACCCACAGCTGCTCATTCCGGAAGTCATCTCTTTGGGTTTCCCCCTACCCCGCCG
AAAGAGGTTTCCCCAGATCCGTCAACTACCGGTGCCGCTTCCCCTGCTAGTTCCAGTGCCGGCGGAAGCGCTGCAAGGGGCGA
AGATAAAGATGGAGTGAAATATCAAGTCTCCTTGACAGAATCCATGAAAATGGAGTCCGGTTCTCCACTCAGGCCTGGACTCG
CCACAATGGGTACTCAACCCGCCACCCATCATCCAATACCGACTTATCCTTCATACGTACCAGCCGCCGCTCATGATTATTCATC
AGGTTTGTTTCATCCAGGTGGATTTCTTGGCGGGCCTGCTAGTTCTTTTACACCAAAACAAAGGTCCAAAGCCAGGTCTTGCAG
TGAGGGGCGCGAATGCGTAAATTGCGGCGCTACCGCAACACCACTGTGGCGTCGTGATGGAACAGGACATTATCTTTGTAAC
GCATGCGGGCTGTATCATAAAATGAACGGACAAAATCGGCCCCTGATTAAACCTAAACGTCGCTTGTCTGCTGCAAGGCGGGC
TGGGACGTGCTGCGCCAACTGCCAAACAACCACAACAACACTGTGGCGTAGGAATGCTAATGGCGATCCCGTATGTAATGCAT
GCGGTCTGTATTATAAACTTCATAACGTCAATCGGCCCCTTACTATGAAGAAAGAGGGCATACAAACCCGAAATCGCAAAATG
TCAAATAAATCTAAGAAATCCAAGAAGGGAGCCGAATGTTTTGAAGAACTCAGCAAATGTATGCAAGAAAAGTCCAGCCCGTT
TTCCGCCGCCGCTTTGGCCGGCCATATGGCCCCCGTCGGACATCTGCCACCGTTTTCCCATAGCGGTCATATTCTTCCTACACCC
ACCCCAATTCATCCGTCTAGCTCCCTGTCTTTTGGGCATCCTCATCCTAGCTCAATGGTCACAGCTATGGGTGGCAGCGGCGCC
ACAAACTTCTCTCTGCTAAAGCAAGCAGGTGATGTTGAAGAAAACCCCGGGCCTATGCCGAGGAGCTTTCTCGTTAAATCCAA
GAAAGCGCATACATATCATCAACCTAGGGTCCAAGAGGACGAGCCACTGTGGCCACCCGCTCTCACACCAGTTCCTCGGGATC
AAGCACCTTCTAATTCACCCGTGTTGAGTACCTTGTTTCCGAATCAATGTCTCGATTGGACTAATCTGAAGCGGGAACCAGAAT
TGGAACAAGATCAAAATCTGGCACGCATGGCTCCCGCTCCTGAAGGTCCAATCGTTCTCAGTCGGCCGCAAGACGGCGATTCC
CCGCTTTCTGATAGCCCACCGTTTTATAAACCATCTTTTAGCTGGGATACGCTCGCTACGACATACGGTCATTCCTATAGACAAG
CTCCGTCAACTATGCAAAGCGCTTTTCTCGAACATAGCGTTTCACTTTATGGATCACCCCTCGTCCCTTCCACCGAACCTGCTCTG
GACTTTTCCCTGAGATATTCTCCCGGGATGGACGCCTATCATTGCGTTAAATGTAATAAAGTGTTTTCAACACCGCATGGTCTTG
AGGTCCACGTACGCCGATCACACAGCGGCACTCGCCCGTTTGCATGCGATATTTGTGGAAAGACTTTTGGACATGCCGTCTCTC
TCGAACAACATACACATGTGCATTCACAAGAACGTTCCTTTGAATGTCGAATGTGTGGGAAAGCGTTTAAAAGGTCAAGTACC
CTCAGCACGCATCTTCTTATTCATTCTGATACCCGCCCTTATCCATGTCAATTTTGTGGGAAACGCTTTCATCAGAAAAGTGATA
TGAAGAAACATACGTATATTCATACCGGGGAAAAGCCACATAAATGTCAAGTATGTGGCAAAGCATTTTCCCAAAGTTCTAATT
TGATAACGCATAGTCGAAAACATACTGGGTTTAAACCATTTTCCTGCGAACTCTGTACTAAGGGATTTCAAAGGAAAGTAGATC
TTAGAAGGCATAGGGAATCCCAACATAACCTGAAAGGCTCCGGCGAGGGCAGGGGAAGTCTTCTAACATGCGGGGACGTGG
AGGAAAATCCCGGCCCAATGATGTTTTCCGGGTTTAATGCCGATTATGAAGCATCCAGTTCTAGGTGTAGTTCTGCAAGTCCCG
CGGGCGACTCACTGTCATATTATCATAGCCCGGCTGATAGCTTTTCTTCTATGGGGTCACCCGTGAATGCCCAAGATTTCTGTAC
TGATTTGGCAGTGAGCTCAGCTAATTTTATCCCTACTGTGACCGCTATAAGCACTTCTCCTGATCTCCAATGGCTCGTCCAACCA
GCACTGGTTAGTTCAGTCGCTCCCAGTCAAACACGGGCACCCCATCCGTTTGGGGTTCCAGCACCTTCAGCAGGCGCCTATAGC
CGGGCCGGAGTCGTAAAGACAATGACTGGTGGGAGAGCCCAATCAATCGGAAGAAGAGGAAAAGTCGAGCAACTTAGCCCT
GAGGAAGAGGAAAAGAGACGCATTAGGCGCGAGCGGAACAAAATGGCCGCCGCTAAGTGTCGAAATAGGCGTCGTGAACTC
ACAGACACCCTGCAGGCCGAAACCGATCAGCTTGAGGACGAGAAAAGTGCACTGCAAACTGAAATCGCTAATCTTCTCAAAG
AGAAAGAGAAGCTGGAATTTATTCTCGCTGCACATCGTCCAGCATGTAAAATTCCCGACGATCTCGGATTTCCGGAAGAAATG
AGCGTAGCCAGTCTCGACCTTACCGGTGGACTCCCTGAAGTCGCGACACCTGAATCCGAAGAAGCGTTTACATTGCCCTTGCT
GAACGATCCGGAACCAAAACCAAGCGTCGAGCCCGTGAAAAGTATATCATCTATGGAACTTAAAACTGAACCTTTCGACGATT
TTCTCTTTCCTGCTTCCAGCCGCCCATCAGGGTCAGAAACCGCTAGATCTGTCCCCGATATGGATCTGAGCGGAAGCTTTTACG
CTGCCGATTGGGAACCACTCCATTCAGGTAGTCTTGGCATGGGCCCTATGGCGACCGAACTTGAACCTCTTTGTACACCCGTTG
TAACTTGCACCCCATCCTGTACGGCCTATACCTCAAGTTTTGTATTTACATATCCTGAAGCCGATTCTTTTCCATCCTGCGCCGCC
GCGCATCGAAAAGGGTCTTCATCTAACGAACCATCTTCCGATAGTTTGAGTAGTCCAACCCTTCTTGCTCTCTAAGGATCCATTC
GATATCAAGCTTATCGATAATCAACCTCTGGATTACAAAATTTGTGAAAGATTGACTGGTATTCTTAACTATGTTGCTCCTTTTA
CGCTATGTGGATACGCTGCTTTAATGCCTTTGTATCATGCTATTGCTTCCCGTATGGCTTTCATTTTCTCCTCCTTGTATAAATCCT
GGTTGCTGTCTCTTTATGAGGAGTTGTGGCCCGTTGTCAGGCAACGTGGCGTGGTGTGCACTGTGTTTGCTGACGCAACCCCC
ACTGGTTGGGGCATTGCCACCACCTGTCAGCTCCTTTCCGGGACTTTCGCTTTCCCCCTCCCTATTGCCACGGCGGAACTCATCG
CCGCCTGCCTTGCCCGCTGCTGGACAGGGGCTCGGCTGTTGGGCACTGACAATTCCGTGGTGTTGTCGGGGAAATCATCGTCC
TTTCCTTGGCTGCTCGCCTGTGTTGCCACCTGGATTCTGCGCGGGACGTCCTTCTGCTACGTCCCTTCGGCCCTCAATCCAGCGG
ACCTTCCTTCCCGCGGCCTGCTGCCGGCTCTGCGGCCTCTTCCGCGTCTTCGCCTTCGCCCTCAGACGAGTCGGATCTCCCTTTG
GGCCGCCTCCCCGCATCGATACCGTCGACCTCGAGACCTAGAAAAACATGGAGCAATCACAAGTAGCAATACAGCAGCTACCA
ATGCTGATTGTGCCTGGCTAGAAGCACAAGAGGAGGAGGAGGTGGGTTTTCCAGTCACACCTCAGGTACCTTTAAGACCAAT
GACTTACAAGGCAGCTGTAGATCTTAGCCACTTTTTAAAAGAAAAGGGGGGACTGGAAGGGCTAATTCACTCCCAACGAAGA
CAAGATATCCTTGATCTGTGGATCTACCACACACAAGGCTACTTCCCTGATTGGCAGAACTACACACCAGGGCCAGGGATCAG
ATATCCACTGACCTTTGGATGGTGCTACAAGCTAGTACCAGTTGAGCAAGAGAAGGTAGAAGAAGCCAATGAAGGAGAGAAC
ACCCGCTTGTTACACCCTGTGAGCCTGCATGGGATGGATGACCCGGAGAGAGAAGTATTAGAGTGGAGGTTTGACAGCCGCC
TAGCATTTCATCACATGGCCCGAGAGCTGCATCCGGACTGTACTGGGTCTCTCTGGTTAGACCAGATCTGAGCCTGGGAGCTCT
CTGGCTAACTAGGGAACCCACTGCTTAAGCCTCAATAAAGCTTGCCTTGAGTGCTTCAAGTAGTGTGTGCCCGTCTGTTGTGTG
ACTCTGGTAACTAGAGATCCCTCAGACCCTTTTAGTCAGTGTGGAAAATCTCTAGCAGGGCCCGTTTAAACCCGCTGATCAGCC
TCGACTGTGCCTTCTAGTTGCCAGCCATCTGTTGTTTGCCCCTCCCCCGTGCCTTCCTTGACCCTGGAAGGTGCCACTCCCACTG
TCCTTTCCTAATAAAATGAGGAAATTGCATCGCATTGTCTGAGTAGGTGTCATTCTATTCTGGGGGGTGGGGTGGGGCAGGAC
AGCAAGGGGGAGGATTGGGAAGACAATAGCAGGCATGTGAGCAAAAGGCCAGCAAAAGGCCAGGAACCGTAAAAAGGCCG
CGTTGCTGGCGTTTTTCCATAGGCTCCGCCCCCCTGACGAGCATCACAAAAATCGACGCTCAAGTCAGAGGTGGCGAAACCCG
ACAGGACTATAAAGATACCAGGCGTTTCCCCCTGGAAGCTCCCTCGTGCGCTCTCCTGTTCCGACCCTGCCGCTTACCGGATAC
CTGTCCGCCTTTCTCCCTTCGGGAAGCGTGGCGCTTTCTCATAGCTCACGCTGTAGGTATCTCAGTTCGGTGTAGGTCGTTCGCT
CCAAGCTGGGCTGTGTGCACGAACCCCCCGTTCAGCCCGACCGCTGCGCCTTATCCGGTAACTATCGTCTTGAGTCCAACCCGG
TAAGACACGACTTATCGCCACTGGCAGCAGCCACTGGTAACAGGATTAGCAGAGCGAGGTATGTAGGCGGTGCTACAGAGTT
CTTGAAGTGGTGGCCTAACTACGGCTACACTAGAAGAACAGTATTTGGTATCTGCGCTCTGCTGAAGCCAGTTACCTTCGGAA
AAAGAGTTGGTAGCTCTTGATCCGGCAAACAAACCACCGCTGGTAGCGGTGGTTTTTTTGTTTGCAAGCAGCAGATTACGCGC
AGAAAAAAAGGATCTCAAGAAGATCCTTTGATCTTTTCTACGGGGTCTGACGCTCAGTGGAACGAAAACTCACGTTAAGGGAT
TTTGGTCATGAGATTATCAAAAAGGATCTTCACCTAGATCCTTTTAAATTAAAAATGAAGTTTTAAATCAATCTAAAGTATATAT
GAGTAAACTTGGTCTGACAGTTACCAATGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCAGTGCTGCAATGATACCGCGAGACCCA
CGCTCACCGGCTCCAGATTTATCAGCAATAAACCAGCCAGCCGGAAGGGCCGAGCGCAGAAGTGGTCCTGCAACTTTATCCGC
CTCCATCCAGTCTATTAATTGTTGCCGGGAAGCTAGAGTAAGTAGTTCGCCAGTTAATAGTTTGCGCAACGTTGTTGCCATTGC
TACAGGCATCGTGGTGTCACGCTCGTCGTTTGGTATGGCTTCATTCAGCTCCGGTTCCCAACGATCAAGGCGAGTTACATGATC
CCCCATGTTGTGCAAAAAAGCGGTTAGCTCCTTCGGTCCTCCGATCGTTGTCAGAAGTAAGTTGGCCGCAGTGTTATCACTCAT
GGTTATGGCAGCACTGCATAATTCTCTTACTGTCATGCCATCCGTAAGATGCTTTTCTGTGACTGGTGAGTACTCAACCAAGTCA
TTCTGAGAATAGTGTATGCGGCGACCGAGTTGCTCTTGCCCGGCGTCAATACGGGATAATACCGCGCCACATAGCAGAACTTT
AAAAGTGCTCATCATTGGAAAACGTTCTTCGGGGCGAAAACTCTCAAGGATCTTACCGCTGTTGAGATCCAGTTCGATGTAACC
CACTCGTGCACCCAACTGATCTTCAGCATCTTTTACTTTCACCAGCGTTTCTGGGTGAGCAAAAACAGGAAGGCAAAATGCCGC
AAAAAAGGGAATAAGGGCGACACGGAAATGTTGAATACTCATACTCTTCCTTTTTCAATATTATTGAAGCATTTATCAGGGTTA
TTGTCTCATGAGCGGATACATATTTGAATGTATTTAGAAAAATAAACAAATAGGGGTTCCGCGCACATTTCCCCGAAAAGTGCC
ACCTGACG
SEQ ID No. 21-FUW-GaFoGi (vector 2)
TCGACGGATCGGGAGATCTCCCGATCCCCTATGGTGCACTCTCAGTACAATCTGCTCTGATGCCGCATAGTTAAGCCAGTATCT
GCTCCCTGCTTGTGTGTTGGAGGTCGCTGAGTAGTGCGCGAGCAAAATTTAAGCTACAACAAGGCAAGGCTTGACCGACAATT
GCATGAAGAATCTGCTTAGGGTTAGGCGTTTTGCGCTGCTTCGCGATGTACGGGCCAGATATACGCGTTGACATTGATTATTG
ACTAGTTATTAATAGTAATCAATTACGGGGTCATTAGTTCATAGCCCATATATGGAGTTCCGCGTTACATAACTTACGGTAAAT
GGCCCGCCTGGCTGACCGCCCAACGACCCCCGCCCATTGACGTCAATAATGACGTATGTTCCCATAGTAACGCCAATAGGGAC
TTTCCATTGACGTCAATGGGTGGAGTATTTACGGTAAACTGCCCACTTGGCAGTACATCAAGTGTATCATATGCCAAGTACGCC
CCCTATTGACGTCAATGACGGTAAATGGCCCGCCTGGCATTATGCCCAGTACATGACCTTATGGGACTTTCCTACTTGGCAGTA
CATCTACGTATTAGTCATCGCTATTACCATGGTGATGCGGTTTTGGCAGTACATCAATGGGCGTGGATAGCGGTTTGACTCACG
GGGATTTCCAAGTCTCCACCCCATTGACGTCAATGGGAGTTTGTTTTGGCACCAAAATCAACGGGACTTTCCAAAATGTCGTAA
CAACTCCGCCCCATTGACGCAAATGGGCGGTAGGCGTGTACGGTGGGAGGTCTATATAAGCAGCGCGTTTTGCCTGTACTGG
GTCTCTCTGGTTAGACCAGATCTGAGCCTGGGAGCTCTCTGGCTAACTAGGGAACCCACTGCTTAAGCCTCAATAAAGCTTGCC
TTGAGTGCTTCAAGTAGTGTGTGCCCGTCTGTTGTGTGACTCTGGTAACTAGAGATCCCTCAGACCCTTTTAGTCAGTGTGGAA
AATCTCTAGCAGTGGCGCCCGAACAGGGACTTGAAAGCGAAAGGGAAACCAGAGGAGCTCTCTCGACGCAGGACTCGGCTTG
CTGAAGCGCGCACGGCAAGAGGCGAGGGGCGGCGACTGGTGAGTACGCCAAAAATTTTGACTAGCGGAGGCTAGAAGGAG
AGAGATGGGTGCGAGAGCGTCAGTATTAAGCGGGGGAGAATTAGATCGCGATGGGAAAAAATTCGGTTAAGGCCAGGGGG
AAAGAAAAAATATAAATTAAAACATATAGTATGGGCAAGCAGGGAGCTAGAACGATTCGCAGTTAATCCTGGCCTGTTAGAA
ACATCAGAAGGCTGTAGACAAATACTGGGACAGCTACAACCATCCCTTCAGACAGGATCAGAAGAACTTAGATCATTATATAA
TACAGTAGCAACCCTCTATTGTGTGCATCAAAGGATAGAGATAAAAGACACCAAGGAAGCTTTAGACAAGATAGAGGAAGAG
CAAAACAAAAGTAAGACCACCGCACAGCAAGCGGCCGCTGATCTTCAGACCTGGAGGAGGAGATATGAGGGACAATTGGAG
AAGTGAATTATATAAATATAAAGTAGTAAAAATTGAACCATTAGGAGTAGCACCCACCAAGGCAAAGAGAAGAGTGGTGCAG
AGAGAAAAAAGAGCAGTGGGAATAGGAGCTTTGTTCCTTGGGTTCTTGGGAGCAGCAGGAAGCACTATGGGCGCAGCGTCA
ATGACGCTGACGGTACAGGCCAGACAATTATTGTCTGGTATAGTGCAGCAGCAGAACAATTTGCTGAGGGCTATTGAGGCGC
AACAGCATCTGTTGCAACTCACAGTCTGGGGCATCAAGCAGCTCCAGGCAAGAATCCTGGCTGTGGAAAGATACCTAAAGGAT
CAACAGCTCCTGGGGATTTGGGGTTGCTCTGGAAAACTCATTTGCACCACTGCTGTGCCTTGGAATGCTAGTTGGAGTAATAA
ATCTCTGGAACAGATTTGGAATCACACGACCTGGATGGAGTGGGACAGAGAAATTAACAATTACACAAGCTTAATACACTCCT
TAATTGAAGAATCGCAAAACCAGCAAGAAAAGAATGAACAAGAATTATTGGAATTAGATAAATGGGCAAGTTTGTGGAATTG
GTTTAACATAACAAATTGGCTGTGGTATATAAAATTATTCATAATGATAGTAGGAGGCTTGGTAGGTTTAAGAATAGTTTTTGC
TGTACTTTCTATAGTGAATAGAGTTAGGCAGGGATATTCACCATTATCGTTTCAGACCCACCTCCCAACCCCGAGGGGACCCGA
CAGGCCCGAAGGAATAGAAGAAGAAGGTGGAGAGAGAGACAGAGACAGATCCATTCGATTAGTGAACGGATCGGCACTGC
GTGCGCCAATTCTGCAGACAAATGGCAGTATTCATCCACAATTTTAAAAGAAAAGGGGGGATTGGGGGGTACAGTGCAGGGG
AAAGAATAGTAGACATAATAGCAACAGACATACAAACTAAAGAATTACAAAAACAAATTACAAAAATTCAAAATTTTCGGGTT
TATTACAGGGACAGCAGAGATCCAGTTTGGTTAATTAACCCGTGTCGGCTCCAGATCTGGCCTCCGCGCCGGGTTTTGGCGCC
TCCCGCGGGCGCCCCCCTCCTCACGGCGAGCGCTGCCACGTCAGACGAAGGGCGCAGCGAGCGTCCTGATCCTTCCGCCCGG
ACGCTCAGGACAGCGGCCCGCTGCTCATAAGACTCGGCCTTAGAACCCCAGTATCAGCAGAAGGACATTTTAGGACGGGACTT
GGGTGACTCTAGGGCACTGGTTTTCTTTCCAGAGAGCGGAACAGGCGAGGAAAAGTAGTCCCTTCTCGGCGATTCTGCGGAG
GGATCTCCGTGGGGCGGTGAACGCCGATGATTATATAAGGACGCGCCGGGTGTGGCACAGCTAGTTCCGTCGCAGCCGGGAT
TTGGGTCGCGGTTCTTGTTTGTGGATCGCTGTGATCGTCACTTGGTGAGTAGCGGGCTGCTGGGCTGGCCGGGGCTTTCGTGG
CCGCCGGGCCGCTCGGTGGGACGGAAGCGTGTGGAGAGACCGCCAAGGGCTGTAGTCTGGGTCCGCGAGCAAGGTTGCCCT
GAACTGGGGGTTGGGGGGAGCGCAGCAAAATGGCGGCTGTTCCCGAGTCTTGAATGGAAGACGCTTGTGAGGCGGGCTGTG
AGGTCGTTGAAACAAGGTGGGGGGCATGGTGGGCGGCAAGAACCCAAGGTCTTGAGGCCTTCGCTAATGCGGGAAAGCTCT
TATTCGGGTGAGATGGGCTGGGGCACCATCTGGGGACCCTGACGTGAAGTTTGTCACTGACTGGAGAACTCGGTTTGTCGTCT
GTTGCGGGGGCGGCAGTTATGGCGGTGCCGTTGGGCAGTGCACCCGTACCTTTGGGAGCGCGCGCCCTCGTCGTGTCGTGAC
GTCACCCGTTCTGTTGGCTTATAATGCAGGGTGGGGCCACCTGCCGGTAGGTGTGCGGTAGGCTTTTCTCCGTCGCAGGACGC
AGGGTTCGGGCCTAGGGTAGGCTCTCCTGAATCGACAGGCGCCGGACCTCTGGTGAGGGGAGGGATAAGTGAGGCGTCAGT
TTCTTTGGTCGGTTTTATGTACCTATCTTCTTAAGTAGCTGAAGCTCCGGTTTTGAACTATGCGCTCGGGGTTGGCGAGTGTGTT
TTGTGAAGTTTTTTAGGCACCTTTTGAAATGTAATCATTTGGGTCAATATGTAATTTTCAGTGTTAGACTAGTAAATTGTCCGCT
AAATTCTGGCCGTTTTTGGCTTTTTTGTTAGACGAAGCTTGGGCCCGGGATCCATGGAAGTCGCCCCGGAACAACCACGATGG
ATGGCACATCCTGCAGTTCTCAACGCACAACATCCTGATAGTCATCATCCAGGGTTGGCTCATAATTATATGGAGCCTGCCCAA
CTCCTTCCCCCTGATGAAGTTGATGTGTTCTTTAACCATCTGGATTCTCAAGGAAATCCGTATTACGCTAATCCAGCCCATGCAC
GCGCACGTGTTTCATATAGTCCTGCCCATGCGAGGCTTACAGGCGGGCAAATGTGTCGTCCCCATCTCCTGCATTCTCCCGGCC
TGCCGTGGCTTGATGGTGGAAAGGCTGCGCTGAGCGCTGCCGCCGCTCATCATCATAATCCATGGACTGTCAGTCCGTTTTCTA
AAACTCCTTTGCATCCATCTGCAGCCGGTGGACCAGGCGGTCCGCTTTCAGTTTATCCCGGTGCGGGTGGCGGCTCCGGCGGC
GGTTCAGGATCAAGCGTCGCGAGCCTGACTCCAACGGCTGCTCATAGTGGTAGCCATCTGTTTGGGTTTCCCCCAACTCCCCCT
AAAGAGGTCAGCCCAGATCCAAGCACGACCGGCGCCGCTTCCCCTGCATCCAGCTCTGCCGGTGGGAGCGCGGCAAGAGGG
GAAGATAAAGATGGAGTGAAATATCAAGTAAGTTTGACAGAATCAATGAAAATGGAGAGCGGATCTCCTCTCCGGCCCGGGC
TTGCCACAATGGGTACTCAACCAGCCACCCATCATCCTATTCCAACATATCCTAGCTACGTTCCCGCCGCCGCTCATGATTATTC
ATCTGGTCTGTTTCATCCTGGCGGGTTCTTGGGCGGCCCCGCGAGCAGTTTTACACCCAAACAACGTTCTAAAGCCAGGAGCT
GCAGTGAGGGTAGAGAATGCGTTAATTGCGGCGCTACTGCAACACCGCTTTGGCGAAGAGATGGTACTGGTCATTATTTGTGT
AACGCATGCGGTCTGTATCATAAAATGAACGGTCAAAATCGGCCCCTGATTAAACCTAAAAGGCGGCTTAGTGCGGCAAGGC
GTGCAGGAACTTGCTGCGCCAACTGCCAAACAACGACAACTACTCTCTGGAGACGGAATGCAAATGGTGATCCGGTTTGTAAT
GCGTGCGGGCTGTATTATAAATTGCATAACGTCAATCGGCCGCTTACAATGAAGAAAGAGGGAATACAAACCAGAAATCGCA
AAATGAGCAATAAATCTAAGAAATCTAAGAAGGGAGCAGAATGTTTTGAGGAACTCAGCAAATGTATGCAAGAAAAGTCCAG
CCCTTTCTCCGCTGCAGCTTTGGCGGGGCATATGGCTCCCGTCGGTCATCTTCCACCGTTTTCTCATAGCGGTCATATTCTTCCTA
CGCCCACCCCAATACATCCTTCTTCATCTTTGTCATTTGGACATCCTCATCCATCTTCTATGGTCACTGCAATGGGCGGCAGCGG
CGCCACAAACTTCTCTCTGCTAAAGCAAGCAGGTGATGTTGAAGAAAACCCCGGGCCTATGATGTTTTCAGGGTTTAATGCCG
ATTATGAAGCCAGTTCTTCAAGATGTTCCTCAGCTTCCCCCGCAGGCGACTCCTTGTCCTATTATCATTCTCCAGCTGATAGCTTT
AGTTCCATGGGGTCCCCCGTTAATGCCCAAGACTTTTGTACTGATCTCGCGGTTAGCAGCGCAAATTTTATACCGACTGTGACA
GCAATATCAACATCTCCAGATCTTCAATGGCTCGTTCAACCTGCACTGGTGAGCTCTGTCGCACCTTCACAAACGCGGGCTCCA
CATCCATTTGGCGTTCCTGCACCAAGTGCAGGTGCCTATAGCCGCGCAGGGGTGGTTAAAACAATGACTGGTGGAAGAGCCC
AATCCATCGGGCGCCGGGGAAAAGTAGAGCAACTGTCCCCCGAGGAAGAGGAAAAGCGTCGTATTAGGCGCGAGCGGAACA
AAATGGCGGCTGCAAAGTGTAGAAATAGGCGCCGCGAATTGACAGACACCCTGCAGGCAGAAACCGATCAGCTCGAGGACG
AAAAGTCCGCCCTTCAAACTGAAATCGCAAATTTGCTTAAAGAGAAAGAGAAGTTGGAATTTATACTCGCTGCCCATCGGCCA
GCATGTAAAATACCCGACGATTTGGGTTTTCCCGAGGAAATGTCAGTAGCCTCTCTCGACCTTACGGGCGGACTTCCCGAAGT
GGCGACTCCTGAAAGTGAAGAAGCATTTACACTTCCCCTGCTGAACGATCCAGAACCTAAACCTTCCGTCGAGCCCGTAAAATC
AATTTCTTCCATGGAATTGAAAACAGAACCTTTCGACGATTTTCTCTTTCCTGCCAGTTCACGCCCATCCGGGAGTGAAACCGCT
CGAAGCGTTCCGGATATGGATCTTAGCGGCAGCTTTTACGCTGCTGATTGGGAACCGCTTCATAGCGGGTCATTGGGTATGGG
ACCAATGGCTACCGAACTTGAACCATTGTGTACCCCAGTTGTGACATGCACCCCTTCTTGTACCGCGTATACTTCAAGCTTTGTA
TTTACATATCCTGAAGCCGATTCATTTCCAAGTTGCGCCGCCGCTCATAGGAAAGGGTCTAGTAGTAACGAACCAAGTTCAGAT
TCACTGTCCAGCCCTACATTGTTGGCTCTCGGCTCCGGCGAGGGCAGGGGAAGTCTTCTAACATGCGGGGACGTGGAGGAAA
ATCCCGGCCCAATGCCCCGGAGTTTTCTTGTTAAATCCAAGAAAGCCCATACATATCATCAACCTCGCGTCCAAGAGGACGAGC
CACTGTGGCCCCCAGCTCTGACGCCTGTACCTCGGGATCAAGCACCGTCTAATTCTCCCGTGCTGTCAACCCTGTTTCCTAATCA
ATGTCTTGATTGGACTAATCTTAAGCGCGAACCAGAACTCGAACAAGATCAAAATCTGGCACGGATGGCTCCTGCCCCCGAAG
GACCAATCGTACTCTCTCGGCCTCAAGACGGCGATAGTCCCTTGTCAGATTCACCCCCATTTTATAAACCATCCTTTAGTTGGGA
TACACTCGCTACTACATACGGTCATTCATATAGACAAGCTCCATCTACAATGCAATCCGCGTTTCTCGAACATAGCGTGAGTCTT
TATGGTAGCCCACTGGTTCCAAGTACCGAACCGGCACTGGATTTCTCACTTCGTTATAGCCCTGGGATGGACGCTTATCATTGC
GTCAAATGTAATAAAGTGTTTAGTACGCCCCATGGACTGGAAGTCCACGTAAGGCGGAGTCACTCCGGCACTAGACCTTTTGC
ATGCGATATATGTGGGAAGACTTTTGGTCATGCCGTTTCCCTTGAACAACATACTCATGTGCATTCTCAAGAAAGATCCTTTGA
ATGTCGGATGTGTGGAAAAGCATTTAAAAGGTCATCAACTCTCAGCACTCATTTGCTGATTCATTCTGATACCCGCCCATATCCT
TGTCAATTTTGTGGTAAAAGGTTTCATCAGAAAAGCGATATGAAGAAACATACCTACATTCATACTGGAGAGAAGCCCCATAA
ATGTCAAGTTTGTGGTAAGGCATTTTCACAATCTTCTAACCTGATAACACATTCCCGGAAACATACTGGATTTAAACCATTTTCT
TGCGAACTCTGTACAAAGGGTTTTCAAAGAAAAGTAGATCTCCGTAGGCATCGTGAATCACAACATAACCTGAAATAAGGATC
CATTCGATATCAAGCTTATCGATAATCAACCTCTGGATTACAAAATTTGTGAAAGATTGACTGGTATTCTTAACTATGTTGCTCC
TTTTACGCTATGTGGATACGCTGCTTTAATGCCTTTGTATCATGCTATTGCTTCCCGTATGGCTTTCATTTTCTCCTCCTTGTATAA
ATCCTGGTTGCTGTCTCTTTATGAGGAGTTGTGGCCCGTTGTCAGGCAACGTGGCGTGGTGTGCACTGTGTTTGCTGACGCAA
CCCCCACTGGTTGGGGCATTGCCACCACCTGTCAGCTCCTTTCCGGGACTTTCGCTTTCCCCCTCCCTATTGCCACGGCGGAACT
CATCGCCGCCTGCCTTGCCCGCTGCTGGACAGGGGCTCGGCTGTTGGGCACTGACAATTCCGTGGTGTTGTCGGGGAAATCAT
CGTCCTTTCCTTGGCTGCTCGCCTGTGTTGCCACCTGGATTCTGCGCGGGACGTCCTTCTGCTACGTCCCTTCGGCCCTCAATCC
AGCGGACCTTCCTTCCCGCGGCCTGCTGCCGGCTCTGCGGCCTCTTCCGCGTCTTCGCCTTCGCCCTCAGACGAGTCGGATCTC
CCTTTGGGCCGCCTCCCCGCATCGATACCGTCGACCTCGAGACCTAGAAAAACATGGAGCAATCACAAGTAGCAATACAGCAG
CTACCAATGCTGATTGTGCCTGGCTAGAAGCACAAGAGGAGGAGGAGGTGGGTTTTCCAGTCACACCTCAGGTACCTTTAAGA
CCAATGACTTACAAGGCAGCTGTAGATCTTAGCCACTTTTTAAAAGAAAAGGGGGGACTGGAAGGGCTAATTCACTCCCAACG
AAGACAAGATATCCTTGATCTGTGGATCTACCACACACAAGGCTACTTCCCTGATTGGCAGAACTACACACCAGGGCCAGGGA
TCAGATATCCACTGACCTTTGGATGGTGCTACAAGCTAGTACCAGTTGAGCAAGAGAAGGTAGAAGAAGCCAATGAAGGAGA
GAACACCCGCTTGTTACACCCTGTGAGCCTGCATGGGATGGATGACCCGGAGAGAGAAGTATTAGAGTGGAGGTTTGACAGC
CGCCTAGCATTTCATCACATGGCCCGAGAGCTGCATCCGGACTGTACTGGGTCTCTCTGGTTAGACCAGATCTGAGCCTGGGA
GCTCTCTGGCTAACTAGGGAACCCACTGCTTAAGCCTCAATAAAGCTTGCCTTGAGTGCTTCAAGTAGTGTGTGCCCGTCTGTT
GTGTGACTCTGGTAACTAGAGATCCCTCAGACCCTTTTAGTCAGTGTGGAAAATCTCTAGCAGGGCCCGTTTAAACCCGCTGAT
CAGCCTCGACTGTGCCTTCTAGTTGCCAGCCATCTGTTGTTTGCCCCTCCCCCGTGCCTTCCTTGACCCTGGAAGGTGCCACTCC
CACTGTCCTTTCCTAATAAAATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAATCGACGCTCAAGTCAGAGGTGGCGAA
ACCCGACAGGACTATAAAGATACCAGGCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTGTAGGTATCTCAGTTCGGTGTAGGTCG
TTCGCTCCAAGCTGGGCTGTGTGCACGAACCCCCCGTTCAGCCCGACCGCTGCGCCTTATCCGGTAACTATCGTCTTGAGTCCA
ACCCGGTAAGACACGACTTATCGCCACTGGCAGCAGCCACTGGTAACAGGATTAGCAGAGCGAGGTATGTAGGCGGTGCTAC
AGAGTTCTTGAAGTGGTGGCCTAACTACGGCTACACTAGAAGAACAGTATTTGGTATCTGCGCTCTGCTGAAGCCAGTTACCTT
CGGAAAAAGAGTTGGTAGCTCTTGATCCGGCAAACAAACCACCGCTGGTAGCGGTGGTTTTTTTGTTTGCAAGCAGCAGATTA
CGCGCAGAAAAAAAGGATCTCAAGAAGATCCTTTGATCTTTTCTACGGGGTCTGACGCTCAGTGGAACGAAAACTCACGTTAA
GGGATTTTGGTCATGAGATTATCAAAAAGGATCTTCACCTAGATCCTTTTAAATTAAAAATGAAGTTTTAAATCAATCTAAAGTA
TATATGAGTAAACTTGGTCTGACAGTTACCAATGCTTAATCAGTGAGGCACCTATCTCAGCGATCTGTCTATTTCGTTCATCCAT
AGTTGCCTGACTCCCCGTCGTGTAGATAACTACGATACGGGAGGGCTTACCATCTGGCCCCAGTGCTGCAATGATACCGCGAG
ACCCACGCTCACCGGCTCCAGATTTATCAGCAATAAACCAGCCAGCCGGAAGGGCCGAGCGCAGAAGTGGTCCTGCAACTTTA
TCCGCCTCCATCCAGTCTATTAATTGTTGCCGGGAAGCTAGAGTAAGTAGTTCGCCAGTTAATAGTTTGCGCAACGTTGTTGCC
ATTGCTACAGGCATCGTGGTGTCACGCTCGTCGTTTGGTATGGCTTCATTCAGCTCCGGTTCCCAACGATCAAGGCGAGTTACA
TGATCCCCCATGTTGTGCAAAAAAGCGGTTAGCTCCTTCGGTCCTCCGATCGTTGTCAGAAGTAAGTTGGCCGCAGTGTTATCA
CTCATGGTTATGGCAGCACTGCATAATTCTCTTACTGTCATGCCATCCGTAAGATGCTTTTCTGTGACTGGTGAGTACTCAACCA
AGTCATTCTGAGAATAGTGTATGCGGCGACCGAGTTGCTCTTGCCCGGCGTCAATACGGGATAATACCGCGCCACATAGCAGA
ACTTTAAAAGTGCTCATCATTGGAAAACGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTGGGTGAGCAAAAACAGGAAGGCAAAAT
GCCGCAAAAAAGGGAATAAGGGCGACACGGAAATGTTGAATACTCATACTCTTCCTTTTTCAATATTATTGAAGCATTTATCAG
GGTTATTGTCTCATGAGCGGATACATATTTGAATGTATTTAGAAAAATAAACAAATAGGGGTTCCGCGCACATTTCCCCGAAAA
GTGCCACCTGACG
SEQ ID No. 22-FUW-GiGaFo (vector 3)
tcgacggatcgggagatctcccgatcccctatggtgcactctcagtacaatctgctctgatgccgcatagttaagccagtatctgctccctgcttgtgtgttggaggtcgc
tgagtagtgcgcgagcaaaatttaagctacaacaaggcaaggcttgaccgacaattgcatgaagaatctgcttagggttaggcgttttgcgctgcttcgcgatgtacg
ggccagatatacgcgttgacattgattattgactagttattaatagtaatcaattacggggtcattagttcatagcccatatatggagttccgcgttacataacttacggt
aaatggcccgcctggctgaccgcccaacgacccccgcccattgacgtcaataatgacgtatgttcccatagtaacgccaatagggactttccattgacgtcaatgggt
ggagtatttacggtaaactgcccacttggcagtacatcaagtgtatcatatgccaagtacgccccctattgacgtcaatgacggtaaatggcccgcctggcattatgcc
cagtacatgaccttatgggactttcctacttggcagtacatctacgtattagtcatcgctattaccatggtgatgcggttttggcagtacatcaatgggcgtggatagcgg
tttgactcacggggatttccaagtctccaccccattgacgtcaatgggagtttgttttggcaccaaaatcaacgggactttccaaaatgtcgtaacaactccgccccatt
gacgcaaatgggcggtaggcgtgtacggtgggaggtctatataagcagcgcgttttgcctgtactgggtctctctggttagaccagatctgagcctgggagctctctgg
ctaactagggaacccactgcttaagcctcaataaagcttgccttgagtgcttcaagtagtgtgtgcccgtctgttgtgtgactctggtaactagagatccctcagaccctt
ttagtcagtgtggaaaatctctagcagtggcgcccgaacagggacttgaaagcgaaagggaaaccagaggagctctctcgacgcaggactcggcttgctgaagcgc
gcacggcaagaggcgaggggcggcgactggtgagtacgccaaaaattttgactagcggaggctagaaggagagagatgggtgcgagagcgtcagtattaagcgg
gggagaattagatcgcgatgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaagaacttagatcattatataat
acagtagcaaccctctattgtgtgcatcaaaggatagagataaaagacaccaaggaagctttagacaagatagaggaagagcaaaacaaaagtaagaccaccgc
acagcaagcggccgctgatcttcagacctggaggaggagatatgagggacaattggagaagtgaattatataaatataaagtagtaaaaattgaaccattaggagt
agcacccaccaaggcaaagagaagagtggtgcagagagaaaaaagagcagtgggaataggagctttgttccttgggttcttgggagcagcaggaagcactatggg
cgcagcgtcaatgacgctgacggtacaggccagacaattattgtctggtatagtgcagcagcagaacaatttgctgagggctattgaggcgcaacagcatctgttgca
actcacagtctggggcatcaagcagctccaggcaagaatcctggctgtggaaagatacctaaaggatcaacagctcctggggatttggggttgctctggaaaactcat
ttgcaccactgctgtgccttggaatgctagttggagtaataaatctctggaacagatttggaatcacacgacctggatggagtgggacagagaaattaacaattacac
aagcttaatacactccttaattgaagaatcgcaaaaccagcaagaaaagaatgaacaagaattattggaattagataaatgggcaagtttgtggaattggtttaaca
taacaaattggctgtggtatataaaattattcataatgatagtaggaggcttggtaggtttaagaatagtttttgctgtactttctatagtgaatagagttaggcagggat
attcaccattatcgtttcagacccacctcccaaccccgaggggacccgacaggcccgaaggaatagaagaagaaggtggagagagagacagagacagatccattc
gattagtgaacggatcggcactgcgtgcgccaattctgcagacaaatggcagtattcatccacaattttaaaagaaaaggggggattggggggtacagtgcagggg
aaagaatagtagacataatagcaacagacatacaaactaaagaattacaaaaacaaattacaaaaattcaaaattttcgggtttattacagggacagcagagatcc
agtttggttaattaacccgtgtcggctccagatctggcctccgcgccgggttttggcgcctcccgcgggcgcccccctcctcacggcgagcgctgccacgtcagacgaa
gggcgcagcgagcgtcctgatccttccgcccggacgctcaggacagcggcccgctgctcataagactcggccttagaaccccagtatcagcagaaggacattttagg
acgggacttgggtgactctagggcactggttttctttccagagagcggaacaggcgaggaaaagtagtcccttctcggcgattctgcggagggatctccgtggggcgg
tgaacgccgatgattatataaggacgcgccgggtgtggcacagctagttccgtcgcagccgggatttgggtcgcggttcttgtttgtggatcgctgtgatcgtcacttgg
tgagtagcgggctgctgggctggccggggctttcgtggccgccgggccgctcggtgggacggaagcgtgtggagagaccgccaagggctgtagtctgggtccgcgag
caaggttgccctgaactgggggttggggggagcgcagcaaaatggcggctgttcccgagtcttgaatggaagacgcttgtgaggcgggctgtgaggtcgttgaaaca
aggtggggggcatggtgggcggcaagaacccaaggtcttgaggccttcgctaatgcgggaaagctcttattcgggtgagatgggctggggcaccatctggggaccct
gacgtgaagtttgtcactgactggagaactcggtttgtcgtctgttgcgggggcggcagttatggcggtgccgttgggcagtgcacccgtacctttgggagcgcgcgcc
ctcgtcgtgtcgtgacgtcacccgttctgttggcttataatgcagggtggggccacctgccggtaggtgtgcggtaggcttttctccgtcgcaggacgcagggttcgggc
ctagggtaggctctcctgaatcgacaggcgccggacctctggtgaggggagggataagtgaggcgtcagtttctttggtcggttttatgtacctatcttcttaagtagct
gaagctccggttttgaactatgcgctcggggttggcgagtgtgttttgtgaagttttttaggcaccttttgaaatgtaatcatttgggtcaatatgtaattttcagtgttaga
ctagtaaattgtccgctaaattctggccgtttttggcttttttgttagacgaagcttgggcccgggatccATGCCCAGAAGCTTTCTCGTCAAATCAAAGAA
AGCCCATACATATCATCAACCTCGGGTTCAAGAGGACGAGCCTTTGTGGCCACCAGCTCTCACACCAGTCCCTCGGGATCAAGC
CCCTTCAAATAGTCCAGTACTCTCTACACTCTTTCCTAATCAATGTTTGGATTGGACAAATCTGAAGCGGGAACCAGAACTTGAA
CAAGATCAAAATCTGGCACGGATGGCACCTGCGCCTGAAGGACCAATCGTCTTGTCTAGGCCTCAAGACGGCGATAGCCCGCT
CAGCGATTCCCCACCGTTTTATAAACCCAGTTTTAGCTGGGATACACTGGCTACGACATACGGACATTCATATAGGCAAGCTCC
TAGTACTATGCAATCCGCATTTCTCGAACATAGCGTGTCTTTGTATGGGAGCCCGCTCGTACCTTCTACCGAACCTGCGCTGGA
TTTTAGTCTGCGTTATAGCCCTGGGATGGACGCCTATCATTGCGTCAAATGTAATAAAGTCTTTAGCACTCCCCATGGCCTGGA
AGTCCACGTCCGGCGAAGTCACTCTGGTACTAGACCATTTGCTTGCGATATTTGTGGAAAGACTTTTGGACATGCCGTCAGTCT
TGAACAACATACACATGTGCATTCTCAAGAACGGTCCTTTGAATGTCGGATGTGTGGGAAAGCTTTTAAAAGAAGCTCTACTCT
CTCAACGCATCTCTTGATTCATAGCGATACACGTCCTTATCCATGTCAATTTTGTGGAAAACGCTTCCATCAAAAGTCAGATATG
AAGAAACATACTTATATTCATACTGGCGAGAAACCACACAAATGTCAAGTTTGCGGCAAAGCATTTTCCCAATCCTCAAATCTG
ATTACTCATTCCAGAAAACATACTGGGTTTAAACCATTTTCCTGCGAACTTTGTACGAAGGGTTTTCAAAGGAAAGTTGATTTG
CGACGCCATAGAGAATCCCAACATAACCTGAAAGGCAGCGGCGCCACAAACTTCTCTCTGCTAAAGCAAGCAGGTGATGTTGA
AGAAAACCCCGGGCCTATGGAAGTTGCCCCTGAACAACCAAGATGGATGGCCCATCCCGCGGTCCTCAACGCCCAACATCCAG
ATTCTCATCATCCCGGTCTTGCCCATAATTATATGGAGCCTGCCCAACTTTTGCCCCCAGATGAAGTAGATGTATTCTTCAACCA
TCTGGATTCCCAAGGAAATCCTTATTACGCAAATCCAGCCCATGCACGCGCCAGGGTGAGCTATTCTCCAGCCCATGCAAGGCT
TACGGGCGGACAAATGTGTAGACCTCATCTGCTCCATAGTCCAGGCCTGCCATGGCTTGATGGCGGTAAGGCCGCGCTTAGCG
CTGCAGCAGCTCATCATCATAATCCATGGACTGTCTCCCCATTTAGCAAAACACCGCTTCATCCTTCTGCAGCAGGTGGGCCGG
GCGGACCTCTGAGCGTATATCCTGGCGCAGGTGGCGGCTCCGGCGGCGGGTCAGGAAGTAGTGTCGCGAGCCTGACTCCAAC
CGCTGCTCATAGCGGGAGTCATCTGTTTGGGTTTCCGCCAACACCCCCTAAAGAGGTATCCCCAGATCCATCTACTACCGGTGC
CGCCAGTCCCGCAAGCTCCAGTGCAGGCGGCAGCGCTGCTAGAGGTGAAGATAAAGATGGGGTGAAATATCAAGTTTCCCTC
ACCGAAAGTATGAAAATGGAGTCCGGAAGCCCACTTCGACCTGGTCTTGCCACCATGGGAACACAACCCGCCACCCATCATCC
TATACCAACGTATCCAAGCTACGTCCCCGCTGCAGCTCATGATTATTCATCAGGGCTGTTTCATCCTGGTGGATTCTTGGGCGG
TCCTGCTTCAAGTTTTACTCCGAAACAACGGTCTAAAGCCAGGAGCTGCTCCGAGGGAAGGGAATGCGTGAATTGCGGAGCA
ACCGCTACTCCCCTTTGGCGAAGGGATGGTACTGGTCATTATCTTTGTAACGCATGCGGGCTGTATCATAAAATGAACGGCCA
AAATCGGCCCCTGATTAAACCAAAACGGAGACTTTCCGCTGCACGGCGTGCTGGGACATGCTGCGCTAACTGCCAAACCACCA
CTACAACTCTCTGGCGGCGGAATGCTAATGGTGATCCAGTGTGTAATGCTTGCGGGCTGTATTACAAACTTCATAACGTGAATC
GCCCTCTTACGATGAAGAAAGAGGGAATTCAAACGCGTAATCGTAAAATGTCAAATAAATCAAAGAAATCTAAGAAGGGTGC
TGAATGTTTTGAGGAACTCAGCAAATGTATGCAAGAGAAAAGCAGCCCTTTTAGCGCGGCGGCGCTTGCAGGGCATATGGCC
CCAGTAGGTCATCTGCCCCCGTTTTCTCATAGCGGTCATATTCTCCCAACCCCAACACCGATACATCCAAGCAGCTCCTTGTCTTT
TGGGCATCCTCATCCAAGCTCAATGGTCACAGCAATGGGCGGCTCCGGCGAGGGCAGGGGAAGTCTTCTAACATGCGGGGAC
GTGGAGGAAAATCCCGGCCCAATGATGTTTTCAGGGTTTAATGCCGATTATGAAGCCAGTTCATCTCGATGTTCATCAGCCAGC
CCTGCTGGCGACTCACTGAGCTATTATCATTCTCCAGCTGATTCATTTTCTTCTATGGGGAGCCCAGTAAATGCACAAGACTTTT
GTACCGATCTCGCAGTTTCAAGCGCGAATTTTATCCCAACCGTGACCGCTATTAGCACTTCTCCAGATCTCCAATGGCTTGTCCA
ACCGGCTCTGGTGAGTAGCGTCGCTCCGAGCCAAACGAGGGCACCCCATCCGTTTGGTGTACCAGCACCAAGCGCAGGAGCA
TATTCTCGCGCGGGTGTAGTCAAAACAATGACTGGCGGGCGCGCCCAAAGTATCGGTCGCAGAGGTAAAGTAGAGCAACTGA
GCCCCGAGGAAGAGGAAAAGCGACGGATTAGACGTGAGCGGAACAAAATGGCCGCCGCTAAGTGTCGGAATAGACGACGA
GAACTTACAGACACTCTGCAGGCCGAAACCGATCAGTTGGAAGACGAAAAGTCCGCACTCCAAACAGAAATCGCGAATCTTTT
GAAAGAGAAAGAGAAGCTCGAATTTATTCTTGCCGCACATCGCCCCGCGTGTAAAATACCCGACGATCTTGGATTTCCTGAGG
AAATGTCCGTTGCAAGCCTGGACCTCACCGGCGGACTTCCGGAAGTCGCAACACCCGAAAGTGAAGAAGCTTTTACTCTTCCC
CTGCTGAACGATCCCGAACCTAAACCTTCTGTTGAGCCCGTTAAATCAATAAGTAGTATGGAACTCAAAACTGAACCATTCGAC
GATTTTCTGTTTCCCGCTTCCAGTAGACCAAGCGGGAGCGAAACTGCACGATCTGTCCCGGATATGGATCTGTCAGGCTCATTT
TACGCGGCCGATTGGGAACCACTCCATTCCGGATCTCTCGGCATGGGACCAATGGCTACCGAACTTGAACCACTTTGTACCCCC
GTCGTAACGTGCACACCATCCTGTACCGCGTATACTAGCTCTTTTGTATTTACTTATCCGGAAGCGGATTCATTTCCAAGTTGCG
CCGCCGCTCATAGAAAAGGGTCTTCATCTAACGAACCGTCTTCCGATTCCCTGAGTAGTCCTACCCTTTTGGCATTGTAAggatcc
attcgatatcaagcttatcgataatcaacctctggattacaaaatttgtgaaagattgactggtattcttaactatgttgctccttttacgctatgtggatacgctgcttta
atgcctttgtatcatgctattgcttcccgtatggctttcattttctcctccttgtataaatcctggttgctgtctctttatgaggagttgtggcccgttgtcaggcaacgtggc
gtggtgtgcactgtgtttgctgacgcaacccccactggttggggcattgccaccacctgtcagctcctttccgggactttcgctttccccctccctattgccacggcggaa
ctcatcgccgcctgccttgcccgctgctggacaggggctcggctgttgggcactgacaattccgtggtgttgtcggggaaatcatcgtcctttccttggctgctcgcctgt
gttgccacctggattctgcgcgggacgtccttctgctacgtcccttcggccctcaatccagcggaccttccttcccgcggcctgctgccggctctgcggcctcttccgcgtc
ttcgccttcgccctcagacgagtcggatctccctttgggccgcctccccgcatcgataccgtcgacctcgagacctagaaaaacatggagcaatcacaagtagcaata
cagcagctaccaatgctgattgtgcctggctagaagcacaagaggaggaggaggtgggttttccagtcacacctcaggtacctttaagaccaatgacttacaaggca
gctgtagatcttagccactttttaaaagaaaaggggggactggaagggctaattcactcccaacgaagacaagatatccttgatctgtggatctaccacacacaaggc
tacttccctgattggcagaactacacaccagggccagggatcagatatccactgacctttggatggtgctacaagctagtaccagttgagcaagagaaggtagaaga
agccaatgaaggagagaacacccgcttgttacaccctgtgagcctgcatgggatggatgacccggagagagaagtattagagtggaggtttgacagccgcctagca
tttcatcacatggcccgagagctgcatccggactgtactgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccctcagacccttttagtcagtgtggaaaatctctagcag
ggcccgtttaaacccgctgatcagcctcgactgtgccttctagttgccagccatctgttgtttgcccctcccccgtgccttccttgaccctggaaggtgccactcccactgt
cctttcctaataaaatgaggaaattgcatcgcattgtctgagtaggtgtcattctattctggggggtggggggggcaggacagcaagggggaggattgggaagaca
atagcaggcatgtgagcaaaaggccagcaaaaggccaggaaccgtaaaaaggccgcgttgctggcgtttttccataggctccgcccccctgacgagcatcacaaaa
atcgacgctcaagtcagaggtggcgaaacccgacaggactataaagataccaggcgtttccccctggaagctccctcgtgcgctctcctgttccgaccctgccgcttac
cggatacctgtccgcctttctcccttcgggaagcgtggcgctttctcatagctcacgctgtaggtatctcagttcggtgtaggtcgttcgctccaagctgggctgtgtgca
cgaaccccccgttcagcccgaccgctgcgccttatccggtaactatcgtcttgagtccaacccggtaagacacgacttatcgccactggcagcagccactggtaacag
gattagcagagcgaggtatgtaggcggtgctacagagttcttgaagtggtggcctaactacggctacactagaagaacagtatttggtatctgcgctctgctgaagcc
agttaccttcggaaaaagagttggtagctcttgatccggcaaacaaaccaccgctggtagcggtggtttttttgtttgcaagcagcagattacgcgcagaaaaaaagg
atctcaagaagatcctttgatcttttctacggggtctgacgctcagtggaacgaaaactcacgttaagggattttggtcatgagattatcaaaaaggatcttcacctaga
tccttttaaattaaaaatgaagttttaaatcaatctaaagtatatatgagtaaacttggtctgacagttaccaatgcttaatcagtgaggcacctatctcagcgatctgtc
tatttcgttcatccatagttgcctgactccccgtcgtgtagataactacgatacgggagggcttaccatctggccccagtgctgcaatgataccgcgagacccacgctca
ccggctccagatttatcagcaataaaccagccagccggaagggccgagcgcagaagtggtcctgcaactttatccgcctccatccagtctattaattgttgccgggaa
gctagagtaagtagttcgccagttaatagtttgcgcaacgttgttgccattgctacaggcatcgtggtgtcacgctcgtcgtttggtatggcttcattcagctccggttccc
aacgatcaaggcgagttacatgatcccccatgttgtgcaaaaaagcggttagctccttcggtcctccgatcgttgtcagaagtaagttggccgcagtgttatcactcatg
gttatggcagcactgcataattctcttactgtcatgccatccgtaagatgcttttctgtgactggtgagtactcaaccaagtcattctgagaatagtgtatgcggcgaccg
agttgctcttgcccggcgtcaatacgggataataccgcgccacatagcagaactttaaaagtgctcatcattggaaaacgttcttcggggcgaaaactctcaaggatct
taccgctgttgagatccagttcgatgtaacccactcgtgcacccaactgatcttcagcatcttttactttcaccagcgtttctgggtgagcaaaaacaggaaggcaaaat
gccgcaaaaaagggaataagggcgacacggaaatgttgaatactcatactcttcctttttcaatattattgaagcatttatcagggttattgtctcatgagcggatacat
atttgaatgtatttagaaaaataaacaaataggggttccgcgcacatttccccgaaaagtgccacctgacg
SEQ ID No. 23-FUW-GiFoGa (vector 4)
tcgacggatcgggagatctcccgatcccctatggtgcactctcagtacaatctgctctgatgccgcatagttaagccagtatctgctccctgcttgtgtgttggaggtcgc
tgagtagtgcgcgagcaaaatttaagctacaacaaggcaaggcttgaccgacaattgcatgaagaatctgcttagggttaggcgttttgcgctgcttcgcgatgtacg
ggccagatatacgcgttgacattgattattgactagttattaatagtaatcaattacggggtcattagttcatagcccatatatggagttccgcgttacataacttacggt
aaatggcccgcctggctgaccgcccaacgacccccgcccattgacgtcaataatgacgtatgttcccatagtaacgccaatagggactttccattgacgtcaatgggt
ggagtatttacggtaaactgcccacttggcagtacatcaagtgtatcatatgccaagtacgccccctattgacgtcaatgacggtaaatggcccgcctggcattatgcc
cagtacatgaccttatgggactttcctacttggcagtacatctacgtattagtcatcgctattaccatggtgatgcggttttggcagtacatcaatgggcgtggatagcgg
tttgactcacggggatttccaagtctccaccccattgacgtcaatgggagtttgttttggcaccaaaatcaacgggactttccaaaatgtcgtaacaactccgccccatt
gacgcaaatgggcggtaggcgtgtacggtgggaggtctatataagcagcgcgttttgcctgtactgggtctctctggttagaccagatctgagcctgggagctctctgg
ctaactagggaacccactgcttaagcctcaataaagcttgccttgagtgcttcaagtagtgtgtgcccgtctgttgtgtgactctggtaactagagatccctcagaccctt
ttagtcagtgtggaaaatctctagcagtggcgcccgaacagggacttgaaagcgaaagggaaaccagaggagctctctcgacgcaggactcggcttgctgaagcgc
gcacggcaagaggcgaggggcggcgactggtgagtacgccaaaaattttgactagcggaggctagaaggagagagatgggtgcgagagcgtcagtattaagcgg
gggagaattagatcgcgatgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaagaacttagatcattatataat
acagtagcaaccctctattgtgtgcatcaaaggatagagataaaagacaccaaggaagctttagacaagatagaggaagagcaaaacaaaagtaagaccaccgc
acagcaagcggccgctgatcttcagacctggaggaggagatatgagggacaattggagaagtgaattatataaatataaagtagtaaaaattgaaccattaggagt
agcacccaccaaggcaaagagaagagtggtgcagagagaaaaaagagcagtgggaataggagctttgttccttgggttcttgggagcagcaggaagcactatggg
cgcagcgtcaatgacgctgacggtacaggccagacaattattgtctggtatagtgcagcagcagaacaatttgctgagggctattgaggcgcaacagcatctgttgca
actcacagtctggggcatcaagcagctccaggcaagaatcctggctgtggaaagatacctaaaggatcaacagctcctggggatttggggttgctctggaaaactcat
ttgcaccactgctgtgccttggaatgctagttggagtaataaatctctggaacagatttggaatcacacgacctggatggagtgggacagagaaattaacaattacac
aagcttaatacactccttaattgaagaatcgcaaaaccagcaagaaaagaatgaacaagaattattggaattagataaatgggcaagtttgtggaattggtttaaca
taacaaattggctgtggtatataaaattattcataatgatagtaggaggcttggtaggtttaagaatagtttttgctgtactttctatagtgaatagagttaggcagggat
attcaccattatcgtttcagacccacctcccaaccccgaggggacccgacaggcccgaaggaatagaagaagaaggtggagagagagacagagacagatccattc
gattagtgaacggatcggcactgcgtgcgccaattctgcagacaaatggcagtattcatccacaattttaaaagaaaaggggggattggggggtacagtgcagggg
aaagaatagtagacataatagcaacagacatacaaactaaagaattacaaaaacaaattacaaaaattcaaaattttcgggtttattacagggacagcagagatcc
agtttggttaattaacccgtgtcggctccagatctggcctccgcgccgggttttggcgcctcccggggcgcccccctcctcacggcgagcgctgccacgtcagacgaa
gggcgcagcgagcgtcctgatccttccgcccggacgctcaggacagcggcccgctgctcataagactcggccttagaaccccagtatcagcagaaggacattttagg
acgggacttgggtgactctagggcactggttttctttccagagagcggaacaggcgaggaaaagtagtcccttctcggcgattctgcggagggatctccgtggggcgg
tgaacgccgatgattatataaggacgcgccgggtgtggcacagctagttccgtcgcagccgggatttgggtcgcggttcttgtttgtggatcgctgtgatcgtcacttgg
tgagtagcgggctgctgggctggccggggctttcgtggccgccgggccgctcggtgggacggaagcgtgtggagagaccgccaagggctgtagtctgggtccgcgag
caaggttgccctgaactgggggttggggggagcgcagcaaaatggcggctgttcccgagtcttgaatggaagacgcttgtgaggcgggctgtgaggtcgttgaaaca
aggtggggggcatggtgggcggcaagaacccaaggtcttgaggccttcgctaatgcgggaaagctcttattcgggtgagatgggctggggcaccatctggggaccct
gacgtgaagtttgtcactgactggagaactcggtttgtcgtctgttgcgggggcggcagttatggcggtgccgttgggcagtgcacccgtacctttgggagcgcgcgcc
ctcgtcgtgtcgtgacgtcacccgttctgttggcttataatgcagggtggggccacctgccggtaggtgtgcggtaggcttttctccgtcgcaggacgcagggttcgggc
ctagggtaggctctcctgaatcgacaggcgccggacctctggtgaggggagggataagtgaggcgtcagtttctttggtcggttttatgtacctatcttcttaagtagct
gaagctccggttttgaactatgcgctcggggttggcgagtgtgttttgtgaagttttttaggcaccttttgaaatgtaatcatttgggtcaatatgtaattttcagtgttaga
ctagtaaattgtccgctaaattctggccgtttttggcttttttgttagacgaagcttgggcccgggatccATGCCGAGATCATTTCTCGTCAAAAGTAAGAA
GGCGCATACTTATCATCAACCTCGCGTTCAAGAGGACGAGCCCTTGTGGCCCCCAGCGTTGACACCTGTTCCTCGCGATCAAGC
ACCCTCTAATAGTCCCGTGCTCTCAACCCTGTTTCCTAATCAATGTTTGGATTGGACAAATCTGAAGCGTGAACCTGAATTGGA
ACAAGATCAAAATCTTGCACGCATGGCGCCAGCGCCTGAAGGTCCAATCGTCCTCAGTAGACCACAAGACGGAGATAGCCCGT
TGAGCGATTCTCCACCCTTTTATAAACCATCCTTTAGTTGGGATACACTGGCAACGACATACGGACATAGTTATAGGCAAGCTC
CATCTACTATGCAATCTGCGTTTCTGGAACATAGCGTTTCTCTTTATGGGTCTCCGCTGGTTCCATCAACAGAACCTGCGCTGGA
TTTTAGCCTGAGATATAGTCCTGGGATGGACGCATATCATTGCGTAAAATGTAATAAAGTGTTTAGCACACCACATGGCCTGG
AAGTCCACGTTCGGCGGAGCCACAGCGGCACAAGGCCATTTGCATGCGATATTTGTGGGAAGACGTTTGGACATGCCGTTTCC
CTCGAACAACATACACATGTTCATAGCCAAGAAAGATCATTTGAATGTAGAATGTGTGGGAAAGCATTTAAACGGTCTAGCAC
ACTCTCTACGCATCTCCTGATTCATAGCGATACTCGCCCATATCCTTGTCAATTTTGCGGGAAAAGATTTCATCAAAAGTCAGAT
ATGAAGAAGCATACGTATATTCATACCGGGGAGAAACCACATAAATGTCAAGTCTGTGGCAAAGCCTTTTCCCAGTCATCTAAT
TTGATAACACATAGTAGGAAACATACCGGTTTTAAACCGTTTTCTTGCGAACTCTGTACAAAGGGTTTTCAAAGAAAAGTTGAT
CTCCGTAGACATAGGGAATCCCAACATAACCTGAAAGGCAGCGGCGCCACAAACTTCTCTCTGCTAAAGCAAGCAGGTGATGT
TGAAGAAAACCCCGGGCCTATGATGTTTAGCGGATTTAATGCCGATTATGAAGCAAGCTCAAGCAGGTGTTCCTCAGCTTCTCC
CGCTGGAGACTCTCTGTCATATTATCATAGCCCAGCCGATAGCTTTTCTTCCATGGGTTCCCCAGTTAATGCTCAAGATTTCTGT
ACCGATCTCGCGGTGAGTAGCGCAAATTTTATACCAACCGTGACCGCTATTTCTACATCTCCCGATCTTCAATGGTTGGTCCAAC
CTGCTCTGGTGAGTTCTGTCGCACCCTCTCAAACTCGGGCACCGCATCCCTTTGGCGTACCAGCACCAAGCGCCGGAGCCTATA
GCCGGGCAGGGGTGGTCAAAACTATGACCGGTGGGCGTGCCCAATCCATAGGACGCCGCGGGAAAGTCGAGCAATTGAGCC
CGGAAGAAGAGGAAAAGCGGCGCATTCGCCGGGAGCGAAACAAAATGGCCGCCGCTAAGTGTCGTAATAGACGCCGCGAAC
TCACCGACACGTTGCAGGCTGAAACCGATCAGCTCGAGGACGAGAAAAGTGCGCTCCAAACGGAAATCGCTAATCTTCTCAAA
GAAAAGGAGAAGCTTGAATTTATTTTGGCCGCCCATCGCCCCGCTTGTAAAATACCGGACGATCTTGGGTTTCCCGAGGAAAT
GAGTGTCGCCTCTCTGGACCTCACCGGCGGATTGCCTGAAGTGGCTACACCAGAATCCGAAGAAGCGTTTACGCTCCCCCTGC
TGAACGATCCCGAACCAAAACCATCCGTTGAGCCAGTGAAATCAATATCATCCATGGAACTTAAAACAGAACCTTTCGACGATT
TTCTGTTTCCTGCCAGCTCACGCCCGAGCGGTTCAGAAACCGCGAGGTCTGTACCCGATATGGATCTGTCAGGTTCATTTTACG
CTGCCGATTGGGAACCCTTGCATAGCGGTTCTTTGGGCATGGGACCTATGGCTACCGAACTCGAACCTCTCTGTACACCTGTCG
TGACATGCACACCTTCTTGTACCGCCTATACCTCAAGTTTTGTGTTTACTTATCCAGAAGCCGATAGCTTTCCTAGTTGCGCTGCC
GCACATCGTAAAGGAAGTTCCTCAAACGAACCGAGTTCCGATTCTCTTAGTAGCCCAACCCTTTTGGCACTTGGCTCCGGCGAG
GGCAGGGGAAGTCTTCTAACATGCGGGGACGTGGAGGAAAATCCCGGCCCAATGGAAGTCGCCCCAGAACAACCTAGGTGG
ATGGCCCATCCTGCAGTACTCAACGCCCAACATCCTGATAGTCATCATCCCGGATTGGCACATAATTATATGGAGCCGGCTCAA
CTCCTTCCCCCGGATGAAGTCGATGTTTTCTTTAACCATTTGGATAGTCAAGGAAATCCATATTACGCGAATCCTGCCCATGCCC
GTGCCAGGGTGAGCTATTCCCCTGCCCATGCTAGACTCACTGGTGGTCAAATGTGTAGGCCCCATCTTCTCCATTCCCCTGGAC
TGCCTTGGTTGGATGGCGGAAAGGCTGCTCTGTCCGCTGCCGCCGCACATCATCATAATCCTTGGACAGTCAGTCCTTTTAGCA
AAACACCACTCCATCCTTCTGCTGCCGGCGGACCCGGCGGTCCTCTGTCAGTTTATCCGGGCGCTGGTGGCGGCTCTGGTGGT
GGATCAGGTTCTTCCGTCGCAAGCCTGACTCCCACTGCCGCGCATTCCGGTAGCCATCTCTTTGGTTTTCCCCCTACACCCCCAA
AGGAAGTCTCCCCCGATCCCTCAACTACTGGCGCGGCTAGTCCTGCGTCCTCTTCTGCCGGCGGCAGCGCTGCTCGCGGTGAA
GATAAAGATGGAGTGAAATATCAAGTTAGCCTCACCGAATCTATGAAAATGGAGAGCGGATCTCCTTTGCGTCCCGGGTTGGC
CACCATGGGTACACAACCAGCCACCCATCATCCTATTCCTACTTATCCGAGCTACGTACCTGCTGCCGCACATGATTATTCATCT
GGGCTGTTTCATCCAGGCGGGTTTCTCGGTGGCCCCGCAAGCTCATTTACTCCCAAACAAAGGTCCAAAGCGCGGAGCTGCAG
TGAGGGGAGAGAATGCGTGAATTGCGGCGCAACCGCTACGCCCTTGTGGAGAAGGGATGGAACGGGGCATTATCTCTGTAA
CGCATGCGGGTTGTATCATAAAATGAACGGCCAAAATCGCCCCTTGATAAAACCAAAGAGACGGTTGTCAGCTGCACGACGC
GCTGGAACTTGCTGCGCCAACTGCCAAACCACCACAACAACATTGTGGCGGCGTAATGCAAATGGCGATCCCGTGTGTAATGC
TTGCGGTCTGTATTATAAATTGCATAACGTGAATAGACCCCTCACTATGAAGAAAGAGGGCATACAAACCCGCAATCGCAAAA
TGTCAAATAAAAGCAAGAAGTCTAAGAAGGGCGCCGAATGTTTTGAAGAATTGTCTAAATGTATGCAAGAAAAGAGCAGCCC
ATTTTCCGCCGCCGCACTCGCAGGCCATATGGCCCCAGTAGGTCATCTGCCCCCTTTTAGTCATAGCGGGCATATTTTGCCTACG
CCAACACCGATTCATCCTAGCAGCTCACTGTCTTTTGGGCATCCACATCCAAGTTCTATGGTCACGGCTATGGGTTGAggatccatt
cgatatcaagcttatcgataatcaacctctggattacaaaatttgtgaaagattgactggtattcttaactatgttgctccttttacgctatgtggatacgctgctttaatg
cctttgtatcatgctattgcttcccgtatggctttcattttctcctccttgtataaatcctggttgctgtctctttatgaggagttgtggcccgttgtcaggcaacgtggcgtg
gtgtgcactgtgtttgctgacgcaacccccactggttggggcattgccaccacctgtcagctcctttccgggactttcgctttccccctccctattgccacggcggaactc
atcgccgcctgccttgcccgctgctggacaggggctcggctgttgggcactgacaattccgtggtgttgtcggggaaatcatcgtcctttccttggctgctcgcctgtgtt
gccacctggattctgcgcgggacgtccttctgctacgtcccttcggccctcaatccagcggaccttccttcccgcggcctgctgccggctctgcggcctcttccgcgtcttc
gccttcgccctcagacgagtcggatctccctttgggccgcctccccgcatcgataccgtcgacctcgagacctagaaaaacatggagcaatcacaagtagcaatacag
cagctaccaatgctgattgtgcctggctagaagcacaagaggaggaggaggtgggttttccagtcacacctcaggtacctttaagaccaatgacttacaaggcagctg
tagatcttagccactttttaaaagaaaaggggggactggaagggctaattcactcccaacgaagacaagatatccttgatctgtggatctaccacacacaaggctact
tccctgattggcagaactacacaccagggccagggatcagatatccactgacctttggatggtgctacaagctagtaccagttgagcaagagaaggtagaagaagcc
aatgaaggagagaacacccgcttgttacaccctgtgagcctgcatgggatggatgacccggagagagaagtattagagtggaggtttgacagccgcctagcatttca
tcacatggcccgagagctgcatccggactgtactgggtctctctggttagaccagatctgagcctgggagctctctggctaactagggaacccactgcttaagcctcaa
taaagcttgccttgagtgcttcaagtagtgtgtgcccgtctgttgtgtgactctggtaactagagatccctcagacccttttagtcagtgtggaaaatctctagcagggcc
cgtttaaacccgctgatcagcctcgactgtgccttctagttgccagccatctgttgtttgcccctcccccgtgccttccttgaccctggaaggtgccactcccactgtccttt
cctaataaaatgaggaaattgcatcgcattgtctgagtaggtgtcattctattctggggggggggtggggcaggacagcaagggggaggattgggaagacaatagc
aggcatgtgagcaaaaggccagcaaaaggccaggaaccgtaaaaaggccgcgttgctggcgtttttccataggctccgcccccctgacgagcatcacaaaaatcga
cgctcaagtcagaggtggcgaaacccgacaggactataaagataccaggcgtttccccctggaagctccctcgtgcgctctcctgttccgaccctgccgcttaccggat
acctgtccgcctttctcccttcgggaagcgtggcgctttctcatagctcacgctgtaggtatctcagttcggtgtaggtcgttcgctccaagctgggctgtgtgcacgaac
cccccgttcagcccgaccgctgcgccttatccggtaactatcgtcttgagtccaacccggtaagacacgacttatcgccactggcagcagccactggtaacaggattag
cagagcgaggtatgtaggcggtgctacagagttcttgaagtggtggcctaactacggctacactagaagaacagtatttggtatctgcgctctgctgaagccagttacc
ttcggaaaaagagttggtagctcttgatccggcaaacaaaccaccgctggtagcggtggtttttttgtttgcaagcagcagattacgcgcagaaaaaaaggatctcaa
gaagatcctttgatcttttctacggggtctgacgctcagtggaacgaaaactcacgttaagggattttggtcatgagattatcaaaaaggatcttcacctagatcctttta
aattaaaaatgaagttttaaatcaatctaaagtatatatgagtaaacttggtctgacagttaccaatgcttaatcagtgaggcacctatctcagcgatctgtctatttcgt
tcatccatagttgcctgactccccgtcgtgtagataactacgatacgggagggcttaccatctggccccagtgctgcaatgataccgcgagacccacgctcaccggctc
cagatttatcagcaataaaccagccagccggaagggccgagcgcagaagtggtcctgcaactttatccgcctccatccagtctattaattgttgccgggaagctagag
taagtagttcgccagttaatagtttgcgcaacgttgttgccattgctacaggcatcgtggtgtcacgctcgtcgtttggtatggcttcattcagctccggttcccaacgatc
aaggcgagttacatgatcccccatgttgtgcaaaaaagcggttagctccttcggtcctccgatcgttgtcagaagtaagttggccgcagtgttatcactcatggttatgg
cagcactgcataattctcttactgtcatgccatccgtaagatgcttttctgtgactggtgagtactcaaccaagtcattctgagaatagtgtatgcggcgaccgagttgct
cttgcccggcgtcaatacgggataataccgcgccacatagcagaactttaaaagtgctcatcattggaaaacgttcttcggggcgaaaactctcaaggatcttaccgc
tgttgagatccagttcgatgtaacccactcgtgcacccaactgatcttcagcatcttttactttcaccagcgtttctgggtgagcaaaaacaggaaggcaaaatgccgc
aaaaaagggaataagggcgacacggaaatgttgaatactcatactcttcctttttcaatattattgaagcatttatcagggttattgtctcatgagcggatacatatttg
aatgtatttagaaaaataaacaaataggggttccgcgcacatttccccgaaaagtgccacctgacg
SEQ ID No. 24-FUW-FoGaGi (vector 5)
tcgacggatcgggagatctcccgatcccctatggtgcactctcagtacaatctgctctgatgccgcatagttaagccagtatctgctccctgcttgtgtgttggaggtcgc
tgagtagtgcgcgagcaaaatttaagctacaacaaggcaaggcttgaccgacaattgcatgaagaatctgcttagggttaggcgttttgcgctgcttcgcgatgtacg
ggccagatatacgcgttgacattgattattgactagttattaatagtaatcaattacggggtcattagttcatagcccatatatggagttccgcgttacataacttacggt
aaatggcccgcctggctgaccgcccaacgacccccgcccattgacgtcaataatgacgtatgttcccatagtaacgccaatagggactttccattgacgtcaatgggt
ggagtatttacggtaaactgcccacttggcagtacatcaagtgtatcatatgccaagtacgccccctattgacgtcaatgacggtaaatggcccgcctggcattatgcc
cagtacatgaccttatgggactttcctacttggcagtacatctacgtattagtcatcgctattaccatggtgatgcggttttggcagtacatcaatgggcgtggatagcgg
tttgactcacggggatttccaagtctccaccccattgacgtcaatgggagtttgttttggcaccaaaatcaacgggactttccaaaatgtcgtaacaactccgccccatt
gacgcaaatgggcggtaggcgtgtacggtgggaggtctatataagcagcgcgttttgcctgtactgggtctctctggttagaccagatctgagcctgggagctctctgg
ctaactagggaacccactgcttaagcctcaataaagcttgccttgagtgcttcaagtagtgtgtgcccgtctgttgtgtgactctggtaactagagatccctcagaccctt
ttagtcagtgtggaaaatctctagcagtggcgcccgaacagggacttgaaagcgaaagggaaaccagaggagctctctcgacgcaggactcggcttgctgaagcgc
gcacggcaagaggcgaggggcggcgactggtgagtacgccaaaaattttgactagcggaggctagaaggagagagatgggtgcgagagcgtcagtattaagcgg
gggagaattagatcgcgatgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaagaacttagatcattatataat
acagtagcaaccctctattgtgtgcatcaaaggatagagataaaagacaccaaggaagctttagacaagatagaggaagagcaaaacaaaagtaagaccaccgc
acagcaagcggccgctgatcttcagacctggaggaggagatatgagggacaattggagaagtgaattatataaatataaagtagtaaaaattgaaccattaggagt
agcacccaccaaggcaaagagaagagtggtgcagagagaaaaaagagcagtgggaataggagctttgttccttgggttcttgggagcagcaggaagcactatggg
cgcagcgtcaatgacgctgacggtacaggccagacaattattgtctggtatagtgcagcagcagaacaatttgctgagggctattgaggcgcaacagcatctgttgca
actcacagtctggggcatcaagcagctccaggcaagaatcctggctgtggaaagatacctaaaggatcaacagctcctggggatttggggttgctctggaaaactcat
ttgcaccactgctgtgccttggaatgctagttggagtaataaatctctggaacagatttggaatcacacgacctggatggagtgggacagagaaattaacaattacac
aagcttaatacactccttaattgaagaatcgcaaaaccagcaagaaaagaatgaacaagaattattggaattagataaatgggcaagtttgtggaattggtttaaca
taacaaattggctgtggtatataaaattattcataatgatagtaggaggcttggtaggtttaagaatagtttttgctgtactttctatagtgaatagagttaggcagggat
attcaccattatcgtttcagacccacctcccaaccccgaggggacccgacaggcccgaaggaatagaagaagaaggtggagagagagacagagacagatccattc
gattagtgaacggatcggcactgcgtgcgccaattctgcagacaaatggcagtattcatccacaattttaaaagaaaaggggggattggggggtacagtgcagggg
aaagaatagtagacataatagcaacagacatacaaactaaagaattacaaaaacaaattacaaaaattcaaaattttcgggtttattacagggacagcagagatcc
agtttggttaattaacccgtgtcggctccagatctggcctccgcgccgggttttggcgcctcccgcgggcgcccccctcctcacggcgagcgctgccacgtcagacgaa
gggcgcagcgagcgtcctgatccttccgcccggacgctcaggacagcggcccgctgctcataagactcggccttagaaccccagtatcagcagaaggacattttagg
acgggacttgggtgactctagggcactggttttctttccagagagcggaacaggcgaggaaaagtagtcccttctcggcgattctgcggagggatctccgtggggcgg
tgaacgccgatgattatataaggacgcgccgggtgtggcacagctagttccgtcgcagccgggatttgggtcgcggttcttgtttgtggatcgctgtgatcgtcacttgg
tgagtagcgggctgctgggctggccggggctttcgtggccgccgggccgctcggtgggacggaagcgtgtggagagaccgccaagggctgtagtctgggtccgcgag
caaggttgccctgaactgggggttggggggagcgcagcaaaatggcggctgttcccgagtcttgaatggaagacgcttgtgaggcgggctgtgaggtcgttgaaaca
aggtggggggcatggtgggcggcaagaacccaaggtcttgaggccttcgctaatgcgggaaagctcttattcgggtgagatgggctggggcaccatctggggaccct
gacgtgaagtttgtcactgactggagaactcggtttgtcgtctgttgcgggggcggcagttatggcggtgccgttgggcagtgcacccgtacctttgggagcgcgcgcc
ctcgtcgtgtcgtgacgtcacccgttctgttggcttataatgcagggtggggccacctgccggtaggtgtgcggtaggcttttctccgtcgcaggacgcagggttcgggc
ctagggtaggctctcctgaatcgacaggcgccggacctctggtgaggggagggataagtgaggcgtcagtttctttggtcggttttatgtacctatcttcttaagtagct
gaagctccggttttgaactatgcgctcggggttggcgagtgtgttttgtgaagttttttaggcaccttttgaaatgtaatcatttgggtcaatatgtaattttcagtgttaga
ctagtaaattgtccgctaaattctggccgtttttggcttttttgttagacgaagcttgggcccgggatccATGATGTTTTCAGGATTTAATGCTGATTATGA
AGCCAGCTCTAGTAGGTGTTCCTCTGCAAGCCCAGCTGGTGACTCCCTGTCATATTATCATAGTCCTGCTGATAGCTTTAGTTCT
ATGGGATCTCCAGTGAATGCCCAAGACTTTTGTACAGATCTCGCAGTAAGCAGCGCAAATTTTATCCCGACTGTTACCGCTATT
TCCACTTCACCCGATCTCCAATGGCTTGTCCAACCAGCGCTGGTGAGTAGTGTTGCTCCGAGCCAAACACGGGCTCCCCATCCC
TTTGGGGTGCCTGCTCCGAGCGCAGGCGCATATAGCCGTGCCGGAGTGGTGAAAACAATGACCGGCGGACGGGCACAATCTA
TCGGGCGTAGAGGTAAAGTCGAGCAACTGAGTCCTGAGGAAGAGGAAAAGAGACGGATTAGGGGGAGCGCAACAAAATG
GCAGCCGCAAAGTGTCGAAATAGAAGACGCGAACTCACGGACACCCTGCAGGCAGAAACTGATCAGCTTGAGGACGAAAAG
AGTGCCCTGCAAACTGAAATCGCAAATCTTCTCAAAGAAAAGGAGAAGCTGGAATTTATTCTCGCCGCACATAGGCCCGCTTG
TAAAATTCCCGACGATCTCGGATTTCCTGAGGAAATGAGCGTTGCCTCACTGGACCTTACAGGCGGGTTGCCCGAAGTGGCTA
CTCCCGAATCAGAAGAAGCATTTACTCTTCCCCTGCTGAACGATCCCGAACCAAAACCATCCGTTGAGCCGGTGAAATCTATTA
GTTCAATGGAATTGAAAACTGAACCTTTCGACGATTTTCTCTTTCCTGCCTCTAGCCGACCATCAGGGTCCGAAACGGCTCGTA
GTGTACCTGATATGGATCTGAGCGGTAGCTTTTACGCCGCCGATTGGGAACCTCTCCATTCCGGTTCATTGGGCATGGGACCG
ATGGCTACTGAATTGGAACCGCTCTGTACCCCAGTAGTGACATGCACACCTTCATGTACAGCATATACATCAAGCTTTGTGTTT
ACTTATCCGGAAGCCGATTCTTTTCCTTCTTGCGCTGCCGCTCATAGGAAAGGCTCCTCTTCTAACGAACCAAGTAGTGATTCAC
TGTCATCCCCTACACTTTTGGCTCTTGGCAGCGGCGCCACAAACTTCTCTCTGCTAAAGCAAGCAGGTGATGTTGAAGAAAACC
CCGGGCCTATGGAAGTAGCCCCTGAACAACCCAGATGGATGGCTCATCCAGCAGTATTGAACGCACAACATCCAGATTCCCAT
CATCCAGGATTGGCCCATAATTATATGGAGCCAGCCCAACTCTTGCCACCTGATGAAGTCGATGTTTTCTTTAACCATCTGGATA
GTCAAGGTAATCCTTATTACGCTAATCCAGCACATGCACGCGCTCGGGTTTCATATTCCCCTGCCCATGCAAGGCTTACAGGCG
GACAAATGTGTCGTCCTCATCTGCTCCATTCCCCAGGCCTTCCATGGCTTGATGGCGGGAAGGCGGCGCTTTCAGCGGCGGCT
GCACATCATCATAATCCGTGGACTGTCTCTCCTTTTAGTAAAACTCCGCTTCATCCGAGCGCAGCAGGCGGACCTGGCGGACCC
CTGTCAGTCTATCCTGGTGCCGGTGGCGGCTCCGGCGGTGGGTCCGGTTCCAGCGTTGCAAGCTTGACACCAACGGCGGCTCA
TTCAGGATCACATTTGTTTGGTTTTCCCCCAACCCCGCCGAAAGAGGTCAGTCCCGATCCAAGTACAACCGGAGCAGCAAGTCC
CGCAAGCTCATCTGCCGGCGGCTCCGCCGCTAGAGGCGAAGATAAAGATGGGGTAAAATATCAAGTCTCTCTTACCGAATCCA
TGAAAATGGAGTCTGGATCTCCTCTCCGGCCCGGGCTCGCGACCATGGGGACACAACCCGCAACCCATCATCCAATTCCAACTT
ATCCAAGCTACGTTCCCGCTGCAGCACATGATTATTCATCTGGGCTGTTTCATCCGGGCGGATTTCTCGGTGGCCCAGCATCTTC
TTTTACTCCCAAACAACGGTCTAAAGCAAGGTCTTGCAGTGAGGGTAGGGAATGCGTGAATTGCGGAGCTACTGCAACTCCCC
TGTGGCGTCGCGATGGGACAGGTCATTATCTTTGTAACGCTTGCGGACTTTATCATAAAATGAACGGCCAAAATCGCCCGCTG
ATTAAACCTAAACGCAGGTTGTCAGCGGCTCGCAGGGGGGGGACATGCTGCGCCAACTGCCAAACCACCACTACAACACTGTG
GCGAAGAAATGCAAATGGCGATCCCGTGTGTAATGCTTGCGGGCTTTATTATAAACTCCATAACGTCAATCGGCCTCTTACAAT
GAAGAAAGAGGGAATTCAAACCAGGAATAGGAAAATGTCAAATAAAAGCAAGAAATCAAAGAAGGGCGCCGAATGTTTTGA
AGAATTGTCTAAATGTATGCAAGAGAAATCTTCACCGTTTTCCGCCGCCGCGTTGGCCGGTCATATGGCTCCAGTCGGACATCT
TCCGCCATTTTCACATAGTGGGCATATTCTTCCAACACCCACACCTATTCATCCTTCATCTTCTCTGTCATTTGGACATCCTCATCC
TAGCTCCATGGTTACTGCAATGGGCGGCTCCGGCGAGGGCAGGGGAAGTCTTCTAACATGCGGGGACGTGGAGGAAAATCC
CGGCCCAATGCCCCGAAGTTTTCTCGTTAAATCAAAGAAAGCACATACTTATCATCAACCTCGCGTCCAAGAGGACGAGCCACT
TTGGCCACCCGCTCTGACACCTGTCCCGAGGGATCAAGCGCCGTCCAATTCACCCGTGTTGTCTACCCTCTTTCCCAATCAATGT
TTGGATTGGACAAATTTGAAGAGGGAACCAGAACTTGAACAAGATCAAAATCTTGCGCGGATGGCGCCTGCCCCGGAAGGGC
CAATCGTCCTCAGCCGGCCGCAAGACGGCGATTCCCCTCTTAGCGATAGCCCACCATTTTATAAACCCAGTTTTAGCTGGGATA
CTCTGGCTACTACTTACGGACATTCATATAGACAAGCGCCTTCAACGATGCAAAGCGCATTTCTTGAACATAGTGTGTCCCTTTA
TGGTTCCCCACTCGTTCCTTCCACAGAACCAGCTCTGGATTTCTCACTTCGATATTCTCCCGGGATGGACGCTTATCATTGCGTA
AAATGTAATAAAGTGTTTTCTACACCGCATGGTCTGGAAGTCCACGTACGCCGTAGTCACTCCGGCACAAGGCCTTTTGCGTGC
GATATTTGTGGGAAGACGTTTGGTCATGCCGTATCACTCGAACAACATACACATGTGCATTCTCAAGAACGGAGTTTTGAATGT
AGAATGTGTGGTAAAGCTTTTAAAAGGTCATCAACTTTGAGTACTCATCTCTTGATACACAGCGATACAAGGCCGTATCCATGT
CAATTTTGTGGAAAACGGTTTCATCAAAAGTCAGATATGAAGAAACATACATATATTCATACCGGGGAAAAGCCCCATAAATG
TCAAGTCTGTGGTAAAGCGTTTTCTCAATCTAGTAATCTGATTACACATTCTCGTAAACATACTGGGTTTAAACCTTTCTCTTGCG
AACTTTGTACAAAGGGATTTCAACGGAAAGTCGATCTTCGTAGACATAGAGAATCCCAACATAACCTTAAATAAggatccattcgat
atcaagcttatcgataatcaacctctggattacaaaatttgtgaaagattgactggtattcttaactatgttgctccttttacgctatgtggatacgctgctttaatgccttt
gtatcatgctattgcttcccgtatggctttcattttctcctccttgtataaatcctggttgctgtctctttatgaggagttgtggcccgttgtcaggcaacgtggcgtggtgtg
cactgtgtttgctgacgcaacccccactggttggggcattgccaccacctgtcagctcctttccgggactttcgctttccccctccctattgccacggcggaactcatcgc
cgcctgccttgcccgctgctggacaggggctcggctgttgggcactgacaattccgtggtgttgtcggggaaatcatcgtcctttccttggctgctcgcctgtgttgccac
ctggattctgcgcgggacgtccttctgctacgtcccttcggccctcaatccagcggaccttccttcccgcggcctgctgccggctctgcggcctcttccgcgtcttcgcctt
cgccctcagacgagtcggatctccctttgggccgcctccccgcatcgataccgtcgacctcgagacctagaaaaacatggagcaatcacaagtagcaatacagcagc
taccaatgctgattgtgcctggctagaagcacaagaggaggaggaggtgggttttccagtcacacctcaggtacctttaagaccaatgacttacaaggcagctgtaga
tcttagccactttttaaaagaaaaggggggactggaagggctaattcactcccaacgaagacaagatatccttgatctgtggatctaccacacacaaggctacttccct
gattggcagaactacacaccagggccagggatcagatatccactgacctttggatggtgctacaagctagtaccagttgagcaagagaaggtagaagaagccaatg
aaggagagaacacccgcttgttacaccctgtgagcctgcatgggatggatgacccggagagagaagtattagagtggaggtttgacagccgcctagcatttcatcac
atggcccgagagctgcatccggactgtactgggtctctctggttagaccagatctgagcctgggagctctctggctaactagggaacccactgcttaagcctcaataaa
gcttgccttgagtgcttcaagtagtgtgtgcccgtctgttgtgtgactctggtaactagagatccctcagacccttttagtcagtgtggaaaatctctagcagggcccgttt
aaacccgctgatcagcctcgactgtgccttctagttgccagccatctgttgtttgcccctcccccgtgccttccttgaccctggaaggtgccactcccactgtcctttccta
ataaaatgaggaaattgcatcgcattgtctgagtaggtgtcattctattctggggggtggggtggggcaggacagcaagggggaggattgggaagacaatagcagg
catgtgagcaaaaggccagcaaaaggccaggaaccgtaaaaaggccgcgttgctggcgtttttccataggctccgcccccctgacgagcatcacaaaaatcgacgc
tcaagtcagaggtggcgaaacccgacaggactataaagataccaggcgtttccccctggaagctccctcgtgcgctctcctgttccgaccctgccgcttaccggatacc
tgtccgcctttctcccttcgggaagcgtggcgctttctcatagctcacgctgtaggtatctcagttcggtgtaggtcgttcgctccaagctgggctgtgtgcacgaacccc
ccgttcagcccgaccgctgcgccttatccggtaactatcgtcttgagtccaacccggtaagacacgacttatcgccactggcagcagccactggtaacaggattagca
gagcgaggtatgtaggcggtgctacagagttcttgaagtggtggcctaactacggctacactagaagaacagtatttggtatctgcgctctgctgaagccagttacctt
cggaaaaagagttggtagctcttgatccggcaaacaaaccaccgctggtagcggtggtttttttgtttgcaagcagcagattacgcgcagaaaaaaaggatctcaag
aagatcctttgatcttttctacggggtctgacgctcagtggaacgaaaactcacgttaagggattttggtcatgagattatcaaaaaggatcttcacctagatccttttaa
attaaaaatgaagttttaaatcaatctaaagtatatatgagtaaacttggtctgacagttaccaatgcttaatcagtgaggcacctatctcagcgatctgtctatttcgtt
catccatagttgcctgactccccgtcgtgtagataactacgatacgggagggcttaccatctggccccagtgctgcaatgataccgcgagacccacgctcaccggctcc
agatttatcagcaataaaccagccagccggaagggccgagcgcagaagtggtcctgcaactttatccgcctccatccagtctattaattgttgccgggaagctagagt
aagtagttcgccagttaatagtttgcgcaacgttgttgccattgctacaggcatcgtggtgtcacgctcgtcgtttggtatggcttcattcagctccggttcccaacgatc
aaggcgagttacatgatcccccatgttgtgcaaaaaagcggttagctccttcggtcctccgatcgttgtcagaagtaagttggccgcagtgttatcactcatggttatgg
cagcactgcataattctcttactgtcatgccatccgtaagatgcttttctgtgactggtgagtactcaaccaagtcattctgagaatagtgtatgcggcgaccgagttgct
cttgcccggcgtcaatacgggataataccgcgccacatagcagaactttaaaagtgctcatcattggaaaacgttcttcggggcgaaaactctcaaggatcttaccgc
tgttgagatccagttcgatgtaacccactcgtgcacccaactgatcttcagcatcttttactttcaccagcgtttctgggtgagcaaaaacaggaaggcaaaatgccgc
aaaaaagggaataagggcgacacggaaatgttgaatactcatactcttcctttttcaatattattgaagcatttatcagggttattgtctcatgagcggatacatatttg
aatgtatttagaaaaataaacaaataggggttccgcgcacatttccccgaaaagtgccacctgacg
SEQ ID No. 25-FUW-FoGiGa (vector 6)
tcgacggatcgggagatctcccgatcccctatggtgcactctcagtacaatctgctctgatgccgcatagttaagccagtatctgctccctgcttgtgtgttggaggtcgc
tgagtagtgcgcgagcaaaatttaagctacaacaaggcaaggcttgaccgacaattgcatgaagaatctgcttagggttaggcgttttgcgctgcttcgcgatgtacg
ggccagatatacgcgttgacattgattattgactagttattaatagtaatcaattacggggtcattagttcatagcccatatatggagttccgcgttacataacttacggt
aaatggcccgcctggctgaccgcccaacgacccccgcccattgacgtcaataatgacgtatgttcccatagtaacgccaatagggactttccattgacgtcaatgggt
ggagtatttacggtaaactgcccacttggcagtacatcaagtgtatcatatgccaagtacgccccctattgacgtcaatgacggtaaatggcccgcctggcattatgcc
cagtacatgaccttatgggactttcctacttggcagtacatctacgtattagtcatcgctattaccatggtgatgcggttttggcagtacatcaatgggcgtggatagcgg
tttgactcacggggatttccaagtctccaccccattgacgtcaatgggagtttgttttggcaccaaaatcaacgggactttccaaaatgtcgtaacaactccgccccatt
gacgcaaatgggcggtaggcgtgtacggtgggaggtctatataagcagcgcgttttgcctgtactgggtctctctggttagaccagatctgagcctgggagctctctgg
ctaactagggaacccactgcttaagcctcaataaagcttgccttgagtgcttcaagtagtgtgtgcccgtctgttgtgtgactctggtaactagagatccctcagaccctt
ttagtcagtgtggaaaatctctagcagtggcgcccgaacagggacttgaaagcgaaagggaaaccagaggagctctctcgacgcaggactcggcttgctgaagcgc
gcacggcaagaggcgaggggcggcgactggtgagtacgccaaaaattttgactagcggaggctagaaggagagagatgggtgcgagagcgtcagtattaagcgg
gggagaattagatcgcgatgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaagaacttagatcattatataat
acagtagcaaccctctattgtgtgcatcaaaggatagagataaaagacaccaaggaagctttagacaagatagaggaagagcaaaacaaaagtaagaccaccgc
acagcaagcggccgctgatcttcagacctggaggaggagatatgagggacaattggagaagtgaattatataaatataaagtagtaaaaattgaaccattaggagt
agcacccaccaaggcaaagagaagagtggtgcagagagaaaaaagagcagtgggaataggagctttgttccttgggttcttgggagcagcaggaagcactatggg
cgcagcgtcaatgacgctgacggtacaggccagacaattattgtctggtatagtgcagcagcagaacaatttgctgagggctattgaggcgcaacagcatctgttgca
actcacagtctggggcatcaagcagctccaggcaagaatcctggctgtggaaagatacctaaaggatcaacagctcctggggatttggggttgctctggaaaactcat
ttgcaccactgctgtgccttggaatgctagttggagtaataaatctctggaacagatttggaatcacacgacctggatggagtgggacagagaaattaacaattacac
aagcttaatacactccttaattgaagaatcgcaaaaccagcaagaaaagaatgaacaagaattattggaattagataaatgggcaagtttgtggaattggtttaaca
taacaaattggctgtggtatataaaattattcataatgatagtaggaggcttggtaggtttaagaatagtttttgctgtactttctatagtgaatagagttaggcagggat
attcaccattatcgtttcagacccacctcccaaccccgaggggacccgacaggcccgaaggaatagaagaagaaggtggagagagagacagagacagatccattc
gattagtgaacggatcggcactgcgtgcgccaattctgcagacaaatggcagtattcatccacaattttaaaagaaaaggggggattggggggtacagtgcagggg
aaagaatagtagacataatagcaacagacatacaaactaaagaattacaaaaacaaattacaaaaattcaaaattttcgggtttattacagggacagcagagatcc
agtttggttaattaacccgtgtcggctccagatctggcctccgcgccgggttttggcgcctcccgcgggcgcccccctcctcacggcgagcgctgccacgtcagacgaa
gggcgcagcgagcgtcctgatccttccgcccggacgctcaggacagcggcccgctgctcataagactcggccttagaaccccagtatcagcagaaggacattttagg
acgggacttgggtgactctagggcactggttttctttccagagagcggaacaggcgaggaaaagtagtcccttctcggcgattctgcggagggatctccgtggggcgg
tgaacgccgatgattatataaggacgcgccgggtgtggcacagctagttccgtcgcagccgggatttgggtcgcggttcttgtttgtggatcgctgtgatcgtcacttgg
tgagtagcgggctgctgggctggccggggctttcgtggccgccgggccgctcggtgggacggaagcgtgtggagagaccgccaagggctgtagtctgggtccgcgag
caaggttgccctgaactgggggttggggggagcgcagcaaaatggcggctgttcccgagtcttgaatggaagacgcttgtgaggcgggctgtgaggtcgttgaaaca
aggtggggggcatggtgggcggcaagaacccaaggtcttgaggccttcgctaatgcgggaaagctcttattcgggtgagatgggctggggcaccatctggggaccct
gacgtgaagtttgtcactgactggagaactcggtttgtcgtctgttgcgggggcggcagttatggcggtgccgttgggcagtgcacccgtacctttgggagcgcgcgcc
ctcgtcgtgtcgtgacgtcacccgttctgttggcttataatgcagggtggggccacctgccggtaggtgtgcggtaggcttttctccgtcgcaggacgcagggttcgggc
ctagggtaggctctcctgaatcgacaggcgccggacctctggtgaggggagggataagtgaggcgtcagtttctttggtcggttttatgtacctatcttcttaagtagct
gaagctccggttttgaactatgcgctcggggttggcgagtgtgttttgtgaagttttttaggcaccttttgaaatgtaatcatttgggtcaatatgtaattttcagtgttaga
ctagtaaattgtccgctaaattctggccgtttttggcttttttgttagacgaagcttgggcccgggatccATGATGTTTAGCGGATTTAATGCCGATTATGA
AGCAAGCTCATCTCGATGTTCATCTGCAAGCCCAGCTGGTGACTCACTGAGCTATTATCATAGCCCGGCTGATTCATTTTCATCT
ATGGGTTCCCCCGTTAATGCCCAAGACTTTTGTACCGATCTCGCAGTGAGCAGCGCAAATTTTATCCCTACCGTGACCGCTATAT
CAACTAGCCCCGATTTGCAATGGCTTGTCCAACCAGCGTTGGTGTCAAGCGTTGCACCCAGCCAAACTCGCGCTCCCCATCCCT
TTGGCGTGCCAGCTCCGAGCGCAGGCGCCTATTCACGGGCAGGGGTAGTCAAAACAATGACCGGCGGACGCGCTCAATCCAT
CGGAAGAAGAGGGAAAGTCGAGCAACTCTCCCCCGAGGAAGAGGAAAAGCGACGCATTAGAAGAGAGCGGAACAAAATGG
CCGCCGCTAAGTGTCGGAATAGACGCCGCGAACTCACCGACACTCTGCAGGCCGAAACCGATCAGCTGGAAGACGAAAAGAG
TGCCCTTCAAACTGAAATCGCAAATCTCTTGAAAGAAAAGGAGAAGCTGGAATTTATTCTTGCTGCACATCGTCCAGCATGTAA
AATTCCGGACGATTTGGGGTTTCCCGAGGAAATGTCCGTTGCCTCTCTCGACTTGACCGGTGGGTTGCCTGAAGTAGCTACACC
CGAAAGTGAAGAAGCGTTTACTCTTCCATTGCTTAACGATCCAGAACCAAAACCAAGCGTTGAGCCCGTAAAATCTATTTCTTC
AATGGAACTCAAAACTGAACCATTCGACGATTTTCTCTTTCCTGCTTCTAGTAGACCATCTGGCTCCGAAACCGCACGATCTGTA
CCTGATATGGATCTGTCCGGAAGCTTTTACGCCGCCGATTGGGAACCACTCCATTCCGGTTCACTCGGAATGGGACCTATGGCT
ACGGAATTGGAACCTCTTTGTACCCCAGTTGTAACTTGCACCCCATCATGTACCGCCTATACCTCTAGCTTTGTATTTACGTATCC
TGAAGCCGATAGCTTTCCATCTTGCGCCGCCGCGCATAGGAAAGGATCTTCAAGTAACGAACCCAGTTCCGATAGTTTGAGTTC
TCCGACACTTCTCGCTCTTGGCAGCGGCGCCACAAACTTCTCTCTGCTAAAGCAAGCAGGTGATGTTGAAGAAAACCCCGGGC
CTATGCCCCGTTCTTTCTTGGTAAAATCCAAGAAGGCGCATACATATCATCAACCACGGGTTCAAGAGGACGAGCCACTGTGG
CCGCCAGCTCTGACTCCCGTCCCTCGGGATCAAGCCCCCTCTAATTCTCCCGTGCTGTCAACATTGTTTCCTAATCAATGTTTGG
ATTGGACTAATCTGAAGCGCGAACCAGAATTGGAACAAGATCAAAATCTCGCTCGGATGGCTCCTGCTCCGGAAGGTCCTATC
GTTCTTTCAAGACCACAAGACGGCGATTCACCCCTCTCAGATTCTCCCCCTTTCTATAAACCCAGCTTTAGTTGGGATACTCTGG
CAACCACATACGGGCATTCTTATCGCCAAGCGCCAAGCACAATGCAAAGTGCATTTCTCGAACATTCAGTATCTCTTTATGGAT
CTCCCCTGGTCCCTTCTACCGAACCAGCGCTTGACTTTTCCTTGAGGTATAGTCCCGGGATGGACGCATATCATTGCGTAAAAT
GTAATAAAGTTTTCAGCACGCCCCATGGATTGGAAGTCCACGTCAGGCGATCTCACTCTGGCACAAGACCTTTTGCATGCGATA
TTTGTGGTAAGACGTTTGGACATGCCGTTTCACTCGAACAACATACACATGTTCATAGCCAAGAACGTTCATTTGAATGTAGAA
TGTGTGGAAAAGCATTTAAAAGGAGCTCTACCCTTTCTACACATTTGCTGATTCATAGCGATACCCGACCTTATCCGTGTCAATT
TTGTGGAAAACGATTTCATCAGAAATCAGATATGAAGAAGCATACATATATACATACCGGGGAGAAACCCCATAAATGTCAAG
TCTGTGGCAAAGCATTTTCTCAATCAAGCAATCTGATTACTCATTCCAGAAAACATACTGGATTTAAACCCTTTTCATGCGAACT
TTGTACTAAGGGATTTCAACGGAAAGTTGATTTGAGGAGACATCGAGAAAGTCAACATAACCTGAAAGGCTCCGGCGAGGGC
AGGGGAAGTCTTCTAACATGCGGGGACGTGGAGGAAAATCCCGGCCCAATGGAAGTTGCCCCGGAACAACCCCGTTGGATG
GCCCATCCTGCAGTACTTAACGCCCAACATCCGGATTCCCATCATCCCGGACTCGCTCATAATTATATGGAGCCGGCTCAACTCT
TGCCACCCGATGAAGTCGATGTGTTCTTTAACCATCTGGATTCTCAAGGGAATCCATATTACGCAAATCCAGCACATGCCCGAG
CCAGAGTTAGCTATTCACCTGCCCATGCAAGATTGACTGGTGGACAAATGTGTCGGCCCCATCTGCTCCATTCACCAGGGCTCC
CATGGCTTGATGGCGGTAAGGCTGCGCTGAGCGCTGCCGCCGCACATCATCATAATCCATGGACAGTATCTCCCTTTTCTAAAA
CTCCCCTTCATCCTTCTGCAGCCGGTGGACCCGGCGGACCTTTGAGTGTTTATCCTGGCGCAGGCGGCGGTTCCGGCGGCGGG
AGTGGAAGCAGTGTCGCGAGTCTGACGCCCACTGCTGCACATTCAGGAAGTCATCTGTTTGGGTTTCCCCCAACTCCACCCAAA
GAGGTTTCCCCAGATCCAAGTACAACCGGCGCAGCCAGTCCCGCTTCCTCTAGCGCCGGCGGCTCAGCTGCTAGAGGGGAAG
ATAAAGATGGTGTTAAATATCAAGTTAGCCTTACTGAATCTATGAAAATGGAGTCAGGGAGCCCGCTCAGACCCGGGCTGGCG
ACGATGGGTACACAACCGGCCACTCATCATCCTATTCCGACGTATCCGAGTTACGTTCCCGCCGCCGCTCATGATTATTCTTCCG
GCCTGTTTCATCCAGGCGGTTTTCTTGGCGGCCCCGCGAGCTCATTTACGCCCAAACAAAGGAGTAAAGCCAGATCTTGCTCTG
AGGGGAGAGAATGCGTGAATTGCGGCGCGACGGCAACTCCCTTGTGGCGCCGCGATGGTACTGGGCATTATCTCTGTAACGC
ATGCGGATTGTATCATAAAATGAACGGTCAAAATAGGCCTCTGATAAAACCTAAACGGCGCCTTAGTGCGGCAAGGCGTGCA
GGAACATGCTGCGCTAACTGCCAAACAACTACAACTACTTTGTGGCGGCGCAATGCGAATGGTGATCCCGTGTGTAATGCTTG
CGGATTGTATTATAAACTCCATAACGTGAATCGTCCTCTCACTATGAAGAAGGAAGGAATTCAAACGCGCAATAGGAAAATGT
CTAATAAAAGCAAGAAGTCCAAGAAGGGCGCTGAATGTTTTGAAGAACTCAGCAAATGTATGCAAGAGAAATCCAGTCCATTT
TCAGCCGCCGCACTTGCAGGTCATATGGCTCCCGTCGGGCATTTGCCACCGTTTTCCCATAGCGGTCATATTCTCCCAACCCCTA
CTCCAATACATCCTTCTAGCTCTCTGAGTTTTGGGCATCCTCATCCCAGTTCCATGGTCACAGCGATGGGTTGAggatccattcgatat
caagcttatcgataatcaacctctggattacaaaatttgtgaaagattgactggtattcttaactatgttgctccttttacgctatgtggatacgctgctttaatgcctttgt
atcatgctattgcttcccgtatggctttcattttctcctccttgtataaatcctggttgctgtctctttatgaggagttgtggcccgttgtcaggcaacgtggcgtggtgtgca
ctgtgtttgctgacgcaacccccactggttggggcattgccaccacctgtcagctcctttccgggactttcgctttccccctccctattgccacggcggaactcatcgccg
cctgccttgcccgctgctggacaggggctcggctgttgggcactgacaattccgtggtgttgtcggggaaatcatcgtcctttccttggctgctcgcctgtgttgccacct
ggattctgcgcgggacgtccttctgctacgtcccttcggccctcaatccagcggaccttccttcccgcggcctgctgccggctctgcggcctcttccgcgtcttcgccttcg
ccctcagacgagtcggatctccctttgggccgcctccccgcatcgataccgtcgacctcgagacctagaaaaacatggagcaatcacaagtagcaatacagcagcta
ccaatgctgattgtgcctggctagaagcacaagaggaggaggaggtgggttttccagtcacacctcaggtacctttaagaccaatgacttacaaggcagctgtagatc
ttagccactttttaaaagaaaaggggggactggaagggctaattcactcccaacgaagacaagatatccttgatctgtggatctaccacacacaaggctacttccctg
attggcagaactacacaccagggccagggatcagatatccactgacctttggatggtgctacaagctagtaccagttgagcaagagaaggtagaagaagccaatga
aggagagaacacccgcttgttacaccctgtgagcctgcatgggatggatgacccggagagagaagtattagagtggaggtttgacagccgcctagcatttcatcaca
tggcccgagagctgcatccggactgtactgggtctctctggttagaccagatctgagcctgggagctctctggctaactagggaacccactgcttaagcctcaataaag
cttgccttgagtgcttcaagtagtgtgtgcccgtctgttgtgtgactctggtaactagagatccctcagacccttttagtcagtgtggaaaatctctagcagggcccgttta
aacccgctgatcagcctcgactgtgccttctagttgccagccatctgttgtttgcccctcccccgtgccttccttgaccctggaaggtgccactcccactgtcctttcctaat
aaaatgaggaaattgcatcgcattgtctgagtaggtgtcattctattctggggggtggggggggcaggacagcaagggggaggattgggaagacaatagcaggca
tgtgagcaaaaggccagcaaaaggccaggaaccgtaaaaaggccgcgttgctggcgtttttccataggctccgcccccctgacgagcatcacaaaaatcgacgctc
aagtcagaggtggcgaaacccgacaggactataaagataccaggcgtttccccctggaagctccctcgtgcgctctcctgttccgaccctgccgcttaccggatacctg
tccgcctttctcccttcgggaagcgtggcgctttctcatagctcacgctgtaggtatctcagttcggtgtaggtcgttcgctccaagctgggctgtgtgcacgaacccccc
gttcagcccgaccgctgcgccttatccggtaactatcgtcttgagtccaacccggtaagacacgacttatcgccactggcagcagccactggtaacaggattagcaga
gcgaggtatgtaggcggtgctacagagttcttgaagtggtggcctaactacggctacactagaagaacagtatttggtatctgcgctctgctgaagccagttaccttcg
gaaaaagagttggtagctcttgatccggcaaacaaaccaccgctggtagcggtggtttttttgtttgcaagcagcagattacgcgcagaaaaaaaggatctcaagaa
gatcctttgatcttttctacggggtctgacgctcagtggaacgaaaactcacgttaagggattttggtcatgagattatcaaaaaggatcttcacctagatccttttaaat
taaaaatgaagttttaaatcaatctaaagtatatatgagtaaacttggtctgacagttaccaatgcttaatcagtgaggcacctatctcagcgatctgtctatttcgttca
tccatagttgcctgactccccgtcgtgtagataactacgatacgggagggcttaccatctggccccagtgctgcaatgataccgcgagacccacgctcaccggctccag
atttatcagcaataaaccagccagccggaagggccgagcgcagaagtggtcctgcaactttatccgcctccatccagtctattaattgttgccgggaagctagagtaa
gtagttcgccagttaatagtttgcgcaacgttgttgccattgctacaggcatcgtggtgtcacgctcgtcgtttggtatggcttcattcagctccggttcccaacgatcaa
ggcgagttacatgatcccccatgttgtgcaaaaaagcggttagctccttcggtcctccgatcgttgtcagaagtaagttggccgcagtgttatcactcatggttatggca
gcactgcataattctcttactgtcatgccatccgtaagatgcttttctgtgactggtgagtactcaaccaagtcattctgagaatagtgtatgcggcgaccgagttgctct
tgcccggcgtcaatacgggataataccgcgccacatagcagaactttaaaagtgctcatcattggaaaacgttcttcggggcgaaaactctcaaggatcttaccgctg
ttgagatccagttcgatgtaacccactcgtgcacccaactgatcttcagcatcttttactttcaccagcgtttctgggtgagcaaaaacaggaaggcaaaatgccgcaa
aaaagggaataagggcgacacggaaatgttgaatactcatactcttcctttttcaatattattgaagcatttatcagggttattgtctcatgagcggatacatatttgaat
gtatttagaaaaataaacaaataggggttccgcgcacatttccccgaaaagtgccacctgacg
SEQ ID No. 26-GATA2 (from vector 1)
ATGGAAGTCGCTCCTGAACAACCACGGTGGATGGCTCATCCTGCAGTTCTTAACGCTCAACATCCAGATAGCCATCATCCTGGG
CTCGCTCATAATTATATGGAGCCTGCCCAACTCCTTCCCCCAGATGAAGTAGATGTATTCTTTAACCATCTTGATAGCCAAGGAA
ATCCTTATTACGCAAATCCTGCCCATGCCAGAGCCAGAGTAAGCTATTCACCAGCCCATGCAAGGCTTACTGGTGGACAAATGT
GTCGGCCCCATCTGCTGCATTCCCCCGGCCTGCCGTGGTTGGATGGCGGAAAGGCGGCTCTGAGTGCGGCGGCTGCACATCAT
CATAATCCTTGGACAGTCTCACCTTTCTCTAAAACTCCCCTTCATCCTAGTGCAGCCGGTGGTCCAGGTGGTCCGCTGTCCGTTT
ATCCCGGCGCCGGCGGCGGAAGTGGCGGCGGAAGTGGCAGTAGCGTAGCTTCATTGACACCCACAGCTGCTCATTCCGGAAG
TCATCTCTTTGGGTTTCCCCCTACCCCGCCGAAAGAGGTTTCCCCAGATCCGTCAACTACCGGTGCCGCTTCCCCTGCTAGTTCC
AGTGCCGGCGGAAGCGCTGCAAGGGGCGAAGATAAAGATGGAGTGAAATATCAAGTCTCCTTGACAGAATCCATGAAAATG
GAGTCCGGTTCTCCACTCAGGCCTGGACTCGCCACAATGGGTACTCAACCCGCCACCCATCATCCAATACCGACTTATCCTTCAT
ACGTACCAGCCGCCGCTCATGATTATTCATCAGGTTTGTTTCATCCAGGTGGATTTCTTGGCGGGCCTGCTAGTTCTTTTACACC
AAAACAAAGGTCCAAAGCCAGGTCTTGCAGTGAGGGGCGCGAATGCGTAAATTGCGGCGCTACCGCAACACCACTGTGGCGT
CGTGATGGAACAGGACATTATCTTTGTAACGCATGCGGGCTGTATCATAAAATGAACGGACAAAATCGGCCCCTGATTAAACC
TAAACGTCGCTTGTCTGCTGCAAGGCGGGCTGGGACGTGCTGCGCCAACTGCCAAACAACCACAACAACACTGTGGCGTAGG
AATGCTAATGGCGATCCCGTATGTAATGCATGCGGTCTGTATTATAAACTTCATAACGTCAATCGGCCCCTTACTATGAAGAAA
GAGGGCATACAAACCCGAAATCGCAAAATGTCAAATAAATCTAAGAAATCCAAGAAGGGAGCCGAATGTTTTGAAGAACTCA
GCAAATGTATGCAAGAAAAGTCCAGCCCGTTTTCCGCCGCCGCTTTGGCCGGCCATATGGCCCCCGTCGGACATCTGCCACCG
TTTTCCCATAGCGGTCATATTCTTCCTACACCCACCCCAATTCATCCGTCTAGCTCCCTGTCTTTTGGGCATCCTCATCCTAGCTCA
ATGGTCACAGCTATGGGT
SEQ ID No. 27-GATA2 (from vector 2)
ATGGAAGTCGCCCCGGAACAACCACGATGGATGGCACATCCTGCAGTTCTCAACGCACAACATCCTGATAGTCATCATCCAGG
GTTGGCTCATAATTATATGGAGCCTGCCCAACTCCTTCCCCCTGATGAAGTTGATGTGTTCTTTAACCATCTGGATTCTCAAGGA
AATCCGTATTACGCTAATCCAGCCCATGCACGCGCACGTGTTTCATATAGTCCTGCCCATGCGAGGCTTACAGGCGGGCAAAT
GTGTCGTCCCCATCTCCTGCATTCTCCCGGCCTGCCGTGGCTTGATGGTGGAAAGGCTGCGCTGAGCGCTGCCGCCGCTCATCA
TCATAATCCATGGACTGTCAGTCCGTTTTCTAAAACTCCTTTGCATCCATCTGCAGCCGGTGGACCAGGCGGTCCGCTTTCAGTT
TATCCCGGTGCGGGTGGCGGCTCCGGCGGCGGTTCAGGATCAAGCGTCGCGAGCCTGACTCCAACGGCTGCTCATAGTGGTA
GCCATCTGTTTGGGTTTCCCCCAACTCCCCCTAAAGAGGTCAGCCCAGATCCAAGCACGACCGGCGCCGCTTCCCCTGCATCCA
GCTCTGCCGGTGGGAGCGCGGCAAGAGGGGAAGATAAAGATGGAGTGAAATATCAAGTAAGTTTGACAGAATCAATGAAAA
TGGAGAGCGGATCTCCTCTCCGGCCCGGGCTTGCCACAATGGGTACTCAACCAGCCACCCATCATCCTATTCCAACATATCCTA
GCTACGTTCCCGCCGCCGCTCATGATTATTCATCTGGTCTGTTTCATCCTGGCGGGTTCTTGGGCGGCCCCGCGAGCAGTTTTA
CACCCAAACAACGTTCTAAAGCCAGGAGCTGCAGTGAGGGTAGAGAATGCGTTAATTGCGGCGCTACTGCAACACCGCTTTG
GCGAAGAGATGGTACTGGTCATTATTTGTGTAACGCATGCGGTCTGTATCATAAAATGAACGGTCAAAATCGGCCCCTGATTA
AACCTAAAAGGCGGCTTAGTGCGGCAAGGCGTGCAGGAACTTGCTGCGCCAACTGCCAAACAACGACAACTACTCTCTGGAG
ACGGAATGCAAATGGTGATCCGGTTTGTAATGCGTGCGGGCTGTATTATAAATTGCATAACGTCAATCGGCCGCTTACAATGA
AGAAAGAGGGAATACAAACCAGAAATCGCAAAATGAGCAATAAATCTAAGAAATCTAAGAAGGGAGCAGAATGTTTTGAGG
AACTCAGCAAATGTATGCAAGAAAAGTCCAGCCCTTTCTCCGCTGCAGCTTTGGCGGGGCATATGGCTCCCGTCGGTCATCTTC
CACCGTTTTCTCATAGCGGTCATATTCTTCCTACGCCCACCCCAATACATCCTTCTTCATCTTTGTCATTTGGACATCCTCATCCAT
CTTCTATGGTCACTGCAATGGGC
SEQ ID No. 28-GATA2 (from vector 3)
ATGGAAGTTGCCCCTGAACAACCAAGATGGATGGCCCATCCCGCGGTCCTCAACGCCCAACATCCAGATTCTCATCATCCCGGT
CTTGCCCATAATTATATGGAGCCTGCCCAACTTTTGCCCCCAGATGAAGTAGATGTATTCTTCAACCATCTGGATTCCCAAGGAA
ATCCTTATTACGCAAATCCAGCCCATGCACGCGCCAGGGTGAGCTATTCTCCAGCCCATGCAAGGCTTACGGGCGGACAAATG
TGTAGACCTCATCTGCTCCATAGTCCAGGCCTGCCATGGCTTGATGGCGGTAAGGCCGCGCTTAGCGCTGCAGCAGCTCATCA
TCATAATCCATGGACTGTCTCCCCATTTAGCAAAACACCGCTTCATCCTTCTGCAGCAGGTGGGCCGGGCGGACCTCTGAGCGT
ATATCCTGGCGCAGGTGGCGGCTCCGGCGGCGGGTCAGGAAGTAGTGTCGCGAGCCTGACTCCAACCGCTGCTCATAGCGGG
AGTCATCTGTTTGGGTTTCCGCCAACACCCCCTAAAGAGGTATCCCCAGATCCATCTACTACCGGTGCCGCCAGTCCCGCAAGC
TCCAGTGCAGGCGGCAGCGCTGCTAGAGGTGAAGATAAAGATGGGGTGAAATATCAAGTTTCCCTCACCGAAAGTATGAAAA
TGGAGTCCGGAAGCCCACTTCGACCTGGTCTTGCCACCATGGGAACACAACCCGCCACCCATCATCCTATACCAACGTATCCAA
GCTACGTCCCCGCTGCAGCTCATGATTATTCATCAGGGCTGTTTCATCCTGGTGGATTCTTGGGCGGTCCTGCTTCAAGTTTTAC
TCCGAAACAACGGTCTAAAGCCAGGAGCTGCTCCGAGGGAAGGGAATGCGTGAATTGCGGAGCAACCGCTACTCCCCTTTGG
CGAAGGGATGGTACTGGTCATTATCTTTGTAACGCATGCGGGCTGTATCATAAAATGAACGGCCAAAATCGGCCCCTGATTAA
ACCAAAACGGAGACTTTCCGCTGCACGGCGTGCTGGGACATGCTGCGCTAACTGCCAAACCACCACTACAACTCTCTGGCGGC
GGAATGCTAATGGTGATCCAGTGTGTAATGCTTGCGGGCTGTATTACAAACTTCATAACGTGAATCGCCCTCTTACGATGAAG
AAAGAGGGAATTCAAACGCGTAATCGTAAAATGTCAAATAAATCAAAGAAATCTAAGAAGGGTGCTGAATGTTTTGAGGAAC
TCAGCAAATGTATGCAAGAGAAAAGCAGCCCTTTTAGCGCGGCGGCGCTTGCAGGGCATATGGCCCCAGTAGGTCATCTGCCC
CCGTTTTCTCATAGCGGTCATATTCTCCCAACCCCAACACCGATACATCCAAGCAGCTCCTTGTCTTTTGGGCATCCTCATCCAA
GCTCAATGGTCACAGCAATGGGC
SEQ ID No. 29-GATA2 (from vector 4)
ATGGAAGTCGCCCCAGAACAACCTAGGTGGATGGCCCATCCTGCAGTACTCAACGCCCAACATCCTGATAGTCATCATCCCGG
ATTGGCACATAATTATATGGAGCCGGCTCAACTCCTTCCCCCGGATGAAGTCGATGTTTTCTTTAACCATTTGGATAGTCAAGG
AAATCCATATTACGCGAATCCTGCCCATGCCCGTGCCAGGGTGAGCTATTCCCCTGCCCATGCTAGACTCACTGGTGGTCAAAT
GTGTAGGCCCCATCTTCTCCATTCCCCTGGACTGCCTTGGTTGGATGGCGGAAAGGCTGCTCTGTCCGCTGCCGCCGCACATCA
TCATAATCCTTGGACAGTCAGTCCTTTTAGCAAAACACCACTCCATCCTTCTGCTGCCGGCGGACCCGGCGGTCCTCTGTCAGTT
TATCCGGGCGCTGGTGGCGGCTCTGGTGGTGGATCAGGTTCTTCCGTCGCAAGCCTGACTCCCACTGCCGCGCATTCCGGTAG
CCATCTCTTTGGTTTTCCCCCTACACCCCCAAAGGAAGTCTCCCCCGATCCCTCAACTACTGGCGCGGCTAGTCCTGCGTCCTCTT
CTGCCGGCGGCAGCGCTGCTCGCGGTGAAGATAAAGATGGAGTGAAATATCAAGTTAGCCTCACCGAATCTATGAAAATGGA
GAGCGGATCTCCTTTGCGTCCCGGGTTGGCCACCATGGGTACACAACCAGCCACCCATCATCCTATTCCTACTTATCCGAGCTA
CGTACCTGCTGCCGCACATGATTATTCATCTGGGCTGTTTCATCCAGGCGGGTTTCTCGGTGGCCCCGCAAGCTCATTTACTCCC
AAACAAAGGTCCAAAGCGCGGAGCTGCAGTGAGGGGAGAGAATGCGTGAATTGCGGCGCAACCGCTACGCCCTTGTGGAGA
AGGGATGGAACGGGGCATTATCTCTGTAACGCATGCGGGTTGTATCATAAAATGAACGGCCAAAATCGCCCCTTGATAAAACC
AAAGAGACGGTTGTCAGCTGCACGACGCGCTGGAACTTGCTGCGCCAACTGCCAAACCACCACAACAACATTGTGGCGGCGT
AATGCAAATGGCGATCCCGTGTGTAATGCTTGCGGTCTGTATTATAAATTGCATAACGTGAATAGACCCCTCACTATGAAGAAA
GAGGGCATACAAACCCGCAATCGCAAAATGTCAAATAAAAGCAAGAAGTCTAAGAAGGGCGCCGAATGTTTTGAAGAATTGT
CTAAATGTATGCAAGAAAAGAGCAGCCCATTTTCCGCCGCCGCACTCGCAGGCCATATGGCCCCAGTAGGTCATCTGCCCCCTT
TTAGTCATAGCGGGCATATTTTGCCTACGCCAACACCGATTCATCCTAGCAGCTCACTGTCTTTTGGGCATCCACATCCAAGTTC
TATGGTCACGGCTATGGGTTGA
SEQ ID No. 30-GATA2 (from vector 5)
ATGGAAGTAGCCCCTGAACAACCCAGATGGATGGCTCATCCAGCAGTATTGAACGCACAACATCCAGATTCCCATCATCCAGG
ATTGGCCCATAATTATATGGAGCCAGCCCAACTCTTGCCACCTGATGAAGTCGATGTTTTCTTTAACCATCTGGATAGTCAAGG
TAATCCTTATTACGCTAATCCAGCACATGCACGCGCTCGGGTTTCATATTCCCCTGCCCATGCAAGGCTTACAGGCGGACAAAT
GTGTCGTCCTCATCTGCTCCATTCCCCAGGCCTTCCATGGCTTGATGGCGGGAAGGCGGCGCTTTCAGCGGCGGCTGCACATC
ATCATAATCCGTGGACTGTCTCTCCTTTTAGTAAAACTCCGCTTCATCCGAGCGCAGCAGGCGGACCTGGCGGACCCCTGTCAG
TCTATCCTGGTGCCGGTGGCGGCTCCGGCGGTGGGTCCGGTTCCAGCGTTGCAAGCTTGACACCAACGGCGGCTCATTCAGGA
TCACATTTGTTTGGTTTTCCCCCAACCCCGCCGAAAGAGGTCAGTCCCGATCCAAGTACAACCGGAGCAGCAAGTCCCGCAAGC
TCATCTGCCGGCGGCTCCGCCGCTAGAGGCGAAGATAAAGATGGGGTAAAATATCAAGTCTCTCTTACCGAATCCATGAAAAT
GGAGTCTGGATCTCCTCTCCGGCCCGGGCTCGCGACCATGGGGACACAACCCGCAACCCATCATCCAATTCCAACTTATCCAAG
CTACGTTCCCGCTGCAGCACATGATTATTCATCTGGGCTGTTTCATCCGGGGGGATTTCTCGGTGGCCCAGCATCTTCTTTTACT
CCCAAACAACGGTCTAAAGCAAGGTCTTGCAGTGAGGGTAGGGAATGCGTGAATTGCGGAGCTACTGCAACTCCCCTGTGGC
GTCGCGATGGGACAGGTCATTATCTTTGTAACGCTTGCGGACTTTATCATAAAATGAACGGCCAAAATCGCCCGCTGATTAAAC
CTAAACGCAGGTTGTCAGCGGCTCGCAGGGGGGGGACATGCTGCGCCAACTGCCAAACCACCACTACAACACTGTGGCGAAG
AAATGCAAATGGCGATCCCGTGTGTAATGCTTGCGGGCTTTATTATAAACTCCATAACGTCAATCGGCCTCTTACAATGAAGAA
AGAGGGAATTCAAACCAGGAATAGGAAAATGTCAAATAAAAGCAAGAAATCAAAGAAGGGCGCCGAATGTTTTGAAGAATT
GTCTAAATGTATGCAAGAGAAATCTTCACCGTTTTCCGCCGCCGCGTTGGCCGGTCATATGGCTCCAGTCGGACATCTTCCGCC
ATTTTCACATAGTGGGCATATTCTTCCAACACCCACACCTATTCATCCTTCATCTTCTCTGTCATTTGGACATCCTCATCCTAGCTC
CATGGTTACTGCAATGGGC
SEQ ID No. 31-GATA2 (from vector 6)
ATGGAAGTTGCCCCGGAACAACCCCGTTGGATGGCCCATCCTGCAGTACTTAACGCCCAACATCCGGATTCCCATCATCCCGGA
CTCGCTCATAATTATATGGAGCCGGCTCAACTCTTGCCACCCGATGAAGTCGATGTGTTCTTTAACCATCTGGATTCTCAAGGG
AATCCATATTACGCAAATCCAGCACATGCCCGAGCCAGAGTTAGCTATTCACCTGCCCATGCAAGATTGACTGGTGGACAAAT
GTGTCGGCCCCATCTGCTCCATTCACCAGGGCTCCCATGGCTTGATGGCGGTAAGGCTGCGCTGAGCGCTGCCGCCGCACATC
ATCATAATCCATGGACAGTATCTCCCTTTTCTAAAACTCCCCTTCATCCTTCTGCAGCCGGTGGACCCGGCGGACCTTTGAGTGT
TTATCCTGGCGCAGGCGGCGGTTCCGGCGGCGGGAGTGGAAGCAGTGTCGCGAGTCTGACGCCCACTGCTGCACATTCAGGA
AGTCATCTGTTTGGGTTTCCCCCAACTCCACCCAAAGAGGTTTCCCCAGATCCAAGTACAACCGGCGCAGCCAGTCCCGCTTCC
TCTAGCGCCGGCGGCTCAGCTGCTAGAGGGGAAGATAAAGATGGTGTTAAATATCAAGTTAGCCTTACTGAATCTATGAAAAT
GGAGTCAGGGAGCCCGCTCAGACCCGGGCTGGCGACGATGGGTACACAACCGGCCACTCATCATCCTATTCCGACGTATCCG
AGTTACGTTCCCGCCGCCGCTCATGATTATTCTTCCGGCCTGTTTCATCCAGGCGGTTTTCTTGGCGGCCCCGCGAGCTCATTTA
CGCCCAAACAAAGGAGTAAAGCCAGATCTTGCTCTGAGGGGAGAGAATGCGTGAATTGCGGCGCGACGGCAACTCCCTTGTG
GCGCCGCGATGGTACTGGGCATTATCTCTGTAACGCATGCGGATTGTATCATAAAATGAACGGTCAAAATAGGCCTCTGATAA
AACCTAAACGGCGCCTTAGTGCGGCAAGGCGTGCAGGAACATGCTGCGCTAACTGCCAAACAACTACAACTACTTTGTGGCGG
CGCAATGCGAATGGTGATCCCGTGTGTAATGCTTGCGGATTGTATTATAAACTCCATAACGTGAATCGTCCTCTCACTATGAAG
AAGGAAGGAATTCAAACGCGCAATAGGAAAATGTCTAATAAAAGCAAGAAGTCCAAGAAGGGCGCTGAATGTTTTGAAGAAC
TCAGCAAATGTATGCAAGAGAAATCCAGTCCATTTTCAGCCGCCGCACTTGCAGGTCATATGGCTCCCGTCGGGCATTTGCCAC
CGTTTTCCCATAGCGGTCATATTCTCCCAACCCCTACTCCAATACATCCTTCTAGCTCTCTGAGTTTTGGGCATCCTCATCCCAGT
TCCATGGTCACAGCGATGGGTTGA
SEQ ID No. 32-GFI1B (from vector 1)
ATGCCGAGGAGCTTTCTCGTTAAATCCAAGAAAGCGCATACATATCATCAACCTAGGGTCCAAGAGGACGAGCCACTGTGGCC
ACCCGCTCTCACACCAGTTCCTCGGGATCAAGCACCTTCTAATTCACCCGTGTTGAGTACCTTGTTTCCGAATCAATGTCTCGAT
TGGACTAATCTGAAGCGGGAACCAGAATTGGAACAAGATCAAAATCTGGCACGCATGGCTCCCGCTCCTGAAGGTCCAATCGT
TCTCAGTCGGCCGCAAGACGGCGATTCCCCGCTTTCTGATAGCCCACCGTTTTATAAACCATCTTTTAGCTGGGATACGCTCGCT
ACGACATACGGTCATTCCTATAGACAAGCTCCGTCAACTATGCAAAGCGCTTTTCTCGAACATAGCGTTTCACTTTATGGATCAC
CCCTCGTCCCTTCCACCGAACCTGCTCTGGACTTTTCCCTGAGATATTCTCCCGGGATGGACGCCTATCATTGCGTTAAATGTAA
TAAAGTGTTTTCAACACCGCATGGTCTTGAGGTCCACGTACGCCGATCACACAGCGGCACTCGCCCGTTTGCATGCGATATTTG
TGGAAAGACTTTTGGACATGCCGTCTCTCTCGAACAACATACACATGTGCATTCACAAGAACGTTCCTTTGAATGTCGAATGTG
TGGGAAAGCGTTTAAAAGGTCAAGTACCCTCAGCACGCATCTTCTTATTCATTCTGATACCCGCCCTTATCCATGTCAATTTTGT
GGGAAACGCTTTCATCAGAAAAGTGATATGAAGAAACATACGTATATTCATACCGGGGAAAAGCCACATAAATGTCAAGTATG
TGGCAAAGCATTTTCCCAAAGTTCTAATTTGATAACGCATAGTCGAAAACATACTGGGTTTAAACCATTTTCCTGCGAACTCTGT
ACTAAGGGATTTCAAAGGAAAGTAGATCTTAGAAGGCATAGGGAATCCCAACATAACCTGAAA
SEQ ID No. 33-GFI1B (from vector 2)
ATGCCCCGGAGTTTTCTTGTTAAATCCAAGAAAGCCCATACATATCATCAACCTCGCGTCCAAGAGGACGAGCCACTGTGGCCC
CCAGCTCTGACGCCTGTACCTCGGGATCAAGCACCGTCTAATTCTCCCGTGCTGTCAACCCTGTTTCCTAATCAATGTCTTGATT
GGACTAATCTTAAGCGCGAACCAGAACTCGAACAAGATCAAAATCTGGCACGGATGGCTCCTGCCCCCGAAGGACCAATCGTA
CTCTCTCGGCCTCAAGACGGCGATAGTCCCTTGTCAGATTCACCCCCATTTTATAAACCATCCTTTAGTTGGGATACACTCGCTA
CTACATACGGTCATTCATATAGACAAGCTCCATCTACAATGCAATCCGCGTTTCTCGAACATAGCGTGAGTCTTTATGGTAGCCC
ACTGGTTCCAAGTACCGAACCGGCACTGGATTTCTCACTTCGTTATAGCCCTGGGATGGACGCTTATCATTGCGTCAAATGTAA
TAAAGTGTTTAGTACGCCCCATGGACTGGAAGTCCACGTAAGGCGGAGTCACTCCGGCACTAGACCTTTTGCATGCGATATAT
GTGGGAAGACTTTTGGTCATGCCGTTTCCCTTGAACAACATACTCATGTGCATTCTCAAGAAAGATCCTTTGAATGTCGGATGT
GTGGAAAAGCATTTAAAAGGTCATCAACTCTCAGCACTCATTTGCTGATTCATTCTGATACCCGCCCATATCCTTGTCAATTTTG
TGGTAAAAGGTTTCATCAGAAAAGCGATATGAAGAAACATACCTACATTCATACTGGAGAGAAGCCCCATAAATGTCAAGTTT
GTGGTAAGGCATTTTCACAATCTTCTAACCTGATAACACATTCCCGGAAACATACTGGATTTAAACCATTTTCTTGCGAACTCTG
TACAAAGGGTTTTCAAAGAAAAGTAGATCTCCGTAGGCATCGTGAATCACAACATAACCTGAAATAA
SEQ ID No. 34-GFI1B (from vector 3)
ATGCCCAGAAGCTTTCTCGTCAAATCAAAGAAAGCCCATACATATCATCAACCTCGGGTTCAAGAGGACGAGCCTTTGTGGCC
ACCAGCTCTCACACCAGTCCCTCGGGATCAAGCCCCTTCAAATAGTCCAGTACTCTCTACACTCTTTCCTAATCAATGTTTGGATT
GGACAAATCTGAAGCGGGAACCAGAACTTGAACAAGATCAAAATCTGGCACGGATGGCACCTGCGCCTGAAGGACCAATCGT
CTTGTCTAGGCCTCAAGACGGCGATAGCCCGCTCAGCGATTCCCCACCGTTTTATAAACCCAGTTTTAGCTGGGATACACTGGC
TACGACATACGGACATTCATATAGGCAAGCTCCTAGTACTATGCAATCCGCATTTCTCGAACATAGCGTGTCTTTGTATGGGAG
CCCGCTCGTACCTTCTACCGAACCTGCGCTGGATTTTAGTCTGCGTTATAGCCCTGGGATGGACGCCTATCATTGCGTCAAATG
TAATAAAGTCTTTAGCACTCCCCATGGCCTGGAAGTCCACGTCCGGCGAAGTCACTCTGGTACTAGACCATTTGCTTGCGATAT
TTGTGGAAAGACTTTTGGACATGCCGTCAGTCTTGAACAACATACACATGTGCATTCTCAAGAACGGTCCTTTGAATGTCGGAT
GTGTGGGAAAGCTTTTAAAAGAAGCTCTACTCTCTCAACGCATCTCTTGATTCATAGCGATACACGTCCTTATCCATGTCAATTT
TGTGGAAAACGCTTCCATCAAAAGTCAGATATGAAGAAACATACTTATATTCATACTGGCGAGAAACCACACAAATGTCAAGT
TTGCGGCAAAGCATTTTCCCAATCCTCAAATCTGATTACTCATTCCAGAAAACATACTGGGTTTAAACCATTTTCCTGCGAACTT
TGTACGAAGGGTTTTCAAAGGAAAGTTGATTTGCGACGCCATAGAGAATCCCAACATAACCTGAAA
SEQ ID No. 35-GFI1B (from vector 4)
ATGCCGAGATCATTTCTCGTCAAAAGTAAGAAGGCGCATACTTATCATCAACCTCGCGTTCAAGAGGACGAGCCCTTGTGGCC
CCCAGCGTTGACACCTGTTCCTCGCGATCAAGCACCCTCTAATAGTCCCGTGCTCTCAACCCTGTTTCCTAATCAATGTTTGGAT
TGGACAAATCTGAAGCGTGAACCTGAATTGGAACAAGATCAAAATCTTGCACGCATGGCGCCAGCGCCTGAAGGTCCAATCGT
CCTCAGTAGACCACAAGACGGAGATAGCCCGTTGAGCGATTCTCCACCCTTTTATAAACCATCCTTTAGTTGGGATACACTGGC
AACGACATACGGACATAGTTATAGGCAAGCTCCATCTACTATGCAATCTGCGTTTCTGGAACATAGCGTTTCTCTTTATGGGTCT
CCGCTGGTTCCATCAACAGAACCTGCGCTGGATTTTAGCCTGAGATATAGTCCTGGGATGGACGCATATCATTGCGTAAAATGT
AATAAAGTGTTTAGCACACCACATGGCCTGGAAGTCCACGTTCGGCGGAGCCACAGCGGCACAAGGCCATTTGCATGCGATAT
TTGTGGGAAGACGTTTGGACATGCCGTTTCCCTCGAACAACATACACATGTTCATAGCCAAGAAAGATCATTTGAATGTAGAAT
GTGTGGGAAAGCATTTAAACGGTCTAGCACACTCTCTACGCATCTCCTGATTCATAGCGATACTCGCCCATATCCTTGTCAATTT
TGCGGGAAAAGATTTCATCAAAAGTCAGATATGAAGAAGCATACGTATATTCATACCGGGGAGAAACCACATAAATGTCAAGT
CTGTGGCAAAGCCTTTTCCCAGTCATCTAATTTGATAACACATAGTAGGAAACATACCGGTTTTAAACCGTTTTCTTGCGAACTC
TGTACAAAGGGTTTTCAAAGAAAAGTTGATCTCCGTAGACATAGGGAATCCCAACATAACCTGAAA
SEQ ID No. 36-GFI1B (from vector 5)
ATGCCCCGAAGTTTTCTCGTTAAATCAAAGAAAGCACATACTTATCATCAACCTCGCGTCCAAGAGGACGAGCCACTTTGGCCA
CCCGCTCTGACACCTGTCCCGAGGGATCAAGCGCCGTCCAATTCACCCGTGTTGTCTACCCTCTTTCCCAATCAATGTTTGGATT
GGACAAATTTGAAGAGGGAACCAGAACTTGAACAAGATCAAAATCTTGCGCGGATGGCGCCTGCCCCGGAAGGGCCAATCGT
CCTCAGCCGGCCGCAAGACGGCGATTCCCCTCTTAGCGATAGCCCACCATTTTATAAACCCAGTTTTAGCTGGGATACTCTGGC
TACTACTTACGGACATTCATATAGACAAGCGCCTTCAACGATGCAAAGCGCATTTCTTGAACATAGTGTGTCCCTTTATGGTTCC
CCACTCGTTCCTTCCACAGAACCAGCTCTGGATTTCTCACTTCGATATTCTCCCGGGATGGACGCTTATCATTGCGTAAAATGTA
ATAAAGTGTTTTCTACACCGCATGGTCTGGAAGTCCACGTACGCCGTAGTCACTCCGGCACAAGGCCTTTTGCGTGCGATATTT
GTGGGAAGACGTTTGGTCATGCCGTATCACTCGAACAACATACACATGTGCATTCTCAAGAACGGAGTTTTGAATGTAGAATG
TGTGGTAAAGCTTTTAAAAGGTCATCAACTTTGAGTACTCATCTCTTGATACACAGCGATACAAGGCCGTATCCATGTCAATTTT
GTGGAAAACGGTTTCATCAAAAGTCAGATATGAAGAAACATACATATATTCATACCGGGGAAAAGCCCCATAAATGTCAAGTC
TGTGGTAAAGCGTTTTCTCAATCTAGTAATCTGATTACACATTCTCGTAAACATACTGGGTTTAAACCTTTCTCTTGCGAACTTTG
TACAAAGGGATTTCAACGGAAAGTCGATCTTCGTAGACATAGAGAATCCCAACATAACCTTAAATAA
SEQ ID No. 37-GFI1B (from vector 6)
ATGCCCCGTTCTTTCTTGGTAAAATCCAAGAAGGCGCATACATATCATCAACCACGGGTTCAAGAGGACGAGCCACTGTGGCC
GCCAGCTCTGACTCCCGTCCCTCGGGATCAAGCCCCCTCTAATTCTCCCGTGCTGTCAACATTGTTTCCTAATCAATGTTTGGATT
GGACTAATCTGAAGCGCGAACCAGAATTGGAACAAGATCAAAATCTCGCTCGGATGGCTCCTGCTCCGGAAGGTCCTATCGTT
CTTTCAAGACCACAAGACGGCGATTCACCCCTCTCAGATTCTCCCCCTTTCTATAAACCCAGCTTTAGTTGGGATACTCTGGCAA
CCACATACGGGCATTCTTATCGCCAAGCGCCAAGCACAATGCAAAGTGCATTTCTCGAACATTCAGTATCTCTTTATGGATCTCC
CCTGGTCCCTTCTACCGAACCAGCGCTTGACTTTTCCTTGAGGTATAGTCCCGGGATGGACGCATATCATTGCGTAAAATGTAA
TAAAGTTTTCAGCACGCCCCATGGATTGGAAGTCCACGTCAGGCGATCTCACTCTGGCACAAGACCTTTTGCATGCGATATTTG
TGGTAAGACGTTTGGACATGCCGTTTCACTCGAACAACATACACATGTTCATAGCCAAGAACGTTCATTTGAATGTAGAATGTG
TGGAAAAGCATTTAAAAGGAGCTCTACCCTTTCTACACATTTGCTGATTCATAGCGATACCCGACCTTATCCGTGTCAATTTTGT
GGAAAACGATTTCATCAGAAATCAGATATGAAGAAGCATACATATATACATACCGGGGAGAAACCCCATAAATGTCAAGTCTG
TGGCAAAGCATTTTCTCAATCAAGCAATCTGATTACTCATTCCAGAAAACATACTGGATTTAAACCCTTTTCATGCGAACTTTGT
ACTAAGGGATTTCAACGGAAAGTTGATTTGAGGAGACATCGAGAAAGTCAACATAACCTGAAA
SEQ ID No. 38-FOS (from vector 1)
ATGATGTTTTCCGGGTTTAATGCCGATTATGAAGCATCCAGTTCTAGGTGTAGTTCTGCAAGTCCCGCGGGCGACTCACTGTCA
TATTATCATAGCCCGGCTGATAGCTTTTCTTCTATGGGGTCACCCGTGAATGCCCAAGATTTCTGTACTGATTTGGCAGTGAGCT
CAGCTAATTTTATCCCTACTGTGACCGCTATAAGCACTTCTCCTGATCTCCAATGGCTCGTCCAACCAGCACTGGTTAGTTCAGT
CGCTCCCAGTCAAACACGGGCACCCCATCCGTTTGGGGTTCCAGCACCTTCAGCAGGCGCCTATAGCCGGGCCGGAGTCGTAA
AGACAATGACTGGTGGGAGAGCCCAATCAATCGGAAGAAGAGGAAAAGTCGAGCAACTTAGCCCTGAGGAAGAGGAAAAG
AGACGCATTAGGCGCGAGCGGAACAAAATGGCCGCCGCTAAGTGTCGAAATAGGCGTCGTGAACTCACAGACACCCTGCAGG
CCGAAACCGATCAGCTTGAGGACGAGAAAAGTGCACTGCAAACTGAAATCGCTAATCTTCTCAAAGAGAAAGAGAAGCTGGA
ATTTATTCTCGCTGCACATCGTCCAGCATGTAAAATTCCCGACGATCTCGGATTTCCGGAAGAAATGAGCGTAGCCAGTCTCGA
CCTTACCGGTGGACTCCCTGAAGTCGCGACACCTGAATCCGAAGAAGCGTTTACATTGCCCTTGCTGAACGATCCGGAACCAA
AACCAAGCGTCGAGCCCGTGAAAAGTATATCATCTATGGAACTTAAAACTGAACCTTTCGACGATTTTCTCTTTCCTGCTTCCAG
CCGCCCATCAGGGTCAGAAACCGCTAGATCTGTCCCCGATATGGATCTGAGCGGAAGCTTTTACGCTGCCGATTGGGAACCAC
TCCATTCAGGTAGTCTTGGCATGGGCCCTATGGCGACCGAACTTGAACCTCTTTGTACACCCGTTGTAACTTGCACCCCATCCTG
TACGGCCTATACCTCAAGTTTTGTATTTACATATCCTGAAGCCGATTCTTTTCCATCCTGCGCCGCCGCGCATCGAAAAGGGTCT
TCATCTAACGAACCATCTTCCGATAGTTTGAGTAGTCCAACCCTTCTTGCTCTCTAA
SEQ ID No. 39-FOS (from vector 2)
ATGATGTTTTCAGGGTTTAATGCCGATTATGAAGCCAGTTCTTCAAGATGTTCCTCAGCTTCCCCCGCAGGCGACTCCTTGTCCT
ATTATCATTCTCCAGCTGATAGCTTTAGTTCCATGGGGTCCCCCGTTAATGCCCAAGACTTTTGTACTGATCTCGCGGTTAGCAG
CGCAAATTTTATACCGACTGTGACAGCAATATCAACATCTCCAGATCTTCAATGGCTCGTTCAACCTGCACTGGTGAGCTCTGTC
GCACCTTCACAAACGCGGGCTCCACATCCATTTGGCGTTCCTGCACCAAGTGCAGGTGCCTATAGCCGCGCAGGGGTGGTTAA
AACAATGACTGGTGGAAGAGCCCAATCCATCGGGCGCCGGGGAAAAGTAGAGCAACTGTCCCCCGAGGAAGAGGAAAAGCG
TCGTATTAGGCGCGAGCGGAACAAAATGGCGGCTGCAAAGTGTAGAAATAGGCGCCGCGAATTGACAGACACCCTGCAGGC
AGAAACCGATCAGCTCGAGGACGAAAAGTCCGCCCTTCAAACTGAAATCGCAAATTTGCTTAAAGAGAAAGAGAAGTTGGAA
TTTATACTCGCTGCCCATCGGCCAGCATGTAAAATACCCGACGATTTGGGTTTTCCCGAGGAAATGTCAGTAGCCTCTCTCGAC
CTTACGGGCGGACTTCCCGAAGTGGCGACTCCTGAAAGTGAAGAAGCATTTACACTTCCCCTGCTGAACGATCCAGAACCTAA
ACCTTCCGTCGAGCCCGTAAAATCAATTTCTTCCATGGAATTGAAAACAGAACCTTTCGACGATTTTCTCTTTCCTGCCAGTTCA
CGCCCATCCGGGAGTGAAACCGCTCGAAGCGTTCCGGATATGGATCTTAGCGGCAGCTTTTACGCTGCTGATTGGGAACCGCT
TCATAGCGGGTCATTGGGTATGGGACCAATGGCTACCGAACTTGAACCATTGTGTACCCCAGTTGTGACATGCACCCCTTCTTG
TACCGCGTATACTTCAAGCTTTGTATTTACATATCCTGAAGCCGATTCATTTCCAAGTTGCGCCGCCGCTCATAGGAAAGGGTCT
AGTAGTAACGAACCAAGTTCAGATTCACTGTCCAGCCCTACATTGTTGGCTCTC
SEQ ID No. 40-FOS (from vector 3)
ATGATGTTTTCAGGGTTTAATGCCGATTATGAAGCCAGTTCATCTCGATGTTCATCAGCCAGCCCTGCTGGCGACTCACTGAGC
TATTATCATTCTCCAGCTGATTCATTTTCTTCTATGGGGAGCCCAGTAAATGCACAAGACTTTTGTACCGATCTCGCAGTTTCAA
GCGCGAATTTTATCCCAACCGTGACCGCTATTAGCACTTCTCCAGATCTCCAATGGCTTGTCCAACCGGCTCTGGTGAGTAGCG
TCGCTCCGAGCCAAACGAGGGCACCCCATCCGTTTGGTGTACCAGCACCAAGCGCAGGAGCATATTCTCGCGCGGGTGTAGTC
AAAACAATGACTGGCGGGCGCGCCCAAAGTATCGGTCGCAGAGGTAAAGTAGAGCAACTGAGCCCCGAGGAAGAGGAAAA
GCGACGGATTAGACGTGAGCGGAACAAAATGGCCGCCGCTAAGTGTCGGAATAGACGACGAGAACTTACAGACACTCTGCA
GGCCGAAACCGATCAGTTGGAAGACGAAAAGTCCGCACTCCAAACAGAAATCGCGAATCTTTTGAAAGAGAAAGAGAAGCTC
GAATTTATTCTTGCCGCACATCGCCCCGCGTGTAAAATACCCGACGATCTTGGATTTCCTGAGGAAATGTCCGTTGCAAGCCTG
GACCTCACCGGCGGACTTCCGGAAGTCGCAACACCCGAAAGTGAAGAAGCTTTTACTCTTCCCCTGCTGAACGATCCCGAACCT
AAACCTTCTGTTGAGCCCGTTAAATCAATAAGTAGTATGGAACTCAAAACTGAACCATTCGACGATTTTCTGTTTCCCGCTTCCA
GTAGACCAAGCGGGAGCGAAACTGCACGATCTGTCCCGGATATGGATCTGTCAGGCTCATTTTACGCGGCCGATTGGGAACC
ACTCCATTCCGGATCTCTCGGCATGGGACCAATGGCTACCGAACTTGAACCACTTTGTACCCCCGTCGTAACGTGCACACCATC
CTGTACCGCGTATACTAGCTCTTTTGTATTTACTTATCCGGAAGCGGATTCATTTCCAAGTTGCGCCGCCGCTCATAGAAAAGG
GTCTTCATCTAACGAACCGTCTTCCGATTCCCTGAGTAGTCCTACCCTTTTGGCATTGTAA
SEQ ID No. 41-FOS (from vector 4)
ATGATGTTTAGCGGATTTAATGCCGATTATGAAGCAAGCTCAAGCAGGTGTTCCTCAGCTTCTCCCGCTGGAGACTCTCTGTCA
TATTATCATAGCCCAGCCGATAGCTTTTCTTCCATGGGTTCCCCAGTTAATGCTCAAGATTTCTGTACCGATCTCGCGGTGAGTA
GCGCAAATTTTATACCAACCGTGACCGCTATTTCTACATCTCCCGATCTTCAATGGTTGGTCCAACCTGCTCTGGTGAGTTCTGT
CGCACCCTCTCAAACTCGGGCACCGCATCCCTTTGGCGTACCAGCACCAAGCGCCGGAGCCTATAGCCGGGCAGGGGTGGTC
AAAACTATGACCGGTGGGCGTGCCCAATCCATAGGACGCCGCGGGAAAGTCGAGCAATTGAGCCCGGAAGAAGAGGAAAAG
CGGCGCATTCGCCGGGAGCGAAACAAAATGGCCGCCGCTAAGTGTCGTAATAGACGCCGCGAACTCACCGACACGTTGCAGG
CTGAAACCGATCAGCTCGAGGACGAGAAAAGTGCGCTCCAAACGGAAATCGCTAATCTTCTCAAAGAAAAGGAGAAGCTTGA
ATTTATTTTGGCCGCCCATCGCCCCGCTTGTAAAATACCGGACGATCTTGGGTTTCCCGAGGAAATGAGTGTCGCCTCTCTGGA
CCTCACCGGCGGATTGCCTGAAGTGGCTACACCAGAATCCGAAGAAGCGTTTACGCTCCCCCTGCTGAACGATCCCGAACCAA
AACCATCCGTTGAGCCAGTGAAATCAATATCATCCATGGAACTTAAAACAGAACCTTTCGACGATTTTCTGTTTCCTGCCAGCTC
ACGCCCGAGCGGTTCAGAAACCGCGAGGTCTGTACCCGATATGGATCTGTCAGGTTCATTTTACGCTGCCGATTGGGAACCCT
TGCATAGCGGTTCTTTGGGCATGGGACCTATGGCTACCGAACTCGAACCTCTCTGTACACCTGTCGTGACATGCACACCTTCTT
GTACCGCCTATACCTCAAGTTTTGTGTTTACTTATCCAGAAGCCGATAGCTTTCCTAGTTGCGCTGCCGCACATCGTAAAGGAA
GTTCCTCAAACGAACCGAGTTCCGATTCTCTTAGTAGCCCAACCCTTTTGGCACTT
SEQ ID No. 42-FOS (from vector 5)
ATGATGTTTTCAGGATTTAATGCTGATTATGAAGCCAGCTCTAGTAGGTGTTCCTCTGCAAGCCCAGCTGGTGACTCCCTGTCA
TATTATCATAGTCCTGCTGATAGCTTTAGTTCTATGGGATCTCCAGTGAATGCCCAAGACTTTTGTACAGATCTCGCAGTAAGCA
GCGCAAATTTTATCCCGACTGTTACCGCTATTTCCACTTCACCCGATCTCCAATGGCTTGTCCAACCAGCGCTGGTGAGTAGTGT
TGCTCCGAGCCAAACACGGGCTCCCCATCCCTTTGGGGTGCCTGCTCCGAGCGCAGGCGCATATAGCCGTGCCGGAGTGGTG
AAAACAATGACCGGCGGACGGGCACAATCTATCGGGCGTAGAGGTAAAGTCGAGCAACTGAGTCCTGAGGAAGAGGAAAAG
AGACGGATTAGGCGGGAGCGCAACAAAATGGCAGCCGCAAAGTGTCGAAATAGAAGACGCGAACTCACGGACACCCTGCAG
GCAGAAACTGATCAGCTTGAGGACGAAAAGAGTGCCCTGCAAACTGAAATCGCAAATCTTCTCAAAGAAAAGGAGAAGCTGG
AATTTATTCTCGCCGCACATAGGCCCGCTTGTAAAATTCCCGACGATCTCGGATTTCCTGAGGAAATGAGCGTTGCCTCACTGG
ACCTTACAGGCGGGTTGCCCGAAGTGGCTACTCCCGAATCAGAAGAAGCATTTACTCTTCCCCTGCTGAACGATCCCGAACCAA
AACCATCCGTTGAGCCGGTGAAATCTATTAGTTCAATGGAATTGAAAACTGAACCTTTCGACGATTTTCTCTTTCCTGCCTCTAG
CCGACCATCAGGGTCCGAAACGGCTCGTAGTGTACCTGATATGGATCTGAGCGGTAGCTTTTACGCCGCCGATTGGGAACCTC
TCCATTCCGGTTCATTGGGCATGGGACCGATGGCTACTGAATTGGAACCGCTCTGTACCCCAGTAGTGACATGCACACCTTCAT
GTACAGCATATACATCAAGCTTTGTGTTTACTTATCCGGAAGCCGATTCTTTTCCTTCTTGCGCTGCCGCTCATAGGAAAGGCTC
CTCTTCTAACGAACCAAGTAGTGATTCACTGTCATCCCCTACACTTTTGGCTCTT
SEQ ID No. 43-FOS (from vector 6)
ATGATGTTTAGCGGATTTAATGCCGATTATGAAGCAAGCTCATCTCGATGTTCATCTGCAAGCCCAGCTGGTGACTCACTGAGC
TATTATCATAGCCCGGCTGATTCATTTTCATCTATGGGTTCCCCCGTTAATGCCCAAGACTTTTGTACCGATCTCGCAGTGAGCA
GCGCAAATTTTATCCCTACCGTGACCGCTATATCAACTAGCCCCGATTTGCAATGGCTTGTCCAACCAGCGTTGGTGTCAAGCG
TTGCACCCAGCCAAACTCGCGCTCCCCATCCCTTTGGCGTGCCAGCTCCGAGCGCAGGCGCCTATTCACGGGCAGGGGTAGTC
AAAACAATGACCGGCGGACGCGCTCAATCCATCGGAAGAAGAGGGAAAGTCGAGCAACTCTCCCCCGAGGAAGAGGAAAAG
CGACGCATTAGAAGAGAGCGGAACAAAATGGCCGCCGCTAAGTGTCGGAATAGACGCCGCGAACTCACCGACACTCTGCAGG
CCGAAACCGATCAGCTGGAAGACGAAAAGAGTGCCCTTCAAACTGAAATCGCAAATCTCTTGAAAGAAAAGGAGAAGCTGGA
ATTTATTCTTGCTGCACATCGTCCAGCATGTAAAATTCCGGACGATTTGGGGTTTCCCGAGGAAATGTCCGTTGCCTCTCTCGAC
TTGACCGGTGGGTTGCCTGAAGTAGCTACACCCGAAAGTGAAGAAGCGTTTACTCTTCCATTGCTTAACGATCCAGAACCAAA
ACCAAGCGTTGAGCCCGTAAAATCTATTTCTTCAATGGAACTCAAAACTGAACCATTCGACGATTTTCTCTTTCCTGCTTCTAGT
AGACCATCTGGCTCCGAAACCGCACGATCTGTACCTGATATGGATCTGTCCGGAAGCTTTTACGCCGCCGATTGGGAACCACTC
CATTCCGGTTCACTCGGAATGGGACCTATGGCTACGGAATTGGAACCTCTTTGTACCCCAGTTGTAACTTGCACCCCATCATGT
ACCGCCTATACCTCTAGCTTTGTATTTACGTATCCTGAAGCCGATAGCTTTCCATCTTGCGCCGCCGCGCATAGGAAAGGATCTT
CAAGTAACGAACCCAGTTCCGATAGTTTGAGTTCTCCGACACTTCTCGCTCTT
SEQ ID No. 44-GaGiFo
ATGGAAGTCGCTCCTGAACAACCACGGTGGATGGCTCATCCTGCAGTTCTTAACGCTCAACATCCAGATAGCCATCATCCTGGG
CTCGCTCATAATTATATGGAGCCTGCCCAACTCCTTCCCCCAGATGAAGTAGATGTATTCTTTAACCATCTTGATAGCCAAGGAA
ATCCTTATTACGCAAATCCTGCCCATGCCAGAGCCAGAGTAAGCTATTCACCAGCCCATGCAAGGCTTACTGGTGGACAAATGT
GTCGGCCCCATCTGCTGCATTCCCCCGGCCTGCCGTGGTTGGATGGCGGAAAGGCGGCTCTGAGTGCGGCGGCTGCACATCAT
CATAATCCTTGGACAGTCTCACCTTTCTCTAAAACTCCCCTTCATCCTAGTGCAGCCGGTGGTCCAGGTGGTCCGCTGTCCGTTT
ATCCCGGCGCCGGCGGCGGAAGTGGCGGCGGAAGTGGCAGTAGCGTAGCTTCATTGACACCCACAGCTGCTCATTCCGGAAG
TCATCTCTTTGGGTTTCCCCCTACCCCGCCGAAAGAGGTTTCCCCAGATCCGTCAACTACCGGTGCCGCTTCCCCTGCTAGTTCC
AGTGCCGGCGGAAGCGCTGCAAGGGGCGAAGATAAAGATGGAGTGAAATATCAAGTCTCCTTGACAGAATCCATGAAAATG
GAGTCCGGTTCTCCACTCAGGCCTGGACTCGCCACAATGGGTACTCAACCCGCCACCCATCATCCAATACCGACTTATCCTTCAT
ACGTACCAGCCGCCGCTCATGATTATTCATCAGGTTTGTTTCATCCAGGTGGATTTCTTGGCGGGCCTGCTAGTTCTTTTACACC
AAAACAAAGGTCCAAAGCCAGGTCTTGCAGTGAGGGGCGCGAATGCGTAAATTGCGGCGCTACCGCAACACCACTGTGGCGT
CGTGATGGAACAGGACATTATCTTTGTAACGCATGCGGGCTGTATCATAAAATGAACGGACAAAATCGGCCCCTGATTAAACC
TAAACGTCGCTTGTCTGCTGCAAGGGGGCTGGGACGTGCTGCGCCAACTGCCAAACAACCACAACAACACTGTGGCGTAGG
AATGCTAATGGCGATCCCGTATGTAATGCATGCGGTCTGTATTATAAACTTCATAACGTCAATCGGCCCCTTACTATGAAGAAA
GAGGGCATACAAACCCGAAATCGCAAAATGTCAAATAAATCTAAGAAATCCAAGAAGGGAGCCGAATGTTTTGAAGAACTCA
GCAAATGTATGCAAGAAAAGTCCAGCCCGTTTTCCGCCGCCGCTTTGGCCGGCCATATGGCCCCCGTCGGACATCTGCCACCG
TTTTCCCATAGCGGTCATATTCTTCCTACACCCACCCCAATTCATCCGTCTAGCTCCCTGTCTTTTGGGCATCCTCATCCTAGCTCA
ATGGTCACAGCTATGGGTGGCAGCGGCGCCACAAACTTCTCTCTGCTAAAGCAAGCAGGTGATGTTGAAGAAAACCCCGGGC
CTATGCCGAGGAGCTTTCTCGTTAAATCCAAGAAAGCGCATACATATCATCAACCTAGGGTCCAAGAGGACGAGCCACTGTGG
CCACCCGCTCTCACACCAGTTCCTCGGGATCAAGCACCTTCTAATTCACCCGTGTTGAGTACCTTGTTTCCGAATCAATGTCTCG
ATTGGACTAATCTGAAGCGGGAACCAGAATTGGAACAAGATCAAAATCTGGCACGCATGGCTCCCGCTCCTGAAGGTCCAATC
GTTCTCAGTCGGCCGCAAGACGGCGATTCCCCGCTTTCTGATAGCCCACCGTTTTATAAACCATCTTTTAGCTGGGATACGCTC
GCTACGACATACGGTCATTCCTATAGACAAGCTCCGTCAACTATGCAAAGCGCTTTTCTCGAACATAGCGTTTCACTTTATGGAT
CACCCCTCGTCCCTTCCACCGAACCTGCTCTGGACTTTTCCCTGAGATATTCTCCCGGGATGGACGCCTATCATTGCGTTAAATG
TAATAAAGTGTTTTCAACACCGCATGGTCTTGAGGTCCACGTACGCCGATCACACAGCGGCACTCGCCCGTTTGCATGCGATAT
TTGTGGAAAGACTTTTGGACATGCCGTCTCTCTCGAACAACATACACATGTGCATTCACAAGAACGTTCCTTTGAATGTCGAAT
GTGTGGGAAAGCGTTTAAAAGGTCAAGTACCCTCAGCACGCATCTTCTTATTCATTCTGATACCCGCCCTTATCCATGTCAATTT
TGTGGGAAACGCTTTCATCAGAAAAGTGATATGAAGAAACATACGTATATTCATACCGGGGAAAAGCCACATAAATGTCAAGT
ATGTGGCAAAGCATTTTCCCAAAGTTCTAATTTGATAACGCATAGTCGAAAACATACTGGGTTTAAACCATTTTCCTGCGAACTC
TGTACTAAGGGATTTCAAAGGAAAGTAGATCTTAGAAGGCATAGGGAATCCCAACATAACCTGAAAGGCTCCGGCGAGGGCA
GGGGAAGTCTTCTAACATGCGGGGACGTGGAGGAAAATCCCGGCCCAATGATGTTTTCCGGGTTTAATGCCGATTATGAAGC
ATCCAGTTCTAGGTGTAGTTCTGCAAGTCCCGCGGGCGACTCACTGTCATATTATCATAGCCCGGCTGATAGCTTTTCTTCTATG
GGGTCACCCGTGAATGCCCAAGATTTCTGTACTGATTTGGCAGTGAGCTCAGCTAATTTTATCCCTACTGTGACCGCTATAAGC
ACTTCTCCTGATCTCCAATGGCTCGTCCAACCAGCACTGGTTAGTTCAGTCGCTCCCAGTCAAACACGGGCACCCCATCCGTTTG
GGGTTCCAGCACCTTCAGCAGGCGCCTATAGCCGGGCCGGAGTCGTAAAGACAATGACTGGTGGGAGAGCCCAATCAATCGG
AAGAAGAGGAAAAGTCGAGCAACTTAGCCCTGAGGAAGAGGAAAAGAGACGCATTAGGCGCGAGCGGAACAAAATGGCCG
CCGCTAAGTGTCGAAATAGGCGTCGTGAACTCACAGACACCCTGCAGGCCGAAACCGATCAGCTTGAGGACGAGAAAAGTGC
ACTGCAAACTGAAATCGCTAATCTTCTCAAAGAGAAAGAGAAGCTGGAATTTATTCTCGCTGCACATCGTCCAGCATGTAAAAT
TCCCGACGATCTCGGATTTCCGGAAGAAATGAGCGTAGCCAGTCTCGACCTTACCGGTGGACTCCCTGAAGTCGCGACACCTG
AATCCGAAGAAGCGTTTACATTGCCCTTGCTGAACGATCCGGAACCAAAACCAAGCGTCGAGCCCGTGAAAAGTATATCATCT
ATGGAACTTAAAACTGAACCTTTCGACGATTTTCTCTTTCCTGCTTCCAGCCGCCCATCAGGGTCAGAAACCGCTAGATCTGTCC
CCGATATGGATCTGAGCGGAAGCTTTTACGCTGCCGATTGGGAACCACTCCATTCAGGTAGTCTTGGCATGGGCCCTATGGCG
ACCGAACTTGAACCTCTTTGTACACCCGTTGTAACTTGCACCCCATCCTGTACGGCCTATACCTCAAGTTTTGTATTTACATATCC
TGAAGCCGATTCTTTTCCATCCTGCGCCGCCGCGCATCGAAAAGGGTCTTCATCTAACGAACCATCTTCCGATAGTTTGAGTAG
TCCAACCCTTCTTGCTCTCTAA
SEQ ID No. 45-GaFoGi
ATGGAAGTCGCCCCGGAACAACCACGATGGATGGCACATCCTGCAGTTCTCAACGCACAACATCCTGATAGTCATCATCCAGG
GTTGGCTCATAATTATATGGAGCCTGCCCAACTCCTTCCCCCTGATGAAGTTGATGTGTTCTTTAACCATCTGGATTCTCAAGGA
AATCCGTATTACGCTAATCCAGCCCATGCACGCGCACGTGTTTCATATAGTCCTGCCCATGCGAGGCTTACAGGCGGGCAAAT
GTGTCGTCCCCATCTCCTGCATTCTCCCGGCCTGCCGTGGCTTGATGGTGGAAAGGCTGCGCTGAGCGCTGCCGCCGCTCATCA
TCATAATCCATGGACTGTCAGTCCGTTTTCTAAAACTCCTTTGCATCCATCTGCAGCCGGTGGACCAGGCGGTCCGCTTTCAGTT
TATCCCGGTGCGGGTGGCGGCTCCGGCGGCGGTTCAGGATCAAGCGTCGCGAGCCTGACTCCAACGGCTGCTCATAGTGGTA
GCCATCTGTTTGGGTTTCCCCCAACTCCCCCTAAAGAGGTCAGCCCAGATCCAAGCACGACCGGCGCCGCTTCCCCTGCATCCA
GCTCTGCCGGTGGGAGCGCGGCAAGAGGGGAAGATAAAGATGGAGTGAAATATCAAGTAAGTTTGACAGAATCAATGAAAA
TGGAGAGCGGATCTCCTCTCCGGCCCGGGCTTGCCACAATGGGTACTCAACCAGCCACCCATCATCCTATTCCAACATATCCTA
GCTACGTTCCCGCCGCCGCTCATGATTATTCATCTGGTCTGTTTCATCCTGGCGGGTTCTTGGGCGGCCCCGCGAGCAGTTTTA
CACCCAAACAACGTTCTAAAGCCAGGAGCTGCAGTGAGGGTAGAGAATGCGTTAATTGCGGCGCTACTGCAACACCGCTTTG
GCGAAGAGATGGTACTGGTCATTATTTGTGTAACGCATGCGGTCTGTATCATAAAATGAACGGTCAAAATCGGCCCCTGATTA
AACCTAAAAGGCGGCTTAGTGCGGCAAGGCGTGCAGGAACTTGCTGCGCCAACTGCCAAACAACGACAACTACTCTCTGGAG
ACGGAATGCAAATGGTGATCCGGTTTGTAATGCGTGCGGGCTGTATTATAAATTGCATAACGTCAATCGGCCGCTTACAATGA
AGAAAGAGGGAATACAAACCAGAAATCGCAAAATGAGCAATAAATCTAAGAAATCTAAGAAGGGAGCAGAATGTTTTGAGG
AACTCAGCAAATGTATGCAAGAAAAGTCCAGCCCTTTCTCCGCTGCAGCTTTGGCGGGGCATATGGCTCCCGTCGGTCATCTTC
CACCGTTTTCTCATAGCGGTCATATTCTTCCTACGCCCACCCCAATACATCCTTCTTCATCTTTGTCATTTGGACATCCTCATCCAT
CTTCTATGGTCACTGCAATGGGCGGCAGCGGCGCCACAAACTTCTCTCTGCTAAAGCAAGCAGGTGATGTTGAAGAAAACCCC
GGGCCTATGATGTTTTCAGGGTTTAATGCCGATTATGAAGCCAGTTCTTCAAGATGTTCCTCAGCTTCCCCCGCAGGCGACTCC
TTGTCCTATTATCATTCTCCAGCTGATAGCTTTAGTTCCATGGGGTCCCCCGTTAATGCCCAAGACTTTTGTACTGATCTCGCGGT
TAGCAGCGCAAATTTTATACCGACTGTGACAGCAATATCAACATCTCCAGATCTTCAATGGCTCGTTCAACCTGCACTGGTGAG
CTCTGTCGCACCTTCACAAACGCGGGCTCCACATCCATTTGGCGTTCCTGCACCAAGTGCAGGTGCCTATAGCCGCGCAGGGG
TGGTTAAAACAATGACTGGTGGAAGAGCCCAATCCATCGGGCGCCGGGGAAAAGTAGAGCAACTGTCCCCCGAGGAAGAGG
AAAAGCGTCGTATTAGGCGCGAGCGGAACAAAATGGCGGCTGCAAAGTGTAGAAATAGGCGCCGCGAATTGACAGACACCC
TGCAGGCAGAAACCGATCAGCTCGAGGACGAAAAGTCCGCCCTTCAAACTGAAATCGCAAATTTGCTTAAAGAGAAAGAGAA
GTTGGAATTTATACTCGCTGCCCATCGGCCAGCATGTAAAATACCCGACGATTTGGGTTTTCCCGAGGAAATGTCAGTAGCCTC
TCTCGACCTTACGGGCGGACTTCCCGAAGTGGCGACTCCTGAAAGTGAAGAAGCATTTACACTTCCCCTGCTGAACGATCCAG
AACCTAAACCTTCCGTCGAGCCCGTAAAATCAATTTCTTCCATGGAATTGAAAACAGAACCTTTCGACGATTTTCTCTTTCCTGC
CAGTTCACGCCCATCCGGGAGTGAAACCGCTCGAAGCGTTCCGGATATGGATCTTAGCGGCAGCTTTTACGCTGCTGATTGGG
AACCGCTTCATAGCGGGTCATTGGGTATGGGACCAATGGCTACCGAACTTGAACCATTGTGTACCCCAGTTGTGACATGCACC
CCTTCTTGTACCGCGTATACTTCAAGCTTTGTATTTACATATCCTGAAGCCGATTCATTTCCAAGTTGCGCCGCCGCTCATAGGA
AAGGGTCTAGTAGTAACGAACCAAGTTCAGATTCACTGTCCAGCCCTACATTGTTGGCTCTCGGCTCCGGCGAGGGCAGGGGA
AGTCTTCTAACATGCGGGGACGTGGAGGAAAATCCCGGCCCAATGCCCCGGAGTTTTCTTGTTAAATCCAAGAAAGCCCATAC
ATATCATCAACCTCGCGTCCAAGAGGACGAGCCACTGTGGCCCCCAGCTCTGACGCCTGTACCTCGGGATCAAGCACCGTCTA
ATTCTCCCGTGCTGTCAACCCTGTTTCCTAATCAATGTCTTGATTGGACTAATCTTAAGCGCGAACCAGAACTCGAACAAGATCA
AAATCTGGCACGGATGGCTCCTGCCCCCGAAGGACCAATCGTACTCTCTCGGCCTCAAGACGGCGATAGTCCCTTGTCAGATTC
ACCCCCATTTTATAAACCATCCTTTAGTTGGGATACACTCGCTACTACATACGGTCATTCATATAGACAAGCTCCATCTACAATG
CAATCCGCGTTTCTCGAACATAGCGTGAGTCTTTATGGTAGCCCACTGGTTCCAAGTACCGAACCGGCACTGGATTTCTCACTT
CGTTATAGCCCTGGGATGGACGCTTATCATTGCGTCAAATGTAATAAAGTGTTTAGTACGCCCCATGGACTGGAAGTCCACGT
AAGGCGGAGTCACTCCGGCACTAGACCTTTTGCATGCGATATATGTGGGAAGACTTTTGGTCATGCCGTTTCCCTTGAACAACA
TACTCATGTGCATTCTCAAGAAAGATCCTTTGAATGTCGGATGTGTGGAAAAGCATTTAAAAGGTCATCAACTCTCAGCACTCA
TTTGCTGATTCATTCTGATACCCGCCCATATCCTTGTCAATTTTGTGGTAAAAGGTTTCATCAGAAAAGCGATATGAAGAAACAT
ACCTACATTCATACTGGAGAGAAGCCCCATAAATGTCAAGTTTGTGGTAAGGCATTTTCACAATCTTCTAACCTGATAACACATT
CCCGGAAACATACTGGATTTAAACCATTTTCTTGCGAACTCTGTACAAAGGGTTTTCAAAGAAAAGTAGATCTCCGTAGGCATC
GTGAATCACAACATAACCTGAAATAA
SEQ ID No. 46-GiGaFo
ATGCCCAGAAGCTTTCTCGTCAAATCAAAGAAAGCCCATACATATCATCAACCTCGGGTTCAAGAGGACGAGCCTTTGTGGCC
ACCAGCTCTCACACCAGTCCCTCGGGATCAAGCCCCTTCAAATAGTCCAGTACTCTCTACACTCTTTCCTAATCAATGTTTGGATT
GGACAAATCTGAAGCGGGAACCAGAACTTGAACAAGATCAAAATCTGGCACGGATGGCACCTGCGCCTGAAGGACCAATCGT
CTTGTCTAGGCCTCAAGACGGCGATAGCCCGCTCAGCGATTCCCCACCGTTTTATAAACCCAGTTTTAGCTGGGATACACTGGC
TACGACATACGGACATTCATATAGGCAAGCTCCTAGTACTATGCAATCCGCATTTCTCGAACATAGCGTGTCTTTGTATGGGAG
CCCGCTCGTACCTTCTACCGAACCTGCGCTGGATTTTAGTCTGCGTTATAGCCCTGGGATGGACGCCTATCATTGCGTCAAATG
TAATAAAGTCTTTAGCACTCCCCATGGCCTGGAAGTCCACGTCCGGCGAAGTCACTCTGGTACTAGACCATTTGCTTGCGATAT
TTGTGGAAAGACTTTTGGACATGCCGTCAGTCTTGAACAACATACACATGTGCATTCTCAAGAACGGTCCTTTGAATGTCGGAT
GTGTGGGAAAGCTTTTAAAAGAAGCTCTACTCTCTCAACGCATCTCTTGATTCATAGCGATACACGTCCTTATCCATGTCAATTT
TGTGGAAAACGCTTCCATCAAAAGTCAGATATGAAGAAACATACTTATATTCATACTGGCGAGAAACCACACAAATGTCAAGT
TTGCGGCAAAGCATTTTCCCAATCCTCAAATCTGATTACTCATTCCAGAAAACATACTGGGTTTAAACCATTTTCCTGCGAACTT
TGTACGAAGGGTTTTCAAAGGAAAGTTGATTTGCGACGCCATAGAGAATCCCAACATAACCTGAAAGGCAGCGGCGCCACAA
ACTTCTCTCTGCTAAAGCAAGCAGGTGATGTTGAAGAAAACCCCGGGCCTATGGAAGTTGCCCCTGAACAACCAAGATGGATG
GCCCATCCCGCGGTCCTCAACGCCCAACATCCAGATTCTCATCATCCCGGTCTTGCCCATAATTATATGGAGCCTGCCCAACTTT
TGCCCCCAGATGAAGTAGATGTATTCTTCAACCATCTGGATTCCCAAGGAAATCCTTATTACGCAAATCCAGCCCATGCACGCG
CCAGGGTGAGCTATTCTCCAGCCCATGCAAGGCTTACGGGCGGACAAATGTGTAGACCTCATCTGCTCCATAGTCCAGGCCTG
CCATGGCTTGATGGCGGTAAGGCCGCGCTTAGCGCTGCAGCAGCTCATCATCATAATCCATGGACTGTCTCCCCATTTAGCAAA
ACACCGCTTCATCCTTCTGCAGCAGGTGGGCCGGGCGGACCTCTGAGCGTATATCCTGGCGCAGGTGGCGGCTCCGGCGGCG
GGTCAGGAAGTAGTGTCGCGAGCCTGACTCCAACCGCTGCTCATAGCGGGAGTCATCTGTTTGGGTTTCCGCCAACACCCCCT
AAAGAGGTATCCCCAGATCCATCTACTACCGGTGCCGCCAGTCCCGCAAGCTCCAGTGCAGGCGGCAGCGCTGCTAGAGGTG
AAGATAAAGATGGGGTGAAATATCAAGTTTCCCTCACCGAAAGTATGAAAATGGAGTCCGGAAGCCCACTTCGACCTGGTCTT
GCCACCATGGGAACACAACCCGCCACCCATCATCCTATACCAACGTATCCAAGCTACGTCCCCGCTGCAGCTCATGATTATTCAT
CAGGGCTGTTTCATCCTGGTGGATTCTTGGGCGGTCCTGCTTCAAGTTTTACTCCGAAACAACGGTCTAAAGCCAGGAGCTGCT
CCGAGGGAAGGGAATGCGTGAATTGCGGAGCAACCGCTACTCCCCTTTGGCGAAGGGATGGTACTGGTCATTATCTTTGTAAC
GCATGCGGGCTGTATCATAAAATGAACGGCCAAAATCGGCCCCTGATTAAACCAAAACGGAGACTTTCCGCTGCACGGCGTGC
TGGGACATGCTGCGCTAACTGCCAAACCACCACTACAACTCTCTGGCGGCGGAATGCTAATGGTGATCCAGTGTGTAATGCTT
GCGGGCTGTATTACAAACTTCATAACGTGAATCGCCCTCTTACGATGAAGAAAGAGGGAATTCAAACGCGTAATCGTAAAATG
TCAAATAAATCAAAGAAATCTAAGAAGGGTGCTGAATGTTTTGAGGAACTCAGCAAATGTATGCAAGAGAAAAGCAGCCCTTT
TAGCGCGGCGGCGCTTGCAGGGCATATGGCCCCAGTAGGTCATCTGCCCCCGTTTTCTCATAGCGGTCATATTCTCCCAACCCC
AACACCGATACATCCAAGCAGCTCCTTGTCTTTTGGGCATCCTCATCCAAGCTCAATGGTCACAGCAATGGGCGGCTCCGGCGA
GGGCAGGGGAAGTCTTCTAACATGCGGGGACGTGGAGGAAAATCCCGGCCCAATGATGTTTTCAGGGTTTAATGCCGATTAT
GAAGCCAGTTCATCTCGATGTTCATCAGCCAGCCCTGCTGGCGACTCACTGAGCTATTATCATTCTCCAGCTGATTCATTTTCTT
CTATGGGGAGCCCAGTAAATGCACAAGACTTTTGTACCGATCTCGCAGTTTCAAGCGCGAATTTTATCCCAACCGTGACCGCTA
TTAGCACTTCTCCAGATCTCCAATGGCTTGTCCAACCGGCTCTGGTGAGTAGCGTCGCTCCGAGCCAAACGAGGGCACCCCATC
CGTTTGGTGTACCAGCACCAAGCGCAGGAGCATATTCTCGCGCGGGTGTAGTCAAAACAATGACTGGCGGGCGCGCCCAAAG
TATCGGTCGCAGAGGTAAAGTAGAGCAACTGAGCCCCGAGGAAGAGGAAAAGCGACGGATTAGACGTGAGCGGAACAAAAT
GGCCGCCGCTAAGTGTCGGAATAGACGACGAGAACTTACAGACACTCTGCAGGCCGAAACCGATCAGTTGGAAGACGAAAA
GTCCGCACTCCAAACAGAAATCGCGAATCTTTTGAAAGAGAAAGAGAAGCTCGAATTTATTCTTGCCGCACATCGCCCCGCGT
GTAAAATACCCGACGATCTTGGATTTCCTGAGGAAATGTCCGTTGCAAGCCTGGACCTCACCGGCGGACTTCCGGAAGTCGCA
ACACCCGAAAGTGAAGAAGCTTTTACTCTTCCCCTGCTGAACGATCCCGAACCTAAACCTTCTGTTGAGCCCGTTAAATCAATA
AGTAGTATGGAACTCAAAACTGAACCATTCGACGATTTTCTGTTTCCCGCTTCCAGTAGACCAAGCGGGAGCGAAACTGCACG
ATCTGTCCCGGATATGGATCTGTCAGGCTCATTTTACGCGGCCGATTGGGAACCACTCCATTCCGGATCTCTCGGCATGGGACC
AATGGCTACCGAACTTGAACCACTTTGTACCCCCGTCGTAACGTGCACACCATCCTGTACCGCGTATACTAGCTCTTTTGTATTT
ACTTATCCGGAAGCGGATTCATTTCCAAGTTGCGCCGCCGCTCATAGAAAAGGGTCTTCATCTAACGAACCGTCTTCCGATTCC
CTGAGTAGTCCTACCCTTTTGGCATTGTAA
SEQ ID No. 47-GiFoGa
ATGCCGAGATCATTTCTCGTCAAAAGTAAGAAGGCGCATACTTATCATCAACCTCGCGTTCAAGAGGACGAGCCCTTGTGGCC
CCCAGCGTTGACACCTGTTCCTCGCGATCAAGCACCCTCTAATAGTCCCGTGCTCTCAACCCTGTTTCCTAATCAATGTTTGGAT
TGGACAAATCTGAAGCGTGAACCTGAATTGGAACAAGATCAAAATCTTGCACGCATGGCGCCAGCGCCTGAAGGTCCAATCGT
CCTCAGTAGACCACAAGACGGAGATAGCCCGTTGAGCGATTCTCCACCCTTTTATAAACCATCCTTTAGTTGGGATACACTGGC
AACGACATACGGACATAGTTATAGGCAAGCTCCATCTACTATGCAATCTGCGTTTCTGGAACATAGCGTTTCTCTTTATGGGTCT
CCGCTGGTTCCATCAACAGAACCTGCGCTGGATTTTAGCCTGAGATATAGTCCTGGGATGGACGCATATCATTGCGTAAAATGT
AATAAAGTGTTTAGCACACCACATGGCCTGGAAGTCCACGTTCGGCGGAGCCACAGCGGCACAAGGCCATTTGCATGCGATAT
TTGTGGGAAGACGTTTGGACATGCCGTTTCCCTCGAACAACATACACATGTTCATAGCCAAGAAAGATCATTTGAATGTAGAAT
GTGTGGGAAAGCATTTAAACGGTCTAGCACACTCTCTACGCATCTCCTGATTCATAGCGATACTCGCCCATATCCTTGTCAATTT
TGCGGGAAAAGATTTCATCAAAAGTCAGATATGAAGAAGCATACGTATATTCATACCGGGGAGAAACCACATAAATGTCAAGT
CTGTGGCAAAGCCTTTTCCCAGTCATCTAATTTGATAACACATAGTAGGAAACATACCGGTTTTAAACCGTTTTCTTGCGAACTC
TGTACAAAGGGTTTTCAAAGAAAAGTTGATCTCCGTAGACATAGGGAATCCCAACATAACCTGAAAGGCAGCGGCGCCACAA
ACTTCTCTCTGCTAAAGCAAGCAGGTGATGTTGAAGAAAACCCCGGGCCTATGATGTTTAGCGGATTTAATGCCGATTATGAA
GCAAGCTCAAGCAGGTGTTCCTCAGCTTCTCCCGCTGGAGACTCTCTGTCATATTATCATAGCCCAGCCGATAGCTTTTCTTCCA
TGGGTTCCCCAGTTAATGCTCAAGATTTCTGTACCGATCTCGCGGTGAGTAGCGCAAATTTTATACCAACCGTGACCGCTATTTC
TACATCTCCCGATCTTCAATGGTTGGTCCAACCTGCTCTGGTGAGTTCTGTCGCACCCTCTCAAACTCGGGCACCGCATCCCTTT
GGCGTACCAGCACCAAGCGCCGGAGCCTATAGCCGGGCAGGGGTGGTCAAAACTATGACCGGTGGGCGTGCCCAATCCATA
GGACGCCGCGGGAAAGTCGAGCAATTGAGCCCGGAAGAAGAGGAAAAGCGGCGCATTCGCCGGGAGCGAAACAAAATGGC
CGCCGCTAAGTGTCGTAATAGACGCCGCGAACTCACCGACACGTTGCAGGCTGAAACCGATCAGCTCGAGGACGAGAAAAGT
GCGCTCCAAACGGAAATCGCTAATCTTCTCAAAGAAAAGGAGAAGCTTGAATTTATTTTGGCCGCCCATCGCCCCGCTTGTAAA
ATACCGGACGATCTTGGGTTTCCCGAGGAAATGAGTGTCGCCTCTCTGGACCTCACCGGCGGATTGCCTGAAGTGGCTACACC
AGAATCCGAAGAAGCGTTTACGCTCCCCCTGCTGAACGATCCCGAACCAAAACCATCCGTTGAGCCAGTGAAATCAATATCATC
CATGGAACTTAAAACAGAACCTTTCGACGATTTTCTGTTTCCTGCCAGCTCACGCCCGAGCGGTTCAGAAACCGCGAGGTCTGT
ACCCGATATGGATCTGTCAGGTTCATTTTACGCTGCCGATTGGGAACCCTTGCATAGCGGTTCTTTGGGCATGGGACCTATGGC
TACCGAACTCGAACCTCTCTGTACACCTGTCGTGACATGCACACCTTCTTGTACCGCCTATACCTCAAGTTTTGTGTTTACTTATC
CAGAAGCCGATAGCTTTCCTAGTTGCGCTGCCGCACATCGTAAAGGAAGTTCCTCAAACGAACCGAGTTCCGATTCTCTTAGTA
GCCCAACCCTTTTGGCACTTGGCTCCGGCGAGGGCAGGGGAAGTCTTCTAACATGCGGGGACGTGGAGGAAAATCCCGGCCC
AATGGAAGTCGCCCCAGAACAACCTAGGTGGATGGCCCATCCTGCAGTACTCAACGCCCAACATCCTGATAGTCATCATCCCG
GATTGGCACATAATTATATGGAGCCGGCTCAACTCCTTCCCCCGGATGAAGTCGATGTTTTCTTTAACCATTTGGATAGTCAAG
GAAATCCATATTACGCGAATCCTGCCCATGCCCGTGCCAGGGTGAGCTATTCCCCTGCCCATGCTAGACTCACTGGTGGTCAAA
TGTGTAGGCCCCATCTTCTCCATTCCCCTGGACTGCCTTGGTTGGATGGCGGAAAGGCTGCTCTGTCCGCTGCCGCCGCACATC
ATCATAATCCTTGGACAGTCAGTCCTTTTAGCAAAACACCACTCCATCCTTCTGCTGCCGGCGGACCCGGCGGTCCTCTGTCAGT
TTATCCGGGCGCTGGTGGCGGCTCTGGTGGTGGATCAGGTTCTTCCGTCGCAAGCCTGACTCCCACTGCCGCGCATTCCGGTA
GCCATCTCTTTGGTTTTCCCCCTACACCCCCAAAGGAAGTCTCCCCCGATCCCTCAACTACTGGCGCGGCTAGTCCTGCGTCCTC
TTCTGCCGGCGGCAGCGCTGCTCGCGGTGAAGATAAAGATGGAGTGAAATATCAAGTTAGCCTCACCGAATCTATGAAAATG
GAGAGCGGATCTCCTTTGCGTCCCGGGTTGGCCACCATGGGTACACAACCAGCCACCCATCATCCTATTCCTACTTATCCGAGC
TACGTACCTGCTGCCGCACATGATTATTCATCTGGGCTGTTTCATCCAGGCGGGTTTCTCGGTGGCCCCGCAAGCTCATTTACTC
CCAAACAAAGGTCCAAAGCGCGGAGCTGCAGTGAGGGGAGAGAATGCGTGAATTGCGGCGCAACCGCTACGCCCTTGTGGA
GAAGGGATGGAACGGGGCATTATCTCTGTAACGCATGCGGGTTGTATCATAAAATGAACGGCCAAAATCGCCCCTTGATAAA
ACCAAAGAGACGGTTGTCAGCTGCACGACGCGCTGGAACTTGCTGCGCCAACTGCCAAACCACCACAACAACATTGTGGCGG
CGTAATGCAAATGGCGATCCCGTGTGTAATGCTTGCGGTCTGTATTATAAATTGCATAACGTGAATAGACCCCTCACTATGAAG
AAAGAGGGCATACAAACCCGCAATCGCAAAATGTCAAATAAAAGCAAGAAGTCTAAGAAGGGCGCCGAATGTTTTGAAGAAT
TGTCTAAATGTATGCAAGAAAAGAGCAGCCCATTTTCCGCCGCCGCACTCGCAGGCCATATGGCCCCAGTAGGTCATCTGCCCC
CTTTTAGTCATAGCGGGCATATTTTGCCTACGCCAACACCGATTCATCCTAGCAGCTCACTGTCTTTTGGGCATCCACATCCAAG
TTCTATGGTCACGGCTATGGGTTGA
SEQ ID No. 48-FoGaGi
ATGATGTTTTCAGGATTTAATGCTGATTATGAAGCCAGCTCTAGTAGGTGTTCCTCTGCAAGCCCAGCTGGTGACTCCCTGTCA
TATTATCATAGTCCTGCTGATAGCTTTAGTTCTATGGGATCTCCAGTGAATGCCCAAGACTTTTGTACAGATCTCGCAGTAAGCA
GCGCAAATTTTATCCCGACTGTTACCGCTATTTCCACTTCACCCGATCTCCAATGGCTTGTCCAACCAGCGCTGGTGAGTAGTGT
TGCTCCGAGCCAAACACGGGCTCCCCATCCCTTTGGGGTGCCTGCTCCGAGCGCAGGCGCATATAGCCGTGCCGGAGTGGTG
AAAACAATGACCGGCGGACGGGCACAATCTATCGGGCGTAGAGGTAAAGTCGAGCAACTGAGTCCTGAGGAAGAGGAAAAG
AGACGGATTAGGCGGGAGCGCAACAAAATGGCAGCCGCAAAGTGTCGAAATAGAAGACGCGAACTCACGGACACCCTGCAG
GCAGAAACTGATCAGCTTGAGGACGAAAAGAGTGCCCTGCAAACTGAAATCGCAAATCTTCTCAAAGAAAAGGAGAAGCTGG
AATTTATTCTCGCCGCACATAGGCCCGCTTGTAAAATTCCCGACGATCTCGGATTTCCTGAGGAAATGAGCGTTGCCTCACTGG
ACCTTACAGGCGGGTTGCCCGAAGTGGCTACTCCCGAATCAGAAGAAGCATTTACTCTTCCCCTGCTGAACGATCCCGAACCAA
AACCATCCGTTGAGCCGGTGAAATCTATTAGTTCAATGGAATTGAAAACTGAACCTTTCGACGATTTTCTCTTTCCTGCCTCTAG
CCGACCATCAGGGTCCGAAACGGCTCGTAGTGTACCTGATATGGATCTGAGCGGTAGCTTTTACGCCGCCGATTGGGAACCTC
TCCATTCCGGTTCATTGGGCATGGGACCGATGGCTACTGAATTGGAACCGCTCTGTACCCCAGTAGTGACATGCACACCTTCAT
GTACAGCATATACATCAAGCTTTGTGTTTACTTATCCGGAAGCCGATTCTTTTCCTTCTTGCGCTGCCGCTCATAGGAAAGGCTC
CTCTTCTAACGAACCAAGTAGTGATTCACTGTCATCCCCTACACTTTTGGCTCTTGGCAGCGGCGCCACAAACTTCTCTCTGCTA
AAGCAAGCAGGTGATGTTGAAGAAAACCCCGGGCCTATGGAAGTAGCCCCTGAACAACCCAGATGGATGGCTCATCCAGCAG
TATTGAACGCACAACATCCAGATTCCCATCATCCAGGATTGGCCCATAATTATATGGAGCCAGCCCAACTCTTGCCACCTGATG
AAGTCGATGTTTTCTTTAACCATCTGGATAGTCAAGGTAATCCTTATTACGCTAATCCAGCACATGCACGCGCTCGGGTTTCATA
TTCCCCTGCCCATGCAAGGCTTACAGGCGGACAAATGTGTCGTCCTCATCTGCTCCATTCCCCAGGCCTTCCATGGCTTGATGG
CGGGAAGGCGGCGCTTTCAGCGGCGGCTGCACATCATCATAATCCGTGGACTGTCTCTCCTTTTAGTAAAACTCCGCTTCATCC
GAGCGCAGCAGGCGGACCTGGCGGACCCCTGTCAGTCTATCCTGGTGCCGGTGGCGGCTCCGGCGGTGGGTCCGGTTCCAGC
GTTGCAAGCTTGACACCAACGGCGGCTCATTCAGGATCACATTTGTTTGGTTTTCCCCCAACCCCGCCGAAAGAGGTCAGTCCC
GATCCAAGTACAACCGGAGCAGCAAGTCCCGCAAGCTCATCTGCCGGCGGCTCCGCCGCTAGAGGCGAAGATAAAGATGGG
GTAAAATATCAAGTCTCTCTTACCGAATCCATGAAAATGGAGTCTGGATCTCCTCTCCGGCCCGGGCTCGCGACCATGGGGAC
ACAACCCGCAACCCATCATCCAATTCCAACTTATCCAAGCTACGTTCCCGCTGCAGCACATGATTATTCATCTGGGCTGTTTCAT
CCGGGCGGATTTCTCGGTGGCCCAGCATCTTCTTTTACTCCCAAACAACGGTCTAAAGCAAGGTCTTGCAGTGAGGGTAGGGA
ATGCGTGAATTGCGGAGCTACTGCAACTCCCCTGTGGCGTCGCGATGGGACAGGTCATTATCTTTGTAACGCTTGCGGACTTTA
TCATAAAATGAACGGCCAAAATCGCCCGCTGATTAAACCTAAACGCAGGTTGTCAGCGGCTCGCAGGGGGGGGACATGCTGC
GCCAACTGCCAAACCACCACTACAACACTGTGGCGAAGAAATGCAAATGGCGATCCCGTGTGTAATGCTTGCGGGCTTTATTA
TAAACTCCATAACGTCAATCGGCCTCTTACAATGAAGAAAGAGGGAATTCAAACCAGGAATAGGAAAATGTCAAATAAAAGCA
AGAAATCAAAGAAGGGCGCCGAATGTTTTGAAGAATTGTCTAAATGTATGCAAGAGAAATCTTCACCGTTTTCCGCCGCCGCG
TTGGCCGGTCATATGGCTCCAGTCGGACATCTTCCGCCATTTTCACATAGTGGGCATATTCTTCCAACACCCACACCTATTCATC
CTTCATCTTCTCTGTCATTTGGACATCCTCATCCTAGCTCCATGGTTACTGCAATGGGCGGCTCCGGCGAGGGCAGGGGAAGTC
TTCTAACATGCGGGGACGTGGAGGAAAATCCCGGCCCAATGCCCCGAAGTTTTCTCGTTAAATCAAAGAAAGCACATACTTAT
CATCAACCTCGCGTCCAAGAGGACGAGCCACTTTGGCCACCCGCTCTGACACCTGTCCCGAGGGATCAAGCGCCGTCCAATTC
ACCCGTGTTGTCTACCCTCTTTCCCAATCAATGTTTGGATTGGACAAATTTGAAGAGGGAACCAGAACTTGAACAAGATCAAAA
TCTTGCGCGGATGGCGCCTGCCCCGGAAGGGCCAATCGTCCTCAGCCGGCCGCAAGACGGCGATTCCCCTCTTAGCGATAGCC
CACCATTTTATAAACCCAGTTTTAGCTGGGATACTCTGGCTACTACTTACGGACATTCATATAGACAAGCGCCTTCAACGATGCA
AAGCGCATTTCTTGAACATAGTGTGTCCCTTTATGGTTCCCCACTCGTTCCTTCCACAGAACCAGCTCTGGATTTCTCACTTCGAT
ATTCTCCCGGGATGGACGCTTATCATTGCGTAAAATGTAATAAAGTGTTTTCTACACCGCATGGTCTGGAAGTCCACGTACGCC
GTAGTCACTCCGGCACAAGGCCTTTTGCGTGCGATATTTGTGGGAAGACGTTTGGTCATGCCGTATCACTCGAACAACATACAC
ATGTGCATTCTCAAGAACGGAGTTTTGAATGTAGAATGTGTGGTAAAGCTTTTAAAAGGTCATCAACTTTGAGTACTCATCTCT
TGATACACAGCGATACAAGGCCGTATCCATGTCAATTTTGTGGAAAACGGTTTCATCAAAAGTCAGATATGAAGAAACATACA
TATATTCATACCGGGGAAAAGCCCCATAAATGTCAAGTCTGTGGTAAAGCGTTTTCTCAATCTAGTAATCTGATTACACATTCTC
GTAAACATACTGGGTTTAAACCTTTCTCTTGCGAACTTTGTACAAAGGGATTTCAACGGAAAGTCGATCTTCGTAGACATAGAG
AATCCCAACATAACCTTAAATAA
SEQ ID No. 49-FoGiGa
ATGATGTTTAGCGGATTTAATGCCGATTATGAAGCAAGCTCATCTCGATGTTCATCTGCAAGCCCAGCTGGTGACTCACTGAGC
TATTATCATAGCCCGGCTGATTCATTTTCATCTATGGGTTCCCCCGTTAATGCCCAAGACTTTTGTACCGATCTCGCAGTGAGCA
GCGCAAATTTTATCCCTACCGTGACCGCTATATCAACTAGCCCCGATTTGCAATGGCTTGTCCAACCAGCGTTGGTGTCAAGCG
TTGCACCCAGCCAAACTCGCGCTCCCCATCCCTTTGGCGTGCCAGCTCCGAGCGCAGGCGCCTATTCACGGGCAGGGGTAGTC
AAAACAATGACCGGCGGACGCGCTCAATCCATCGGAAGAAGAGGGAAAGTCGAGCAACTCTCCCCCGAGGAAGAGGAAAAG
CGACGCATTAGAAGAGAGCGGAACAAAATGGCCGCCGCTAAGTGTCGGAATAGACGCCGCGAACTCACCGACACTCTGCAGG
CCGAAACCGATCAGCTGGAAGACGAAAAGAGTGCCCTTCAAACTGAAATCGCAAATCTCTTGAAAGAAAAGGAGAAGCTGGA
ATTTATTCTTGCTGCACATCGTCCAGCATGTAAAATTCCGGACGATTTGGGGTTTCCCGAGGAAATGTCCGTTGCCTCTCTCGAC
TTGACCGGTGGGTTGCCTGAAGTAGCTACACCCGAAAGTGAAGAAGCGTTTACTCTTCCATTGCTTAACGATCCAGAACCAAA
ACCAAGCGTTGAGCCCGTAAAATCTATTTCTTCAATGGAACTCAAAACTGAACCATTCGACGATTTTCTCTTTCCTGCTTCTAGT
AGACCATCTGGCTCCGAAACCGCACGATCTGTACCTGATATGGATCTGTCCGGAAGCTTTTACGCCGCCGATTGGGAACCACTC
CATTCCGGTTCACTCGGAATGGGACCTATGGCTACGGAATTGGAACCTCTTTGTACCCCAGTTGTAACTTGCACCCCATCATGT
ACCGCCTATACCTCTAGCTTTGTATTTACGTATCCTGAAGCCGATAGCTTTCCATCTTGCGCCGCCGCGCATAGGAAAGGATCTT
CAAGTAACGAACCCAGTTCCGATAGTTTGAGTTCTCCGACACTTCTCGCTCTTGGCAGCGGCGCCACAAACTTCTCTCTGCTAA
AGCAAGCAGGTGATGTTGAAGAAAACCCCGGGCCTATGCCCCGTTCTTTCTTGGTAAAATCCAAGAAGGCGCATACATATCAT
CAACCACGGGTTCAAGAGGACGAGCCACTGTGGCCGCCAGCTCTGACTCCCGTCCCTCGGGATCAAGCCCCCTCTAATTCTCCC
GTGCTGTCAACATTGTTTCCTAATCAATGTTTGGATTGGACTAATCTGAAGCGCGAACCAGAATTGGAACAAGATCAAAATCTC
GCTCGGATGGCTCCTGCTCCGGAAGGTCCTATCGTTCTTTCAAGACCACAAGACGGCGATTCACCCCTCTCAGATTCTCCCCCTT
TCTATAAACCCAGCTTTAGTTGGGATACTCTGGCAACCACATACGGGCATTCTTATCGCCAAGCGCCAAGCACAATGCAAAGTG
CATTTCTCGAACATTCAGTATCTCTTTATGGATCTCCCCTGGTCCCTTCTACCGAACCAGCGCTTGACTTTTCCTTGAGGTATAGT
CCCGGGATGGACGCATATCATTGCGTAAAATGTAATAAAGTTTTCAGCACGCCCCATGGATTGGAAGTCCACGTCAGGCGATC
TCACTCTGGCACAAGACCTTTTGCATGCGATATTTGTGGTAAGACGTTTGGACATGCCGTTTCACTCGAACAACATACACATGT
TCATAGCCAAGAACGTTCATTTGAATGTAGAATGTGTGGAAAAGCATTTAAAAGGAGCTCTACCCTTTCTACACATTTGCTGAT
TCATAGCGATACCCGACCTTATCCGTGTCAATTTTGTGGAAAACGATTTCATCAGAAATCAGATATGAAGAAGCATACATATAT
ACATACCGGGGAGAAACCCCATAAATGTCAAGTCTGTGGCAAAGCATTTTCTCAATCAAGCAATCTGATTACTCATTCCAGAAA
ACATACTGGATTTAAACCCTTTTCATGCGAACTTTGTACTAAGGGATTTCAACGGAAAGTTGATTTGAGGAGACATCGAGAAA
GTCAACATAACCTGAAAGGCTCCGGCGAGGGCAGGGGAAGTCTTCTAACATGCGGGGACGTGGAGGAAAATCCCGGCCCAA
TGGAAGTTGCCCCGGAACAACCCCGTTGGATGGCCCATCCTGCAGTACTTAACGCCCAACATCCGGATTCCCATCATCCCGGAC
TCGCTCATAATTATATGGAGCCGGCTCAACTCTTGCCACCCGATGAAGTCGATGTGTTCTTTAACCATCTGGATTCTCAAGGGA
ATCCATATTACGCAAATCCAGCACATGCCCGAGCCAGAGTTAGCTATTCACCTGCCCATGCAAGATTGACTGGTGGACAAATGT
GTCGGCCCCATCTGCTCCATTCACCAGGGCTCCCATGGCTTGATGGCGGTAAGGCTGCGCTGAGCGCTGCCGCCGCACATCAT
CATAATCCATGGACAGTATCTCCCTTTTCTAAAACTCCCCTTCATCCTTCTGCAGCCGGTGGACCCGGCGGACCTTTGAGTGTTT
ATCCTGGCGCAGGCGGCGGTTCCGGCGGCGGGAGTGGAAGCAGTGTCGCGAGTCTGACGCCCACTGCTGCACATTCAGGAA
GTCATCTGTTTGGGTTTCCCCCAACTCCACCCAAAGAGGTTTCCCCAGATCCAAGTACAACCGGCGCAGCCAGTCCCGCTTCCT
CTAGCGCCGGCGGCTCAGCTGCTAGAGGGGAAGATAAAGATGGTGTTAAATATCAAGTTAGCCTTACTGAATCTATGAAAATG
GAGTCAGGGAGCCCGCTCAGACCCGGGCTGGCGACGATGGGTACACAACCGGCCACTCATCATCCTATTCCGACGTATCCGA
GTTACGTTCCCGCCGCCGCTCATGATTATTCTTCCGGCCTGTTTCATCCAGGCGGTTTTCTTGGCGGCCCCGCGAGCTCATTTAC
GCCCAAACAAAGGAGTAAAGCCAGATCTTGCTCTGAGGGGAGAGAATGCGTGAATTGCGGCGCGACGGCAACTCCCTTGTGG
CGCCGCGATGGTACTGGGCATTATCTCTGTAACGCATGCGGATTGTATCATAAAATGAACGGTCAAAATAGGCCTCTGATAAA
ACCTAAACGGCGCCTTAGTGCGGCAAGGCGTGCAGGAACATGCTGCGCTAACTGCCAAACAACTACAACTACTTTGTGGCGGC
GCAATGCGAATGGTGATCCCGTGTGTAATGCTTGCGGATTGTATTATAAACTCCATAACGTGAATCGTCCTCTCACTATGAAGA
AGGAAGGAATTCAAACGCGCAATAGGAAAATGTCTAATAAAAGCAAGAAGTCCAAGAAGGGCGCTGAATGTTTTGAAGAACT
CAGCAAATGTATGCAAGAGAAATCCAGTCCATTTTCAGCCGCCGCACTTGCAGGTCATATGGCTCCCGTCGGGCATTTGCCACC
GTTTTCCCATAGCGGTCATATTCTCCCAACCCCTACTCCAATACATCCTTCTAGCTCTCTGAGTTTTGGGCATCCTCATCCCAGTT
CCATGGTCACAGCGATGGGTTGA

1. A construct or a vector for reprogramming stem cells, differentiated cells, or mixtures thereof into hemogenic and/or hematopoietic stem cell-like cells, wherein the construct or the vector encodes a peptide comprising a combination of at least three isolated or synthetic transcription factors wherein the peptide sequence is at least 90% homologous to one of a list consisting of: SEQ ID No. 11, SEQ ID No. 12, SEQ ID No. 13, SEQ ID No. 14, SEQ ID No. 15, SEQ ID No. 16, and mixtures thereof.

2. (canceled)

3. (canceled)

4. (canceled)

5. The construct or the vector according to claim 1 comprising a sequence at least 90% homologous to one of a list consisting of: SEQ ID No. 44, SEQ ID No. 45, SEQ ID No. 46, SEQ ID No. 47, SEQ ID No. 48; SEQ ID No. 49.

6. (canceled)

7. (canceled)

8. The construct or the vector according to claim 1 with a sequence at least 90% homologous to one of a list consisting of: SEQ ID No. 20, SEQ ID. No 21, SEQ ID No. 22, SEQ ID No. 23, SEQ ID No. 24, SEQ ID No. 25, or combinations thereof.

9. (canceled)

10. (canceled)

11. (canceled)

12. The construct or the vector according to claim 1 wherein the combination of three isolated or synthetic transcription factors is encoded in the following sequential order from 5′ to 3′: GATA2, GFI1B, FOS; or GATA2, FOS, GFI1B; or GFI1B, GATA2, FOS; or GFI1B, FOS, GATA2; FOS, GATA2, GFI1B; or FOS, GFI1B, GATA2.

13. (canceled)

14. The construct or the vector according to claim 1 for use in medicine or as medicament.

15. A composition comprising the construct or vector described in claim 1 for reprogramming stem cells, differentiated cells, or mixtures thereof into hemogenic and/or hematopoietic stem cell-like cells.

16. The composition according of claim 15, further comprising a combination of at least three transcription factors encoded by an isolated or synthetic sequence at least 90% homologous to one a list consisting of: SEQ ID No. 44, SEQ ID No. 45, SEQ ID No. 46, SEQ ID No. 47, SEQ ID No. 48, SEQ ID No. 49.

17. (canceled)

18. (canceled)

19. (canceled)

20. (canceled)

21. The composition according to claim 15 for reprogramming or inducing factor of a cell selected from a list consisting of: fibroblast, differentiated cell, and mixtures thereof, into hemogenic and/or hematopoietic stem cell-like cells.

22. The composition according to claim 15, for use in veterinary or human medicine.

23. (canceled)

24. A method for reprogramming or inducing a stem cell or a differentiated cell into hemogenic and/or hematopoietic stem cell-like cells, comprising the following steps:
transducing a cell selected from a list consisting of: stem cell or a differentiated cell, and mixtures thereof, with one or more vectors comprising a nucleic acid sequence encoding an amino_acid sequence at least 90% homologous, to a sequence from a list consisting of SEQ ID No. 11, SEQ ID No. 12, SEQ ID No. 13, SEQ ID No. 14, SEQ ID No. 15, SEQ ID No. 16, and mixtures thereof; and
culturing the transduced cell in a cell media that supports growth of hemogenic and/or hematopoietic stem cell-like cells.

25. (canceled)

26. (canceled)

27. (canceled)

28. (canceled)

29. (canceled)

30. (canceled)

31. The method according to claim 24, wherein the pluripotent stem cell, multipotent stem cell, or differentiated cell, is selected from the group consisting of: an endoderm derived cell, a mesoderm derived cell, or an ectoderm derived cell, a multipotent stem cell including mesenchymal stem cell, a hematopoietic stem cell, an intestinal stem cell, a cell line, or fibroblast cell.

32. (canceled)

33. (canceled)

34. (canceled)

35. (canceled)

36. An induced hemogenic and/or hematopoietic stem cell-like cell obtained by the method described in claim 24.

37. A composition comprising an induced hemogenic and/or hematopoietic stem cell-like cell described in claim 36, or mixtures thereof, in a therapeutically effective amount and a pharmaceutically acceptable excipient.

38. The composition according to claim 37 for use in veterinary or human medicine.

39. The composition according to 38 claim 37 for use in stem cell or bone marrow transplantation, immunotherapy, hemotherapy, treatment or therapy of neurodegenerative diseases, drug screening, disease modelling of blood-related diseases, treatment or therapy of autoimmune diseases, treatment or therapy of immunodeficiency, treatment or therapy of cancer, or the treatment or therapy of an infectious diseases.

40. The composition according to claim 37, further comprising an anti-viral, an analgesic, an anti-inflammatory agent, a chemotherapy agent, a radiotherapy agent, an antibiotic, a diuretic, or mixtures thereof.

41. (canceled)

42. The composition according to claim 37, wherein the composition is an injectable formulation.

43. (canceled)

44. (canceled)

45. (canceled)

46. (canceled)

47. A kit comprising one or more of the following components:
the construct or vector of claim 1; and
induced hemogenic and/or hematopoietic stem cell-like cells, wherein the cells have been induced into their current state by transducing stem cells, differentiated cells, or mixtures thereof, with the construct or vector of claim 1, then culturing the cells in cell media that supports growth of hemogenic and/or hematopoietic stem cell-like cells.

48. The construct or the vector according to claim 1 for use in the treatment or therapy of diseases related to stem cell or bone marrow transplantation, immunotherapy, hemotherapy, treatment or therapy of neurodegenerative diseases, drug screening, disease modelling of blood-related diseases, treatment or therapy of autoimmune diseases, treatment or therapy of immunodeficiency, treatment or therapy of cancer, or the treatment or therapy of an infectious diseases.

49. The composition according to claim 15 for use in bone marrow and stem cell transplantation, in immunotherapy, or in autoimmune diseases, immunodeficiency, or in neurodegenerative or ageing diseases, or in cancer or in infectious diseases, or as a drug screening or disease model.


SEQUENCE LISTING (49 sequences)

SEQ ID NO: 1 - DNA, 1443 bp, Homo sapiens
  source 1..1443: mol_type = genomic DNA
atggaggtggcgcccgagcagccgcgctggatggcgcacccggccgtgctgaatgcgcagcaccccgactcacaccacccgggcctggcgcacaactacatggaacccgcgcagctgctgcctccagacgaggtggacgtcttcttcaatcacctcgactcgcagggcaacccctactatgccaaccccgctcacgcgcgggcgcgcgtctcctacagccccgcgcacgcccgcctgaccggaggccagatgtgccgcccacacttgttgcacagcccgggtttgccctggctggacgggggcaaagcagccctctctgccgctgcggcccaccaccacaacccctggaccgtgagccccttctccaagacgccactgcacccctcagctgctggaggccctggaggcccactctctgtgtacccaggggctgggggtgggagcgggggaggcagcgggagctcagtggcctccctcacccctacagcagcccactctggctcccaccttttcggcttcccacccacgccacccaaagaagtgtctcctgaccctagcaccacgggggctgcgtctccagcctcatcttccgcggggggtagtgcagcccgaggagaggacaaggacggcgtcaagtaccaggtgtcactgacggagagcatgaagatggaaagtggcagtcccctgcgcccaggcctagctactatgggcacccagcctgctacacaccaccccatccccacctacccctcctatgtgccggcggctgcccacgactacagcagcggactcttccaccccggaggcttcctggggggaccggcctccagcttcacccctaagcagcgcagcaaggctcgttcctgttcagaaggccgggagtgtgtcaactgtggggccacagccacccctctctggcggcgggacggcaccggccactacctgtgcaatgcctgtggcctctaccacaagatgaatgggcagaaccgaccactcatcaagcccaagcgaagactgtcggccgccagaagagccggcacctgttgtgcaaattgtcagacgacaaccaccaccttatggcgccgaaacgccaacggggaccctgtctgcaacgcctgtggcctctactacaagctgcacaatgttaacaggccactgaccatgaagaaggaagggatccagactcggaaccggaagatgtccaacaagtccaagaagagcaagaaaggggcggagtgcttcgaggagctgtcaaagtgcatgcaggagaagtcatcccccttcagtgcagctgccctggctggacacatggcacctgtgggccacctcccgcccttcagccactccggacacatcctgcccactccgacgcccatccacccctcctccagcctctccttcggccacccccacccgtccagcatggtgaccgccatgggctag

SEQ ID NO: 2 - DNA, 993 bp, Homo sapiens
  source 1..993: mol_type = genomic DNA
atgccacgctccttcctggtgaagagcaagaaggctcacacctaccaccagccccgtgtgcaggaagatgaaccgctctggcctcctgcccttaccccggtgcccagagaccaggctccaagcaacagccctgtccttagcactctattcccaaaccagtgcctggactggaccaacctcaaacgagagccggagctggagcaggaccagaacttggccaggatggccccggcaccagagggccccattgtgctgtcccgaccccaggatggggactctccactgtccgactcacccccattctacaagcctagcttctcctgggacaccttggccacaacctatggccacagctaccggcaggccccctccaccatgcagtcagccttcctggagcactccgtcagcctgtacggcagtcctcttgtgcccagcactgagcccgccttggacttcagcctccgctactccccaggcatggatgcgtaccactgtgtgaagtgcaacaaggtcttctccacccctcacgggctcgaagtgcatgtgcgacgctcccatagtgggacccggcccttcgcctgtgacatctgcggcaaaaccttcggccacgctgtgagcctggagcagcacacgcacgtccactcccaggagcgcagcttcgagtgccgcatgtgcggcaaggccttcaagcgctcgtccacgctgtccacccacctgctcatccactcagacacgcggccctacccctgccagttctgcggcaagcgtttccaccagaagtccgacatgaagaagcacacctacatccacacaggtgagaagccgcacaagtgccaggtgtgcggaaaggccttcagccagagctccaacctcatcacccacagccgcaagcacacaggcttcaagcccttcagctgtgagctgtgcaccaaaggcttccagcgcaaggtggacctgcggcggcaccgcgagagccagcacaatctcaagtga

SEQ ID NO: 3 - DNA, 1143 bp, Homo sapiens
  source 1..1143: mol_type = genomic DNA
atgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggacttctgcacggacctggccgtctccagtgccaacttcattcccacggtcactgccatctcgaccagtccggacctgcagtggctggtgcagcccgccctcgtctcctccgtggccccatcgcagaccagagcccctcaccctttcggagtccccgccccctccgctggggcttactccagggctggcgttgtgaagaccatgacaggaggccgagcgcagagcattggcaggaggggcaaggtggaacagttatctccagaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtga

SEQ ID NO: 4 - DNA, 57 bp, synthetic construct
  source 1..57: mol_type = other DNA
gccacaaacttctctctgctaaagcaagcaggtgatgttgaagaaaaccccgggcct

SEQ ID NO: 5 - DNA, 54 bp, synthetic construct
  source 1..54: mol_type = other DNA
gagggcaggggaagtcttctaacatgcggggacgtggaggaaaatcccggccca

SEQ ID NO: 6 - protein, 480 aa, Homo sapiens
MEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMG

SEQ ID NO: 7 - protein, 330 aa, Homo sapiens
MPRSFLVKSKKAHTYHQPRVQEDEPLWPPALTPVPRDQAPSNSPVLSTLFPNQCLDWTNLKREPELEQDQNLARMAPAPEGPIVLSRPQDGDSPLSDSPPFYKPSFSWDTLATTYGHSYRQAPSTMQSAFLEHSVSLYGSPLVPSTEPALDFSLRYSPGMDAYHCVKCNKVFSTPHGLEVHVRRSHSGTRPFACDICGKTFGHAVSLEQHTHVHSQERSFECRMCGKAFKRSSTLSTHLLIHSDTRPYPCQFCGKRFHQKSDMKKHTYIHTGEKPHKCQVCGKAFSQSSNLITHSRKHTGFKPFSCELCTKGFQRKVDLRRHRESQHNLK

SEQ ID NO: 8 - protein, 380 aa, Homo sapiens
MMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLAL

SEQ ID NO: 9 - protein, 19 aa, synthetic construct
ATNFSLLKQAGDVEENPGP

SEQ ID NO: 10 - protein, 18 aa, synthetic construct
EGRGSLLTCGDVEENPGP

SEQ ID NO: 11 - protein, 1233 aa, synthetic construct
MEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMGGSGATNFSLLKQAGDVEENPGPMPRSFLVKSKKAHTYHQPRVQEDEPLWPPALTPVPRDQAPSNSPVLSTLFPNQCLDWTNLKREPELEQDQNLARMAPAPEGPIVLSRPQDGDSPLSDSPPFYKPSFSWDTLATTYGHSYRQAPSTMQSAFLEHSVSLYGSPLVPSTEPALDFSLRYSPGMDAYHCVKCNKVFSTPHGLEVHVRRSHSGTRPFACDICGKTFGHAVSLEQHTHVHSQERSFECRMCGKAFKRSSTLSTHLLIHSDTRPYPCQFCGKRFHQKSDMKKHTYIHTGEKPHKCQVCGKAFSQSSNLITHSRKHTGFKPFSCELCTKGFQRKVDLRRHRESQHNLKGSGEGRGSLLTCGDVEENPGPMMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLAL

SEQ ID NO: 12 - protein, 1233 aa, synthetic construct
MEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMGGSGATNFSLLKQAGDVEENPGPMMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLALGSGEGRGSLLTCGDVEENPGPMPRSFLVKSKKAHTYHQPRVQEDEPLWPPALTPVPRDQAPSNSPVLSTLFPNQCLDWTNLKREPELEQDQNLARMAPAPEGPIVLSRPQDGDSPLSDSPPFYKPSFSWDTLATTYGHSYRQAPSTMQSAFLEHSVSLYGSPLVPSTEPALDFSLRYSPGMDAYHCVKCNKVFSTPHGLEVHVRRSHSGTRPFACDICGKTFGHAVSLEQHTHVHSQERSFECRMCGKAFKRSSTLSTHLLIHSDTRPYPCQFCGKRFHQKSDMKKHTYIHTGEKPHKCQVCGKAFSQSSNLITHSRKHTGFKPFSCELCTKGFQRKVDLRRHRESQHNLK

SEQ ID NO: 13 - protein, 1233 aa, synthetic construct
MPRSFLVKSKKAHTYHQPRVQEDEPLWPPALTPVPRDQAPSNSPVLSTLFPNQCLDWTNLKREPELEQDQNLARMAPAPEGPIVLSRPQDGDSPLSDSPPFYKPSFSWDTLATTYGHSYRQAPSTMQSAFLEHSVSLYGSPLVPSTEPALDFSLRYSPGMDAYHCVKCNKVFSTPHGLEVHVRRSHSGTRPFACDICGKTFGHAVSLEQHTHVHSQERSFECRMCGKAFKRSSTLSTHLLIHSDTRPYPCQFCGKRFHQKSDMKKHTYIHTGEKPHKCQVCGKAFSQSSNLITHSRKHTGFKPFSCELCTKGFQRKVDLRRHRESQHNLKGSGATNFSLLKQAGDVEENPGPMEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMGGSGEGRGSLLTCGDVEENPGPMMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLAL

SEQ ID NO: 14 - protein, 1233 aa, synthetic construct
MPRSFLVKSKKAHTYHQPRVQEDEPLWPPALTPVPRDQAPSNSPVLSTLFPNQCLDWTNLKREPELEQDQNLARMAPAPEGPIVLSRPQDGDSPLSDSPPFYKPSFSWDTLATTYGHSYRQAPSTMQSAFLEHSVSLYGSPLVPSTEPALDFSLRYSPGMDAYHCVKCNKVFSTPHGLEVHVRRSHSGTRPFACDICGKTFGHAVSLEQHTHVHSQERSFECRMCGKAFKRSSTLSTHLLIHSDTRPYPCQFCGKRFHQKSDMKKHTYIHTGEKPHKCQVCGKAFSQSSNLITHSRKHTGFKPFSCELCTKGFQRKVDLRRHRESQHNLKGSGATNFSLLKQAGDVEENPGPMMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLALGSGEGRGSLLTCGDVEENPGPMEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMG

SEQ ID NO: 15 - protein, 1233 aa, synthetic construct
MMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLALGSGATNFSLLKQAGDVEENPGPMEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMGGSGEGRGSLLTCGDVEENPGPMPRSFLVKSKKAHTYHQPRVQEDEPLWPPALTPVPRDQAPSNSPVLSTLFPNQCLDWTNLKREPELEQDQNLARMAPAPEGPIVLSRPQDGDSPLSDSPPFYKPSFSWDTLATTYGHSYRQAPSTMQSAFLEHSVSLYGSPLVPSTEPALDFSLRYSPGMDAYHCVKCNKVFSTPHGLEVHVRRSHSGTRPFACDICGKTFGHAVSLEQHTHVHSQERSFECRMCGKAFKRSSTLSTHLLIHSDTRPYPCQFCGKRFHQKSDMKKHTYIHTGEKPHKCQVCGKAFSQSSNLITHSRKHTGFKPFSCELCTKGFQRKVDLRRHRESQHNLK

SEQ ID NO: 16 - protein, 1233 aa, synthetic construct
MMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLALGSGATNFSLLKQAGDVEENPGPMPRSFLVKSKKAHTYHQPRVQEDEPLWPPALTPVPRDQAPSNSPVLSTLFPNQCLDWTNLKREPELEQDQNLARMAPAPEGPIVLSRPQDGDSPLSDSPPFYKPSFSWDTLATTYGHSYRQAPSTMQSAFLEHSVSLYGSPLVPSTEPALDFSLRYSPGMDAYHCVKCNKVFSTPHGLEVHVRRSHSGTRPFACDICGKTFGHAVSLEQHTHVHSQERSFECRMCGKAFKRSSTLSTHLLIHSDTRPYPCQFCGKRFHQKSDMKKHTYIHTGEKPHKCQVCGKAFSQSSNLITHSRKHTGFKPFSCELCTKGFQRKVDLRRHRESQHNLKGSGEGRGSLLTCGDVEENPGPMEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMG

SEQ ID NO: 17 - DNA, 7210 bp, synthetic construct
  source 1..7210: mol_type = other DNA
tcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggcccgggatccattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacg

SEQ ID NO: 18 - DNA, 10709 bp, synthetic construct
  source 1..10709: mol_type = other DNA
ttgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgccrggggggggggggggggggggggggggggggggggsgrggsggrgrggkgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgccycgggccggggrgggctcgggggrggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgggccgcgttgacgcgcacggcaagaggcgaggggcggcgactggtgagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagctcgaaacaaccggtacctctagaactatagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgaca

SEQ ID NO: 19 - DNA, 5822 bp, synthetic construct
  source 1..5822: mol_type = other DNA
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 20 - DNA, 10918 bp, synthetic construct
  source 1..10918: mol_type = other DNA
tcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggcccgggatccatggaagtcgctcctgaacaaccacggtggatggctcatcctgcagttcttaacgctcaacatccagatagccatcatcctgggctcgctcataattatatggagcctgcccaactccttcccccagatgaagtagatgtattctttaaccatcttgatagccaaggaaatccttattacgcaaatcctgcccatgccagagccagagtaagctattcaccagcccatgcaaggcttactggtggacaaatgtgtcggccccatctgctgcattcccccggcctgccgtggttggatggcggaaaggcggctctgagtgcggcggctgcacatcatcataatccttggacagtctcacctttctctaaaactccccttcatcctagtgcagccggtggtccaggtggtccgctgtccgtttatcccggcgccggcggcggaagtggcggcggaagtggcagtagcgtagcttcattgacacccacagctgctcattccggaagtcatctctttgggtttccccctaccccgccgaaagaggtttccccagatccgtcaactaccggtgccgcttcccctgctagttccagtgccggcggaagcgctgcaaggggcgaagataaagatggagtgaaatatcaagtctccttgacagaatccatgaaaatggagtccggttctccactcaggcctggactcgccacaatgggtactcaacccgccacccatcatccaataccgacttatccttcatacgtaccagccgccgctcatgattattcatcaggtttgtttcatccaggtggatttcttggcgggcctgctagttcttttacaccaaaacaaaggtccaaagccaggtcttgcagtgaggggcgcgaatgcgtaaattgcggcgctaccgcaacaccactgtggcgtcgtgatggaacaggacattatctttgtaacgcatgcgggctgtatcataaaatgaacggacaaaatcggcccctgattaaacctaaacgtcgcttgtctgctgcaaggcgggctgggacgtgctgcgccaactgccaaacaaccacaacaacactgtggcgtaggaatgctaatggcgatcccgtatgtaatgcatgcggtctgtattataaacttcataacgtcaatcggccccttactatgaagaaagagggcatacaaacccgaaatcgcaaaatgtcaaataaatctaagaaatccaagaagggagccgaatgttttgaagaactcagcaaatgtatgcaagaaaagtccagcccgttttccgccgccgctttggccggccatatggcccccgtcggacatctgccaccgttttcccatagcggtcatattcttcctacacccaccccaattcatccgtctagctccctgtcttttgggcatcctcatcctagctcaatggtcacagctatgggtggcagcggcgccacaaacttctctctgctaaagcaagcaggtgatgttgaagaaaaccccgggcctatgccgaggagctttctcgttaaatccaagaaagcgcatacatatcatcaacctagggtccaagaggacgagccactgtggccacccgctctcacaccagttcctcgggatcaagcaccttctaattcacccgtgttgagtaccttgtttccgaatcaatgtctcgattggactaatctgaagcgggaaccagaattggaacaagatcaaaatctggcacgcatggctcccgctcctgaaggtccaatcgttctcagtcggccgcaagacggcgattccccgctttctgatagcccaccgttttataaaccatcttttagctgggatacgctcgctacgacatacggtcattcctatagacaagctccgtcaactatgcaaagcgcttttctcgaacatagcgtttcactttatggatcacccctcgtcccttccaccgaacctgctctggacttttccctgagatattctcccgggatggacgcctatcattgcgttaaatgtaataaagtgttttcaacaccgcatggtcttgaggtccacgtacgccgatcacacagcggcactcgcccgtttgcatgcgatatttgtggaaagacttttggacatgccgtctctctcgaacaacatacacatgtgcattcacaagaacgttcctttgaatgtcgaatgtgtgggaaagcgtttaaaaggtcaagtaccctcagcacgcatcttcttattcattctgatacccgcccttatccatgtcaattttgtgggaaacgctttcatcagaaaagtgatatgaagaaacatacgtatattcataccggggaaaagccacataaatgtcaagtatgtggcaaagcattttcccaaagttctaatttgataacgcatagtcgaaaacatactgggtttaaaccattttcctgcgaactctgtactaagggatttcaaaggaaagtagatcttagaaggcatagggaatcccaacataacctgaaaggctccggcgagggcaggggaagtcttctaacatgcggggacgtggaggaaaatcccggcccaatgatgttttccgggtttaatgccgattatgaagcatccagttctaggtgtagttctgcaagtcccgcgggcgactcactgtcatattatcatagcccggctgatagcttttcttctatggggtcacccgtgaatgcccaagatttctgtactgatttggcagtgagctcagctaattttatccctactgtgaccgctataagcacttctcctgatctccaatggctcgtccaaccagcactggttagttcagtcgctcccagtcaaacacgggcaccccatccgtttggggttccagcaccttcagcaggcgcctatagccgggccggagtcgtaaagacaatgactggtgggagagcccaatcaatcggaagaagaggaaaagtcgagcaacttagccctgaggaagaggaaaagagacgcattaggcgcgagcggaacaaaatggccgccgctaagtgtcgaaataggcgtcgtgaactcacagacaccctgcaggccgaaaccgatcagcttgaggacgagaaaagtgcactgcaaactgaaatcgctaatcttctcaaagagaaagagaagctggaatttattctcgctgcacatcgtccagcatgtaaaattcccgacgatctcggatttccggaagaaatgagcgtagccagtctcgaccttaccggtggactccctgaagtcgcgacacctgaatccgaagaagcgtttacattgcccttgctgaacgatccggaaccaaaaccaagcgtcgagcccgtgaaaagtatatcatctatggaacttaaaactgaacctttcgacgattttctctttcctgcttccagccgcccatcagggtcagaaaccgctagatctgtccccgatatggatctgagcggaagcttttacgctgccgattgggaaccactccattcaggtagtcttggcatgggccctatggcgaccgaacttgaacctctttgtacacccgttgtaacttgcaccccatcctgtacggcctatacctcaagttttgtatttacatatcctgaagccgattcttttccatcctgcgccgccgcgcatcgaaaagggtcttcatctaacgaaccatcttccgatagtttgagtagtccaacccttcttgctctctaaggatccattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacg

SEQ ID NO: 21 - DNA, 10918 bp, synthetic construct
  source 1..10918: mol_type = other DNA
tcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggcccgggatccatggaagtcgccccggaacaaccacgatggatggcacatcctgcagttctcaacgcacaacatcctgatagtcatcatccagggttggctcataattatatggagcctgcccaactccttccccctgatgaagttgatgtgttctttaaccatctggattctcaaggaaatccgtattacgctaatccagcccatgcacgcgcacgtgtttcatatagtcctgcccatgcgaggcttacaggcgggcaaatgtgtcgtccccatctcctgcattctcccggcctgccgtggcttgatggtggaaaggctgcgctgagcgctgccgccgctcatcatcataatccatggactgtcagtccgttttctaaaactcctttgcatccatctgcagccggtggaccaggcggtccgctttcagtttatcccggtgcgggtggcggctccggcggcggttcaggatcaagcgtcgcgagcctgactccaacggctgctcatagtggtagccatctgtttgggtttcccccaactccccctaaagaggtcagcccagatccaagcacgaccggcgccgcttcccctgcatccagctctgccggtgggagcgcggcaagaggggaagataaagatggagtgaaatatcaagtaagtttgacagaatcaatgaaaatggagagcggatctcctctccggcccgggcttgccacaatgggtactcaaccagccacccatcatcctattccaacatatcctagctacgttcccgccgccgctcatgattattcatctggtctgtttcatcctggcgggttcttgggcggccccgcgagcagttttacacccaaacaacgttctaaagccaggagctgcagtgagggtagagaatgcgttaattgcggcgctactgcaacaccgctttggcgaagagatggtactggtcattatttgtgtaacgcatgcggtctgtatcataaaatgaacggtcaaaatcggcccctgattaaacctaaaaggcggcttagtgcggcaaggcgtgcaggaacttgctgcgccaactgccaaacaacgacaactactctctggagacggaatgcaaatggtgatccggtttgtaatgcgtgcgggctgtattataaattgcataacgtcaatcggccgcttacaatgaagaaagagggaatacaaaccagaaatcgcaaaatgagcaataaatctaagaaatctaagaagggagcagaatgttttgaggaactcagcaaatgtatgcaagaaaagtccagccctttctccgctgcagctttggcggggcatatggctcccgtcggtcatcttccaccgttttctcatagcggtcatattcttcctacgcccaccccaatacatccttcttcatctttgtcatttggacatcctcatccatcttctatggtcactgcaatgggcggcagcggcgccacaaacttctctctgctaaagcaagcaggtgatgttgaagaaaaccccgggcctatgatgttttcagggtttaatgccgattatgaagccagttcttcaagatgttcctcagcttcccccgcaggcgactccttgtcctattatcattctccagctgatagctttagttccatggggtcccccgttaatgcccaagacttttgtactgatctcgcggttagcagcgcaaattttataccgactgtgacagcaatatcaacatctccagatcttcaatggctcgttcaacctgcactggtgagctctgtcgcaccttcacaaacgcgggctccacatccatttggcgttcctgcaccaagtgcaggtgcctatagccgcgcaggggtggttaaaacaatgactggtggaagagcccaatccatcgggcgccggggaaaagtagagcaactgtcccccgaggaagaggaaaagcgtcgtattaggcgcgagcggaacaaaatggcggctgcaaagtgtagaaataggcgccgcgaattgacagacaccctgcaggcagaaaccgatcagctcgaggacgaaaagtccgcccttcaaactgaaatcgcaaatttgcttaaagagaaagagaagttggaatttatactcgctgcccatcggccagcatgtaaaatacccgacgatttgggttttcccgaggaaatgtcagtagcctctctcgaccttacgggcggacttcccgaagtggcgactcctgaaagtgaagaagcatttacacttcccctgctgaacgatccagaacctaaaccttccgtcgagcccgtaaaatcaatttcttccatggaattgaaaacagaacctttcgacgattttctctttcctgccagttcacgcccatccgggagtgaaaccgctcgaagcgttccggatatggatcttagcggcagcttttacgctgctgattgggaaccgcttcatagcgggtcattgggtatgggaccaatggctaccgaacttgaaccattgtgtaccccagttgtgacatgcaccccttcttgtaccgcgtatacttcaagctttgtatttacatatcctgaagccgattcatttccaagttgcgccgccgctcataggaaagggtctagtagtaacgaaccaagttcagattcactgtccagccctacattgttggctctcggctccggcgagggcaggggaagtcttctaacatgcggggacgtggaggaaaatcccggcccaatgccccggagttttcttgttaaatccaagaaagcccatacatatcatcaacctcgcgtccaagaggacgagccactgtggcccccagctctgacgcctgtacctcgggatcaagcaccgtctaattctcccgtgctgtcaaccctgtttcctaatcaatgtcttgattggactaatcttaagcgcgaaccagaactcgaacaagatcaaaatctggcacggatggctcctgcccccgaaggaccaatcgtactctctcggcctcaagacggcgatagtcccttgtcagattcacccccattttataaaccatcctttagttgggatacactcgctactacatacggtcattcatatagacaagctccatctacaatgcaatccgcgtttctcgaacatagcgtgagtctttatggtagcccactggttccaagtaccgaaccggcactggatttctcacttcgttatagccctgggatggacgcttatcattgcgtcaaatgtaataaagtgtttagtacgccccatggactggaagtccacgtaaggcggagtcactccggcactagaccttttgcatgcgatatatgtgggaagacttttggtcatgccgtttcccttgaacaacatactcatgtgcattctcaagaaagatcctttgaatgtcggatgtgtggaaaagcatttaaaaggtcatcaactctcagcactcatttgctgattcattctgatacccgcccatatccttgtcaattttgtggtaaaaggtttcatcagaaaagcgatatgaagaaacatacctacattcatactggagagaagccccataaatgtcaagtttgtggtaaggcattttcacaatcttctaacctgataacacattcccggaaacatactggatttaaaccattttcttgcgaactctgtacaaagggttttcaaagaaaagtagatctccgtaggcatcgtgaatcacaacataacctgaaataaggatccattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacg

SEQ ID NO: 22 - DNA, 10918 bp, synthetic construct
  source 1..10918: mol_type = other DNA
tcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggcccgggatccatgcccagaagctttctcgtcaaatcaaagaaagcccatacatatcatcaacctcgggttcaagaggacgagcctttgtggccaccagctctcacaccagtccctcgggatcaagccccttcaaatagtccagtactctctacactctttcctaatcaatgtttggattggacaaatctgaagcgggaaccagaacttgaacaagatcaaaatctggcacggatggcacctgcgcctgaaggaccaatcgtcttgtctaggcctcaagacggcgatagcccgctcagcgattccccaccgttttataaacccagttttagctgggatacactggctacgacatacggacattcatataggcaagctcctagtactatgcaatccgcatttctcgaacatagcgtgtctttgtatgggagcccgctcgtaccttctaccgaacctgcgctggattttagtctgcgttatagccctgggatggacgcctatcattgcgtcaaatgtaataaagtctttagcactccccatggcctggaagtccacgtccggcgaagtcactctggtactagaccatttgcttgcgatatttgtggaaagacttttggacatgccgtcagtcttgaacaacatacacatgtgcattctcaagaacggtcctttgaatgtcggatgtgtgggaaagcttttaaaagaagctctactctctcaacgcatctcttgattcatagcgatacacgtccttatccatgtcaattttgtggaaaacgcttccatcaaaagtcagatatgaagaaacatacttatattcatactggcgagaaaccacacaaatgtcaagtttgcggcaaagcattttcccaatcctcaaatctgattactcattccagaaaacatactgggtttaaaccattttcctgcgaactttgtacgaagggttttcaaaggaaagttgatttgcgacgccatagagaatcccaacataacctgaaaggcagcggcgccacaaacttctctctgctaaagcaagcaggtgatgttgaagaaaaccccgggcctatggaagttgcccctgaacaaccaagatggatggcccatcccgcggtcctcaacgcccaacatccagattctcatcatcccggtcttgcccataattatatggagcctgcccaacttttgcccccagatgaagtagatgtattcttcaaccatctggattcccaaggaaatccttattacgcaaatccagcccatgcacgcgccagggtgagctattctccagcccatgcaaggcttacgggcggacaaatgtgtagacctcatctgctccatagtccaggcctgccatggcttgatggcggtaaggccgcgcttagcgctgcagcagctcatcatcataatccatggactgtctccccatttagcaaaacaccgcttcatccttctgcagcaggtgggccgggcggacctctgagcgtatatcctggcgcaggtggcggctccggcggcgggtcaggaagtagtgtcgcgagcctgactccaaccgctgctcatagcgggagtcatctgtttgggtttccgccaacaccccctaaagaggtatccccagatccatctactaccggtgccgccagtcccgcaagctccagtgcaggcggcagcgctgctagaggtgaagataaagatggggtgaaatatcaagtttccctcaccgaaagtatgaaaatggagtccggaagcccacttcgacctggtcttgccaccatgggaacacaacccgccacccatcatcctataccaacgtatccaagctacgtccccgctgcagctcatgattattcatcagggctgtttcatcctggtggattcttgggcggtcctgcttcaagttttactccgaaacaacggtctaaagccaggagctgctccgagggaagggaatgcgtgaattgcggagcaaccgctactcccctttggcgaagggatggtactggtcattatctttgtaacgcatgcgggctgtatcataaaatgaacggccaaaatcggcccctgattaaaccaaaacggagactttccgctgcacggcgtgctgggacatgctgcgctaactgccaaaccaccactacaactctctggcggcggaatgctaatggtgatccagtgtgtaatgcttgcgggctgtattacaaacttcataacgtgaatcgccctcttacgatgaagaaagagggaattcaaacgcgtaatcgtaaaatgtcaaataaatcaaagaaatctaagaagggtgctgaatgttttgaggaactcagcaaatgtatgcaagagaaaagcagcccttttagcgcggcggcgcttgcagggcatatggccccagtaggtcatctgcccccgttttctcatagcggtcatattctcccaaccccaacaccgatacatccaagcagctccttgtcttttgggcatcctcatccaagctcaatggtcacagcaatgggcggctccggcgagggcaggggaagtcttctaacatgcggggacgtggaggaaaatcccggcccaatgatgttttcagggtttaatgccgattatgaagccagttcatctcgatgttcatcagccagccctgctggcgactcactgagctattatcattctccagctgattcattttcttctatggggagcccagtaaatgcacaagacttttgtaccgatctcgcagtttcaagcgcgaattttatcccaaccgtgaccgctattagcacttctccagatctccaatggcttgtccaaccggctctggtgagtagcgtcgctccgagccaaacgagggcaccccatccgtttggtgtaccagcaccaagcgcaggagcatattctcgcgcgggtgtagtcaaaacaatgactggcgggcgcgcccaaagtatcggtcgcagaggtaaagtagagcaactgagccccgaggaagaggaaaagcgacggattagacgtgagcggaacaaaatggccgccgctaagtgtcggaatagacgacgagaacttacagacactctgcaggccgaaaccgatcagttggaagacgaaaagtccgcactccaaacagaaatcgcgaatcttttgaaagagaaagagaagctcgaatttattcttgccgcacatcgccccgcgtgtaaaatacccgacgatcttggatttcctgaggaaatgtccgttgcaagcctggacctcaccggcggacttccggaagtcgcaacacccgaaagtgaagaagcttttactcttcccctgctgaacgatcccgaacctaaaccttctgttgagcccgttaaatcaataagtagtatggaactcaaaactgaaccattcgacgattttctgtttcccgcttccagtagaccaagcgggagcgaaactgcacgatctgtcccggatatggatctgtcaggctcattttacgcggccgattgggaaccactccattccggatctctcggcatgggaccaatggctaccgaacttgaaccactttgtacccccgtcgtaacgtgcacaccatcctgtaccgcgtatactagctcttttgtatttacttatccggaagcggattcatttccaagttgcgccgccgctcatagaaaagggtcttcatctaacgaaccgtcttccgattccctgagtagtcctacccttttggcattgtaaggatccattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacg

SEQ ID NO: 23 - DNA, 10918 bp, synthetic construct
  source 1..10918: mol_type = other DNA
tcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggcccgggatccatgccgagatcatttctcgtcaaaagtaagaaggcgcatacttatcatcaacctcgcgttcaagaggacgagcccttgtggcccccagcgttgacacctgttcctcgcgatcaagcaccctctaatagtcccgtgctctcaaccctgtttcctaatcaatgtttggattggacaaatctgaagcgtgaacctgaattggaacaagatcaaaatcttgcacgcatggcgccagcgcctgaaggtccaatcgtcctcagtagaccacaagacggagatagcccgttgagcgattctccacccttttataaaccatcctttagttgggatacactggcaacgacatacggacatagttataggcaagctccatctactatgcaatctgcgtttctggaacatagcgtttctctttatgggtctccgctggttccatcaacagaacctgcgctggattttagcctgagatatagtcctgggatggacgcatatcattgcgtaaaatgtaataaagtgtttagcacaccacatggcctggaagtccacgttcggcggagccacagcggcacaaggccatttgcatgcgatatttgtgggaagacgtttggacatgccgtttccctcgaacaacatacacatgttcatagccaagaaagatcatttgaatgtagaatgtgtgggaaagcatttaaacggtctagcacactctctacgcatctcctgattcatagcgatactcgcccatatccttgtcaattttgcgggaaaagatttcatcaaaagtcagatatgaagaagcatacgtatattcataccggggagaaaccacataaatgtcaagtctgtggcaaagccttttcccagtcatctaatttgataacacatagtaggaaacataccggttttaaaccgttttcttgcgaactctgtacaaagggttttcaaagaaaagttgatctccgtagacatagggaatcccaacataacctgaaaggcagcggcgccacaaacttctctctgctaaagcaagcaggtgatgttgaagaaaaccccgggcctatgatgtttagcggatttaatgccgattatgaagcaagctcaagcaggtgttcctcagcttctcccgctggagactctctgtcatattatcatagcccagccgatagcttttcttccatgggttccccagttaatgctcaagatttctgtaccgatctcgcggtgagtagcgcaaattttataccaaccgtgaccgctatttctacatctcccgatcttcaatggttggtccaacctgctctggtgagttctgtcgcaccctctcaaactcgggcaccgcatccctttggcgtaccagcaccaagcgccggagcctatagccgggcaggggtggtcaaaactatgaccggtgggcgtgcccaatccataggacgccgcgggaaagtcgagcaattgagcccggaagaagaggaaaagcggcgcattcgccgggagcgaaacaaaatggccgccgctaagtgtcgtaatagacgccgcgaactcaccgacacgttgcaggctgaaaccgatcagctcgaggacgagaaaagtgcgctccaaacggaaatcgctaatcttctcaaagaaaaggagaagcttgaatttattttggccgcccatcgccccgcttgtaaaataccggacgatcttgggtttcccgaggaaatgagtgtcgcctctctggacctcaccggcggattgcctgaagtggctacaccagaatccgaagaagcgtttacgctccccctgctgaacgatcccgaaccaaaaccatccgttgagccagtgaaatcaatatcatccatggaacttaaaacagaacctttcgacgattttctgtttcctgccagctcacgcccgagcggttcagaaaccgcgaggtctgtacccgatatggatctgtcaggttcattttacgctgccgattgggaacccttgcatagcggttctttgggcatgggacctatggctaccgaactcgaacctctctgtacacctgtcgtgacatgcacaccttcttgtaccgcctatacctcaagttttgtgtttacttatccagaagccgatagctttcctagttgcgctgccgcacatcgtaaaggaagttcctcaaacgaaccgagttccgattctcttagtagcccaacccttttggcacttggctccggcgagggcaggggaagtcttctaacatgcggggacgtggaggaaaatcccggcccaatggaagtcgccccagaacaacctaggtggatggcccatcctgcagtactcaacgcccaacatcctgatagtcatcatcccggattggcacataattatatggagccggctcaactccttcccccggatgaagtcgatgttttctttaaccatttggatagtcaaggaaatccatattacgcgaatcctgcccatgcccgtgccagggtgagctattcccctgcccatgctagactcactggtggtcaaatgtgtaggccccatcttctccattcccctggactgccttggttggatggcggaaaggctgctctgtccgctgccgccgcacatcatcataatccttggacagtcagtccttttagcaaaacaccactccatccttctgctgccggcggacccggcggtcctctgtcagtttatccgggcgctggtggcggctctggtggtggatcaggttcttccgtcgcaagcctgactcccactgccgcgcattccggtagccatctctttggttttccccctacacccccaaaggaagtctcccccgatccctcaactactggcgcggctagtcctgcgtcctcttctgccggcggcagcgctgctcgcggtgaagataaagatggagtgaaatatcaagttagcctcaccgaatctatgaaaatggagagcggatctcctttgcgtcccgggttggccaccatgggtacacaaccagccacccatcatcctattcctacttatccgagctacgtacctgctgccgcacatgattattcatctgggctgtttcatccaggcgggtttctcggtggccccgcaagctcatttactcccaaacaaaggtccaaagcgcggagctgcagtgaggggagagaatgcgtgaattgcggcgcaaccgctacgcccttgtggagaagggatggaacggggcattatctctgtaacgcatgcgggttgtatcataaaatgaacggccaaaatcgccccttgataaaaccaaagagacggttgtcagctgcacgacgcgctggaacttgctgcgccaactgccaaaccaccacaacaacattgtggcggcgtaatgcaaatggcgatcccgtgtgtaatgcttgcggtctgtattataaattgcataacgtgaatagacccctcactatgaagaaagagggcatacaaacccgcaatcgcaaaatgtcaaataaaagcaagaagtctaagaagggcgccgaatgttttgaagaattgtctaaatgtatgcaagaaaagagcagcccattttccgccgccgcactcgcaggccatatggccccagtaggtcatctgcccccttttagtcatagcgggcatattttgcctacgccaacaccgattcatcctagcagctcactgtcttttgggcatccacatccaagttctatggtcacggctatgggttgaggatccattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacg

SEQ ID NO: 24 - DNA, 10918 bp, synthetic construct
  source 1..10918: mol_type = other DNA
tcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggcccgggatccatgatgttttcaggatttaatgctgattatgaagccagctctagtaggtgttcctctgcaagcccagctggtgactccctgtcatattatcatagtcctgctgatagctttagttctatgggatctccagtgaatgcccaagacttttgtacagatctcgcagtaagcagcgcaaattttatcccgactgttaccgctatttccacttcacccgatctccaatggcttgtccaaccagcgctggtgagtagtgttgctccgagccaaacacgggctccccatccctttggggtgcctgctccgagcgcaggcgcatatagccgtgccggagtggtgaaaacaatgaccggcggacgggcacaatctatcgggcgtagaggtaaagtcgagcaactgagtcctgaggaagaggaaaagagacggattaggcgggagcgcaacaaaatggcagccgcaaagtgtcgaaatagaagacgcgaactcacggacaccctgcaggcagaaactgatcagcttgaggacgaaaagagtgccctgcaaactgaaatcgcaaatcttctcaaagaaaaggagaagctggaatttattctcgccgcacataggcccgcttgtaaaattcccgacgatctcggatttcctgaggaaatgagcgttgcctcactggaccttacaggcgggttgcccgaagtggctactcccgaatcagaagaagcatttactcttcccctgctgaacgatcccgaaccaaaaccatccgttgagccggtgaaatctattagttcaatggaattgaaaactgaacctttcgacgattttctctttcctgcctctagccgaccatcagggtccgaaacggctcgtagtgtacctgatatggatctgagcggtagcttttacgccgccgattgggaacctctccattccggttcattgggcatgggaccgatggctactgaattggaaccgctctgtaccccagtagtgacatgcacaccttcatgtacagcatatacatcaagctttgtgtttacttatccggaagccgattcttttccttcttgcgctgccgctcataggaaaggctcctcttctaacgaaccaagtagtgattcactgtcatcccctacacttttggctcttggcagcggcgccacaaacttctctctgctaaagcaagcaggtgatgttgaagaaaaccccgggcctatggaagtagcccctgaacaacccagatggatggctcatccagcagtattgaacgcacaacatccagattcccatcatccaggattggcccataattatatggagccagcccaactcttgccacctgatgaagtcgatgttttctttaaccatctggatagtcaaggtaatccttattacgctaatccagcacatgcacgcgctcgggtttcatattcccctgcccatgcaaggcttacaggcggacaaatgtgtcgtcctcatctgctccattccccaggccttccatggcttgatggcgggaaggcggcgctttcagcggcggctgcacatcatcataatccgtggactgtctctccttttagtaaaactccgcttcatccgagcgcagcaggcggacctggcggacccctgtcagtctatcctggtgccggtggcggctccggcggtgggtccggttccagcgttgcaagcttgacaccaacggcggctcattcaggatcacatttgtttggttttcccccaaccccgccgaaagaggtcagtcccgatccaagtacaaccggagcagcaagtcccgcaagctcatctgccggcggctccgccgctagaggcgaagataaagatggggtaaaatatcaagtctctcttaccgaatccatgaaaatggagtctggatctcctctccggcccgggctcgcgaccatggggacacaacccgcaacccatcatccaattccaacttatccaagctacgttcccgctgcagcacatgattattcatctgggctgtttcatccgggcggatttctcggtggcccagcatcttcttttactcccaaacaacggtctaaagcaaggtcttgcagtgagggtagggaatgcgtgaattgcggagctactgcaactcccctgtggcgtcgcgatgggacaggtcattatctttgtaacgcttgcggactttatcataaaatgaacggccaaaatcgcccgctgattaaacctaaacgcaggttgtcagcggctcgcagggcggggacatgctgcgccaactgccaaaccaccactacaacactgtggcgaagaaatgcaaatggcgatcccgtgtgtaatgcttgcgggctttattataaactccataacgtcaatcggcctcttacaatgaagaaagagggaattcaaaccaggaataggaaaatgtcaaataaaagcaagaaatcaaagaagggcgccgaatgttttgaagaattgtctaaatgtatgcaagagaaatcttcaccgttttccgccgccgcgttggccggtcatatggctccagtcggacatcttccgccattttcacatagtgggcatattcttccaacacccacacctattcatccttcatcttctctgtcatttggacatcctcatcctagctccatggttactgcaatgggcggctccggcgagggcaggggaagtcttctaacatgcggggacgtggaggaaaatcccggcccaatgccccgaagttttctcgttaaatcaaagaaagcacatacttatcatcaacctcgcgtccaagaggacgagccactttggccacccgctctgacacctgtcccgagggatcaagcgccgtccaattcacccgtgttgtctaccctctttcccaatcaatgtttggattggacaaatttgaagagggaaccagaacttgaacaagatcaaaatcttgcgcggatggcgcctgccccggaagggccaatcgtcctcagccggccgcaagacggcgattcccctcttagcgatagcccaccattttataaacccagttttagctgggatactctggctactacttacggacattcatatagacaagcgccttcaacgatgcaaagcgcatttcttgaacatagtgtgtccctttatggttccccactcgttccttccacagaaccagctctggatttctcacttcgatattctcccgggatggacgcttatcattgcgtaaaatgtaataaagtgttttctacaccgcatggtctggaagtccacgtacgccgtagtcactccggcacaaggccttttgcgtgcgatatttgtgggaagacgtttggtcatgccgtatcactcgaacaacatacacatgtgcattctcaagaacggagttttgaatgtagaatgtgtggtaaagcttttaaaaggtcatcaactttgagtactcatctcttgatacacagcgatacaaggccgtatccatgtcaattttgtggaaaacggtttcatcaaaagtcagatatgaagaaacatacatatattcataccggggaaaagccccataaatgtcaagtctgtggtaaagcgttttctcaatctagtaatctgattacacattctcgtaaacatactgggtttaaacctttctcttgcgaactttgtacaaagggatttcaacggaaagtcgatcttcgtagacatagagaatcccaacataaccttaaataaggatccattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacg

SEQ ID NO: 25 - DNA, 10918 bp, synthetic construct
  source 1..10918: mol_type = other DNA
tcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggcccgggatccatgatgtttagcggatttaatgccgattatgaagcaagctcatctcgatgttcatctgcaagcccagctggtgactcactgagctattatcatagcccggctgattcattttcatctatgggttcccccgttaatgcccaagacttttgtaccgatctcgcagtgagcagcgcaaattttatccctaccgtgaccgctatatcaactagccccgatttgcaatggcttgtccaaccagcgttggtgtcaagcgttgcacccagccaaactcgcgctccccatccctttggcgtgccagctccgagcgcaggcgcctattcacgggcaggggtagtcaaaacaatgaccggcggacgcgctcaatccatcggaagaagagggaaagtcgagcaactctcccccgaggaagaggaaaagcgacgcattagaagagagcggaacaaaatggccgccgctaagtgtcggaatagacgccgcgaactcaccgacactctgcaggccgaaaccgatcagctggaagacgaaaagagtgcccttcaaactgaaatcgcaaatctcttgaaagaaaaggagaagctggaatttattcttgctgcacatcgtccagcatgtaaaattccggacgatttggggtttcccgaggaaatgtccgttgcctctctcgacttgaccggtgggttgcctgaagtagctacacccgaaagtgaagaagcgtttactcttccattgcttaacgatccagaaccaaaaccaagcgttgagcccgtaaaatctatttcttcaatggaactcaaaactgaaccattcgacgattttctctttcctgcttctagtagaccatctggctccgaaaccgcacgatctgtacctgatatggatctgtccggaagcttttacgccgccgattgggaaccactccattccggttcactcggaatgggacctatggctacggaattggaacctctttgtaccccagttgtaacttgcaccccatcatgtaccgcctatacctctagctttgtatttacgtatcctgaagccgatagctttccatcttgcgccgccgcgcataggaaaggatcttcaagtaacgaacccagttccgatagtttgagttctccgacacttctcgctcttggcagcggcgccacaaacttctctctgctaaagcaagcaggtgatgttgaagaaaaccccgggcctatgccccgttctttcttggtaaaatccaagaaggcgcatacatatcatcaaccacgggttcaagaggacgagccactgtggccgccagctctgactcccgtccctcgggatcaagccccctctaattctcccgtgctgtcaacattgtttcctaatcaatgtttggattggactaatctgaagcgcgaaccagaattggaacaagatcaaaatctcgctcggatggctcctgctccggaaggtcctatcgttctttcaagaccacaagacggcgattcacccctctcagattctccccctttctataaacccagctttagttgggatactctggcaaccacatacgggcattcttatcgccaagcgccaagcacaatgcaaagtgcatttctcgaacattcagtatctctttatggatctcccctggtcccttctaccgaaccagcgcttgacttttccttgaggtatagtcccgggatggacgcatatcattgcgtaaaatgtaataaagttttcagcacgccccatggattggaagtccacgtcaggcgatctcactctggcacaagaccttttgcatgcgatatttgtggtaagacgtttggacatgccgtttcactcgaacaacatacacatgttcatagccaagaacgttcatttgaatgtagaatgtgtggaaaagcatttaaaaggagctctaccctttctacacatttgctgattcatagcgatacccgaccttatccgtgtcaattttgtggaaaacgatttcatcagaaatcagatatgaagaagcatacatatatacataccggggagaaaccccataaatgtcaagtctgtggcaaagcattttctcaatcaagcaatctgattactcattccagaaaacatactggatttaaacccttttcatgcgaactttgtactaagggatttcaacggaaagttgatttgaggagacatcgagaaagtcaacataacctgaaaggctccggcgagggcaggggaagtcttctaacatgcggggacgtggaggaaaatcccggcccaatggaagttgccccggaacaaccccgttggatggcccatcctgcagtacttaacgcccaacatccggattcccatcatcccggactcgctcataattatatggagccggctcaactcttgccacccgatgaagtcgatgtgttctttaaccatctggattctcaagggaatccatattacgcaaatccagcacatgcccgagccagagttagctattcacctgcccatgcaagattgactggtggacaaatgtgtcggccccatctgctccattcaccagggctcccatggcttgatggcggtaaggctgcgctgagcgctgccgccgcacatcatcataatccatggacagtatctcccttttctaaaactccccttcatccttctgcagccggtggacccggcggacctttgagtgtttatcctggcgcaggcggcggttccggcggcgggagtggaagcagtgtcgcgagtctgacgcccactgctgcacattcaggaagtcatctgtttgggtttcccccaactccacccaaagaggtttccccagatccaagtacaaccggcgcagccagtcccgcttcctctagcgccggcggctcagctgctagaggggaagataaagatggtgttaaatatcaagttagccttactgaatctatgaaaatggagtcagggagcccgctcagacccgggctggcgacgatgggtacacaaccggccactcatcatcctattccgacgtatccgagttacgttcccgccgccgctcatgattattcttccggcctgtttcatccaggcggttttcttggcggccccgcgagctcatttacgcccaaacaaaggagtaaagccagatcttgctctgaggggagagaatgcgtgaattgcggcgcgacggcaactcccttgtggcgccgcgatggtactgggcattatctctgtaacgcatgcggattgtatcataaaatgaacggtcaaaataggcctctgataaaacctaaacggcgccttagtgcggcaaggcgtgcaggaacatgctgcgctaactgccaaacaactacaactactttgtggcggcgcaatgcgaatggtgatcccgtgtgtaatgcttgcggattgtattataaactccataacgtgaatcgtcctctcactatgaagaaggaaggaattcaaacgcgcaataggaaaatgtctaataaaagcaagaagtccaagaagggcgctgaatgttttgaagaactcagcaaatgtatgcaagagaaatccagtccattttcagccgccgcacttgcaggtcatatggctcccgtcgggcatttgccaccgttttcccatagcggtcatattctcccaacccctactccaatacatccttctagctctctgagttttgggcatcctcatcccagttccatggtcacagcgatgggttgaggatccattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacg

SEQ ID NO: 26 - DNA, 1440 bp, synthetic construct
  source 1..1440: mol_type = other DNA
atggaagtcgctcctgaacaaccacggtggatggctcatcctgcagttcttaacgctcaacatccagatagccatcatcctgggctcgctcataattatatggagcctgcccaactccttcccccagatgaagtagatgtattctttaaccatcttgatagccaaggaaatccttattacgcaaatcctgcccatgccagagccagagtaagctattcaccagcccatgcaaggcttactggtggacaaatgtgtcggccccatctgctgcattcccccggcctgccgtggttggatggcggaaaggcggctctgagtgcggcggctgcacatcatcataatccttggacagtctcacctttctctaaaactccccttcatcctagtgcagccggtggtccaggtggtccgctgtccgtttatcccggcgccggcggcggaagtggcggcggaagtggcagtagcgtagcttcattgacacccacagctgctcattccggaagtcatctctttgggtttccccctaccccgccgaaagaggtttccccagatccgtcaactaccggtgccgcttcccctgctagttccagtgccggcggaagcgctgcaaggggcgaagataaagatggagtgaaatatcaagtctccttgacagaatccatgaaaatggagtccggttctccactcaggcctggactcgccacaatgggtactcaacccgccacccatcatccaataccgacttatccttcatacgtaccagccgccgctcatgattattcatcaggtttgtttcatccaggtggatttcttggcgggcctgctagttcttttacaccaaaacaaaggtccaaagccaggtcttgcagtgaggggcgcgaatgcgtaaattgcggcgctaccgcaacaccactgtggcgtcgtgatggaacaggacattatctttgtaacgcatgcgggctgtatcataaaatgaacggacaaaatcggcccctgattaaacctaaacgtcgcttgtctgctgcaaggcgggctgggacgtgctgcgccaactgccaaacaaccacaacaacactgtggcgtaggaatgctaatggcgatcccgtatgtaatgcatgcggtctgtattataaacttcataacgtcaatcggccccttactatgaagaaagagggcatacaaacccgaaatcgcaaaatgtcaaataaatctaagaaatccaagaagggagccgaatgttttgaagaactcagcaaatgtatgcaagaaaagtccagcccgttttccgccgccgctttggccggccatatggcccccgtcggacatctgccaccgttttcccatagcggtcatattcttcctacacccaccccaattcatccgtctagctccctgtcttttgggcatcctcatcctagctcaatggtcacagctatgggt

SEQ ID NO: 27 - DNA, 1440 bp, synthetic construct
  source 1..1440: mol_type = other DNA
atggaagtcgccccggaacaaccacgatggatggcacatcctgcagttctcaacgcacaacatcctgatagtcatcatccagggttggctcataattatatggagcctgcccaactccttccccctgatgaagttgatgtgttctttaaccatctggattctcaaggaaatccgtattacgctaatccagcccatgcacgcgcacgtgtttcatatagtcctgcccatgcgaggcttacaggcgggcaaatgtgtcgtccccatctcctgcattctcccggcctgccgtggcttgatggtggaaaggctgcgctgagcgctgccgccgctcatcatcataatccatggactgtcagtccgttttctaaaactcctttgcatccatctgcagccggtggaccaggcggtccgctttcagtttatcccggtgcgggtggcggctccggcggcggttcaggatcaagcgtcgcgagcctgactccaacggctgctcatagtggtagccatctgtttgggtttcccccaactccccctaaagaggtcagcccagatccaagcacgaccggcgccgcttcccctgcatccagctctgccggtgggagcgcggcaagaggggaagataaagatggagtgaaatatcaagtaagtttgacagaatcaatgaaaatggagagcggatctcctctccggcccgggcttgccacaatgggtactcaaccagccacccatcatcctattccaacatatcctagctacgttcccgccgccgctcatgattattcatctggtctgtttcatcctggcgggttcttgggcggccccgcgagcagttttacacccaaacaacgttctaaagccaggagctgcagtgagggtagagaatgcgttaattgcggcgctactgcaacaccgctttggcgaagagatggtactggtcattatttgtgtaacgcatgcggtctgtatcataaaatgaacggtcaaaatcggcccctgattaaacctaaaaggcggcttagtgcggcaaggcgtgcaggaacttgctgcgccaactgccaaacaacgacaactactctctggagacggaatgcaaatggtgatccggtttgtaatgcgtgcgggctgtattataaattgcataacgtcaatcggccgcttacaatgaagaaagagggaatacaaaccagaaatcgcaaaatgagcaataaatctaagaaatctaagaagggagcagaatgttttgaggaactcagcaaatgtatgcaagaaaagtccagccctttctccgctgcagctttggcggggcatatggctcccgtcggtcatcttccaccgttttctcatagcggtcatattcttcctacgcccaccccaatacatccttcttcatctttgtcatttggacatcctcatccatcttctatggtcactgcaatgggc

SEQ ID NO: 28 - DNA, 1440 bp, synthetic construct
  source 1..1440: mol_type = other DNA
atggaagttgcccctgaacaaccaagatggatggcccatcccgcggtcctcaacgcccaacatccagattctcatcatcccggtcttgcccataattatatggagcctgcccaacttttgcccccagatgaagtagatgtattcttcaaccatctggattcccaaggaaatccttattacgcaaatccagcccatgcacgcgccagggtgagctattctccagcccatgcaaggcttacgggcggacaaatgtgtagacctcatctgctccatagtccaggcctgccatggcttgatggcggtaaggccgcgcttagcgctgcagcagctcatcatcataatccatggactgtctccccatttagcaaaacaccgcttcatccttctgcagcaggtgggccgggcggacctctgagcgtatatcctggcgcaggtggcggctccggcggcgggtcaggaagtagtgtcgcgagcctgactccaaccgctgctcatagcgggagtcatctgtttgggtttccgccaacaccccctaaagaggtatccccagatccatctactaccggtgccgccagtcccgcaagctccagtgcaggcggcagcgctgctagaggtgaagataaagatggggtgaaatatcaagtttccctcaccgaaagtatgaaaatggagtccggaagcccacttcgacctggtcttgccaccatgggaacacaacccgccacccatcatcctataccaacgtatccaagctacgtccccgctgcagctcatgattattcatcagggctgtttcatcctggtggattcttgggcggtcctgcttcaagttttactccgaaacaacggtctaaagccaggagctgctccgagggaagggaatgcgtgaattgcggagcaaccgctactcccctttggcgaagggatggtactggtcattatctttgtaacgcatgcgggctgtatcataaaatgaacggccaaaatcggcccctgattaaaccaaaacggagactttccgctgcacggcgtgctgggacatgctgcgctaactgccaaaccaccactacaactctctggcggcggaatgctaatggtgatccagtgtgtaatgcttgcgggctgtattacaaacttcataacgtgaatcgccctcttacgatgaagaaagagggaattcaaacgcgtaatcgtaaaatgtcaaataaatcaaagaaatctaagaagggtgctgaatgttttgaggaactcagcaaatgtatgcaagagaaaagcagcccttttagcgcggcggcgcttgcagggcatatggccccagtaggtcatctgcccccgttttctcatagcggtcatattctcccaaccccaacaccgatacatccaagcagctccttgtcttttgggcatcctcatccaagctcaatggtcacagcaatgggc

SEQ ID NO: 29 - DNA, 1443 bp, synthetic construct
  source 1..1443: mol_type = other DNA
atggaagtcgccccagaacaacctaggtggatggcccatcctgcagtactcaacgcccaacatcctgatagtcatcatcccggattggcacataattatatggagccggctcaactccttcccccggatgaagtcgatgttttctttaaccatttggatagtcaaggaaatccatattacgcgaatcctgcccatgcccgtgccagggtgagctattcccctgcccatgctagactcactggtggtcaaatgtgtaggccccatcttctccattcccctggactgccttggttggatggcggaaaggctgctctgtccgctgccgccgcacatcatcataatccttggacagtcagtccttttagcaaaacaccactccatccttctgctgccggcggacccggcggtcctctgtcagtttatccgggcgctggtggcggctctggtggtggatcaggttcttccgtcgcaagcctgactcccactgccgcgcattccggtagccatctctttggttttccccctacacccccaaaggaagtctcccccgatccctcaactactggcgcggctagtcctgcgtcctcttctgccggcggcagcgctgctcgcggtgaagataaagatggagtgaaatatcaagttagcctcaccgaatctatgaaaatggagagcggatctcctttgcgtcccgggttggccaccatgggtacacaaccagccacccatcatcctattcctacttatccgagctacgtacctgctgccgcacatgattattcatctgggctgtttcatccaggcgggtttctcggtggccccgcaagctcatttactcccaaacaaaggtccaaagcgcggagctgcagtgaggggagagaatgcgtgaattgcggcgcaaccgctacgcccttgtggagaagggatggaacggggcattatctctgtaacgcatgcgggttgtatcataaaatgaacggccaaaatcgccccttgataaaaccaaagagacggttgtcagctgcacgacgcgctggaacttgctgcgccaactgccaaaccaccacaacaacattgtggcggcgtaatgcaaatggcgatcccgtgtgtaatgcttgcggtctgtattataaattgcataacgtgaatagacccctcactatgaagaaagagggcatacaaacccgcaatcgcaaaatgtcaaataaaagcaagaagtctaagaagggcgccgaatgttttgaagaattgtctaaatgtatgcaagaaaagagcagcccattttccgccgccgcactcgcaggccatatggccccagtaggtcatctgcccccttttagtcatagcgggcatattttgcctacgccaacaccgattcatcctagcagctcactgtcttttgggcatccacatccaagttctatggtcacggctatgggttga

SEQ ID NO: 30 - DNA, 1440 bp, synthetic construct
  source 1..1440: mol_type = other DNA
atggaagtagcccctgaacaacccagatggatggctcatccagcagtattgaacgcacaacatccagattcccatcatccaggattggcccataattatatggagccagcccaactcttgccacctgatgaagtcgatgttttctttaaccatctggatagtcaaggtaatccttattacgctaatccagcacatgcacgcgctcgggtttcatattcccctgcccatgcaaggcttacaggcggacaaatgtgtcgtcctcatctgctccattccccaggccttccatggcttgatggcgggaaggcggcgctttcagcggcggctgcacatcatcataatccgtggactgtctctccttttagtaaaactccgcttcatccgagcgcagcaggcggacctggcggacccctgtcagtctatcctggtgccggtggcggctccggcggtgggtccggttccagcgttgcaagcttgacaccaacggcggctcattcaggatcacatttgtttggttttcccccaaccccgccgaaagaggtcagtcccgatccaagtacaaccggagcagcaagtcccgcaagctcatctgccggcggctccgccgctagaggcgaagataaagatggggtaaaatatcaagtctctcttaccgaatccatgaaaatggagtctggatctcctctccggcccgggctcgcgaccatggggacacaacccgcaacccatcatccaattccaacttatccaagctacgttcccgctgcagcacatgattattcatctgggctgtttcatccgggcggatttctcggtggcccagcatcttcttttactcccaaacaacggtctaaagcaaggtcttgcagtgagggtagggaatgcgtgaattgcggagctactgcaactcccctgtggcgtcgcgatgggacaggtcattatctttgtaacgcttgcggactttatcataaaatgaacggccaaaatcgcccgctgattaaacctaaacgcaggttgtcagcggctcgcagggcggggacatgctgcgccaactgccaaaccaccactacaacactgtggcgaagaaatgcaaatggcgatcccgtgtgtaatgcttgcgggctttattataaactccataacgtcaatcggcctcttacaatgaagaaagagggaattcaaaccaggaataggaaaatgtcaaataaaagcaagaaatcaaagaagggcgccgaatgttttgaagaattgtctaaatgtatgcaagagaaatcttcaccgttttccgccgccgcgttggccggtcatatggctccagtcggacatcttccgccattttcacatagtgggcatattcttccaacacccacacctattcatccttcatcttctctgtcatttggacatcctcatcctagctccatggttactgcaatgggc

SEQ ID NO: 31 - DNA, 1443 bp, synthetic construct
  source 1..1443: mol_type = other DNA
atggaagttgccccggaacaaccccgttggatggcccatcctgcagtacttaacgcccaacatccggattcccatcatcccggactcgctcataattatatggagccggctcaactcttgccacccgatgaagtcgatgtgttctttaaccatctggattctcaagggaatccatattacgcaaatccagcacatgcccgagccagagttagctattcacctgcccatgcaagattgactggtggacaaatgtgtcggccccatctgctccattcaccagggctcccatggcttgatggcggtaaggctgcgctgagcgctgccgccgcacatcatcataatccatggacagtatctcccttttctaaaactccccttcatccttctgcagccggtggacccggcggacctttgagtgtttatcctggcgcaggcggcggttccggcggcgggagtggaagcagtgtcgcgagtctgacgcccactgctgcacattcaggaagtcatctgtttgggtttcccccaactccacccaaagaggtttccccagatccaagtacaaccggcgcagccagtcccgcttcctctagcgccggcggctcagctgctagaggggaagataaagatggtgttaaatatcaagttagccttactgaatctatgaaaatggagtcagggagcccgctcagacccgggctggcgacgatgggtacacaaccggccactcatcatcctattccgacgtatccgagttacgttcccgccgccgctcatgattattcttccggcctgtttcatccaggcggttttcttggcggccccgcgagctcatttacgcccaaacaaaggagtaaagccagatcttgctctgaggggagagaatgcgtgaattgcggcgcgacggcaactcccttgtggcgccgcgatggtactgggcattatctctgtaacgcatgcggattgtatcataaaatgaacggtcaaaataggcctctgataaaacctaaacggcgccttagtgcggcaaggcgtgcaggaacatgctgcgctaactgccaaacaactacaactactttgtggcggcgcaatgcgaatggtgatcccgtgtgtaatgcttgcggattgtattataaactccataacgtgaatcgtcctctcactatgaagaaggaaggaattcaaacgcgcaataggaaaatgtctaataaaagcaagaagtccaagaagggcgctgaatgttttgaagaactcagcaaatgtatgcaagagaaatccagtccattttcagccgccgcacttgcaggtcatatggctcccgtcgggcatttgccaccgttttcccatagcggtcatattctcccaacccctactccaatacatccttctagctctctgagttttgggcatcctcatcccagttccatggtcacagcgatgggttga

SEQ ID NO: 32 - DNA, 990 bp, synthetic construct
  source 1..990: mol_type = other DNA
atgccgaggagctttctcgttaaatccaagaaagcgcatacatatcatcaacctagggtccaagaggacgagccactgtggccacccgctctcacaccagttcctcgggatcaagcaccttctaattcacccgtgttgagtaccttgtttccgaatcaatgtctcgattggactaatctgaagcgggaaccagaattggaacaagatcaaaatctggcacgcatggctcccgctcctgaaggtccaatcgttctcagtcggccgcaagacggcgattccccgctttctgatagcccaccgttttataaaccatcttttagctgggatacgctcgctacgacatacggtcattcctatagacaagctccgtcaactatgcaaagcgcttttctcgaacatagcgtttcactttatggatcacccctcgtcccttccaccgaacctgctctggacttttccctgagatattctcccgggatggacgcctatcattgcgttaaatgtaataaagtgttttcaacaccgcatggtcttgaggtccacgtacgccgatcacacagcggcactcgcccgtttgcatgcgatatttgtggaaagacttttggacatgccgtctctctcgaacaacatacacatgtgcattcacaagaacgttcctttgaatgtcgaatgtgtgggaaagcgtttaaaaggtcaagtaccctcagcacgcatcttcttattcattctgatacccgcccttatccatgtcaattttgtgggaaacgctttcatcagaaaagtgatatgaagaaacatacgtatattcataccggggaaaagccacataaatgtcaagtatgtggcaaagcattttcccaaagttctaatttgataacgcatagtcgaaaacatactgggtttaaaccattttcctgcgaactctgtactaagggatttcaaaggaaagtagatcttagaaggcatagggaatcccaacataacctgaaa

SEQ ID NO: 33 - DNA, 993 bp, synthetic construct
  source 1..993: mol_type = other DNA
atgccccggagttttcttgttaaatccaagaaagcccatacatatcatcaacctcgcgtccaagaggacgagccactgtggcccccagctctgacgcctgtacctcgggatcaagcaccgtctaattctcccgtgctgtcaaccctgtttcctaatcaatgtcttgattggactaatcttaagcgcgaaccagaactcgaacaagatcaaaatctggcacggatggctcctgcccccgaaggaccaatcgtactctctcggcctcaagacggcgatagtcccttgtcagattcacccccattttataaaccatcctttagttgggatacactcgctactacatacggtcattcatatagacaagctccatctacaatgcaatccgcgtttctcgaacatagcgtgagtctttatggtagcccactggttccaagtaccgaaccggcactggatttctcacttcgttatagccctgggatggacgcttatcattgcgtcaaatgtaataaagtgtttagtacgccccatggactggaagtccacgtaaggcggagtcactccggcactagaccttttgcatgcgatatatgtgggaagacttttggtcatgccgtttcccttgaacaacatactcatgtgcattctcaagaaagatcctttgaatgtcggatgtgtggaaaagcatttaaaaggtcatcaactctcagcactcatttgctgattcattctgatacccgcccatatccttgtcaattttgtggtaaaaggtttcatcagaaaagcgatatgaagaaacatacctacattcatactggagagaagccccataaatgtcaagtttgtggtaaggcattttcacaatcttctaacctgataacacattcccggaaacatactggatttaaaccattttcttgcgaactctgtacaaagggttttcaaagaaaagtagatctccgtaggcatcgtgaatcacaacataacctgaaataa

SEQ ID NO: 34 - DNA, 990 bp, synthetic construct
  source 1..990: mol_type = other DNA
atgcccagaagctttctcgtcaaatcaaagaaagcccatacatatcatcaacctcgggttcaagaggacgagcctttgtggccaccagctctcacaccagtccctcgggatcaagccccttcaaatagtccagtactctctacactctttcctaatcaatgtttggattggacaaatctgaagcgggaaccagaacttgaacaagatcaaaatctggcacggatggcacctgcgcctgaaggaccaatcgtcttgtctaggcctcaagacggcgatagcccgctcagcgattccccaccgttttataaacccagttttagctgggatacactggctacgacatacggacattcatataggcaagctcctagtactatgcaatccgcatttctcgaacatagcgtgtctttgtatgggagcccgctcgtaccttctaccgaacctgcgctggattttagtctgcgttatagccctgggatggacgcctatcattgcgtcaaatgtaataaagtctttagcactccccatggcctggaagtccacgtccggcgaagtcactctggtactagaccatttgcttgcgatatttgtggaaagacttttggacatgccgtcagtcttgaacaacatacacatgtgcattctcaagaacggtcctttgaatgtcggatgtgtgggaaagcttttaaaagaagctctactctctcaacgcatctcttgattcatagcgatacacgtccttatccatgtcaattttgtggaaaacgcttccatcaaaagtcagatatgaagaaacatacttatattcatactggcgagaaaccacacaaatgtcaagtttgcggcaaagcattttcccaatcctcaaatctgattactcattccagaaaacatactgggtttaaaccattttcctgcgaactttgtacgaagggttttcaaaggaaagttgatttgcgacgccatagagaatcccaacataacctgaaa

SEQ ID NO: 35 - DNA, 990 bp, synthetic construct
  source 1..990: mol_type = other DNA
atgccgagatcatttctcgtcaaaagtaagaaggcgcatacttatcatcaacctcgcgttcaagaggacgagcccttgtggcccccagcgttgacacctgttcctcgcgatcaagcaccctctaatagtcccgtgctctcaaccctgtttcctaatcaatgtttggattggacaaatctgaagcgtgaacctgaattggaacaagatcaaaatcttgcacgcatggcgccagcgcctgaaggtccaatcgtcctcagtagaccacaagacggagatagcccgttgagcgattctccacccttttataaaccatcctttagttgggatacactggcaacgacatacggacatagttataggcaagctccatctactatgcaatctgcgtttctggaacatagcgtttctctttatgggtctccgctggttccatcaacagaacctgcgctggattttagcctgagatatagtcctgggatggacgcatatcattgcgtaaaatgtaataaagtgtttagcacaccacatggcctggaagtccacgttcggcggagccacagcggcacaaggccatttgcatgcgatatttgtgggaagacgtttggacatgccgtttccctcgaacaacatacacatgttcatagccaagaaagatcatttgaatgtagaatgtgtgggaaagcatttaaacggtctagcacactctctacgcatctcctgattcatagcgatactcgcccatatccttgtcaattttgcgggaaaagatttcatcaaaagtcagatatgaagaagcatacgtatattcataccggggagaaaccacataaatgtcaagtctgtggcaaagccttttcccagtcatctaatttgataacacatagtaggaaacataccggttttaaaccgttttcttgcgaactctgtacaaagggttttcaaagaaaagttgatctccgtagacatagggaatcccaacataacctgaaa

SEQ ID NO: 36 - DNA, 993 bp, synthetic construct
  source 1..993: mol_type = other DNA
atgccccgaagttttctcgttaaatcaaagaaagcacatacttatcatcaacctcgcgtccaagaggacgagccactttggccacccgctctgacacctgtcccgagggatcaagcgccgtccaattcacccgtgttgtctaccctctttcccaatcaatgtttggattggacaaatttgaagagggaaccagaacttgaacaagatcaaaatcttgcgcggatggcgcctgccccggaagggccaatcgtcctcagccggccgcaagacggcgattcccctcttagcgatagcccaccattttataaacccagttttagctgggatactctggctactacttacggacattcatatagacaagcgccttcaacgatgcaaagcgcatttcttgaacatagtgtgtccctttatggttccccactcgttccttccacagaaccagctctggatttctcacttcgatattctcccgggatggacgcttatcattgcgtaaaatgtaataaagtgttttctacaccgcatggtctggaagtccacgtacgccgtagtcactccggcacaaggccttttgcgtgcgatatttgtgggaagacgtttggtcatgccgtatcactcgaacaacatacacatgtgcattctcaagaacggagttttgaatgtagaatgtgtggtaaagcttttaaaaggtcatcaactttgagtactcatctcttgatacacagcgatacaaggccgtatccatgtcaattttgtggaaaacggtttcatcaaaagtcagatatgaagaaacatacatatattcataccggggaaaagccccataaatgtcaagtctgtggtaaagcgttttctcaatctagtaatctgattacacattctcgtaaacatactgggtttaaacctttctcttgcgaactttgtacaaagggatttcaacggaaagtcgatcttcgtagacatagagaatcccaacataaccttaaataa

SEQ ID NO: 37 - DNA, 990 bp, synthetic construct
  source 1..990: mol_type = other DNA
atgccccgttctttcttggtaaaatccaagaaggcgcatacatatcatcaaccacgggttcaagaggacgagccactgtggccgccagctctgactcccgtccctcgggatcaagccccctctaattctcccgtgctgtcaacattgtttcctaatcaatgtttggattggactaatctgaagcgcgaaccagaattggaacaagatcaaaatctcgctcggatggctcctgctccggaaggtcctatcgttctttcaagaccacaagacggcgattcacccctctcagattctccccctttctataaacccagctttagttgggatactctggcaaccacatacgggcattcttatcgccaagcgccaagcacaatgcaaagtgcatttctcgaacattcagtatctctttatggatctcccctggtcccttctaccgaaccagcgcttgacttttccttgaggtatagtcccgggatggacgcatatcattgcgtaaaatgtaataaagttttcagcacgccccatggattggaagtccacgtcaggcgatctcactctggcacaagaccttttgcatgcgatatttgtggtaagacgtttggacatgccgtttcactcgaacaacatacacatgttcatagccaagaacgttcatttgaatgtagaatgtgtggaaaagcatttaaaaggagctctaccctttctacacatttgctgattcatagcgatacccgaccttatccgtgtcaattttgtggaaaacgatttcatcagaaatcagatatgaagaagcatacatatatacataccggggagaaaccccataaatgtcaagtctgtggcaaagcattttctcaatcaagcaatctgattactcattccagaaaacatactggatttaaacccttttcatgcgaactttgtactaagggatttcaacggaaagttgatttgaggagacatcgagaaagtcaacataacctgaaa

SEQ ID NO: 38 - DNA, 1143 bp, synthetic construct
  source 1..1143: mol_type = other DNA
atgatgttttccgggtttaatgccgattatgaagcatccagttctaggtgtagttctgcaagtcccgcgggcgactcactgtcatattatcatagcccggctgatagcttttcttctatggggtcacccgtgaatgcccaagatttctgtactgatttggcagtgagctcagctaattttatccctactgtgaccgctataagcacttctcctgatctccaatggctcgtccaaccagcactggttagttcagtcgctcccagtcaaacacgggcaccccatccgtttggggttccagcaccttcagcaggcgcctatagccgggccggagtcgtaaagacaatgactggtgggagagcccaatcaatcggaagaagaggaaaagtcgagcaacttagccctgaggaagaggaaaagagacgcattaggcgcgagcggaacaaaatggccgccgctaagtgtcgaaataggcgtcgtgaactcacagacaccctgcaggccgaaaccgatcagcttgaggacgagaaaagtgcactgcaaactgaaatcgctaatcttctcaaagagaaagagaagctggaatttattctcgctgcacatcgtccagcatgtaaaattcccgacgatctcggatttccggaagaaatgagcgtagccagtctcgaccttaccggtggactccctgaagtcgcgacacctgaatccgaagaagcgtttacattgcccttgctgaacgatccggaaccaaaaccaagcgtcgagcccgtgaaaagtatatcatctatggaacttaaaactgaacctttcgacgattttctctttcctgcttccagccgcccatcagggtcagaaaccgctagatctgtccccgatatggatctgagcggaagcttttacgctgccgattgggaaccactccattcaggtagtcttggcatgggccctatggcgaccgaacttgaacctctttgtacacccgttgtaacttgcaccccatcctgtacggcctatacctcaagttttgtatttacatatcctgaagccgattcttttccatcctgcgccgccgcgcatcgaaaagggtcttcatctaacgaaccatcttccgatagtttgagtagtccaacccttcttgctctctaa

SEQ ID NO: 39 - DNA, 1140 bp, synthetic construct
  source 1..1140: mol_type = other DNA
atgatgttttcagggtttaatgccgattatgaagccagttcttcaagatgttcctcagcttcccccgcaggcgactccttgtcctattatcattctccagctgatagctttagttccatggggtcccccgttaatgcccaagacttttgtactgatctcgcggttagcagcgcaaattttataccgactgtgacagcaatatcaacatctccagatcttcaatggctcgttcaacctgcactggtgagctctgtcgcaccttcacaaacgcgggctccacatccatttggcgttcctgcaccaagtgcaggtgcctatagccgcgcaggggtggttaaaacaatgactggtggaagagcccaatccatcgggcgccggggaaaagtagagcaactgtcccccgaggaagaggaaaagcgtcgtattaggcgcgagcggaacaaaatggcggctgcaaagtgtagaaataggcgccgcgaattgacagacaccctgcaggcagaaaccgatcagctcgaggacgaaaagtccgcccttcaaactgaaatcgcaaatttgcttaaagagaaagagaagttggaatttatactcgctgcccatcggccagcatgtaaaatacccgacgatttgggttttcccgaggaaatgtcagtagcctctctcgaccttacgggcggacttcccgaagtggcgactcctgaaagtgaagaagcatttacacttcccctgctgaacgatccagaacctaaaccttccgtcgagcccgtaaaatcaatttcttccatggaattgaaaacagaacctttcgacgattttctctttcctgccagttcacgcccatccgggagtgaaaccgctcgaagcgttccggatatggatcttagcggcagcttttacgctgctgattgggaaccgcttcatagcgggtcattgggtatgggaccaatggctaccgaacttgaaccattgtgtaccccagttgtgacatgcaccccttcttgtaccgcgtatacttcaagctttgtatttacatatcctgaagccgattcatttccaagttgcgccgccgctcataggaaagggtctagtagtaacgaaccaagttcagattcactgtccagccctacattgttggctctc

SEQ ID NO: 40 - DNA, 1143 bp, synthetic construct
  source 1..1143: mol_type = other DNA
atgatgttttcagggtttaatgccgattatgaagccagttcatctcgatgttcatcagccagccctgctggcgactcactgagctattatcattctccagctgattcattttcttctatggggagcccagtaaatgcacaagacttttgtaccgatctcgcagtttcaagcgcgaattttatcccaaccgtgaccgctattagcacttctccagatctccaatggcttgtccaaccggctctggtgagtagcgtcgctccgagccaaacgagggcaccccatccgtttggtgtaccagcaccaagcgcaggagcatattctcgcgcgggtgtagtcaaaacaatgactggcgggcgcgcccaaagtatcggtcgcagaggtaaagtagagcaactgagccccgaggaagaggaaaagcgacggattagacgtgagcggaacaaaatggccgccgctaagtgtcggaatagacgacgagaacttacagacactctgcaggccgaaaccgatcagttggaagacgaaaagtccgcactccaaacagaaatcgcgaatcttttgaaagagaaagagaagctcgaatttattcttgccgcacatcgccccgcgtgtaaaatacccgacgatcttggatttcctgaggaaatgtccgttgcaagcctggacctcaccggcggacttccggaagtcgcaacacccgaaagtgaagaagcttttactcttcccctgctgaacgatcccgaacctaaaccttctgttgagcccgttaaatcaataagtagtatggaactcaaaactgaaccattcgacgattttctgtttcccgcttccagtagaccaagcgggagcgaaactgcacgatctgtcccggatatggatctgtcaggctcattttacgcggccgattgggaaccactccattccggatctctcggcatgggaccaatggctaccgaacttgaaccactttgtacccccgtcgtaacgtgcacaccatcctgtaccgcgtatactagctcttttgtatttacttatccggaagcggattcatttccaagttgcgccgccgctcatagaaaagggtcttcatctaacgaaccgtcttccgattccctgagtagtcctacccttttggcattgtaa

SEQ ID NO: 41 - DNA, 1140 bp, synthetic construct
  source 1..1140: mol_type = other DNA
atgatgtttagcggatttaatgccgattatgaagcaagctcaagcaggtgttcctcagcttctcccgctggagactctctgtcatattatcatagcccagccgatagcttttcttccatgggttccccagttaatgctcaagatttctgtaccgatctcgcggtgagtagcgcaaattttataccaaccgtgaccgctatttctacatctcccgatcttcaatggttggtccaacctgctctggtgagttctgtcgcaccctctcaaactcgggcaccgcatccctttggcgtaccagcaccaagcgccggagcctatagccgggcaggggtggtcaaaactatgaccggtgggcgtgcccaatccataggacgccgcgggaaagtcgagcaattgagcccggaagaagaggaaaagcggcgcattcgccgggagcgaaacaaaatggccgccgctaagtgtcgtaatagacgccgcgaactcaccgacacgttgcaggctgaaaccgatcagctcgaggacgagaaaagtgcgctccaaacggaaatcgctaatcttctcaaagaaaaggagaagcttgaatttattttggccgcccatcgccccgcttgtaaaataccggacgatcttgggtttcccgaggaaatgagtgtcgcctctctggacctcaccggcggattgcctgaagtggctacaccagaatccgaagaagcgtttacgctccccctgctgaacgatcccgaaccaaaaccatccgttgagccagtgaaatcaatatcatccatggaacttaaaacagaacctttcgacgattttctgtttcctgccagctcacgcccgagcggttcagaaaccgcgaggtctgtacccgatatggatctgtcaggttcattttacgctgccgattgggaacccttgcatagcggttctttgggcatgggacctatggctaccgaactcgaacctctctgtacacctgtcgtgacatgcacaccttcttgtaccgcctatacctcaagttttgtgtttacttatccagaagccgatagctttcctagttgcgctgccgcacatcgtaaaggaagttcctcaaacgaaccgagttccgattctcttagtagcccaacccttttggcactt

SEQ ID NO: 42 - DNA, 1140 bp, synthetic construct
  source 1..1140: mol_type = other DNA
atgatgttttcaggatttaatgctgattatgaagccagctctagtaggtgttcctctgcaagcccagctggtgactccctgtcatattatcatagtcctgctgatagctttagttctatgggatctccagtgaatgcccaagacttttgtacagatctcgcagtaagcagcgcaaattttatcccgactgttaccgctatttccacttcacccgatctccaatggcttgtccaaccagcgctggtgagtagtgttgctccgagccaaacacgggctccccatccctttggggtgcctgctccgagcgcaggcgcatatagccgtgccggagtggtgaaaacaatgaccggcggacgggcacaatctatcgggcgtagaggtaaagtcgagcaactgagtcctgaggaagaggaaaagagacggattaggcgggagcgcaacaaaatggcagccgcaaagtgtcgaaatagaagacgcgaactcacggacaccctgcaggcagaaactgatcagcttgaggacgaaaagagtgccctgcaaactgaaatcgcaaatcttctcaaagaaaaggagaagctggaatttattctcgccgcacataggcccgcttgtaaaattcccgacgatctcggatttcctgaggaaatgagcgttgcctcactggaccttacaggcgggttgcccgaagtggctactcccgaatcagaagaagcatttactcttcccctgctgaacgatcccgaaccaaaaccatccgttgagccggtgaaatctattagttcaatggaattgaaaactgaacctttcgacgattttctctttcctgcctctagccgaccatcagggtccgaaacggctcgtagtgtacctgatatggatctgagcggtagcttttacgccgccgattgggaacctctccattccggttcattgggcatgggaccgatggctactgaattggaaccgctctgtaccccagtagtgacatgcacaccttcatgtacagcatatacatcaagctttgtgtttacttatccggaagccgattcttttccttcttgcgctgccgctcataggaaaggctcctcttctaacgaaccaagtagtgattcactgtcatcccctacacttttggctctt

SEQ ID NO: 43 - DNA, 1140 bp, synthetic construct
  source 1..1140: mol_type = other DNA
atgatgtttagcggatttaatgccgattatgaagcaagctcatctcgatgttcatctgcaagcccagctggtgactcactgagctattatcatagcccggctgattcattttcatctatgggttcccccgttaatgcccaagacttttgtaccgatctcgcagtgagcagcgcaaattttatccctaccgtgaccgctatatcaactagccccgatttgcaatggcttgtccaaccagcgttggtgtcaagcgttgcacccagccaaactcgcgctccccatccctttggcgtgccagctccgagcgcaggcgcctattcacgggcaggggtagtcaaaacaatgaccggcggacgcgctcaatccatcggaagaagagggaaagtcgagcaactctcccccgaggaagaggaaaagcgacgcattagaagagagcggaacaaaatggccgccgctaagtgtcggaatagacgccgcgaactcaccgacactctgcaggccgaaaccgatcagctggaagacgaaaagagtgcccttcaaactgaaatcgcaaatctcttgaaagaaaaggagaagctggaatttattcttgctgcacatcgtccagcatgtaaaattccggacgatttggggtttcccgaggaaatgtccgttgcctctctcgacttgaccggtgggttgcctgaagtagctacacccgaaagtgaagaagcgtttactcttccattgcttaacgatccagaaccaaaaccaagcgttgagcccgtaaaatctatttcttcaatggaactcaaaactgaaccattcgacgattttctctttcctgcttctagtagaccatctggctccgaaaccgcacgatctgtacctgatatggatctgtccggaagcttttacgccgccgattgggaaccactccattccggttcactcggaatgggacctatggctacggaattggaacctctttgtaccccagttgtaacttgcaccccatcatgtaccgcctatacctctagctttgtatttacgtatcctgaagccgatagctttccatcttgcgccgccgcgcataggaaaggatcttcaagtaacgaacccagttccgatagtttgagttctccgacacttctcgctctt

SEQ ID NO: 44 - DNA, 3702 bp, synthetic construct
  source 1..3702: mol_type = other DNA
atggaagtcgctcctgaacaaccacggtggatggctcatcctgcagttcttaacgctcaacatccagatagccatcatcctgggctcgctcataattatatggagcctgcccaactccttcccccagatgaagtagatgtattctttaaccatcttgatagccaaggaaatccttattacgcaaatcctgcccatgccagagccagagtaagctattcaccagcccatgcaaggcttactggtggacaaatgtgtcggccccatctgctgcattcccccggcctgccgtggttggatggcggaaaggcggctctgagtgcggcggctgcacatcatcataatccttggacagtctcacctttctctaaaactccccttcatcctagtgcagccggtggtccaggtggtccgctgtccgtttatcccggcgccggcggcggaagtggcggcggaagtggcagtagcgtagcttcattgacacccacagctgctcattccggaagtcatctctttgggtttccccctaccccgccgaaagaggtttccccagatccgtcaactaccggtgccgcttcccctgctagttccagtgccggcggaagcgctgcaaggggcgaagataaagatggagtgaaatatcaagtctccttgacagaatccatgaaaatggagtccggttctccactcaggcctggactcgccacaatgggtactcaacccgccacccatcatccaataccgacttatccttcatacgtaccagccgccgctcatgattattcatcaggtttgtttcatccaggtggatttcttggcgggcctgctagttcttttacaccaaaacaaaggtccaaagccaggtcttgcagtgaggggcgcgaatgcgtaaattgcggcgctaccgcaacaccactgtggcgtcgtgatggaacaggacattatctttgtaacgcatgcgggctgtatcataaaatgaacggacaaaatcggcccctgattaaacctaaacgtcgcttgtctgctgcaaggcgggctgggacgtgctgcgccaactgccaaacaaccacaacaacactgtggcgtaggaatgctaatggcgatcccgtatgtaatgcatgcggtctgtattataaacttcataacgtcaatcggccccttactatgaagaaagagggcatacaaacccgaaatcgcaaaatgtcaaataaatctaagaaatccaagaagggagccgaatgttttgaagaactcagcaaatgtatgcaagaaaagtccagcccgttttccgccgccgctttggccggccatatggcccccgtcggacatctgccaccgttttcccatagcggtcatattcttcctacacccaccccaattcatccgtctagctccctgtcttttgggcatcctcatcctagctcaatggtcacagctatgggtggcagcggcgccacaaacttctctctgctaaagcaagcaggtgatgttgaagaaaaccccgggcctatgccgaggagctttctcgttaaatccaagaaagcgcatacatatcatcaacctagggtccaagaggacgagccactgtggccacccgctctcacaccagttcctcgggatcaagcaccttctaattcacccgtgttgagtaccttgtttccgaatcaatgtctcgattggactaatctgaagcgggaaccagaattggaacaagatcaaaatctggcacgcatggctcccgctcctgaaggtccaatcgttctcagtcggccgcaagacggcgattccccgctttctgatagcccaccgttttataaaccatcttttagctgggatacgctcgctacgacatacggtcattcctatagacaagctccgtcaactatgcaaagcgcttttctcgaacatagcgtttcactttatggatcacccctcgtcccttccaccgaacctgctctggacttttccctgagatattctcccgggatggacgcctatcattgcgttaaatgtaataaagtgttttcaacaccgcatggtcttgaggtccacgtacgccgatcacacagcggcactcgcccgtttgcatgcgatatttgtggaaagacttttggacatgccgtctctctcgaacaacatacacatgtgcattcacaagaacgttcctttgaatgtcgaatgtgtgggaaagcgtttaaaaggtcaagtaccctcagcacgcatcttcttattcattctgatacccgcccttatccatgtcaattttgtgggaaacgctttcatcagaaaagtgatatgaagaaacatacgtatattcataccggggaaaagccacataaatgtcaagtatgtggcaaagcattttcccaaagttctaatttgataacgcatagtcgaaaacatactgggtttaaaccattttcctgcgaactctgtactaagggatttcaaaggaaagtagatcttagaaggcatagggaatcccaacataacctgaaaggctccggcgagggcaggggaagtcttctaacatgcggggacgtggaggaaaatcccggcccaatgatgttttccgggtttaatgccgattatgaagcatccagttctaggtgtagttctgcaagtcccgcgggcgactcactgtcatattatcatagcccggctgatagcttttcttctatggggtcacccgtgaatgcccaagatttctgtactgatttggcagtgagctcagctaattttatccctactgtgaccgctataagcacttctcctgatctccaatggctcgtccaaccagcactggttagttcagtcgctcccagtcaaacacgggcaccccatccgtttggggttccagcaccttcagcaggcgcctatagccgggccggagtcgtaaagacaatgactggtgggagagcccaatcaatcggaagaagaggaaaagtcgagcaacttagccctgaggaagaggaaaagagacgcattaggcgcgagcggaacaaaatggccgccgctaagtgtcgaaataggcgtcgtgaactcacagacaccctgcaggccgaaaccgatcagcttgaggacgagaaaagtgcactgcaaactgaaatcgctaatcttctcaaagagaaagagaagctggaatttattctcgctgcacatcgtccagcatgtaaaattcccgacgatctcggatttccggaagaaatgagcgtagccagtctcgaccttaccggtggactccctgaagtcgcgacacctgaatccgaagaagcgtttacattgcccttgctgaacgatccggaaccaaaaccaagcgtcgagcccgtgaaaagtatatcatctatggaacttaaaactgaacctttcgacgattttctctttcctgcttccagccgcccatcagggtcagaaaccgctagatctgtccccgatatggatctgagcggaagcttttacgctgccgattgggaaccactccattcaggtagtcttggcatgggccctatggcgaccgaacttgaacctctttgtacacccgttgtaacttgcaccccatcctgtacggcctatacctcaagttttgtatttacatatcctgaagccgattcttttccatcctgcgccgccgcgcatcgaaaagggtcttcatctaacgaaccatcttccgatagtttgagtagtccaacccttcttgctctctaa

SEQ ID NO: 45 - DNA, 3702 bp, synthetic construct
  source 1..3702: mol_type = other DNA
atggaagtcgccccggaacaaccacgatggatggcacatcctgcagttctcaacgcacaacatcctgatagtcatcatccagggttggctcataattatatggagcctgcccaactccttccccctgatgaagttgatgtgttctttaaccatctggattctcaaggaaatccgtattacgctaatccagcccatgcacgcgcacgtgtttcatatagtcctgcccatgcgaggcttacaggcgggcaaatgtgtcgtccccatctcctgcattctcccggcctgccgtggcttgatggtggaaaggctgcgctgagcgctgccgccgctcatcatcataatccatggactgtcagtccgttttctaaaactcctttgcatccatctgcagccggtggaccaggcggtccgctttcagtttatcccggtgcgggtggcggctccggcggcggttcaggatcaagcgtcgcgagcctgactccaacggctgctcatagtggtagccatctgtttgggtttcccccaactccccctaaagaggtcagcccagatccaagcacgaccggcgccgcttcccctgcatccagctctgccggtgggagcgcggcaagaggggaagataaagatggagtgaaatatcaagtaagtttgacagaatcaatgaaaatggagagcggatctcctctccggcccgggcttgccacaatgggtactcaaccagccacccatcatcctattccaacatatcctagctacgttcccgccgccgctcatgattattcatctggtctgtttcatcctggcgggttcttgggcggccccgcgagcagttttacacccaaacaacgttctaaagccaggagctgcagtgagggtagagaatgcgttaattgcggcgctactgcaacaccgctttggcgaagagatggtactggtcattatttgtgtaacgcatgcggtctgtatcataaaatgaacggtcaaaatcggcccctgattaaacctaaaaggcggcttagtgcggcaaggcgtgcaggaacttgctgcgccaactgccaaacaacgacaactactctctggagacggaatgcaaatggtgatccggtttgtaatgcgtgcgggctgtattataaattgcataacgtcaatcggccgcttacaatgaagaaagagggaatacaaaccagaaatcgcaaaatgagcaataaatctaagaaatctaagaagggagcagaatgttttgaggaactcagcaaatgtatgcaagaaaagtccagccctttctccgctgcagctttggcggggcatatggctcccgtcggtcatcttccaccgttttctcatagcggtcatattcttcctacgcccaccccaatacatccttcttcatctttgtcatttggacatcctcatccatcttctatggtcactgcaatgggcggcagcggcgccacaaacttctctctgctaaagcaagcaggtgatgttgaagaaaaccccgggcctatgatgttttcagggtttaatgccgattatgaagccagttcttcaagatgttcctcagcttcccccgcaggcgactccttgtcctattatcattctccagctgatagctttagttccatggggtcccccgttaatgcccaagacttttgtactgatctcgcggttagcagcgcaaattttataccgactgtgacagcaatatcaacatctccagatcttcaatggctcgttcaacctgcactggtgagctctgtcgcaccttcacaaacgcgggctccacatccatttggcgttcctgcaccaagtgcaggtgcctatagccgcgcaggggtggttaaaacaatgactggtggaagagcccaatccatcgggcgccggggaaaagtagagcaactgtcccccgaggaagaggaaaagcgtcgtattaggcgcgagcggaacaaaatggcggctgcaaagtgtagaaataggcgccgcgaattgacagacaccctgcaggcagaaaccgatcagctcgaggacgaaaagtccgcccttcaaactgaaatcgcaaatttgcttaaagagaaagagaagttggaatttatactcgctgcccatcggccagcatgtaaaatacccgacgatttgggttttcccgaggaaatgtcagtagcctctctcgaccttacgggcggacttcccgaagtggcgactcctgaaagtgaagaagcatttacacttcccctgctgaacgatccagaacctaaaccttccgtcgagcccgtaaaatcaatttcttccatggaattgaaaacagaacctttcgacgattttctctttcctgccagttcacgcccatccgggagtgaaaccgctcgaagcgttccggatatggatcttagcggcagcttttacgctgctgattgggaaccgcttcatagcgggtcattgggtatgggaccaatggctaccgaacttgaaccattgtgtaccccagttgtgacatgcaccccttcttgtaccgcgtatacttcaagctttgtatttacatatcctgaagccgattcatttccaagttgcgccgccgctcataggaaagggtctagtagtaacgaaccaagttcagattcactgtccagccctacattgttggctctcggctccggcgagggcaggggaagtcttctaacatgcggggacgtggaggaaaatcccggcccaatgccccggagttttcttgttaaatccaagaaagcccatacatatcatcaacctcgcgtccaagaggacgagccactgtggcccccagctctgacgcctgtacctcgggatcaagcaccgtctaattctcccgtgctgtcaaccctgtttcctaatcaatgtcttgattggactaatcttaagcgcgaaccagaactcgaacaagatcaaaatctggcacggatggctcctgcccccgaaggaccaatcgtactctctcggcctcaagacggcgatagtcccttgtcagattcacccccattttataaaccatcctttagttgggatacactcgctactacatacggtcattcatatagacaagctccatctacaatgcaatccgcgtttctcgaacatagcgtgagtctttatggtagcccactggttccaagtaccgaaccggcactggatttctcacttcgttatagccctgggatggacgcttatcattgcgtcaaatgtaataaagtgtttagtacgccccatggactggaagtccacgtaaggcggagtcactccggcactagaccttttgcatgcgatatatgtgggaagacttttggtcatgccgtttcccttgaacaacatactcatgtgcattctcaagaaagatcctttgaatgtcggatgtgtggaaaagcatttaaaaggtcatcaactctcagcactcatttgctgattcattctgatacccgcccatatccttgtcaattttgtggtaaaaggtttcatcagaaaagcgatatgaagaaacatacctacattcatactggagagaagccccataaatgtcaagtttgtggtaaggcattttcacaatcttctaacctgataacacattcccggaaacatactggatttaaaccattttcttgcgaactctgtacaaagggttttcaaagaaaagtagatctccgtaggcatcgtgaatcacaacataacctgaaataa

SEQ ID NO: 46 - DNA, 3702 bp, synthetic construct
  source 1..3702: mol_type = other DNA
atgcccagaagctttctcgtcaaatcaaagaaagcccatacatatcatcaacctcgggttcaagaggacgagcctttgtggccaccagctctcacaccagtccctcgggatcaagccccttcaaatagtccagtactctctacactctttcctaatcaatgtttggattggacaaatctgaagcgggaaccagaacttgaacaagatcaaaatctggcacggatggcacctgcgcctgaaggaccaatcgtcttgtctaggcctcaagacggcgatagcccgctcagcgattccccaccgttttataaacccagttttagctgggatacactggctacgacatacggacattcatataggcaagctcctagtactatgcaatccgcatttctcgaacatagcgtgtctttgtatgggagcccgctcgtaccttctaccgaacctgcgctggattttagtctgcgttatagccctgggatggacgcctatcattgcgtcaaatgtaataaagtctttagcactccccatggcctggaagtccacgtccggcgaagtcactctggtactagaccatttgcttgcgatatttgtggaaagacttttggacatgccgtcagtcttgaacaacatacacatgtgcattctcaagaacggtcctttgaatgtcggatgtgtgggaaagcttttaaaagaagctctactctctcaacgcatctcttgattcatagcgatacacgtccttatccatgtcaattttgtggaaaacgcttccatcaaaagtcagatatgaagaaacatacttatattcatactggcgagaaaccacacaaatgtcaagtttgcggcaaagcattttcccaatcctcaaatctgattactcattccagaaaacatactgggtttaaaccattttcctgcgaactttgtacgaagggttttcaaaggaaagttgatttgcgacgccatagagaatcccaacataacctgaaaggcagcggcgccacaaacttctctctgctaaagcaagcaggtgatgttgaagaaaaccccgggcctatggaagttgcccctgaacaaccaagatggatggcccatcccgcggtcctcaacgcccaacatccagattctcatcatcccggtcttgcccataattatatggagcctgcccaacttttgcccccagatgaagtagatgtattcttcaaccatctggattcccaaggaaatccttattacgcaaatccagcccatgcacgcgccagggtgagctattctccagcccatgcaaggcttacgggcggacaaatgtgtagacctcatctgctccatagtccaggcctgccatggcttgatggcggtaaggccgcgcttagcgctgcagcagctcatcatcataatccatggactgtctccccatttagcaaaacaccgcttcatccttctgcagcaggtgggccgggcggacctctgagcgtatatcctggcgcaggtggcggctccggcggcgggtcaggaagtagtgtcgcgagcctgactccaaccgctgctcatagcgggagtcatctgtttgggtttccgccaacaccccctaaagaggtatccccagatccatctactaccggtgccgccagtcccgcaagctccagtgcaggcggcagcgctgctagaggtgaagataaagatggggtgaaatatcaagtttccctcaccgaaagtatgaaaatggagtccggaagcccacttcgacctggtcttgccaccatgggaacacaacccgccacccatcatcctataccaacgtatccaagctacgtccccgctgcagctcatgattattcatcagggctgtttcatcctggtggattcttgggcggtcctgcttcaagttttactccgaaacaacggtctaaagccaggagctgctccgagggaagggaatgcgtgaattgcggagcaaccgctactcccctttggcgaagggatggtactggtcattatctttgtaacgcatgcgggctgtatcataaaatgaacggccaaaatcggcccctgattaaaccaaaacggagactttccgctgcacggcgtgctgggacatgctgcgctaactgccaaaccaccactacaactctctggcggcggaatgctaatggtgatccagtgtgtaatgcttgcgggctgtattacaaacttcataacgtgaatcgccctcttacgatgaagaaagagggaattcaaacgcgtaatcgtaaaatgtcaaataaatcaaagaaatctaagaagggtgctgaatgttttgaggaactcagcaaatgtatgcaagagaaaagcagcccttttagcgcggcggcgcttgcagggcatatggccccagtaggtcatctgcccccgttttctcatagcggtcatattctcccaaccccaacaccgatacatccaagcagctccttgtcttttgggcatcctcatccaagctcaatggtcacagcaatgggcggctccggcgagggcaggggaagtcttctaacatgcggggacgtggaggaaaatcccggcccaatgatgttttcagggtttaatgccgattatgaagccagttcatctcgatgttcatcagccagccctgctggcgactcactgagctattatcattctccagctgattcattttcttctatggggagcccagtaaatgcacaagacttttgtaccgatctcgcagtttcaagcgcgaattttatcccaaccgtgaccgctattagcacttctccagatctccaatggcttgtccaaccggctctggtgagtagcgtcgctccgagccaaacgagggcaccccatccgtttggtgtaccagcaccaagcgcaggagcatattctcgcgcgggtgtagtcaaaacaatgactggcgggcgcgcccaaagtatcggtcgcagaggtaaagtagagcaactgagccccgaggaagaggaaaagcgacggattagacgtgagcggaacaaaatggccgccgctaagtgtcggaatagacgacgagaacttacagacactctgcaggccgaaaccgatcagttggaagacgaaaagtccgcactccaaacagaaatcgcgaatcttttgaaagagaaagagaagctcgaatttattcttgccgcacatcgccccgcgtgtaaaatacccgacgatcttggatttcctgaggaaatgtccgttgcaagcctggacctcaccggcggacttccggaagtcgcaacacccgaaagtgaagaagcttttactcttcccctgctgaacgatcccgaacctaaaccttctgttgagcccgttaaatcaataagtagtatggaactcaaaactgaaccattcgacgattttctgtttcccgcttccagtagaccaagcgggagcgaaactgcacgatctgtcccggatatggatctgtcaggctcattttacgcggccgattgggaaccactccattccggatctctcggcatgggaccaatggctaccgaacttgaaccactttgtacccccgtcgtaacgtgcacaccatcctgtaccgcgtatactagctcttttgtatttacttatccggaagcggattcatttccaagttgcgccgccgctcatagaaaagggtcttcatctaacgaaccgtcttccgattccctgagtagtcctacccttttggcattgtaa

SEQ ID NO: 47 - DNA, 3702 bp, synthetic construct
  source 1..3702: mol_type = other DNA
atgccgagatcatttctcgtcaaaagtaagaaggcgcatacttatcatcaacctcgcgttcaagaggacgagcccttgtggcccccagcgttgacacctgttcctcgcgatcaagcaccctctaatagtcccgtgctctcaaccctgtttcctaatcaatgtttggattggacaaatctgaagcgtgaacctgaattggaacaagatcaaaatcttgcacgcatggcgccagcgcctgaaggtccaatcgtcctcagtagaccacaagacggagatagcccgttgagcgattctccacccttttataaaccatcctttagttgggatacactggcaacgacatacggacatagttataggcaagctccatctactatgcaatctgcgtttctggaacatagcgtttctctttatgggtctccgctggttccatcaacagaacctgcgctggattttagcctgagatatagtcctgggatggacgcatatcattgcgtaaaatgtaataaagtgtttagcacaccacatggcctggaagtccacgttcggcggagccacagcggcacaaggccatttgcatgcgatatttgtgggaagacgtttggacatgccgtttccctcgaacaacatacacatgttcatagccaagaaagatcatttgaatgtagaatgtgtgggaaagcatttaaacggtctagcacactctctacgcatctcctgattcatagcgatactcgcccatatccttgtcaattttgcgggaaaagatttcatcaaaagtcagatatgaagaagcatacgtatattcataccggggagaaaccacataaatgtcaagtctgtggcaaagccttttcccagtcatctaatttgataacacatagtaggaaacataccggttttaaaccgttttcttgcgaactctgtacaaagggttttcaaagaaaagttgatctccgtagacatagggaatcccaacataacctgaaaggcagcggcgccacaaacttctctctgctaaagcaagcaggtgatgttgaagaaaaccccgggcctatgatgtttagcggatttaatgccgattatgaagcaagctcaagcaggtgttcctcagcttctcccgctggagactctctgtcatattatcatagcccagccgatagcttttcttccatgggttccccagttaatgctcaagatttctgtaccgatctcgcggtgagtagcgcaaattttataccaaccgtgaccgctatttctacatctcccgatcttcaatggttggtccaacctgctctggtgagttctgtcgcaccctctcaaactcgggcaccgcatccctttggcgtaccagcaccaagcgccggagcctatagccgggcaggggtggtcaaaactatgaccggtgggcgtgcccaatccataggacgccgcgggaaagtcgagcaattgagcccggaagaagaggaaaagcggcgcattcgccgggagcgaaacaaaatggccgccgctaagtgtcgtaatagacgccgcgaactcaccgacacgttgcaggctgaaaccgatcagctcgaggacgagaaaagtgcgctccaaacggaaatcgctaatcttctcaaagaaaaggagaagcttgaatttattttggccgcccatcgccccgcttgtaaaataccggacgatcttgggtttcccgaggaaatgagtgtcgcctctctggacctcaccggcggattgcctgaagtggctacaccagaatccgaagaagcgtttacgctccccctgctgaacgatcccgaaccaaaaccatccgttgagccagtgaaatcaatatcatccatggaacttaaaacagaacctttcgacgattttctgtttcctgccagctcacgcccgagcggttcagaaaccgcgaggtctgtacccgatatggatctgtcaggttcattttacgctgccgattgggaacccttgcatagcggttctttgggcatgggacctatggctaccgaactcgaacctctctgtacacctgtcgtgacatgcacaccttcttgtaccgcctatacctcaagttttgtgtttacttatccagaagccgatagctttcctagttgcgctgccgcacatcgtaaaggaagttcctcaaacgaaccgagttccgattctcttagtagcccaacccttttggcacttggctccggcgagggcaggggaagtcttctaacatgcggggacgtggaggaaaatcccggcccaatggaagtcgccccagaacaacctaggtggatggcccatcctgcagtactcaacgcccaacatcctgatagtcatcatcccggattggcacataattatatggagccggctcaactccttcccccggatgaagtcgatgttttctttaaccatttggatagtcaaggaaatccatattacgcgaatcctgcccatgcccgtgccagggtgagctattcccctgcccatgctagactcactggtggtcaaatgtgtaggccccatcttctccattcccctggactgccttggttggatggcggaaaggctgctctgtccgctgccgccgcacatcatcataatccttggacagtcagtccttttagcaaaacaccactccatccttctgctgccggcggacccggcggtcctctgtcagtttatccgggcgctggtggcggctctggtggtggatcaggttcttccgtcgcaagcctgactcccactgccgcgcattccggtagccatctctttggttttccccctacacccccaaaggaagtctcccccgatccctcaactactggcgcggctagtcctgcgtcctcttctgccggcggcagcgctgctcgcggtgaagataaagatggagtgaaatatcaagttagcctcaccgaatctatgaaaatggagagcggatctcctttgcgtcccgggttggccaccatgggtacacaaccagccacccatcatcctattcctacttatccgagctacgtacctgctgccgcacatgattattcatctgggctgtttcatccaggcgggtttctcggtggccccgcaagctcatttactcccaaacaaaggtccaaagcgcggagctgcagtgaggggagagaatgcgtgaattgcggcgcaaccgctacgcccttgtggagaagggatggaacggggcattatctctgtaacgcatgcgggttgtatcataaaatgaacggccaaaatcgccccttgataaaaccaaagagacggttgtcagctgcacgacgcgctggaacttgctgcgccaactgccaaaccaccacaacaacattgtggcggcgtaatgcaaatggcgatcccgtgtgtaatgcttgcggtctgtattataaattgcataacgtgaatagacccctcactatgaagaaagagggcatacaaacccgcaatcgcaaaatgtcaaataaaagcaagaagtctaagaagggcgccgaatgttttgaagaattgtctaaatgtatgcaagaaaagagcagcccattttccgccgccgcactcgcaggccatatggccccagtaggtcatctgcccccttttagtcatagcgggcatattttgcctacgccaacaccgattcatcctagcagctcactgtcttttgggcatccacatccaagttctatggtcacggctatgggttga

SEQ ID NO: 48 - DNA, 3702 bp, synthetic construct
  source 1..3702: mol_type = other DNA
atgatgttttcaggatttaatgctgattatgaagccagctctagtaggtgttcctctgcaagcccagctggtgactccctgtcatattatcatagtcctgctgatagctttagttctatgggatctccagtgaatgcccaagacttttgtacagatctcgcagtaagcagcgcaaattttatcccgactgttaccgctatttccacttcacccgatctccaatggcttgtccaaccagcgctggtgagtagtgttgctccgagccaaacacgggctccccatccctttggggtgcctgctccgagcgcaggcgcatatagccgtgccggagtggtgaaaacaatgaccggcggacgggcacaatctatcgggcgtagaggtaaagtcgagcaactgagtcctgaggaagaggaaaagagacggattaggcgggagcgcaacaaaatggcagccgcaaagtgtcgaaatagaagacgcgaactcacggacaccctgcaggcagaaactgatcagcttgaggacgaaaagagtgccctgcaaactgaaatcgcaaatcttctcaaagaaaaggagaagctggaatttattctcgccgcacataggcccgcttgtaaaattcccgacgatctcggatttcctgaggaaatgagcgttgcctcactggaccttacaggcgggttgcccgaagtggctactcccgaatcagaagaagcatttactcttcccctgctgaacgatcccgaaccaaaaccatccgttgagccggtgaaatctattagttcaatggaattgaaaactgaacctttcgacgattttctctttcctgcctctagccgaccatcagggtccgaaacggctcgtagtgtacctgatatggatctgagcggtagcttttacgccgccgattgggaacctctccattccggttcattgggcatgggaccgatggctactgaattggaaccgctctgtaccccagtagtgacatgcacaccttcatgtacagcatatacatcaagctttgtgtttacttatccggaagccgattcttttccttcttgcgctgccgctcataggaaaggctcctcttctaacgaaccaagtagtgattcactgtcatcccctacacttttggctcttggcagcggcgccacaaacttctctctgctaaagcaagcaggtgatgttgaagaaaaccccgggcctatggaagtagcccctgaacaacccagatggatggctcatccagcagtattgaacgcacaacatccagattcccatcatccaggattggcccataattatatggagccagcccaactcttgccacctgatgaagtcgatgttttctttaaccatctggatagtcaaggtaatccttattacgctaatccagcacatgcacgcgctcgggtttcatattcccctgcccatgcaaggcttacaggcggacaaatgtgtcgtcctcatctgctccattccccaggccttccatggcttgatggcgggaaggcggcgctttcagcggcggctgcacatcatcataatccgtggactgtctctccttttagtaaaactccgcttcatccgagcgcagcaggcggacctggcggacccctgtcagtctatcctggtgccggtggcggctccggcggtgggtccggttccagcgttgcaagcttgacaccaacggcggctcattcaggatcacatttgtttggttttcccccaaccccgccgaaagaggtcagtcccgatccaagtacaaccggagcagcaagtcccgcaagctcatctgccggcggctccgccgctagaggcgaagataaagatggggtaaaatatcaagtctctcttaccgaatccatgaaaatggagtctggatctcctctccggcccgggctcgcgaccatggggacacaacccgcaacccatcatccaattccaacttatccaagctacgttcccgctgcagcacatgattattcatctgggctgtttcatccgggcggatttctcggtggcccagcatcttcttttactcccaaacaacggtctaaagcaaggtcttgcagtgagggtagggaatgcgtgaattgcggagctactgcaactcccctgtggcgtcgcgatgggacaggtcattatctttgtaacgcttgcggactttatcataaaatgaacggccaaaatcgcccgctgattaaacctaaacgcaggttgtcagcggctcgcagggcggggacatgctgcgccaactgccaaaccaccactacaacactgtggcgaagaaatgcaaatggcgatcccgtgtgtaatgcttgcgggctttattataaactccataacgtcaatcggcctcttacaatgaagaaagagggaattcaaaccaggaataggaaaatgtcaaataaaagcaagaaatcaaagaagggcgccgaatgttttgaagaattgtctaaatgtatgcaagagaaatcttcaccgttttccgccgccgcgttggccggtcatatggctccagtcggacatcttccgccattttcacatagtgggcatattcttccaacacccacacctattcatccttcatcttctctgtcatttggacatcctcatcctagctccatggttactgcaatgggcggctccggcgagggcaggggaagtcttctaacatgcggggacgtggaggaaaatcccggcccaatgccccgaagttttctcgttaaatcaaagaaagcacatacttatcatcaacctcgcgtccaagaggacgagccactttggccacccgctctgacacctgtcccgagggatcaagcgccgtccaattcacccgtgttgtctaccctctttcccaatcaatgtttggattggacaaatttgaagagggaaccagaacttgaacaagatcaaaatcttgcgcggatggcgcctgccccggaagggccaatcgtcctcagccggccgcaagacggcgattcccctcttagcgatagcccaccattttataaacccagttttagctgggatactctggctactacttacggacattcatatagacaagcgccttcaacgatgcaaagcgcatttcttgaacatagtgtgtccctttatggttccccactcgttccttccacagaaccagctctggatttctcacttcgatattctcccgggatggacgcttatcattgcgtaaaatgtaataaagtgttttctacaccgcatggtctggaagtccacgtacgccgtagtcactccggcacaaggccttttgcgtgcgatatttgtgggaagacgtttggtcatgccgtatcactcgaacaacatacacatgtgcattctcaagaacggagttttgaatgtagaatgtgtggtaaagcttttaaaaggtcatcaactttgagtactcatctcttgatacacagcgatacaaggccgtatccatgtcaattttgtggaaaacggtttcatcaaaagtcagatatgaagaaacatacatatattcataccggggaaaagccccataaatgtcaagtctgtggtaaagcgttttctcaatctagtaatctgattacacattctcgtaaacatactgggtttaaacctttctcttgcgaactttgtacaaagggatttcaacggaaagtcgatcttcgtagacatagagaatcccaacataaccttaaataa

SEQ ID NO: 49 - DNA, 3702 bp, synthetic construct
  source 1..3702: mol_type = other DNA
atgatgtttagcggatttaatgccgattatgaagcaagctcatctcgatgttcatctgcaagcccagctggtgactcactgagctattatcatagcccggctgattcattttcatctatgggttcccccgttaatgcccaagacttttgtaccgatctcgcagtgagcagcgcaaattttatccctaccgtgaccgctatatcaactagccccgatttgcaatggcttgtccaaccagcgttggtgtcaagcgttgcacccagccaaactcgcgctccccatccctttggcgtgccagctccgagcgcaggcgcctattcacgggcaggggtagtcaaaacaatgaccggcggacgcgctcaatccatcggaagaagagggaaagtcgagcaactctcccccgaggaagaggaaaagcgacgcattagaagagagcggaacaaaatggccgccgctaagtgtcggaatagacgccgcgaactcaccgacactctgcaggccgaaaccgatcagctggaagacgaaaagagtgcccttcaaactgaaatcgcaaatctcttgaaagaaaaggagaagctggaatttattcttgctgcacatcgtccagcatgtaaaattccggacgatttggggtttcccgaggaaatgtccgttgcctctctcgacttgaccggtgggttgcctgaagtagctacacccgaaagtgaagaagcgtttactcttccattgcttaacgatccagaaccaaaaccaagcgttgagcccgtaaaatctatttcttcaatggaactcaaaactgaaccattcgacgattttctctttcctgcttctagtagaccatctggctccgaaaccgcacgatctgtacctgatatggatctgtccggaagcttttacgccgccgattgggaaccactccattccggttcactcggaatgggacctatggctacggaattggaacctctttgtaccccagttgtaacttgcaccccatcatgtaccgcctatacctctagctttgtatttacgtatcctgaagccgatagctttccatcttgcgccgccgcgcataggaaaggatcttcaagtaacgaacccagttccgatagtttgagttctccgacacttctcgctcttggcagcggcgccacaaacttctctctgctaaagcaagcaggtgatgttgaagaaaaccccgggcctatgccccgttctttcttggtaaaatccaagaaggcgcatacatatcatcaaccacgggttcaagaggacgagccactgtggccgccagctctgactcccgtccctcgggatcaagccccctctaattctcccgtgctgtcaacattgtttcctaatcaatgtttggattggactaatctgaagcgcgaaccagaattggaacaagatcaaaatctcgctcggatggctcctgctccggaaggtcctatcgttctttcaagaccacaagacggcgattcacccctctcagattctccccctttctataaacccagctttagttgggatactctggcaaccacatacgggcattcttatcgccaagcgccaagcacaatgcaaagtgcatttctcgaacattcagtatctctttatggatctcccctggtcccttctaccgaaccagcgcttgacttttccttgaggtatagtcccgggatggacgcatatcattgcgtaaaatgtaataaagttttcagcacgccccatggattggaagtccacgtcaggcgatctcactctggcacaagaccttttgcatgcgatatttgtggtaagacgtttggacatgccgtttcactcgaacaacatacacatgttcatagccaagaacgttcatttgaatgtagaatgtgtggaaaagcatttaaaaggagctctaccctttctacacatttgctgattcatagcgatacccgaccttatccgtgtcaattttgtggaaaacgatttcatcagaaatcagatatgaagaagcatacatatatacataccggggagaaaccccataaatgtcaagtctgtggcaaagcattttctcaatcaagcaatctgattactcattccagaaaacatactggatttaaacccttttcatgcgaactttgtactaagggatttcaacggaaagttgatttgaggagacatcgagaaagtcaacataacctgaaaggctccggcgagggcaggggaagtcttctaacatgcggggacgtggaggaaaatcccggcccaatggaagttgccccggaacaaccccgttggatggcccatcctgcagtacttaacgcccaacatccggattcccatcatcccggactcgctcataattatatggagccggctcaactcttgccacccgatgaagtcgatgtgttctttaaccatctggattctcaagggaatccatattacgcaaatccagcacatgcccgagccagagttagctattcacctgcccatgcaagattgactggtggacaaatgtgtcggccccatctgctccattcaccagggctcccatggcttgatggcggtaaggctgcgctgagcgctgccgccgcacatcatcataatccatggacagtatctcccttttctaaaactccccttcatccttctgcagccggtggacccggcggacctttgagtgtttatcctggcgcaggcggcggttccggcggcgggagtggaagcagtgtcgcgagtctgacgcccactgctgcacattcaggaagtcatctgtttgggtttcccccaactccacccaaagaggtttccccagatccaagtacaaccggcgcagccagtcccgcttcctctagcgccggcggctcagctgctagaggggaagataaagatggtgttaaatatcaagttagccttactgaatctatgaaaatggagtcagggagcccgctcagacccgggctggcgacgatgggtacacaaccggccactcatcatcctattccgacgtatccgagttacgttcccgccgccgctcatgattattcttccggcctgtttcatccaggcggttttcttggcggccccgcgagctcatttacgcccaaacaaaggagtaaagccagatcttgctctgaggggagagaatgcgtgaattgcggcgcgacggcaactcccttgtggcgccgcgatggtactgggcattatctctgtaacgcatgcggattgtatcataaaatgaacggtcaaaataggcctctgataaaacctaaacggcgccttagtgcggcaaggcgtgcaggaacatgctgcgctaactgccaaacaactacaactactttgtggcggcgcaatgcgaatggtgatcccgtgtgtaatgcttgcggattgtattataaactccataacgtgaatcgtcctctcactatgaagaaggaaggaattcaaacgcgcaataggaaaatgtctaataaaagcaagaagtccaagaagggcgctgaatgttttgaagaactcagcaaatgtatgcaagagaaatccagtccattttcagccgccgcacttgcaggtcatatggctcccgtcgggcatttgccaccgttttcccatagcggtcatattctcccaacccctactccaatacatccttctagctctctgagttttgggcatcctcatcccagttccatggtcacagcgatgggttga